(19) United States
(12) Patent Application Publication
(10) Pub. No.: US 2023/0265455 A1
(43) Pub. Date: Aug. 24, 2023

(54) IMPROVED AAV-MEDIATED X-LINKED RETINOSCHISIS THERAPIES

(71) Applicant: University of Florida Research Foundation, Incorporated, Gainesville, FL (US)

(72) Inventors: Shannon E. Boye, Gainesville, FL (US); Sanford L. Boye, Gainesville, FL (US)

(73) Assignee: University of Florida Research Foundation, Incorporated, Gainesville, FL (US)

(21) Appl. No.: 18/017,726

(22) PCT Filed: Jul. 28, 2021
(86) PCT No.: PCT/US2021/043582

§ 371 Date: Jan. 24, 2023

Related U.S. Application Data
(60) Provisional application No. 63/058,437, filed on Jul. 29, 2020.

(51) Int. Cl. C12N 15/86; C07K 14/005; C07K 14/47; C07K 14/475; A61K 48/00; A61P 27/02; A61K 9/00

(52) U.S. Cl. CPC C12N 15/86; C07K 14/005; C07K 14/47; C07K 14/475; A61K 48/0058; A61P 27/02; A61K 9/0048

(57) ABSTRACT

The present disclosure provides for improved recombinant AAV therapies for the treatment of X-linked retinoschisis (XLRS). These therapies are designed for administration to subjects, such as human subjects, including humans diagnosed with or suffering from XLRS.

### RELATED APPLICATIONS

This application claims the benefit of the filing date of U.S. Provisional Application No. 63/058,437, filed Jul. 29, 2020, the entire contents of which are incorporated by reference.

### GOVERNMENT SUPPORT

This invention was made with government support under R01 EY024280 awarded by the National Institutes of Health. The government has certain rights in the invention.

### BACKGROUND OF THE INVENTION

Retinoschisis is a genetic condition characterized by the splitting of the retina into two layers. There are two forms of this disorder. The more common form, known commonly as senile retinoschisis, typically develops in middle age or beyond and can affect both men and women. The rarer form, X-linked retinoschisis (XLRS) is present at birth and affects boys and young men. The main symptom of XLRS is impaired vision that cannot be improved with eyeglasses. While some people with XLRS may experience progressive vision loss throughout their life, other people may have relatively stable vision throughout their lifetime. XLRS is one type of a broader disorder called macular degeneration, as it primarily affects the macula. XLRS affects about 30,000 men in the United States and European Union. There are no treatments available for patients with XLRS. However, the use of topical dorzolamide and oral acetazolamide in reducing cystic spaces and foveal thickness with a concomitant increase in visual acuity has been reported.

### SUMMARY OF THE INVENTION

The present disclosure provides for improved rAAV therapies for the treatment of XLRS. These therapies are designed for administration to the eyes of subjects, such as human subjects, including humans diagnosed with or suffering from XLRS. The disclosed rAAV vectors may provide for improved retinal structure and function after administration to subjects. The disclosed vectors may provide for amelioration or reversal, e.g., a partial or complete reversal, of the symptoms of retinoschisis.

The disclosed vectors comprise novel synthetic human RS1 transgenes. In some embodiments, the human RS1 transgene (or heterologous nucleic acid) of the disclosed vectors lacks the 5′ and 3′ untranslated regions, or UTRs. In some embodiments, the transgene contains mutations relative to the wild-type sequence that do not result in any amino acid change (i.e., are silent mutations). The present disclosure also provides for the use of photoreceptor-specific promoters, such as the human rhodopsin promoter, and of AAV capsids that display improved retinal transduction efficiency and that exhibits enhanced lateral spread after subretinal injection.

Accordingly, the present disclosure provides nucleic acid vectors comprising a synthetic human RS1 transgene sequence operably controlled by a promoter, such as a human rhodopsin kinase (hGRK1) promoter, that may be encapsidated into a viral particle, such as an AAV particle. It was previously determined that the hGRK1 promoter mediates efficient, photoreceptor-specific expression in non-human primate retina. The disclosed nucleic acid vectors may comprise AAV inverted terminal repeats flanking the RS1 transgene. The disclosed nucleic acid vectors may comprise AAV inverted terminal repeats flanking polynucleotide comprising the RS1 transgene.

XLRS is caused by mutations in a gene on the X chromosome called RS1 which encodes a protein called retinoschisin. Retinoschisin is a structural protein expressed and secreted by photoreceptor and bipolar cells that binds strongly and specifically to the surfaces of many cells in the retina. This protein serves as an adhesive to maintain the structural integrity of the layers of the retina. Without normal retinoschisin protein, the layers of the retina split, inter-cell communication is disrupted, and retinal cells and ultimately vision is lost. XLRS patients typically present with a diminished b-wave in electroretinogram (ERG) measurements of their retina. Patients with nonsense mutations generally have more severe disease than those with missense mutations.

Retinoschisin (RS1) is expressed throughout the neural retina during retinal development. After development and into adulthood, it expressed by photoreceptors. It is a secreted protein that predominantly localizes to inner segments (IS) of rod and cone photoreceptors and, to a lesser extent, the outer plexiform layer. RS1 contains discoidin domains that allow it to form homo-octameric complexes. The exact molecular function of RS1 remains unresolved. RS1 has been shown to directly interact with retina-specific Na/K+ ATPase and is thought to serve as modulator of these ATPase activities. RS1 interacts with L-type voltage-gated calcium channels (LTCCs) and may facilitate the function of LTCCs at the photoreceptor-bipolar cell synapse.

The therapeutic potential of gene therapy-based approaches for the treatment of XLRS is currently being evaluated. In particular, studies assessing the potential safety and effectiveness of adeno-associated virus (AAV) vectors engineered to express RS1 in animal models have been reportes. Several studies have made use of the retinoschisin homolog (Rs1h) knock-out mouse as a clinically relevant model. In this model, schisis lesions may be observed by optical coherence tomography (OCT) and reduced retinal function may be observed by electroretinography.

However, recent gene therapy clinical trials have failed to show biological activity or therapeutic efficacy, ending due to an adverse inflammatory response. AAV mediated gene therapy in XLRS mouse models have only been successful when the RS1-carrying rAAV particles successfully transduce and express transgenes in photoreceptors, but not when RS1 expression is limited to any other retinal cells type, such as Muller cells or retinal ganglion cells. In these cases, therapeutic intervention as late as 7 months of age is able to prevent further photoreceptor degeneration. Thus, there remains a need for novel AAV vectors that may be used in ocular gene therapy treatments of X-linked retinoschisis.

Major advances in the field of gene therapy have been achieved by using viruses to deliver therapeutic genetic material. The AAV vector has attracted considerable attention as a highly effective viral vector for gene therapy due to its low immunogenicity and ability to effectively transduce non-dividing cells. AAV has been shown to infect a variety of cell and tissue types, and significant progress has been made over the last decade to adapt this viral system for use in human gene therapy. Recombinant adeno-associated virus (rAAV) vectors have been used successfully for in vivo gene transfer in numerous pre-clinical animal models of human disease and have been used successfully for long-term expression of a wide variety of therapeutic genes. AAV vectors have also generated long-term clinical benefit in humans when targeted to immune-privileged sites, e.g., ocular delivery for Leber's congenital amaurosis. A major advantage of this vector is its comparatively low immune profile, eliciting only limited inflammatory responses and, in some cases, even directing immune tolerance to transgene products. Nonetheless, the therapeutic efficiency, when targeted to non-immune privileged organs, has been limited in humans due to antibody and CD8+ T cell responses against the viral capsid, while in animal models, adaptive responses to the transgene product have also been reported.

AAV has become the vector of choice for targeting therapeutic genes to the retina. Both naturally occurring and synthetic AAVs have been identified that display retinal tropism. AAV capsid serotype 44.9 efficiently transduces a number of cell types including salivary gland cells, liver cells, and different types of neurons (e.g., cells of the cortex, olfactory bulb, brain stem, and Purkinje cells of the cerebellum). AAV44.9 exhibits comparable in vivo biodistribution to AAV9. Intracerebroventricular injections of this capsid have shown transduction levels in the cortex, olfactory bulb, cerebellum, choroid plexus and brain stem similar to those observed with AAV9. In addition, antibody neutralization studies suggest a lower frequency of neutralizing antibodies to AAV44.9 compared with AAV2. And glycan array studies of AAV44.9 have suggested binding of the capsid to terminal glucose-containing molecules.

The inventors of the present disclosure have used a rational design approach to engineer a new variant by mutagenizing the glutamic acid at residue 531 to aspartic acid. This new serotype variant, AAV44.9(E531D), was evaluated in subretinally injected mice and macaque. Amino acid substitutions at positions corresponding to the E530 position in the AAV2 capsid, such as position 531 in AAVrh.8 and AAV44.9, have been hypothesized to alter transduction efficiency. rAAV particles incorporating the AAV44.9(E531D) capsid variant were found to be capable of highly efficient transduction of rods, cones, and retinal pigment epithelium (“RPE”) following subretinal injection. In addition, AAV44.9(E531D) exhibits increased lateral spread, transducing photoreceptors and retinal pigment epithelium outside the subretinal injection bleb. The increased potency and lateral spread of AAV44.9(E531D) make this variant a promising vector for gene therapies targeted to the retina.

The inventors have applied the novel AAV44.9(E531D) capsid, as well as other AAV capsids, to the context of delivery of retinoschisin-encoding RS1 heterologous nucleic acid to subjects. The present disclosure provides a RS1 nucleic acid for incorporation into rAAV vectors that comprises only the coding region of the RS1 gene, or cDNA, after removal of the 5′ and 3′ untranslated regions of human RS1. The human RS1 transgenes of the disclosure may also comprise one or more (e.g., four) silent mutations in the coding region that do not result in mutations in the encoded human retinoschisin protein sequence. The RS1 transgenes may contain an optimized Kozak sequence that differs from the native Kozak sequence of the wild-type RS1 gene. In some embodiments, CpG islands have been removed from the coding sequence. In some embodiments, the RS1 transgene of any of the disclosed rAAV vectors has been codon-optimized for human expression.

In some aspects, subretinal administration of the disclosed rAAV vectors is provided. Subretinal injection of AAV is commonly used when transgene expression is required in the retinal pigment epithelium (RPE) or the photoreceptors (PR). The subretinal injection creates a temporary bullous detachment, separating the photoreceptor outer segments from the RPE layer. Typically the subretinal injection bleb resolves over the following few days in subjects. Subretinal injection likely has some deleterious effects on the photoreceptors, with such effects conceivably being more severe in a retina already compromised by disease. In particular, the presence of schisis cavities in XLRS patient retinas suggested that alternatives to subretinal injection would need be explored because these retinas were more prone to detachment. However, administration into the vitreous (intravitreal injection) presents its own challenges, including an observed dilution effect, the presence of the inner limiting membrane (ILM) barrier, and severe inflammation owing to the increased biodistribution of AAV to the peripheral circulation when delivered via this route compared to when delivered to the subretinal space. Thus, there remains a need in the art to administer gene therapies to the retinas of XLRS patients subretinally while minimizing adverse effects such as retinal detachment.

Some aspects of this disclosure relate to rAAV particles and vectors comprising a modified AAV44.9 capsid or an AAV5 capsid for treatment of symptoms and conditions of XLRS in the eye. In particular embodiments, this disclosure provides particles comprising an AAV44.9(E531D) capsid that exhibits enhanced lateral spread after subretinal injection to the fovea, wherein detachment of the fovea is minimized. Accordingly, in some embodiments, the disclosure provides rAAV particles comprising a capsid comprising a VP1, VP2, and/or VP3 protein, wherein the rAAV particle further comprises a polynucleotide comprising a heterologous nucleic acid. In some embodiments, the disclosure provides a capsid protein, e.g., a VP1, VP2 or VP3 capsid protein, comprising the amino acid sequence of any one of SEQ ID NO: 1, 2 or 3, respectively. Further AAV vectors may be utilized, e.g., AAV2 vectors such as AAV2(4pMut)ΔHS as well as others described herein. Accordingly, in some aspects, the disclosure provides methods for expressing a human RS1 nucleic acid segment in one or more photoreceptor cells of a mammalian subject, wherein the method comprises subretinally or intravitreally administering to one or both eyes of the subject an rAAV particle as described herein. In particular embodiments, administration is by subretinal injection.

In some aspects, the disclosure provides recombinant AAV (rAAV) vectors that comprise a polynucleotide that comprises a heterologous nucleic acid encoding a retinoschisin protein (e.g., a human retinoschisin protein) and one or more additional elements, such as post-transcriptional regulatory elements. In some embodiments, the element comprises a splice donor region. In some embodiments, the element comprises a splice acceptor region. In some embodiments, the element comprises an SV40 intron. In some embodiments, the element comprises a polyadenylation signal sequence. In some embodiments, the element comprises a promoter. Non-limiting example promoters include a rhodopsin kinase promoter, a rhodopsin promoter, an IRBP promoter, a chimeric human Retinoschisin-IRBP enhancer (RS/IRPB), a red/green cone opsin promoter, a Cone Arrestin promoter, a chimeric IRBP enhancer-cone transducin promoter, a chicken beta actin promoter, and a truncated chimeric chicken beta actin (smCBA) promoter. In some embodiments, the element comprises a WPRE element. In some embodiments, the element comprises one or more inverted terminal repeat sequences. In some embodiments, the element comprises a stuffer sequence. In various embodiments, the heterologous nucleic acid of the vector does not comprise the 5′ untranslated region (UTR) of the (wild-type) gene (or cDNA) encoding human retinoschisin. The 5′ untranslated region of the human retinoschisin gene may comprise, or consist of, the nucleic acid sequence of SEQ ID NO: 39 (AGTTCAGTAAGGTAGAGCTTTGGCCGAGGACGAGGGGAAG). In some embodiments, the heterologous nucleic acid does not comprise the 3′ untranslated region of human retinoschisin. The 3′ untranslated region of human retinoschisin may comprise, or consist of, the nucleic acid sequence of SEQ ID NO: 40

(TGCCTGCCTCAGCTCGGCGCCTGCCAGGGGGTGACTGGCACAGAGCGGG
CCGTAGGGGACCCCCTCACACACCACCGAGATGGACAGGGCTATATTTCG
CAAAGCAATTGTAACTGCAGTGCTGGGTAGATAATTTTTTTTTTTTTAAG
ATATAGCTTTCTGATTTCAATGAAATAAAAATGAACTTATTCCCCACTCA
GGGCCAGAGAAAGTCAGAACAAAGAAAATGTCCCCGAAACGAATTTTCTT
ACAAAAGCCTAAGTAGCAGGGGTAATTTTCTGCTCATTTTTTGTCTCAGT
GATACTGTGAAAGGTGCAGTCTCAGGGGAACACAAAGCAGCCCTGATAAT
TTGAAAATTCATTTGCTTTACCACATTCAAGACAGAAACATACAGTTTCC
TAAAGCCTGGCTTTGAATGCAGAAGGGAGCAGCTCCTCCTAGTTAAGTTT
CCACTAAATCATCGCCAAAGAGGACTTCAGAGCCCTGGGGAGGCAGCTGA
GGGTCTCAAGGGTGACTGGGTGGCAGGGATGAGTGCGGTGGGTGAGAATC
CCGGTGCCCTGAGAGGCTATACGTGACAAATGACCAAAAGCCCAAGGTAG
GGGAGTTTCCTCTGCTCACAGTTCTTACCTTCAAGGCGGATCTGGGCTTC
CACCCTCATGAACACAGGGATTGGGGAGGGACCAGAGCGCCCAATACACA
CAGCTCCATTATGCAATCCATTCCAGCAAATTCCCCGTGTCTGTGGTCAC
CATTTAGGTGATCATACAGGACAGGCTGCACATCTCAGTATATGTAGGGA
CCCCAAATGACCACAACACAGTACAATTGCCCTTTACCTAGGGCTACCAT
TTCCTAGCAAACCAAACATAGTTCGAGAACAGCTGGCCCAGGAGCTACCA
CTGGCTACTCAGAGGAGGCTCATTAGCTGGCTACATGCTTCGCAGGAAGT
GGGAAGGACTCACATCATAAAAAGGACCATGTAGCTTTTTCCCTGAAAGC
TTCTCACCCTCCACCCTCTGCCTTGCAATACGCAAACTGCGCCTGCTCCT
GAAAAGCTCTCTGGGAAGGAATGGGCCTGGCTTTCCGTTCCTGGAGGCGG
CGCCTTAGATTGGGAGGCCTCATTGGCCACTTAGAGCGCAGCCTGAGTTT
CCAGGCCCCTTCCTGGGAGAGGCTGTTAACACGGGGGAGGGGCAGGAGAG
GGATATGGAGAGCAGGTGGTGGAATCAGAGGACGAGGCTGCTCTAAAGAC
TGTTCTGGCCCCAGACACAGGGTAGTCTTTGCTAGCAGCTCATTTCCGAG
TTACTTTTCATTTTCAAATGCCAAGGCAAGTGACTAGACTCGCGCTAATA
CAGTGCTGGACAACACATTCACCTTTTCTGTGAACAGGCAGCCTTCTAAA
AGCCCCAAACATCCTTCTTGATGCTTTGGGGGCTCAATTATTTTATATCC
AACCCAGCATCTTTCTAGTCCCTATGCTGTATGCTTGAACTTCGGAAAAT
GCTTTTCCCCGCCCAATCTTCTCTCAAATATAAACACATCACACAGGGTG
TTGGGGGTGGGGGGGGGGGGTGGGGGGACTTATCCCTGGCCTTAGGACAC
AGGACAAATCTATTTTGGATAGAAATGCCTGAACAGAGACCCTTATTTGG
AAAGGTGAATTAACTTTGGTCACGACATGGACTGTCAGACAAAATGGCAG
TATCCTAAGAGTTAAGGCACATCAAACACAGGAGTCGAGAGAGTGCAGTT
CAGGGAAAAAGGAGAGGAGGAAACAGTGAGGCAGGGAGAAAGGCTTTCCA
AATAAGAGTTCATGTTGGAAACTTTTGTCACGGCTTTATTGAGATTAAGT
TCACATACAATTTGTATCCATTTAAAGTGTACAATTTGATGACTTTTGGT
ATATTCAGAGTTGTGCAACCATTATCACTAGATCAATTTTAGAAAGTTTA
TCACCCCAAAGAGAAATCCTGCACCCATCAGCCAACACTCCCCAACCCAT
CGGCCACCCCAAGCCCTCTGCAACCACGAATCGACTGTCTCTGTAGATTG
GCCTTCTGGACGTTCTACATAAATGAAATCATATAGTATGTGGTATTTCG
TGACTGGCTTCTTTCACTTAGCATAGTGTTTTAAAGTTCATCCACGTTAT
AACATGTGTATCACTATGTCACTTGTCACTCCTTTTTATTGCTGAACATC
ATTGTTCAGTATCATGTCAAGAGCACATTGTTATTTATCCATTCATCCAT
TGATGGATATTTGGGTTTCCACTCTTTAGCTATTATGAATAATGCTGCTA
TGAACATTTGTGTATAA).

In some aspects, the rAAV vectors comprise a polynucleotide that comprises a heterologous nucleic acid comprising a sequence having at least 80% identity, at least 85% identity, at least 90% identity, at least 92% identity, at least 92.5% identity, at least 93% identity, at least 94% identity, at least 95% identity, 98% identity, or 99% identity to the nucleotide sequence of SEQ ID NO: 8. In some embodiments, the polynucleotide of comprises a heterologous nucleic acid comprising at least 92.5% identity to SEQ ID NO: 8. In particular embodiments, the polynucleotide comprises at least 98% identity to SEQ ID NO: 8. In some embodiments, the heterologous nucleic acid comprises a sequence that differs by 5, 10, 15, 20, 25, or more than 25 nucleotides from the nucleotide sequence of SEQ ID NO: 8. In some embodiments, the heterologous nucleic acid comprises the sequence of SEQ ID NO: 8.

In some embodiments, the heterologous nucleic acid may comprise the sequence set forth as SEQ ID NO: 9. The polynucleotide may comprise the sequence of SEQ ID NO: 10. The polynucleotide may comprise a nucleic acid sequence encoding a myc tag (e.g., a c-myc tag). In some embodiments, the polynucleotide comprises a promoter that is capable of expressing the heterologous nucleic acid in one or more photoreceptors or retinal pigment epithelial cells of a mammalian eye. The rAAV particle may comprise a polynucleotide comprising at least a first polynucleotide that comprises a PR- or an RPE-cell-specific promoter operably linked to at least a first heterologous nucleic acid that encodes a therapeutic agent, for a time effective to produce the therapeutic agent in the one or more PR cells or RPE cells of the mammal. In particular embodiments, the promoter is a rhodopsin kinase promoter, such as a human rhodopsin kinase (hGRK1) promoter.

In some aspects, the disclosure provides rAAV particles comprising any of the rAAV vectors described herein. In some embodiments, the capsid encapsidating the rAAV vector in the rAAV particle comprises an AAV44.9 capsid, an AAV44.9(E531D) capsid, an AAV44.9(Y446F+T492V+E531D) capsid, an AAV44.9(Y446F+E531D) capsid, an AAV44.9(T492V+E531D) capsid, an AAV44.9(Y446F+T492V+E531D), an AAV44.9(Y731F) capsid, an AAVS capsid or a variant thereof, an AAV2(4pMut)ΔHS capsid, or an AAV8(Y447F+Y733F+T494V) capsid. In particular embodiments, the rAAV particle comprises an AAV44.9(E531D) capsid. The disclosure also provides compositions comprising any of the rAAV particles disclosed herein. The disclosure also provides cells (such as mammalian cells) comprising any of the rAAV particles disclosed herein.

In some aspects, the disclosure provides methods for transducing a mammalian photoreceptor cell or retinal pigment epithelium cell, the method comprising administering to one or both eyes of a mammal any of the rAAV particles or compositions of rAAV particles disclosed herein. Methods for treating or ameliorating XLRS in a mammal (such as a human), the method comprising administering to one or both eyes of the mammal any of the rAAV particles or compositions disclosed herein in an amount sufficient to treat or ameliorate the one or more symptoms of the retinoschisis in the mammal are also provided. In some embodiments, the disclosed methods comprise subretinal administration to a fovea of one or both eyes of the mammal. In particular embodiments, detachment of the fovea is minimized following subretinal administration. In some embodiments, any of the disclosed methods a) preserves one or more photoreceptor cells, b) restores laminar retinal structure, c) restores one or more rod- and/or cone-mediated functions, d) restores completely or partially visual behavior in one or both eyes, or e) any combination thereof. In particular embodiments, any of the disclosed methods restore laminar retinal structure and/or reduces or eliminates schisis lesions.

### BRIEF DESCRIPTION OF THE DRAWINGS

The following drawings form part of the present specification and are included to demonstrate certain aspects of the present invention. The invention may be better understood by reference to the following description taken in conjunction with the accompanying drawings, in which like reference numerals identify like elements, and in which:

FIG. 1 shows AAV44.9-hGRK1-GFP and AAV44.9(E531D)-hGRK1-GFP exhibit enhanced lateral spread and potency in subretinally injected macaques. Vector delivered at 1×10¹² vg/mL. Initial boundaries of blebs on day of dosing and borders of resulting GFP expression are outlined in white dotted line. Identical vasculature is highlighted in thickened dark lines for reference.

FIG. 2 shows optical coherence tomography (OCT) scans of three subretinal injection blebs created (see negative contrast fundus image on the left) totaling 90 μL of vector volume, following extrafoveal subretinal injection of AAV44.9-hGRK1-GFP (1×10¹² vg/mL) in macaque. OCT allows for longitudinal, in vivo assessments of retinal structure, such as schisis lesions.

FIG. 3 shows OCT images of macaque retinas following extrafoveal subretinal injection of AAV44.9-hGRK1-GFP. Arrows in SLO image (top left) indicate the locations of retinal sections shown in the scans in the lower part of the figure. Sections were stained for cone arrestin and DAPI. The percentage of rods/cones expressing GFP is plotted in each zone. ONL, outer nuclear layer; INL, inner nuclear layer; GCL, ganglion cell layer. Staining for native GFP is indicated with circles, staining for cone arrestin is indicated with triangles, and staining for DAP is indicated with squares, in this figure and throughout the drawings.

FIG. 4 shows OCT images of macaque retinas following extrafoveal subretinal injection of AAV44.9(E531D)-hGRK1-GFP, three blebs totaling 90 μL of vector volume. Arrows in SLO image (top left) indicate the locations of retinal sections shown in the scans in the lower part of the figure. Sections were stained for cone arrestin and DAPI. The percentage of rods/cones expressing GFP is plotted in each zone.

FIGS. 5A-5D show representative OCT images of parafoveal regions of macaque retinas following extrafoveal subretinal injection of AAV44.9(E531D)-hGRK1-GFP and AAV44.9-hGRK1-GFP. Scale bars are in A=40 microns, B=20 microns.

FIGS. 6A and 6B show representative OCT images of perifoveal regions of macaque retinas following these injections.

FIG. 7 shows a schematic of a subretinal injection.

FIGS. 8A-8C show the transduction profile of AAV vectors following subretinal injection (SRI) in Nrl-GFP mice. (FIG. 8A) AAV-mediated mCherry expression in representative fluorescent fundus images (FIG. 8A, top row) and retinal cross sections (FIG. 8A, middle and bottom row) taken at 4 weeks post-injection with 2×10¹² vg/mL. Transduction profile of AAV44.9(E531D)-IRBP/GNAT2-GFP following subretinal injection (SRI) in C57BL6J mice. (FIG. 8B) Six weeks post-injection with 2×10¹² vg/mL (2×10⁹ vg delivered), retinal cross sections were stained with an antibody directed against cone arrestin and counterstained with DAPI. (FIG. 8C) *Kruskal-Wallis tests were performed, followed by a post hoc Dunn's test to make pairwise comparisons between groups. For the non-rod values: H=44.912, df=6, P<0.001. For the rod values: H=85.546, df=6, P<0.001. Scale bars in A, B=50 microns. RPE—retinal pigment epithelium, ONL—outer nuclear layer, INL—inner nuclear layer, GCL-ganglion cell layer.

FIGS. 9A-9E show natural history electroretinogram measurements (ERGs) at various months. FIG. 9A shows natural history ERG at 1 month. FIG. 9B shows natural history ERG at 2 months. FIG. 9C shows natural history ERG at 3 months. FIG. 9D shows natural history ERG at 4 months. FIG. 9E shows natural history ERG at 5 months.

FIG. 10 shows a 1-5 month summary of the natural history ERG.

FIG. 11 shows the quantification of schisis lesions in Rs1 KO mice. The extent of retinoschisis cavities was analyzed using Bioptigen OCT and facilitated by the Diver software. Eight (8) spots are designated by a 3×3 grid. The center spot is the optical nerve (ON) where cavities are not present. The presence, abundance and magnitude of schisis cavities within a 0.5 mm (500 m) segment were used to generate a score. Examples of scans and corresponding scores as well as descriptions of observations used to factor scores are shown in FIG. 12.

FIG. 12 shows the grading system for severity of schisis cavities (0-4), with 0 equaling no schisis cavities and 4 being the highest cavity count.

FIG. 13 shows a graph of retinoschisis cavity count. Cavity score peaks at 2 to 3 months and then improves with time.

FIG. 14 shows the OCT measurements and schisis activity scores associated with the natural history immunohistochemistry (IHC) assay. 1 month old mouse retinal sections stained with monoclonal antibody to RS1. RS1 expression localized to the inner/outer segment junction of photoreceptors in the female heterozygous and wildtype male mice and is absent in male and female RS1 KO mice.

FIGS. 15A-15F show schematics for six cassettes for exemplary vectors of the disclosure. Exemplary vectors include AAV-hGRK1-hRS1syn, which has a length of 1723 bp from the 5′ end of the first ITR to the 3′ end of the second ITR (FIG. 15A), AAV-CBA-hRS1syn, which has a length of 2977 bp from the 5′ end of the first ITR to the 3′ end of the second ITR (FIG. 15B), AAV-hGRK1-hRS1syn-WPREsf, which has a length of 2311 bp from the 5′ end of the first ITR to the 3′ end of the second ITR (FIG. 15C), pTR-X001-3p, which has a length of 4534 bp from the 5′ end of the first ITR to the 3′ end of the second ITR (FIG. 15D), pTR-X001-5p, which has a length of 4528 bp from the 5′ end of the first ITR to the 3′ end of the second ITR (FIG. 15E), and pTR-X002-3p, which ha a length of 4549 bp from the 5′ end of the first ITR to the 3′ end of the second ITR (FIG. 15F). pTR-GRK1-hRS1syn was packaged in AAVS and AAV44.9(E531D). pTR-CBA-hRS1syn vector plasmid (pTR-UF11 backbone) was packaged in AAVS to serve as a control.

FIG. 16 shows OCT measurements (at 1 month post injection).

FIG. 17A shows data for the pilot AAV-RS1 study. Test Injection: Retinoschisis cavity score (1 month post injection). Schisis cavities are completely resolved in all treated eyes. FIG. 17B shows data for the pilot AAV-RS1 study. ERG shown at 1 month post subretinal AAV-hRS1 injection. Preliminary Conclusions: hGRK1 promoter drives therapeutic RS1 expression, and AAV44.9(E531D) vectored construct is therapeutic.

FIGS. 18A-18C show OCT monthly data. FIG. 18A shows OCT monthy data at 1 month and 2 months. FIG. 18B shows OCT monthy data at 3 months and 4 months. FIG. 18C shows OCT monthy data at 5 months and 6 months.

FIG. 19 shows OCT data of the left eye versus the right eye.

FIG. 20 shows ERG measurements at one month.

FIG. 21 shows ERG at two months.

FIG. 22 shows ERG at three months.

FIG. 23 shows ERG at four months.

FIG. 24 shows ERG at five months.

FIG. 25 shows ERG at six months.

FIG. 26 shows a statistic analysis of b waves of the injected virus versus vehicle. ERG of Group 6 and 7 at 1 month post injection.

FIG. 27 shows a statistic analysis of b waves of the injected eyes.

FIG. 28 shows a statistic analysis of b waves of AAV44.9 (E531D) injected eyes.

FIG. 29 shows a statistic analysis of b waves of the AAV5 injected eyes.

FIG. 30 shows a statistic analysis of scotopic a waves of the injected virus relative to the empty vehicle.

FIG. 31 shows IHC measurements at 4 months post injection.

FIG. 32 shows restoration of retinal structure in RS1KO mice treated with rAAV44.9(E531D) containing stuffed cassettes. Quantification of schisis cavity scores in RS1KO mice treated with either vehicle or rAAV44.9(E531D) vectors containing the following cassettes: X001, X001-3p, X001-5p, or X002-3p. All vectors improved retinoschisis scores at both timepoints, except for X001-5p at 1-month post-injection. Nominal descriptive statistical significance was determined with a two-way ANOVA with a Tukey's post-test on the treated eyes.

FIGS. 33A-33B show restoration of retinal function in RS1KO mice treated with rAAV44.9(E531D) containing stuffed cassettes. Average maximum scotopic (left) and photopic (right) b-wave amplitudes in RS1KO mice measured 1- and 2-months after subretinal injection in one eye with either vehicle or rAAV44.9(E531D) containing the following cassettes: X001, X001-3p, X001-5p, or X002-3p. Vector was dosed at either 1×10⁸ vg (FIG. 33A) or 5×10⁸ vg (FIG. 33B). Retinal function in eyes treated with all vectors was improved over untreated control eyes. Nominal descriptive statistical significance was determined with two-way ANOVA with Tukey's post-test on each individual data set. *p<0.05, **p<0.01, ***p<0.001, ****p<0.0001.

FIGS. 34A-34B shows RS1 expression in retinas of RS1KO mice treated with rAAV44.9(E531D)-X002-3p. Representative retinal cross sections from RS1KO mice treated (top) in one eye only either vehicle or rAAV44.9(E531D) containing the X002-3p cassette. Vector was dosed at either 1×10⁸ vg (FIG. 34A) or 5×10⁸ vg (FIG. 34B). Contralateral untreated eyes are shown in the bottom row. All retinas stained with an antibody raised against RS1 (triangles) and counterstained with DAPI (squares).

### DETAILED DESCRIPTION

The present disclosure provides novel rAAV vectors, compositions, methods, for administration of rAAV particles for treatment of XLRS. These vectors are designed for delivery of a therapeutic agent comprising a synthetic retinoschisin gene to mammalian subjects, such as human subjects. Advantageously, the novel methods of rAAV particle administration disclosed herein have improved efficiency in transducing the retina of the mammalian eye, and in particular, in transducing the photoreceptor (PR) and retinal pigment epithelial (RPE) cells in vivo. Specifically, the disclosed rAAV vectors and compositions are capable of lateral spread beyond the site of vector injection, which will lead to efficient transduction of both intact as well as schisis (split) retina while utilizing relatively small volumes or doses of AAV vector. Accordingly, the disclosed methods may reduce risk of additional damage occurring due to surgical intervention near a schisis lesion, while ensuring that highly efficient photoreceptor transduction is achieved. The disclosed methods may thus provide for safe and efficient delivery of the retinoschisin (RS1) gene to photoreceptors. The disclosure also provides cells, such as host cells, containing any of the disclosed rAAV vectors.

In particular embodiments, delivery of this therapeutic agent a) preserves one or more photoreceptor (PR) cells, b) restores laminar retinal structure, c) restores one or more rod- and/or cone-mediated functions, d) restores completely or partially visual behavior in one or both eyes, or e) any combination thereof. In some embodiments, production of the therapeutic agent persists in the one or more photoreceptor cells or the one or more RPE cells substantially for a period of at least six months following an initial administration of the rAAV particle into the one or both eyes of the mammal.

In some embodiments, a polynucleotide comprising a heterologous nucleic acid that is encapsidated into an rAAV particle is provided. In particular embodiments, the heterologous nucleic acid is administered to the subject to provide a functional protein, e.g., human retinoschisin (RS1), to restore, e.g., completely or partially, photoreceptor function to a subject (e.g., a human) as well as restore laminar retinal structure, i.e. reduce schisis lesions. In some embodiments, one or both alleles of a target coding sequence of the subject are silenced by administering an rAAV particle comprising a heterologous nucleic acid disclosed herein to the subject (e.g., to a human suffering from XLRS).

In some aspects, the disclosure provides compositions comprising a rAAV particle and a pharmaceutically acceptable carrier, excipient, diluent and/or buffer. In some aspects, the disclosure provides a method of transducing RPE and photoreceptor cells to modulate expression of the heterologous nucleic acid (or transgene) in a subject, the method comprising administering to the subject, such as a human subject, a composition comprising an rAAV particle as described herein and a pharmaceutically acceptable carrier, excipient, diluent, buffer, and any combination thereof. In some aspects, the disclosure provides a method of treating XLRS in a subject, the method comprising administering a composition to the eye of a subject.

In some aspects, the disclosure provides a composition for use in treating retinal disease and a composition for use in the manufacture of a medicament to treat retinal disease. In some aspects, the disclosure provides a composition comprising an rAAV particle as described herein for use in treatment by subretinally or intravitreally administering to one or both eyes of the mammal.

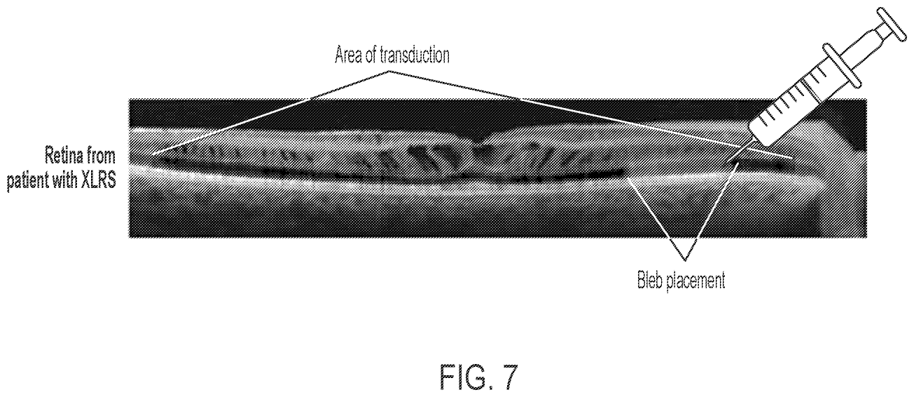

In some aspects, administration of the disclosed rAAV vectors in doses reduced relative to standard clinical doses provided. In a non-limiting example, the administration is subretinal (see FIG. 7). Some research suggests administration of high doses of AAV particles causes inflammation. Systemic administration of high doses of AAV particles that target the liver, neurons and/or muscles have led to deaths of subjects in a handful of clinical trials following excessive inflammation and onset of liver disease. Despite strong proof of concept studies in mouse models for subretinally delivered AAV-RS1 vectors, the first clinical trials utilized intravitreally (IVT) delivered AAVs because the risk:benefit ratio precluded use of subretinal injection for the treatment of XLRS. With the advent of intra-operative optical coherence tomography (OCT), vector placement can be tightly controlled by surgeons. In particular, the vector can be injected to areas beyond the schisis lesions. AAV vectors that can laterally spread beyond the injection site will lead to efficient transduction of both intact as well as schisis retinal areas.

The disclosed rAAV vectors utilize capsid variants that exhibit increased lateral spread and high transduction efficiencies, such as AAV44.9(E531D) and other AAV44.9 variants. The high efficiency and lateral spread mediated by AAV44.9(E531D) allows for smaller injection bleb volumes, thereby further reducing risk by limiting the area of detachment. Accordingly, the use of any of the disclosed rAAV vectors may facilitate reduction of bleb volumes and/or doses for ocular administration necessary to achieve a therapeutic effect in subjects, such as human subjects. Methods of administration of the disclosed rAAV vectors may enable targeting of subretinal injections to areas of the retina (e.g., the retina of an XLRS patient) that do not contain lesions or are relatively intact. These methods may be facilitated by the use of intra-operative OCT to guide the vitreoretinal surgeon's placement of blebs.

Accordingly, the disclosed rAAV particles may be administered in injection bleb volumes of less than 250 μL, less than 200 μL, less than 175 μL, less than 150 μL, less than 125 μL, less than 100 μL, less than 90 μL, less than 75 μL, less than 50 μL, or less than 45 μL. In some embodiments, 0.0001 mL to 10 mL (e.g., 0.0001 mL, 0.001 mL, 0.01 mL, 0.1 mL, 1 mL, 10 mLs) are delivered to the retina of a subject in a dose. In some embodiments, a dose of between 5×10¹⁰ to 1×10¹² vector genomes (vgs)/mL is administered to the retina. In the non-human primate experiments of Examples provided herein, a total of 90 μL (3 individual injection blebs of 30 μL each) was administered subretinally to primate retina (see Example 1). These volumes are substantially smaller than the volumes used in standard clinical application of AAV gene therapy. For example, Luxturna (voretigene neparvovec-rzyl), an rAAV-RPE65 vector, is delivered in a 300 μL subretinal injection. Accordingly, in some embodiments, a single subretinal injection of any of the disclosed vectors may achieve therapeutic effects. In some embodiments, no more than 3-5 subretinal injections may achieve therapeutic effects. In some embodiments, subretinal injections of disclosed rAAV particles or compositions thereo ranging in total volumes of between 30 μL and 300 μL, or more preferably between 30 μL and 150 μL may be administered. In particular embodiments, 3 subretinal injections ranging in bleb volumes of between 10 μL and 10 μL each may be administered.

### Synthetic RS1 Sequences

In some embodiments, the heterologous nucleic acid of any of the polynucleotides of the disclosure has a sequence that has at least 90% identity, at least 95% identity, at least 98%, at least 99% identity, or 100% identity to a nucleotide sequence set forth as SEQ ID NO: 8. This synthetic RS1 transgene lacks 5′ and 3′ untranslated regions relative to the cDNA of wild-type RS1. This sequence is referred to herein is “hRS1syn.” The length of SEQ ID NO: 8 is 684 nucleotides (nt). In some embodiments, the heterologous nucleic acid differs by 5, 10, 15, 20, 25, or more than 25 nucleotides from the nucleotide sequence of SEQ ID NO: 8.

In various embodiments, the heterologous nucleic acid of any of the polynucleotides of the disclosure does not comprise the 5′ untranslated region (UTR) of the (wild-type) gene (or cDNA) encoding human retinoschisin. In some embodiments, the heterologous nucleic acid does not comprise the 3′ UTR of the human retinoschisin gene. In various embodiments, the heterologous nucleic acid does not comprise the 3′ UTR or the 5′ UTR of the human retinoschisin gene. In various embodiments, the heterologous nucleic acid does not comprise truncated versions of the 3′ UTR or the 5′ UTR of the human retinoschisin gene. In some embodiments, the heterologous nucleic acid does not comprise a wild-type RS1 cDNA. In some embodiments, the heterologous nucleic acid consists of an RS1 coding region.

In some embodiments, the heterologous nucleic acid comprises the nucleic acid sequence of any one of SEQ ID NOs: 8, 9 or 10.

A nucleotide sequence encoding a synthetic human retinoschisin 1 (RS1) transgene (SEQ ID NO: 8) are shown below. The start codon is underlined; and the stop codon is bolded.

hRS1syn:
(SEQ ID NO: 8)
ATGTCACGCAAGATAGAAGGCTTTTTGTTATTACTTCTCTTTGGCTATGA
AGCCACATTGGGATTATCGTCTACCGAGGATGAAGGCGAGGACCCATGGT
ATCAAAAAGCCTGCAAGTGCGATTGCCAAGGAGGACCCAATGCTCTGTGG
TCTGCAGGTGCCACCTCCTTGGACTGTATACCAGAATGCCCATATCACAA
GCCTCTGGGTTTCGAGTCAGGGGAGGTCACACCGGACCAGATCACCTGCT
CTAACCCGGAGCAGTATGTGGGCTGGTATTCTTCGTGGACTGCAAACAAG
GCCCGGCTCAACAGTCAAGGCTTTGGGTGTGCCTGGCTCTCCAAGTTCCA
GGACAGTAGCCAGTGGTTACAGATAGATCTGAAGGAGATCAAAGTGATTT
CAGGCATCCTCACCCAGGGGCGCTGTGACATCGATGAGTGGATGACCAAG
TACAGCGTGCAGTACAGGACCGATGAGCGCCTGAACTGGATTTACTACAA
GGACCAGACTGGAAACAACCGGGTCTTCTATGGCAACTCGGACCGCACCT
CCACGGTTCAGAACCTGCTGCGGCCCCCCATCATCTCCCGCTTCATCCGC
CTCATCCCGCTGGGCTGGCACGTCCGCATTGCCATCCGGATGGAGCTGCT
GGAGTGCGTCAGCAAGTGTGCCTGA

By a nucleic acid molecule (e.g., a heterologous nucleic acid, or transgene) comprising a nucleotide sequence having at least, for example, 95% “identity” to a query nucleic acid sequence, it is intended that the nucleotide sequence of the subject nucleic acid molecule is identical to the query sequence except that the subject nucleic acid molecule sequence may include up to five nucleotide alterations per each 100 nucleotides of the query sequence. In other words, to obtain a promoter having a nucleotide sequence at least 95% identical to a reference (query) sequence, up to 5% of the nucleotides in the subject sequence may be inserted, deleted, or substituted with another nucleotide. These alterations of the reference sequence may occur at the 5′ or 3′ ends of the reference sequence or anywhere between those positions, interspersed either individually among nucleotides in the reference sequence or in one or more contiguous groups within the reference sequence.

As a practical matter, whether any particular nucleic acid molecule is at least 80%, 85%, 90%, 95%, 96%, 97%, 98%, or 99% identical to, for instance, the nucleotide sequence of a synthetic RS1 cDNA, can be determined conventionally using known computer programs. Preferred methods for determining the best overall match between a query sequence (a sequence of the present disclosure) and a subject sequence, also referred to as a global sequence alignment, can be determined using the FASTA program analysis described by Pearson and Lipman (1988) and FASTDB and blastn computer programs based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the 5′ and/or 3′ ends, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

In some embodiments, vectors containing any of the polynucleotides as described herein (such as the polynucleotides of SEQ ID NOs: 8-10) may comprise a Kozak sequence immediately 5′ of the start codon sequence (i.e., ATG). In some embodiments, this Kozak sequence comprises the nucleic acid sequence of GCCGCCACC (SEQ ID NO: 41). In some embodiments, the Kozak sequences of any of the disclosed vectors (e.g., those set forth in SEQ ID NOs: 31-35) contain a Kozak sequence having 1, 2, or 3 nucleotides that differ relative to SEQ ID NO: 41. In some embodiments, any of these Kozak sequences constitutes a ribosomal entry site.

In some embodiments, the polynucleotides as described herein may comprise a nucleic acid sequence having 1, 2, 3, 4, 5, 6, 7, 8, 9, 10, 11, 12, 13, 14, 15, 16, 17, or 18 nucleotides that differ relative to the sequence as set forth in any one of SEQ ID NOs: 7-10 and 15-17, e.g., SEQ ID NOs: 8-10, 16 and 17. In some embodiments, the polynucleotides as described herein may comprise a nucleic acid sequence having 1-10 (e.g., 5 or 9) nucleotides that differ relative to the sequence of any one of SEQ ID NOs: 8, 9 and 10. These differences may comprise nucleotides that have been inserted, deleted, or substituted relative to the sequence of any one of SEQ ID NOs: 7-10 and 15-17. In some embodiments, the polynucleotides comprise truncations at the 5′ or 3′ end relative to any one of SEQ ID NOs: 7-10 and 15-17. In some embodiments, the disclosed polynucleotides contain stretches of about 50, about 75, about 100, about 125, about 150, about 175, or about 180 nucleotides in common with the sequence of any one of SEQ ID NOs: 7-10 and 15-17. In some embodiments, the disclosed polynucleotides contain stretches of about 50, about 75, about 100, about 125, about 150, about 175, about 180, about 200, about 300, about 400, about 500, about 600, about 700, about 800, about 900, about 1000, or more than about 1000 nucleotides in common with the sequence of any one of SEQ ID NOs: 16 and 17.

In some embodiments, the disclosed polynucleotides contain stretches of about 50, 75 or more nucleotides in common with any one of SEQ ID NOs: 8-10, 16 and 17 in regions of the sequence in which CpG islands are absent. The polynucleotides of the disclosure may comprise 1, 2, 3, 4, 5, 6, 7, 8, 9, 10, 11, 12 or more than 12 silent mutations that do not result in mutations in the encoded RS1 protein sequence. In some embodiments, the disclosed polynucleotides may comprise between 30 and 40 silent mutations relative to the wild-type RS1 sequence. In some embodiments, a heterologous nucleic acid that varies in identity of up to 30% relative to (i.e., has at least 70% identity to) any of the sequences of SEQ ID NOs: 8-10, 16 and 17 encodes a polypeptide that has at least 90% amino acid sequence identity to a wild-type RS1 protein. In some embodiments, a heterologous nucleic acid that varies in identity of up to 30% relative to any of the sequences of SEQ ID NOs: 8-10, 16 and 17 encodes a polypeptide that has at least 90% amino acid sequence identity to any of the sequences of SEQ ID NOs: 12-14.

In some embodiments, the heterologous nucleic acid of any of the rAAV vectors of the disclosure has a sequence that has at least 75% identity, at least 80% identity, at least 90% identity, at least 95% identity, at least 98%, at least 99% identity, or 100% identity to the nucleotide sequence set forth as SEQ ID NO: 9. The heterologous nucleic acid may comprise SEQ ID NO: 9. A nucleotide sequence encoding a synthetic human retinoschisin 1 (RS1) transgene that has been codon-optimized for human expression and wherein CpG islands have been eliminated (SEQ ID NO: 9) is shown below. The start codon is underlined; and the stop codon is bolded.

(SEQ ID NO: 9)
ATGTCAAGAAAGATAGAAGGCTTTTTGTTATTACTTCTCTTTGGCTATGA
AGCCACATTGGGATTATCGTCTACAGAGGATGAAGGAGAGGACCCATGGT
ATCAAAAAGCCTGCAAGTGTGATTGCCAAGGAGGACCCAATGCTCTGTGG
TCTGCAGGTGCCACCTCCTTGGACTGTATACCAGAATGCCCATATCACAA
GCCTCTGGGTTTTGAGTCAGGGGAGGTCACACCTGACCAGATCACCTGCT
CTAACCCTGAGCAGTATGTGGGCTGGTATTCTTCTTGGACTGCAAACAAG
GCCAGACTCAACAGTCAAGGCTTTGGGTGTGCCTGGCTCTCCAAGTTCCA
GGACAGTAGCCAGTGGTTACAGATAGATCTGAAGGAGATCAAAGTGATTT
CAGGGATCCTCACCCAGGGGAGATGTGACATTGATGAGTGGATGACCAAG
TACTCTGTGCAGTACAGGACAGATGAGCGCCTGAACTGGATTTACTACAA
GGACCAGACTGGAAACAACAGAGTCTTCTATGGCAACTCTGACAGAACCT
CCACAGTTCAGAACCTGCTGAGACCCCCCATCATCTCCAGATTCATCAGA
CTCATCCCACTGGGCTGGCATGTCAGAATTGCCATCAGGATGGAGCTGCT
GGAGTGTGTCAGCAAGTGTGCCTGA

CpG islands are regions of the genome that contain a large number of cytosine-guanine dinucleotide repeats in the 5′→3′ direction. In mammalian genomes, CpG islands usually extend for 300-3000 base pairs. Cytosines in CpG dinucleotides may be, and are often, methylated through cellular epigenetic mechanisms to form 5-methylcytosines. Methylation of these cytosines reduces expression of coding regions of any genes in which these islands appear (e.g., the RS1 gene). In some embodiments, any of the disclosed polynucleotides comprise mutations (e.g., silent mutations) that interrupt thirty-six instances of CpG dinucleotides.

In some embodiments, the polynucleotide of the rAAV vector comprises a myc-tagged polynucleotide. In some embodiments, the heterologous nucleic acid of any of the rAAV nucleic acid vectors of the disclosure has a sequence that has at least 80% identity, at least 75% identity, at least 90% identity, at least 95% identity, at least 98%, at least 99% identity, or 100% identity to the nucleotide sequence set forth as SEQ ID NO: 10. The heterologous nucleic acid may comprise SEQ ID NO: 10. A nucleotide sequence encoding a synthetic myc tagged-human retinoschisin 1 (RS1) transgene (SEQ ID NO: 10) is shown below. The start codon is underlined; and the stop codon is bolded.

(SEQ ID NO: 10)
ATGTCACGCAAGATAGAAGGCTTTTTGTTATTACTTCTCTTTGGCTATGA
AGCCACATTGGGATTATCGGAGCAGAAATTAATCAGTGAGGAAGATCTGT
CTACCGAGGATGAAGGCGAGGACCCATGGTATCAAAAAGCCTGCAAGTGC
GATTGCCAAGGAGGACCCAATGCTCTGTGGTCTGCAGGTGCCACCTCCTT
GGACTGTATACCAGAATGCCCATATCACAAGCCTCTGGGTTTCGAGTCAG
GGGAGGTCACACCGGACCAGATCACCTGCTCTAACCCGGAGCAGTATGTG
GGCTGGTATTCTTCGTGGACTGCAAACAAGGCCCGGCTCAACAGTCAAGG
CTTTGGGTGTGCCTGGCTCTCCAAGTTCCAGGACAGTAGCCAGTGGTTAC
AGATAGATCTGAAGGAGATCAAAGTGATTTCAGGCATCCTCACCCAGGGG
CGCTGTGACATCGATGAGTGGATGACCAAGTACAGCGTGCAGTACAGGAC
CGATGAGCGCCTGAACTGGATTTACTACAAGGACCAGACTGGAAACAACC
GGGTCTTCTATGGCAACTCGGACCGCACCTCCACGGTTCAGAACCTGCTG
CGGCCCCCCATCATCTCCCGCTTCATCCGCCTCATCCCGCTGGGCTGGCA
CGTCCGCATTGCCATCCGGATGGAGCTGCTGGAGTGCGTCAGCAAGTGTG
CCTGA

In various embodiments, the rAAV vectors comprising a heterologous nucleic acid encode human retinoschisin 1 (RS1) protein, or a variant thereof. The amino acid sequences of the RS1 protein variants encoded by each of the nucleotide sequences of SEQ ID NOs: 8-10, respectively, are shown below as SEQ ID NOs: 12-14. In some embodiments, the encoded RS1 protein is a human retinoschisin protein defined by the amino acid sequence of SEQ ID NO: 12. In some embodiments, the encoded RS1 protein may comprise a sequence having 1, 2, 3, 4, 5, 6, 7, 8, 9, 10, 11, 12, 13, 14, 15, 16, 17, or 18 amino acids that differ relative to the sequence of any one of SEQ ID NOs: 12-14. In some embodiments, the encoded RS1 protein may comprise a sequence having 1, 2, 3, 4, 5, 6, 7, 8, 9, 10, 11, 12, 13, 14, 15, 16, 17, or 18 amino acids that differ relative to SEQ ID NO: 12. These differences may comprise amino acids that have been inserted, deleted, or substituted relative to the sequence of any one of SEQ ID NOs: 12-14. In some embodiments, the disclosed rAAV vectors encode a protein having an amino acid sequence having at least 75% identity, at least 80% identity, at least 85% identity, at least 90% identity, at least 92.5% identity, at least 95% identity, at least 98%, at least 99% identity, or 100% identity to any of the amino acid sequences of SEQ ID NOs: 12-14.

Human synthetic RS1
(SEQ ID NO: 12)
MSRKIEGFLLLLLFGYEATLGLSSTEDEGEDPWYQKACKCDCQGGPNALW
SAGATSLDCIPECPYHKPLGFESGEVTPDQITCSNPEQYVGWYSSWTANK
ARLNSQGFGCAWLSKFQDSSQWLQIDLKEIKVISGILTQGRCDIDEWMTK
YSVQYRTDERLNWIYYKDQTGNNRVFYGNSDRTSTVQNLLRPPIISRFIR
LIPLGWHVRIAIRMELLECVSKCA
Human synthetic RS1, CpG islands eliminated
(SEQ ID NO: 13)
MSRKIEGFLLLLLFGYEATLGLSSTEDEGEDPWYQKACKCDCQGGPNALW
SAGATSLDCIPECPYHKPLGFESGEVTPDQITCSNPEQYVGWYSSWTANK
ARLNSQGFGCAWLSKFQDSSQWLQIDLKEIKVISGILTQGRCDIDEWMTK
YSVQYRTDERLNWIYYKDQTGNNRVFYGNSDRTSTVQNLLRPPIISRFIR
LIPLGWHVRIAIRMELLECVSKCA
Human synthetic myc tagged-RS1; C-myc tag
underlined
(SEQ ID NO: 14)
MSRKIEGFLLLLLFGYEATLGLSEQKLISEEDLSTEDEGEDPWYQKACKC
DCQGGPNALWSAGATSLDCIPECPYHKPLGFESGEVTPDQITCSNPEQYV
GWYSSWTANKARLNSQGFGCAWLSKFQDSSQWLQIDLKEIKVISGILTQG
RCDIDEWMTKYSVQYRTDERLNWIYYKDQTGNNRVFYGNSDRTSTVQNLL
RPPIISRFIRLIPLGWHVRIAIRMELLECVSKCA

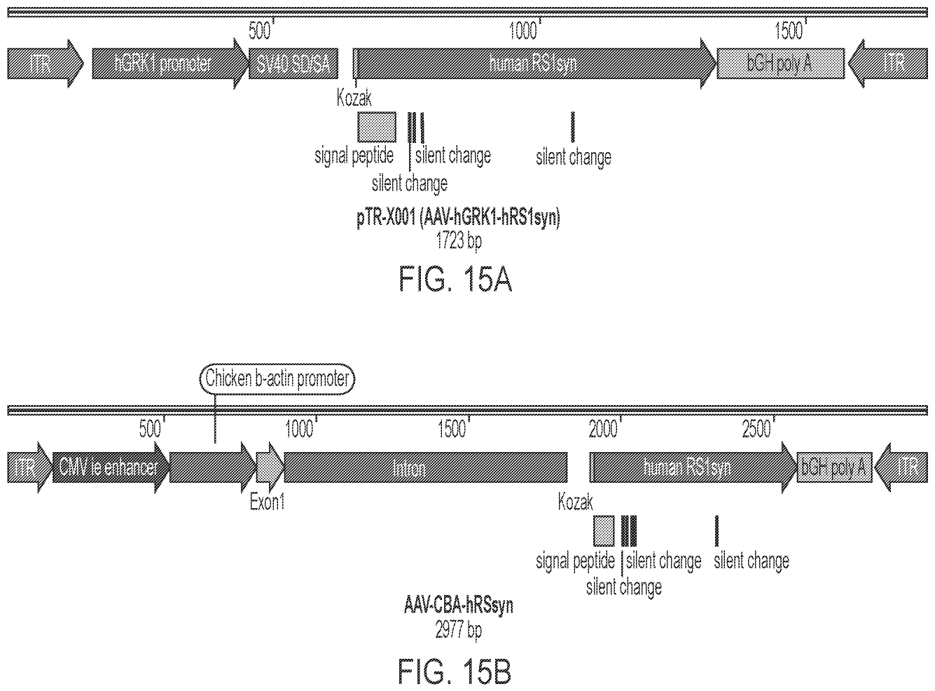

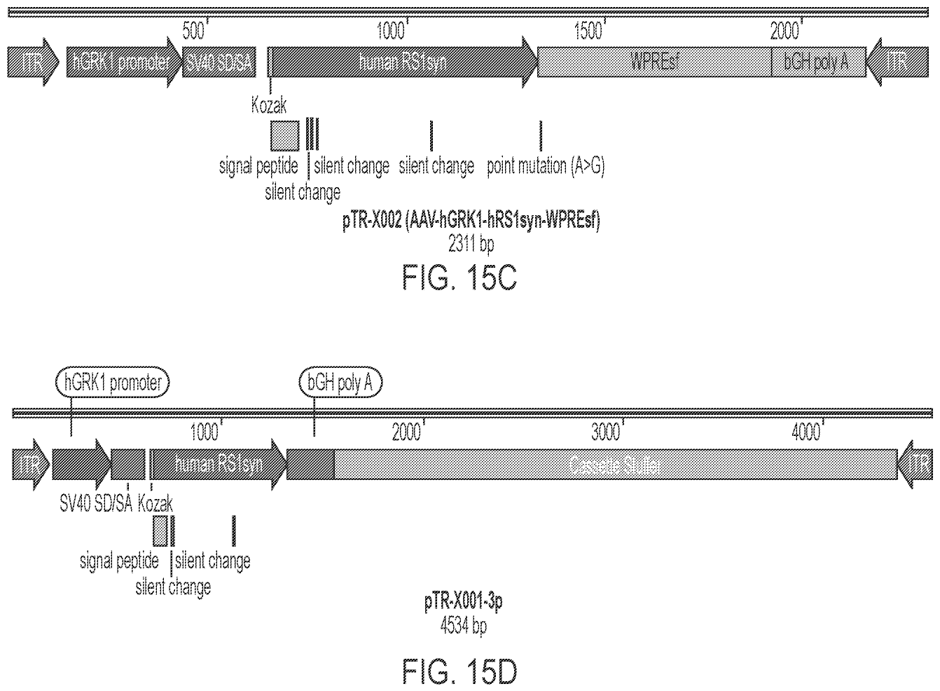

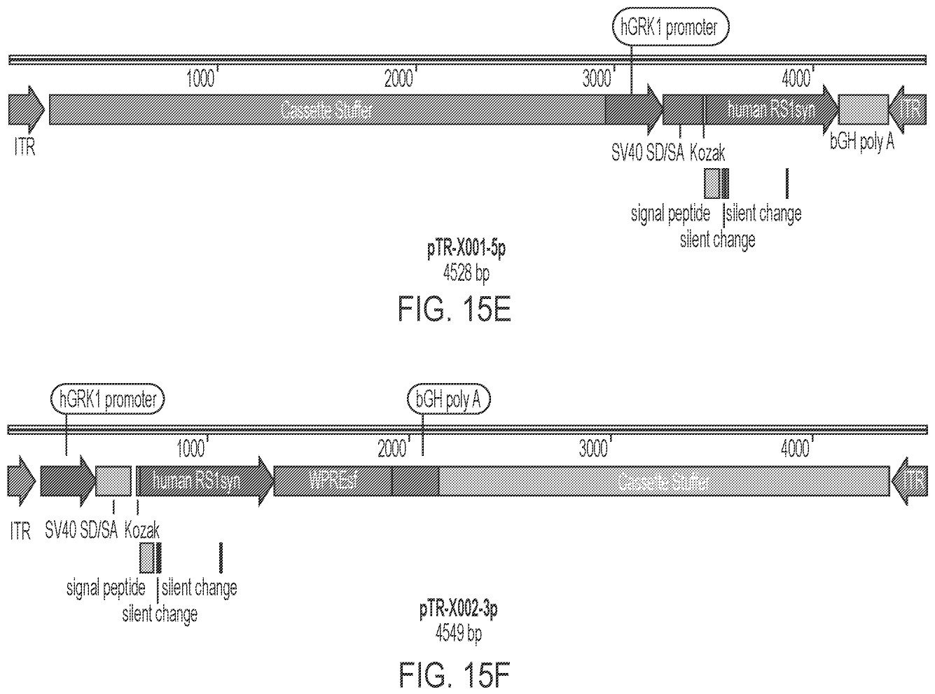

In some embodiments, the nucleic acid vector comprising the heterologous nucleic acid (e.g., RS1) has a length of between 1700 nucleotides (nt, or base pairs (bp)) and about 5000, about 4550, about 4549, about 4535, about 4534, about 4528, about 4525, about 4500, about 3200, about 3000, about 2977, about 2900, about 2800, about 2311, about 2300, about 1725, about 1723, or about 1700 nucleotides from the 5′ beginning of a first ITR to the 3′ end of a second ITR. In some embodiments, the nucleic acid vector has a length of about 1700 to about 1800 (e.g., about 1723) nucleotides from the 5′ beginning of a first ITR to the 3′ end of a second ITR (e.g., FIG. 15A). In some embodiments, the nucleic acid vector has a length of about 2900 to about 3100 (e.g., about 2977) nucleotides from the 5′ beginning of a first ITR to the 3′ end of a second ITR (e.g., FIG. 15B). In some embodiments, the nucleic acid vector has a length of about 2200 to about 2400 (e.g., about 2311) nucleotides from the 5′ beginning of a first ITR to the 3′ end of a second ITR (e.g., FIG. 15C). In some embodiments, the nucleic acid vector has a length of about 4400 to about 4600 (e.g., about 4534) nucleotides from the 5′ beginning of a first ITR to the 3′ end of a second ITR (e.g., FIG. 15D). In some embodiments, the nucleic acid vector has a length of about 4400 to about 4600 (e.g., about 4528) nucleotides from the 5′ beginning of a first ITR to the 3′ end of a second ITR (e.g., FIG. 15E). In some embodiments, the nucleic acid vector has a length of about 4400 to about 4700 (e.g., about 4549) nucleotides from the 5′ beginning of a first ITR to the 3′ end of a second ITR (e.g., FIG. 15F). In some embodiments, the nucleic acid vector comprising the heterologous nucleic acid comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 16-17 and 31-35. In some embodiments, the nucleic acid vector comprising the heterologous nucleic acid comprises the sequence of any one of SEQ ID NOs: 16-17 and 31-35.

In some embodiments, the polynucleotides as described herein may comprise a nucleic acid sequence having 1, 2, 3, 4, 5, 6, 7, 8, 9, 10, 11, 12, 13, 14, 15, 16, 17, or 18 nucleotides that differ relative to the sequence as set forth in any one of SEQ ID NOs: 31-35. These differences may comprise nucleotides that have been inserted, deleted, or substituted relative to the sequence of any one of SEQ ID NOs: 31-35. In some embodiments, the polynucleotides comprise truncations at the 5′ or 3′ end relative to any one of SEQ ID NOs: 31-35. In some embodiments, the disclosed polynucleotides contain stretches of about 50, about 75, about 100, about 125, about 150, about 175, or about 180 nucleotides in common with the sequence of any one of SEQ ID NOs: 31-35. In some embodiments, the disclosed polynucleotides contain stretches of about 50, about 75, about 100, about 125, about 150, about 175, about 180, about 200, about 300, about 400, about 500, about 600, about 700, about 800, about 900, about 1000, or more than about 1000 nucleotides in common with the sequence of any one of SEQ ID NOs: 31-35.

### Splice Donor, Splice Acceptor Regions, and Introns

In some embodiments, the nucleic acid vector comprises an intron. In some embodiments, the nucleic acid vector comprises a splice donor and splice acceptor regions independent of an intron.

In some embodiments, the nucleic acid vector comprises an SV40 intron. In some embodiments, the SV40 intron comprises an SV40 splice donor region. In some embodiments, the SV40 intron comprises an SV40 splice acceptor region. In some embodiments, the SV40 intron comprises SV40 splice donor and splice acceptor regions (SV40 SD/SA) (e.g., see FIGS. 15A-15B and 15D-15F).

The length of the SV40 intron is 99 nucleotides. It was first reported by Ostedgaard et al. that the presence of the SV40 intron between the promoter and the transgene in an AAV expression cassette provided a two-fold increase of transgene expression in lung carcinoma cells, while under the control of a CMV promoter and enhancer (PNAS 2005; 102(8): 2952-2957). Recently, it was shown that positioning an SV40 intron downstream of the expression cassette in a non-viral vector resulted in highest levels of expression of a reporter transgene (in Chinese hamster ovary cells). See Xu et al., J. Cell. Mol. Med. Vol 22, No 4 (2018): 2231-2239. In some embodiments of the disclosed rAAV vectors, the SV40 intron is positioned downstream (3′) of the heterologous nucleic acid. In some embodiments, the SV40 intron is positioned upstream (5′) of the heterologous nucleic acid.

In some embodiments, the SV40 intron comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 20. In some embodiments, the SV40 intron comprises a sequence having at least 90% or 95% identity to SEQ ID NO: 20. In some embodiments, the SV40 intron comprises SEQ ID NO: 20:

TCTAGAGGATCCGGTACTCGAGGAACTGAAAAACCAGAAAGTTAACTGGT
AAGTTTAGTCTTTTTGTCTTTTATTTCAGGTCCCGGATCCGGTGGTGGTG
CAAATCAAAGAACTGCTCCTCAGTGGATGTTGCCTTTACTTCTAGGCCTG
TACGGAAGTGTTAC.

In some embodiments, the SV40 intron contains stretches of about 50, about 75, about 80, about 85, about 95, or about 99 nucleotides in common with the sequence of SEQ ID NO: 20.

In some embodiments, the nucleic acid vector comprises a minute virus of mice (MVM) intron. For instance, the MVM intron having sequence AAGAGGTAAGGGTTTAAGGGATGGTTGGTTGGTGGGGTATTAATGTTTAATTACC TGGAGCACCTGCCTGAAATCACTTTTTTTCAGGTTGG (SEQ ID NO: 21), or a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 21.

In some embodiments, the nucleic acid vector comprises an exon 1 splice site. In some embodiments, the nucleic acid vector comprises an intron 1 splice site.

In some embodiments, the nucleic acid vector comprising the heterologous nucleic acid and an intron, splice acceptor site, and/or splice donor site (e.g., SV40 intron) has a length of between 1700 nucleotides (nt, or base pairs (bp)) and about 5000, about 4550, about 4549, about 4535, about 4534, about 4528, about 4525, about 4500, about 3200, about 3000, about 2977, about 2900, about 2800, about 2311, about 2300, about 1725, about 1723, or about 1700 nucleotides from the 5′ beginning of a first ITR to the 3′ end of a second ITR. In some embodiments, the nucleic acid vector has a length of about 1700 to about 1800 (e.g., about 1723) nucleotides from the 5′ beginning of a first ITR to the 3′ end of a second ITR (e.g., FIG. 15A). In some embodiments, the nucleic acid vector has a length of about 2900 to about 3100 (e.g., about 2977) nucleotides from the 5′ beginning of a first ITR to the 3′ end of a second ITR (e.g., FIG. 15B). In some embodiments, the nucleic acid vector has a length of about 2200 to about 2400 (e.g., about 2311) nucleotides from the 5′ beginning of a first ITR to the 3′ end of a second ITR (e.g., FIG. 15C). In some embodiments, the nucleic acid vector has a length of about 4400 to about 4600 (e.g., about 4534) nucleotides from the 5′ beginning of a first ITR to the 3′ end of a second ITR (e.g., FIG. 15D). In some embodiments, the nucleic acid vector has a length of about 4400 to about 4600 (e.g., about 4528) nucleotides from the 5′ beginning of a first ITR to the 3′ end of a second ITR (e.g., FIG. 15E). In some embodiments, the nucleic acid vector has a length of about 4400 to about 4700 (e.g., about 4549) nucleotides from the 5′ beginning of a first ITR to the 3′ end of a second ITR (e.g., FIG. 15F). In some embodiments, the nucleic acid vector comprising the heterologous nucleic acid and an intron, splice acceptor site, and/or splice donor site (e.g., SV40 intron) comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NO s: 16-17 and 31-35. In some embodiments, the nucleic acid vector comprising the heterologous nucleic acid and an intron, splice acceptor site, and/or splice donor site (e.g., SV40 intron) comprises a sequence of any one of SEQ ID NOs: 16-17 and 31-35.

### Promoters

In some embodiments, the polynucleotide within the rAAV particle comprises regulatory sequences, such as transcription and translation initiation and termination codons, which are specific to the type of host (e.g., bacterium, fungus, plant, or animal) into which the rAAV particle is to be introduced. Preferably, the nucleic acid molecule within the rAAV particle comprises regulatory sequences that are specific to the genus of the host. Most preferably, the molecule comprises regulatory sequences that are specific to the species of the host. The polynucleotide within the rAAV particle may comprise expression control sequences, such as promoters, enhancers, polyadenylation signals, transcription terminators, internal ribosome entry sites (IRES), and the like, that provide for the expression of the heterologous nucleic acid(s) in a host cell. Exemplary expression control sequences are known in the art. In some embodiments, the heterologous nucleic acid is operably linked to one or more regulatory sequences which direct expression of the heterologous nucleic acid in a photoreceptor cell or retinal pigment epithelium cell.

In some embodiments, the polynucleotide of any of the disclosed rAAV vectors comprises a promoter that is capable of expressing the nucleic acid sequence in one or more photoreceptors or retinal pigment epithelial cells of a mammalian eye. In particular embodiments, the disclosure provides a PR- or RPE-cell-specific promoter operably linked to at least a first hetereologous nucleic acid sequence that encodes a therapeutic agent. Exemplary PR- or RPE-cell-specific promoters may comprise a) photoreceptor-specific promoters (active in rod and cone cells), e.g., IRBP promoter (hIRPB, IRBP, IRBP241), rhodopsin kinase promoter (hGRK1, GRK1, GRK, RK), and/or chimeric human Retinoschisin-IRBP enhancer (RS/IRPB); cone-specific promoters, e.g., red/green cone opsin promoter (which may comprise the 2.1 kb (PR2.1) version or 1.7 kb (PR1.7) version, see U.S. Patent Publication No. 2018/0112231, herein incorporated by reference), Cone Arrestin promoter (hCAR, CAR), chimeric IRBP enhancer-cone transducin promoter (IRBP/GNAT2, IRBPe-GNAT2); rod-specific promoters, e.g., human rhodopsin promoter (RHO, RHOP, etc.), human NRL promoter (NRL); or RPE-specific promoters such as RPE65 or Bestrophin/VMD2 (BEST1, BEST, VMD2). In some embodiments, the promoter is a photoreceptor-specific promoter such as an IRBP promoter (hIRPB, IRBP, IRBP241). In some embodiments, the promoter is a rod-specific promoter such as a human rhodopsin promoter (RHO, RHOP).

In exemplary embodiments, the polynucleotide comprises an hGRK1 promoter. In some embodiments, the polynucleotide comprises a CBA promoter. In some embodiments, the polynucleotide comprises a truncated chimeric CBA-CMV promoter (smCBA) promoter, which contains a CMV enhancer and a truncated CBA promoter.

Exemplary vectors of the disclosure that comprise hGRK1 promoters include AAV-hGRK1-hRS1syn (FIG. 15A), AAV-hGRK1-hRS1syn-WPREsf (FIG. 15C), pTR-X001-3p (FIG. 15D), pTR-X001-5p (FIG. 15E), and pTR-X002-3p (FIG. 15F). An exemplary vector of the disclosure that comprise an smCBA promoter is AAV-CBA-hRS1syn (FIG. 15B).

In some embodiments, the promoter of any of the disclosed rAAV vectors comprises a nucleotide sequence that is at least 95%, at least 98%, at least 99%, or 100% identical the sequence of the hGRK1 promoter as set forth in SEQ ID NO: 7:

(SEQ ID NO: 7)
GGGCCCCAGAAGCCTGGTGGTTGTTTGTCCTTCTCAGGGGAAAAGTGAGG
CGGCCCCTTGGAGGAAGGGGCCGGGCAGAATGATCTAATCGGATTCCAAG
CAGCTCAGGGGATTGTCTTTTTCTAGCACCTTCTTGCCACTCCTAAGCGT
CCTCCGTGACCCCGGCTGGGATTTAGCCTGGTGCTGTGTCAGCCCCGGTC
TCCCAGGGGCTTCCCAGTGGTCCCCAGGAACCCTCGACAGGGCCCGGTCT
CTCTCGTCCAGCAAGGGCAGGGACGGGCCACAGGCCAAGGGC

In some embodiments, the disclosure provides constitutive promoters operably linked to at least a first polynucleotide that may comprise rhodopsin, syn1 (synapsin), CMV, CBA, CB, smCBA, CBh, or EF1-alpha promoter.

In some embodiments, the nucleic acid vector comprising the heterologous nucleic acid and promoter has a length of between 1700 nucleotides (nt, or base pairs (bp)) and about 5000, about 4550, about 4549, about 4535, about 4534, about 4528, about 4525, about 4500, about 3200, about 3000, about 2977, about 2900, about 2800, about 2311, about 2300, about 1725, about 1723, or about 1700 nucleotides from the 5′ beginning of a first ITR to the 3′ end of a second ITR. In some embodiments, the nucleic acid vector has a length of about 1700 to about 1800 (e.g., about 1723) nucleotides from the 5′ beginning of a first ITR to the 3′ end of a second ITR (e.g., FIG. 15A). In some embodiments, the nucleic acid vector has a length of about 2900 to about 3100 (e.g., about 2977) nucleotides from the 5′ beginning of a first ITR to the 3′ end of a second ITR (e.g., FIG. 15B). In some embodiments, the nucleic acid vector has a length of about 2200 to about 2400 (e.g., about 2311) nucleotides from the 5′ beginning of a first ITR to the 3′ end of a second ITR (e.g., FIG. 15C). In some embodiments, the nucleic acid vector has a length of about 4400 to about 4600 (e.g., about 4534) nucleotides from the 5′ beginning of a first ITR to the 3′ end of a second ITR (e.g., FIG. 15D). In some embodiments, the nucleic acid vector has a length of about 4400 to about 4600 (e.g., about 4528) nucleotides from the 5′ beginning of a first ITR to the 3′ end of a second ITR (e.g., FIG. 15E). In some embodiments, the nucleic acid vector has a length of about 4400 to about 4700 (e.g., about 4549) nucleotides from the 5′ beginning of a first ITR to the 3′ end of a second ITR (e.g., FIG. 15F). In some embodiments, the nucleic acid vector comprising the heterologous nucleic acid and promoter comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 16-17, 31-35. In some embodiments, the nucleic acid vector comprising the heterologous nucleic acid and promoter comprises a sequence of any one of SEQ ID NOs: 16-17, 31-35.

### Post-Transcription Regulatory Elements

In some embodiments, the nucleic acid vector comprises a post-transcriptional regulatory sequence. In some embodiments, the nucleic acid vector comprises a woodchuck hepatitis virus post-transcription regulatory element (WPRE). The polynucleotide may comprises a WPRE element, such as a WPRE element that comprises the nucleotide sequence of SEQ ID NO: 15. In some embodiments, the WPRE element is positioned 3′ of the heterologous nucleic acid. In some embodiments, the WPRE element is positioned 5′ of the heterologous nucleic acid.

In some embodiments, the nucleic acid vector comprises a WPRE. In some cases, the WPRE is a WPREsf sequence, where the “sf” suffix denotes safe for administration. In some embodiments, the polynucleotide of any of the disclosed rAAV vectors comprises a WPRE element having at least 95%, 98%, or 99% identity to the nucleotide sequence of SEQ ID NO: 15. In some embodiments, the polynucleotide comprises the sequence of SEQ ID NO: 15.

WPREsf sequence:
(SEQ ID NO: 15)
TCGACCTCTGGATTACAAAATTTGTGAAAGATTGACTGGTATTCTTAACT
ATGTTGCTCCTTTTACGCTATGTGGATACGCTGCTTTAATGCCTTTGTAT
CATGCTATTGCTTCCCGTATGGCTTTCATTTTCTCCTCCTTGTATAAATC
CTGGTTGCTGTCTCTTTATGAGGAGTTGTGGCCCGTTGTCAGGCAACGTG
GCGTGGTGTGCACTGTGTTTGCTGACGCAACCCCCACTGGTTGGGGCATT
GCCACCACCTGTCAGCTCCTTTCCGGGACTTTCGCTTTCCCCCTCCCTAT
TGCCACGGCGGAACTCATCGCCGCCTGCCTTGCCCGCTGCTGGACAGGGG
CTCGGCTGTTGGGCACTGACAATTCCGTGGTGTTGTCGGGGAAATCATCG
TCCTTTCCTTGGCTGCTCGCCTGTGTTGCCACCTGGATTCTGCGCGGGAC
GTCCTTCTGCTACGTCCCTTCGGCCCTCAATCCAGCGGACCTTCCTTCCC
GCGGCCTGCTGCCGGCTCTGCGGCCTCTTCCGCGTCTTCGCCTTCGCCCT
CAGACGAGTCGGATCTCCCTTTGGGCCGCCTCCCCGC

In some embodiments, the nucleic acid vector comprising the heterologous nucleic acid and post-transcription regulatory element has a length of between 1700 nucleotides (nt, or base pairs (bp)) and about 5000, about 4550, about 4549, about 4535, about 4534, about 4528, about 4525, about 4500, about 3200, about 3000, about 2977, about 2900, about 2800, about 2311, about 2300, about 1725, about 1723, or about 1700 nucleotides from the 5′ beginning of a first ITR to the 3′ end of a second ITR. In some embodiments, the nucleic acid vector has a length of about 1700 to about 1800 (e.g., about 1723) nucleotides from the 5′ beginning of a first ITR to the 3′ end of a second ITR (e.g., FIG. 15A). In some embodiments, the nucleic acid vector has a length of about 2900 to about 3100 (e.g., about 2977) nucleotides from the 5′ beginning of a first ITR to the 3′ end of a second ITR (e.g., FIG. 15B). In some embodiments, the nucleic acid vector has a length of about 2200 to about 2400 (e.g., about 2311) nucleotides from the 5′ beginning of a first ITR to the 3′ end of a second ITR (e.g., FIG. 15C). In some embodiments, the nucleic acid vector has a length of about 4400 to about 4600 (e.g., about 4534) nucleotides from the 5′ beginning of a first ITR to the 3′ end of a second ITR (e.g., FIG. 15D). In some embodiments, the nucleic acid vector has a length of about 4400 to about 4600 (e.g., about 4528) nucleotides from the 5′ beginning of a first ITR to the 3′ end of a second ITR (e.g., FIG. 15E). In some embodiments, the nucleic acid vector has a length of about 4400 to about 4700 (e.g., about 4549) nucleotides from the 5′ beginning of a first ITR to the 3′ end of a second ITR (e.g., FIG. 15F). In some embodiments, the nucleic acid vector comprising the heterologous nucleic acid and post-transcription regulatory element comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 16-17 and 31-35. In some embodiments, the nucleic acid vector comprising the heterologous nucleic acid and post-transcription regulatory element comprises a sequence of any one of SEQ ID NOs: 16-17 and 31-35.

Polyadenylation (polyA) Signal Sequence

In some embodiments, the nucleic acid vector comprises a polyadenylation (polyA) signal sequence. In some embodiments, the polyadenylation signal is selected from a bovine growth factor hormone (bGH) polyadenylation signal, an SV40 polyadenylation signal, a human growth factor hormone (hGH) polyadenylation signal, and a rabbit beta-globin (rbGlob) polyadenylation signal.

In some embodiments, the vector comprises a bGH polyA signal. In some embodiments, the vector comprises an SV40 polyA signal.

In some embodiments, the vector comprises a bGH polyA signal having a nucleic acid sequence having at least 80%, 85%, 90, 92.5%, 95%, 98% or 99% identity to SEQ ID NO: 19. In some embodiments, the polyA signal of any of the disclosed vectors comprises the nucleic acid sequence of SEQ ID NO: 19.

bGH polyA signal:
(SEQ ID NO: 19)
TCGACTAGAGCTCGCTGATCAGCCTCGACTGTGCCTTCTAGTTGCCAGCC
ATCTGTTGTTTGCCCCTCCCCCGTGCCTTCCTTGACCCTGGAAGGTGCCA
CTCCCACTGTCCTTTCCTAATAAAATGAGGAAATTGCATCGCATTGTCTG
AGTAGGTGTCATTCTATTCTGGGGGGTGGGGTGGGGCAGGACAGCAAGGG
GGAGGATTGGGAAGACAATAGCAGGCAT

In some embodiments, the nucleic acid vector comprising the heterologous nucleic acid and polyA signal has a length of between 1700 nucleotides (nt, or base pairs (bp)) and about 5000, about 4550, about 4549, about 4535, about 4534, about 4528, about 4525, about 4500, about 3200, about 3000, about 2977, about 2900, about 2800, about 2311, about 2300, about 1725, about 1723, or about 1700 nucleotides from the 5′ beginning of a first ITR to the 3′ end of a second ITR. In some embodiments, the nucleic acid vector has a length of about 1700 to about 1800 (e.g., about 1723) nucleotides from the 5′ beginning of a first ITR to the 3′ end of a second ITR (e.g., FIG. 15A). In some embodiments, the nucleic acid vector has a length of about 2900 to about 3100 (e.g., about 2977) nucleotides from the 5′ beginning of a first ITR to the 3′ end of a second ITR (e.g., FIG. 15B). In some embodiments, the nucleic acid vector has a length of about 2200 to about 2400 (e.g., about 2311) nucleotides from the 5′ beginning of a first ITR to the 3′ end of a second ITR (e.g., FIG. 15C). In some embodiments, the nucleic acid vector has a length of about 4400 to about 4600 (e.g., about 4534) nucleotides from the 5′ beginning of a first ITR to the 3′ end of a second ITR (e.g., FIG. 15D). In some embodiments, the nucleic acid vector has a length of about 4400 to about 4600 (e.g., about 4528) nucleotides from the 5′ beginning of a first ITR to the 3′ end of a second ITR (e.g., FIG. 15E). In some embodiments, the nucleic acid vector has a length of about 4400 to about 4700 (e.g., about 4549) nucleotides from the 5′ beginning of a first ITR to the 3′ end of a second ITR (e.g., FIG. 15F). In some embodiments, the nucleic acid vector comprising the heterologous nucleic acid and polyA signal comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 16-17, 31-35. In some embodiments, the nucleic acid vector comprising the heterologous nucleic acid and polyA signal comprises a sequence of any one of SEQ ID NOs: 16-17, 31-35.

### Terminal Repeats

In some embodiments, the nucleic acid vector comprises one or more terminal repeats. In some embodiments, the nucleic acid vector comprises a first inverted terminal repeat (ITR) and a second inverted terminal repeat (ITR). In some embodiments, an inverted terminal repeat comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 22 or 23. In some embodiments, an inverted terminal repeat comprises SEQ ID NO: 22 or 23. In some embodiments, an inverted terminal repeat comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 24 or 25. In some embodiments, an inverted terminal repeat comprises SEQ ID NO: 24 or 25.

Inverted terminal repeat (e.g., first ITR)
(SEQ ID NO: 22)
GGCCACTCCCTCTCTGCGCGCTCGCTCGCTCACTGAGGCCGCCCGGGCAA
AGCCCGGGCGTCGGGCGACCTTTGGTCGCCCGGCCTCAGTGAGCGAGCGA
GCGCGCAGAGAGGGAGTGGCCAACTCCATCACTAGGGGTTCCT
Inverted terminal repeat (e.g., first ITR)
(SEQ ID NO: 23)
TTGGCCACTCCCTCTCTGCGCGCTCGCTCGCTCACTGAGGCCGCCCGGGC
AAAGCCCGGGCGTCGGGCGACCTTTGGTCGCCCGGCCTCAGTGAGCGAGC
GAGCGCGCAGAGAGGGAGTGGCCAACTCCATCACTAGGGGTTCCT
Inverted terminal repeat (e.g., second ITR)
(SEQ ID NO: 24)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGAGAGGGAGTGGCC
Inverted terminal repeat (e.g., second ITR)
(SEQ ID NO: 25)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGAGAGGGAGTGGCCAA

In some embodiments, the nucleic acid vector comprising the heterologous nucleic acid and terminal repeat has a length of between 1700 nucleotides (nt, or base pairs (bp)) and about 5000, about 4550, about 4549, about 4535, about 4534, about 4528, about 4525, about 4500, about 3200, about 3000, about 2977, about 2900, about 2800, about 2311, about 2300, about 1725, about 1723, or about 1700 nucleotides from the 5′ beginning of a first ITR to the 3′ end of a second ITR. In some embodiments, the nucleic acid vector has a length of about 1700 to about 1800 (e.g., about 1723) nucleotides from the 5′ beginning of a first ITR to the 3′ end of a second ITR (e.g., FIG. 15A). In some embodiments, the nucleic acid vector has a length of about 2900 to about 3100 (e.g., about 2977) nucleotides from the 5′ beginning of a first ITR to the 3′ end of a second ITR (e.g., FIG. 15B). In some embodiments, the nucleic acid vector has a length of about 2200 to about 2400 (e.g., about 2311) nucleotides from the 5′ beginning of a first ITR to the 3′ end of a second ITR (e.g., FIG. 15C). In some embodiments, the nucleic acid vector has a length of about 4400 to about 4600 (e.g., about 4534) nucleotides from the 5′ beginning of a first ITR to the 3′ end of a second ITR (e.g., FIG. 15D). In some embodiments, the nucleic acid vector has a length of about 4400 to about 4600 (e.g., about 4528) nucleotides from the 5′ beginning of a first ITR to the 3′ end of a second ITR (e.g., FIG. 15E). In some embodiments, the nucleic acid vector has a length of about 4400 to about 4700 (e.g., about 4549) nucleotides from the 5′ beginning of a first ITR to the 3′ end of a second ITR (e.g., FIG. 15F). In some embodiments, the nucleic acid vector comprising the heterologous nucleic acid and terminal repeat comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 16, 17, and 31-35. In some embodiments, the nucleic acid vector comprising the heterologous nucleic acid and terminal repeat comprises a sequence of any one of SEQ ID NOs: 16, 17, and 31-35.

### Stuffer Sequences

In some embodiments, the rAAV vectors of the disclosure contain one or more stuffer sequences. In some embodiments, a “stuffer” sequence refers to a generic, inert, non-coding sequence that increases the length of the rAAV vectors. In some embodiments, the stuffer sequence increases the length of any of the disclosed rAAV vectors such that the cassette is close to wtAAV genome size (e.g., ˜4.8 kb from ITR to ITR).

In some embodiments, the nucleic acid stuffer sequence has a length of about 100 to about 5000 nucleobases. In some embodiments, the nucleic acid stuffer sequence has a length of about 100 to about 5000, about 100 to about 4000, about 100 to about 3000, or about 100 to about 2000 nucleobases. In some embodiments, the nucleic acid stuffer sequence has a length of about 100 to about 5000, about 500 to about 5000, about 1000 to about 5000, about 2000 to about 5000, or about 3000 to about 5000 nucleobases. In some embodiments, the nucleic acid stuffer sequence has a length of about 1000 to about 5000, about 1050 to about 4500, or about 2000 to about 3000 nucleobases. In some embodiments, the nucleic acid stuffer sequence has a length of about 2822 nucleobases. In some embodiments, the nucleic acid stuffer sequence has a length of about 2820 nucleobases. In some embodiments, the nucleic acid stuffer sequence has a length of about 2249 nucleobases. In some embodiments, the nucleic acid stuffer sequence has a length of about 2235 nucleobases. In some embodiments, the nucleic acid stuffer sequence has a length of about 2219 nucleobases.

In some embodiments, a cassette (from the beginning of a first ITR to the end of a second ITR) comprising the nucleic acid stuffer sequence has a length of about 2000 to about 5000 nucleobases. In some embodiments, the cassette comprising the nucleic acid stuffer sequence has a length of about 3000 to about 5000, about 3500 to about 5000, about 4000 to about 5000, about 4100 to about 5000, about 4200 to about 5000, about 4300 to about 5000, about 4400 to about 5000, about 4500 to about 5000, about 4000 to about 4900, about 4100 to about 4900, about 4200 to about 4900, about 4300 to about 4900, about 4400 to about 4900, about 4000 to about 4800, about 4100 to about 4800, about 4200 to about 4800, about 4300 to about 4800, about 4400 to about 4800, about 4000 to about 4700, about 4100 to about 5000, about 4200 to about 4700, about 4300 to about 4700, about 4400 to about 4700, about 4000 to about 4600, about 4100 to about 4600, about 4200 to about 4600, about 4300 to about 4600, about 4400 to about 4600, about 4000 to about 4500, about 4100 to about 4500, about 4200 to about 4500, about 4300 to about 4500, or about 4400 to about 4500 nucleobases. In some embodiments, the cassette comprising the nucleic acid stuffer sequence has a length of about 4500, 4510, 4520, 4530, 4540, 4550, 4560, 4570, 4580, 4590, or 5000 nucleobases. In some embodiments, the cassette comprising the nucleic acid stuffer sequence has a length of about 4534 nucleobases. In some embodiments, the cassette comprising the nucleic acid stuffer sequence has a length of about 4528 nucleobases. In some embodiments, the cassette comprising the nucleic acid stuffer sequence has a length of about 4549 nucleobases.

In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 26-30. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 26-30. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 26-30.

In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to bases 1-100, 2-101, 3-102, 4-103, 5-104, 6-105, 7-106, 8-107, 9-108, 10-109, 11-110, 12-111, 13-112, 14-113, 15-114, 16-115, 17-116, 18-117, 19-118, 20-119, 21-120, 22-121, 23-122, 24-123, 25-124, 26-125, 27-126, 28-127, 29-128, 30-129, 31-130, 32-131, 33-132, 34-133, 35-134, 36-135, 37-136, 38-137, 39-138, 40-139, 41-140, 42-141, 43-142, 44-143, 45-144, 46-145, 47-146, 48-147, 49-148, 50-149, 51-150, 52-151, 53-152, 54-153, 55-154, 56-155, 57-156, 58-157, 59-158, 60-159, 61-160, 62-161, 63-162, 64-163, 65-164, 66-165, 67-166, 68-167, 69-168, 70-169, 71-170, 72-171, 73-172, 74-173, 75-174, 76-175, 77-176, 78-177, 79-178, 80-179, 81-180, 82-181, 83-182, 84-183, 85-184, 86-185, 87-186, 88-187, 89-188, 90-189, 91-190, 92-191, 93-192, 94-193, 95-194, 96-195, 97-196, 98-197, 99-198, 100-199, 101-200, 102-201, 103-202, 104-203, 105-204, 106-205, 107-206, 108-207, 109-208, 110-209, 111-210, 112-211, 113-212, 114-213, 115-214, 116-215, 117-216, 118-217, 119-218, 120-219, 121-220, 122-221, 123-222, 124-223, 125-224, 126-225, 127-226, 128-227, 129-228, 130-229, 131-230, 132-231, 133-232, 134-233, 135-234, 136-235, 137-236, 138-237, 139-238, 140-239, 141-240, 142-241, 143-242, 144-243, 145-244, 146-245, 147-246, 148-247, 149-248, 150-249, 151-250, 152-251, 153-252, 154-253, 155-254, 156-255, 157-256, 158-257, 159-258, 160-259, 161-260, 162-261, 163-262, 164-263, 165-264, 166-265, 167-266, 168-267, 169-268, 170-269, 171-270, 172-271, 173-272, 174-273, 175-274, 176-275, 177-276, 178-277, 179-278, 180-279, 181-280, 182-281, 183-282, 184-283, 185-284, 186-285, 187-286, 188-287, 189-288, 190-289, 191-290, 192-291, 193-292, 194-293, 195-294, 196-295, 197-296, 198-297, 199-298, 200-299, 201-300, 202-301, 203-302, 204-303, 205-304, 206-305, 207-306, 208-307, 209-308, 210-309, 211-310, 212-311, 213-312, 214-313, 215-314, 216-315, 217-316, 218-317, 219-318, 220-319, 221-320, 222-321, 223-322, 224-323, 225-324, 226-325, 227-326, 228-327, 229-328, 230-329, 231-330, 232-331, 233-332, 234-333, 235-334, 236-335, 237-336, 238-337, 239-338, 240-339, 241-340, 242-341, 243-342, 244-343, 245-344, 246-345, 247-346, 248-347, 249-348, 250-349, 251-350, 252-351, 253-352, 254-353, 255-354, 256-355, 257-356, 258-357, 259-358, 260-359, 261-360, 262-361, 263-362, 264-363, 265-364, 266-365, 267-366, 268-367, 269-368, 270-369, 271-370, 272-371, 273-372, 274-373, 275-374, 276-375, 277-376, 278-377, 279-378, 280-379, 281-380, 282-381, 283-382, 284-383, 285-384, 286-385, 287-386, 288-387, 289-388, 290-389, 291-390, 292-391, 293-392, 294-393, 295-394, 296-395, 297-396, 298-397, 299-398, 300-399, 301-400, 302-401, 303-402, 304-403, 305-404, 306-405, 307-406, 308-407, 309-408, 310-409, 311-410, 312-411, 313-412, 314-413, 315-414, 316-415, 317-416, 318-417, 319-418, 320-419, 321-420, 322-421, 323-422, 324-423, 325-424, 326-425, 327-426, 328-427, 329-428, 330-429, 331-430, 332-431, 333-432, 334-433, 335-434, 336-435, 337-436, 338-437, 339-438, 340-439, 341-440, 342-441, 343-442, 344-443, 345-444, 346-445, 347-446, 348-447, 349-448, 350-449, 351-450, 352-451, 353-452, 354-453, 355-454, 356-455, 357-456, 358-457, 359-458, 360-459, 361-460, 362-461, 363-462, 364-463, 365-464, 366-465, 367-466, 368-467, 369-468, 370-469, 371-470, 372-471, 373-472, 374-473, 375-474, 376-475, 377-476, 378-477, 379-478, 380-479, 381-480, 382-481, 383-482, 384-483, 385-484, 386-485, 387-486, 388-487, 389-488, 390-489, 391-490, 392-491, 393-492, 394-493, 395-494, 396-495, 397-496, 398-497, 399-498, 400-499, 401-500, 402-501, 403-502, 404-503, 405-504, 406-505, 407-506, 408-507, 409-508, 410-509, 411-510, 412-511, 413-512, 414-513, 415-514, 416-515, 417-516, 418-517, 419-518, 420-519, 421-520, 422-521, 423-522, 424-523, 425-524, 426-525, 427-526, 428-527, 429-528, 430-529, 431-530, 432-531, 433-532, 434-533, 435-534, 436-535, 437-536, 438-537, 439-538, 440-539, 441-540, 442-541, 443-542, 444-543, 445-544, 446-545, 447-546, 448-547, 449-548, 450-549, 451-550, 452-551, 453-552, 454-553, 455-554, 456-555, 457-556, 458-557, 459-558, 460-559, 461-560, 462-561, 463-562, 464-563, 465-564, 466-565, 467-566, 468-567, 469-568, 470-569, 471-570, 472-571, 473-572, 474-573, 475-574, 476-575, 477-576, 478-577, 479-578, 480-579, 481-580, 482-581, 483-582, 484-583, 485-584, 486-585, 487-586, 488-587, 489-588, 490-589, 491-590, 492-591, 493-592, 494-593, 495-594, 496-595, 497-596, 498-597, 499-598, 500-599, 501-600, 502-601, 503-602, 504-603, 505-604, 506-605, 507-606, 508-607, 509-608, 510-609, 511-610, 512-611, 513-612, 514-613, 515-614, 516-615, 517-616, 518-617, 519-618, 520-619, 521-620, 522-621, 523-622, 524-623, 525-624, 526-625, 527-626, 528-627, 529-628, 530-629, 531-630, 532-631, 533-632, 534-633, 535-634, 536-635, 537-636, 538-637, 539-638, 540-639, 541-640, 542-641, 543-642, 544-643, 545-644, 546-645, 547-646, 548-647, 549-648, 550-649, 551-650, 552-651, 553-652, 554-653, 555-654, 556-655, 557-656, 558-657, 559-658, 560-659, 561-660, 562-661, 563-662, 564-663, 565-664, 566-665, 567-666, 568-667, 569-668, 570-669, 571-670, 572-671, 573-672, 574-673, 575-674, 576-675, 577-676, 578-677, 579-678, 580-679, 581-680, 582-681, 583-682, 584-683, 585-684, 586-685, 587-686, 588-687, 589-688, 590-689, 591-690, 592-691, 593-692, 594-693, 595-694, 596-695, 597-696, 598-697, 599-698, 600-699, 601-700, 602-701, 603-702, 604-703, 605-704, 606-705, 607-706, 608-707, 609-708, 610-709, 611-710, 612-711, 613-712, 614-713, 615-714, 616-715, 617-716, 618-717, 619-718, 620-719, 621-720, 622-721, 623-722, 624-723, 625-724, 626-725, 627-726, 628-727, 629-728, 630-729, 631-730, 632-731, 633-732, 634-733, 635-734, 636-735, 637-736, 638-737, 639-738, 640-739, 641-740, 642-741, 643-742, 644-743, 645-744, 646-745, 647-746, 648-747, 649-748, 650-749, 651-750, 652-751, 653-752, 654-753, 655-754, 656-755, 657-756, 658-757, 659-758, 660-759, 661-760, 662-761, 663-762, 664-763, 665-764, 666-765, 667-766, 668-767, 669-768, 670-769, 671-770, 672-771, 673-772, 674-773, 675-774, 676-775, 677-776, 678-777, 679-778, 680-779, 681-780, 682-781, 683-782, 684-783, 685-784, 686-785, 687-786, 688-787, 689-788, 690-789, 691-790, 692-791, 693-792, 694-793, 695-794, 696-795, 697-796, 698-797, 699-798, 700-799, 701-800, 702-801, 703-802, 704-803, 705-804, 706-805, 707-806, 708-807, 709-808, 710-809, 711-810, 712-811, 713-812, 714-813, 715-814, 716-815, 717-816, 718-817, 719-818, 720-819, 721-820, 722-821, 723-822, 724-823, 725-824, 726-825, 727-826, 728-827, 729-828, 730-829, 731-830, 732-831, 733-832, 734-833, 735-834, 736-835, 737-836, 738-837, 739-838, 740-839, 741-840, 742-841, 743-842, 744-843, 745-844, 746-845, 747-846, 748-847, 749-848, 750-849, 751-850, 752-851, 753-852, 754-853, 755-854, 756-855, 757-856, 758-857, 759-858, 760-859, 761-860, 762-861, 763-862, 764-863, 765-864, 766-865, 767-866, 768-867, 769-868, 770-869, 771-870, 772-871, 773-872, 774-873, 775-874, 776-875, 777-876, 778-877, 779-878, 780-879, 781-880, 782-881, 783-882, 784-883, 785-884, 786-885, 787-886, 788-887, 789-888, 790-889, 791-890, 792-891, 793-892, 794-893, 795-894, 796-895, 797-896, 798-897, 799-898, 800-899, 801-900, 802-901, 803-902, 804-903, 805-904, 806-905, 807-906, 808-907, 809-908, 810-909, 811-910, 812-911, 813-912, 814-913, 815-914, 816-915, 817-916, 818-917, 819-918, 820-919, 821-920, 822-921, 823-922, 824-923, 825-924, 826-925, 827-926, 828-927, 829-928, 830-929, 831-930, 832-931, 833-932, 834-933, 835-934, 836-935, 837-936, 838-937, 839-938, 840-939, 841-940, 842-941, 843-942, 844-943, 845-944, 846-945, 847-946, 848-947, 849-948, 850-949, 851-950, 852-951, 853-952, 854-953, 855-954, 856-955, 857-956, 858-957, 859-958, 860-959, 861-960, 862-961, 863-962, 864-963, 865-964, 866-965, 867-966, 868-967, 869-968, 870-969, 871-970, 872-971, 873-972, 874-973, 875-974, 876-975, 877-976, 878-977, 879-978, 880-979, 881-980, 882-981, 883-982, 884-983, 885-984, 886-985, 887-986, 888-987, 889-988, 890-989, 891-990, 892-991, 893-992, 894-993, 895-994, 896-995, 897-996, 898-997, 899-998, 900-999, 901-1000, 902-1001, 903-1002, 904-1003, 905-1004, 906-1005, 907-1006, 908-1007, 909-1008, 910-1009, 911-1010, 912-1011, 913-1012, 914-1013, 915-1014, 916-1015, 917-1016, 918-1017, 919-1018, 920-1019, 921-1020, 922-1021, 923-1022, 924-1023, 925-1024, 926-1025, 927-1026, 928-1027, 929-1028, 930-1029, 931-1030, 932-1031, 933-1032, 934-1033, 935-1034, 936-1035, 937-1036, 938-1037, 939-1038, 940-1039, 941-1040, 942-1041, 943-1042, 944-1043, 945-1044, 946-1045, 947-1046, 948-1047, 949-1048, 950-1049, 951-1050, 952-1051, 953-1052, 954-1053, 955-1054, 956-1055, 957-1056, 958-1057, 959-1058, 960-1059, 961-1060, 962-1061, 963-1062, 964-1063, 965-1064, 966-1065, 967-1066, 968-1067, 969-1068, 970-1069, 971-1070, 972-1071, 973-1072, 974-1073, 975-1074, 976-1075, 977-1076, 978-1077, 979-1078, 980-1079, 981-1080, 982-1081, 983-1082, 984-1083, 985-1084, 986-1085, 987-1086, 988-1087, 989-1088, 990-1089, 991-1090, 992-1091, 993-1092, 994-1093, 995-1094, 996-1095, 997-1096, 998-1097, 999-1098, 1000-1099, 1000-1100, 1001-1101, 1002-1102, 1003-1103, 1004-1104, 1005-1105, 1006-1106, 1007-1107, 1008-1108, 1009-1109, 1010-1110, 1011-1111, 1012-1112, 1013-1113, 1014-1114, 1015-1115, 1016-1116, 1017-1117, 1018-1118, 1019-1119, 1020-1120, 1021-1121, 1022-1122, 1023-1123, 1024-1124, 1025-1125, 1026-1126, 1027-1127, 1028-1128, 1029-1129, 1030-1130, 1031-1131, 1032-1132, 1033-1133, 1034-1134, 1035-1135, 1036-1136, 1037-1137, 1038-1138, 1039-1139, 1040-1140, 1041-1141, 1042-1142, 1043-1143, 1044-1144, 1045-1145, 1046-1146, 1047-1147, 1048-1148, 1049-1149, 1050-1150, 1051-1151, 1052-1152, 1053-1153, 1054-1154, 1055-1155, 1056-1156, 1057-1157, 1058-1158, 1059-1159, 1060-1160, 1061-1161, 1062-1162, 1063-1163, 1064-1164, 1065-1165, 1066-1166, 1067-1167, 1068-1168, 1069-1169, 1070-1170, 1071-1171, 1072-1172, 1073-1173, 1074-1174, 1075-1175, 1076-1176, 1077-1177, 1078-1178, 1079-1179, 1080-1180, 1081-1181, 1082-1182, 1083-1183, 1084-1184, 1085-1185, 1086-1186, 1087-1187, 1088-1188, 1089-1189, 1090-1190, 1091-1191, 1092-1192, 1093-1193, 1094-1194, 1095-1195, 1096-1196, 1097-1197, 1098-1198, 1099-1199, 1100-1200, 1101-1201, 1102-1202, 1103-1203, 1104-1204, 1105-1205, 1106-1206, 1107-1207, 1108-1208, 1109-1209, 1110-1210, 1111-1211, 1112-1212, 1113-1213, 1114-1214, 1115-1215, 1116-1216, 1117-1217, 1118-1218, 1119-1219, 1120-1220, 1121-1221, 1122-1222, 1123-1223, 1124-1224, 1125-1225, 1126-1226, 1127-1227, 1128-1228, 1129-1229, 1130-1230, 1131-1231, 1132-1232, 1133-1233, 1134-1234, 1135-1235, 1136-1236, 1137-1237, 1138-1238, 1139-1239, 1140-1240, 1141-1241, 1142-1242, 1143-1243, 1144-1244, 1145-1245, 1146-1246, 1147-1247, 1148-1248, 1149-1249, 1150-1250, 1151-1251, 1152-1252, 1153-1253, 1154-1254, 1155-1255, 1156-1256, 1157-1257, 1158-1258, 1159-1259, 1160-1260, 1161-1261, 1162-1262, 1163-1263, 1164-1264, 1165-1265, 1166-1266, 1167-1267, 1168-1268, 1169-1269, 1170-1270, 1171-1271, 1172-1272, 1173-1273, 1174-1274, 1175-1275, 1176-1276, 1177-1277, 1178-1278, 1179-1279, 1180-1280, 1181-1281, 1182-1282, 1183-1283, 1184-1284, 1185-1285, 1186-1286, 1187-1287, 1188-1288, 1189-1289, 1190-1290, 1191-1291, 1192-1292, 1193-1293, 1194-1294, 1195-1295, 1196-1296, 1197-1297, 1198-1298, 1199-1299, 1200-1300, 1201-1301, 1202-1302, 1203-1303, 1204-1304, 1205-1305, 1206-1306, 1207-1307, 1208-1308, 1209-1309, 1210-1310, 1211-1311, 1212-1312, 1213-1313, 1214-1314, 1215-1315, 1216-1316, 1217-1317, 1218-1318, 1219-1319, 1220-1320, 1221-1321, 1222-1322, 1223-1323, 1224-1324, 1225-1325, 1226-1326, 1227-1327, 1228-1328, 1229-1329, 1230-1330, 1231-1331, 1232-1332, 1233-1333, 1234-1334, 1235-1335, 1236-1336, 1237-1337, 1238-1338, 1239-1339, 1240-1340, 1241-1341, 1242-1342, 1243-1343, 1244-1344, 1245-1345, 1246-1346, 1247-1347, 1248-1348, 1249-1349, 1250-1350, 1251-1351, 1252-1352, 1253-1353, 1254-1354, 1255-1355, 1256-1356, 1257-1357, 1258-1358, 1259-1359, 1260-1360, 1261-1361, 1262-1362, 1263-1363, 1264-1364, 1265-1365, 1266-1366, 1267-1367, 1268-1368, 1269-1369, 1270-1370, 1271-1371, 1272-1372, 1273-1373, 1274-1374, 1275-1375, 1276-1376, 1277-1377, 1278-1378, 1279-1379, 1280-1380, 1281-1381, 1282-1382, 1283-1383, 1284-1384, 1285-1385, 1286-1386, 1287-1387, 1288-1388, 1289-1389, 1290-1390, 1291-1391, 1292-1392, 1293-1393, 1294-1394, 1295-1395, 1296-1396, 1297-1397, 1298-1398, 1299-1399, 1300-1400, 1301-1401, 1302-1402, 1303-1403, 1304-1404, 1305-1405, 1306-1406, 1307-1407, 1308-1408, 1309-1409, 1310-1410, 1311-1411, 1312-1412, 1313-1413, 1314-1414, 1315-1415, 1316-1416, 1317-1417, 1318-1418, 1319-1419, 1320-1420, 1321-1421, 1322-1422, 1323-1423, 1324-1424, 1325-1425, 1326-1426, 1327-1427, 1328-1428, 1329-1429, 1330-1430, 1331-1431, 1332-1432, 1333-1433, 1334-1434, 1335-1435, 1336-1436, 1337-1437, 1338-1438, 1339-1439, 1340-1440, 1341-1441, 1342-1442, 1343-1443, 1344-1444, 1345-1445, 1346-1446, 1347-1447, 1348-1448, 1349-1449, 1350-1450, 1351-1451, 1352-1452, 1353-1453, 1354-1454, 1355-1455, 1356-1456, 1357-1457, 1358-1458, 1359-1459, 1360-1460, 1361-1461, 1362-1462, 1363-1463, 1364-1464, 1365-1465, 1366-1466, 1367-1467, 1368-1468, 1369-1469, 1370-1470, 1371-1471, 1372-1472, 1373-1473, 1374-1474, 1375-1475, 1376-1476, 1377-1477, 1378-1478, 1379-1479, 1380-1480, 1381-1481, 1382-1482, 1383-1483, 1384-1484, 1385-1485, 1386-1486, 1387-1487, 1388-1488, 1389-1489, 1390-1490, 1391-1491, 1392-1492, 1393-1493, 1394-1494, 1395-1495, 1396-1496, 1397-1497, 1398-1498, 1399-1499, 1400-1500, 1401-1501, 1402-1502, 1403-1503, 1404-1504, 1405-1505, 1406-1506, 1407-1507, 1408-1508, 1409-1509, 1410-1510, 1411-1511, 1412-1512, 1413-1513, 1414-1514, 1415-1515, 1416-1516, 1417-1517, 1418-1518, 1419-1519, 1420-1520, 1421-1521, 1422-1522, 1423-1523, 1424-1524, 1425-1525, 1426-1526, 1427-1527, 1428-1528, 1429-1529, 1430-1530, 1431-1531, 1432-1532, 1433-1533, 1434-1534, 1435-1535, 1436-1536, 1437-1537, 1438-1538, 1439-1539, 1440-1540, 1441-1541, 1442-1542, 1443-1543, 1444-1544, 1445-1545, 1446-1546, 1447-1547, 1448-1548, 1449-1549, 1450-1550, 1451-1551, 1452-1552, 1453-1553, 1454-1554, 1455-1555, 1456-1556, 1457-1557, 1458-1558, 1459-1559, 1460-1560, 1461-1561, 1462-1562, 1463-1563, 1464-1564, 1465-1565, 1466-1566, 1467-1567, 1468-1568, 1469-1569, 1470-1570, 1471-1571, 1472-1572, 1473-1573, 1474-1574, 1475-1575, 1476-1576, 1477-1577, 1478-1578, 1479-1579, 1480-1580, 1481-1581, 1482-1582, 1483-1583, 1484-1584, 1485-1585, 1486-1586, 1487-1587, 1488-1588, 1489-1589, 1490-1590, 1491-1591, 1492-1592, 1493-1593, 1494-1594, 1495-1595, 1496-1596, 1497-1597, 1498-1598, 1499-1599, 1500-1600, 1501-1601, 1502-1602, 1503-1603, 1504-1604, 1505-1605, 1506-1606, 1507-1607, 1508-1608, 1509-1609, 1510-1610, 1511-1611, 1512-1612, 1513-1613, 1514-1614, 1515-1615, 1516-1616, 1517-1617, 1518-1618, 1519-1619, 1520-1620, 1521-1621, 1522-1622, 1523-1623, 1524-1624, 1525-1625, 1526-1626, 1527-1627, 1528-1628, 1529-1629, 1530-1630, 1531-1631, 1532-1632, 1533-1633, 1534-1634, 1535-1635, 1536-1636, 1537-1637, 1538-1638, 1539-1639, 1540-1640, 1541-1641, 1542-1642, 1543-1643, 1544-1644, 1545-1645, 1546-1646, 1547-1647, 1548-1648, 1549-1649, 1550-1650, 1551-1651, 1552-1652, 1553-1653, 1554-1654, 1555-1655, 1556-1656, 1557-1657, 1558-1658, 1559-1659, 1560-1660, 1561-1661, 1562-1662, 1563-1663, 1564-1664, 1565-1665, 1566-1666, 1567-1667, 1568-1668, 1569-1669, 1570-1670, 1571-1671, 1572-1672, 1573-1673, 1574-1674, 1575-1675, 1576-1676, 1577-1677, 1578-1678, 1579-1679, 1580-1680, 1581-1681, 1582-1682, 1583-1683, 1584-1684, 1585-1685, 1586-1686, 1587-1687, 1588-1688, 1589-1689, 1590-1690, 1591-1691, 1592-1692, 1593-1693, 1594-1694, 1595-1695, 1596-1696, 1597-1697, 1598-1698, 1599-1699, 1600-1700, 1601-1701, 1602-1702, 1603-1703, 1604-1704, 1605-1705, 1606-1706, 1607-1707, 1608-1708, 1609-1709, 1610-1710, 1611-1711, 1612-1712, 1613-1713, 1614-1714, 1615-1715, 1616-1716, 1617-1717, 1618-1718, 1619-1719, 1620-1720, 1621-1721, 1622-1722, 1623-1723, 1624-1724, 1625-1725, 1626-1726, 1627-1727, 1628-1728, 1629-1729, 1630-1730, 1631-1731, 1632-1732, 1633-1733, 1634-1734, 1635-1735, 1636-1736, 1637-1737, 1638-1738, 1639-1739, 1640-1740, 1641-1741, 1642-1742, 1643-1743, 1644-1744, 1645-1745, 1646-1746, 1647-1747, 1648-1748, 1649-1749, 1650-1750, 1651-1751, 1652-1752, 1653-1753, 1654-1754, 1655-1755, 1656-1756, 1657-1757, 1658-1758, 1659-1759, 1660-1760, 1661-1761, 1662-1762, 1663-1763, 1664-1764, 1665-1765, 1666-1766, 1667-1767, 1668-1768, 1669-1769, 1670-1770, 1671-1771, 1672-1772, 1673-1773, 1674-1774, 1675-1775, 1676-1776, 1677-1777, 1678-1778, 1679-1779, 1680-1780, 1681-1781, 1682-1782, 1683-1783, 1684-1784, 1685-1785, 1686-1786, 1687-1787, 1688-1788, 1689-1789, 1690-1790, 1691-1791, 1692-1792, 1693-1793, 1694-1794, 1695-1795, 1696-1796, 1697-1797, 1698-1798, 1699-1799, 1700-1800, 1701-1801, 1702-1802, 1703-1803, 1704-1804, 1705-1805, 1706-1806, 1707-1807, 1708-1808, 1709-1809, 1710-1810, 1711-1811, 1712-1812, 1713-1813, 1714-1814, 1715-1815, 1716-1816, 1717-1817, 1718-1818, 1719-1819, 1720-1820, 1721-1821, 1722-1822, 1723-1823, 1724-1824, 1725-1825, 1726-1826, 1727-1827, 1728-1828, 1729-1829, 1730-1830, 1731-1831, 1732-1832, 1733-1833, 1734-1834, 1735-1835, 1736-1836, 1737-1837, 1738-1838, 1739-1839, 1740-1840, 1741-1841, 1742-1842, 1743-1843, 1744-1844, 1745-1845, 1746-1846, 1747-1847, 1748-1848, 1749-1849, 1750-1850, 1751-1851, 1752-1852, 1753-1853, 1754-1854, 1755-1855, 1756-1856, 1757-1857, 1758-1858, 1759-1859, 1760-1860, 1761-1861, 1762-1862, 1763-1863, 1764-1864, 1765-1865, 1766-1866, 1767-1867, 1768-1868, 1769-1869, 1770-1870, 1771-1871, 1772-1872, 1773-1873, 1774-1874, 1775-1875, 1776-1876, 1777-1877, 1778-1878, 1779-1879, 1780-1880, 1781-1881, 1782-1882, 1783-1883, 1784-1884, 1785-1885, 1786-1886, 1787-1887, 1788-1888, 1789-1889, 1790-1890, 1791-1891, 1792-1892, 1793-1893, 1794-1894, 1795-1895, 1796-1896, 1797-1897, 1798-1898, 1799-1899, 1800-1900, 1801-1901, 1802-1902, 1803-1903, 1804-1904, 1805-1905, 1806-1906, 1807-1907, 1808-1908, 1809-1909, 1810-1910, 1811-1911, 1812-1912, 1813-1913, 1814-1914, 1815-1915, 1816-1916, 1817-1917, 1818-1918, 1819-1919, 1820-1920, 1821-1921, 1822-1922, 1823-1923, 1824-1924, 1825-1925, 1826-1926, 1827-1927, 1828-1928, 1829-1929, 1830-1930, 1831-1931, 1832-1932, 1833-1933, 1834-1934, 1835-1935, 1836-1936, 1837-1937, 1838-1938, 1839-1939, 1840-1940, 1841-1941, 1842-1942, 1843-1943, 1844-1944, 1845-1945, 1846-1946, 1847-1947, 1848-1948, 1849-1949, 1850-1950, 1851-1951, 1852-1952, 1853-1953, 1854-1954, 1855-1955, 1856-1956, 1857-1957, 1858-1958, 1859-1959, 1860-1960, 1861-1961, 1862-1962, 1863-1963, 1864-1964, 1865-1965, 1866-1966, 1867-1967, 1868-1968, 1869-1969, 1870-1970, 1871-1971, 1872-1972, 1873-1973, 1874-1974, 1875-1975, 1876-1976, 1877-1977, 1878-1978, 1879-1979, 1880-1980, 1881-1981, 1882-1982, 1883-1983, 1884-1984, 1885-1985, 1886-1986, 1887-1987, 1888-1988, 1889-1989, 1890-1990, 1891-1991, 1892-1992, 1893-1993, 1894-1994, 1895-1995, 1896-1996, 1897-1997, 1898-1998, 1899-1999, 1900-2000, 1901-2001, 1902-2002, 1903-2003, 1904-2004, 1905-2005, 1906-2006, 1907-2007, 1908-2008, 1909-2009, 1910-2010, 1911-2011, 1912-2012, 1913-2013, 1914-2014, 1915-2015, 1916-2016, 1917-2017, 1918-2018, 1919-2019, 1920-2020, 1921-2021, 1922-2022, 1923-2023, 1924-2024, 1925-2025, 1926-2026, 1927-2027, 1928-2028, 1929-2029, 1930-2030, 1931-2031, 1932-2032, 1933-2033, 1934-2034, 1935-2035, 1936-2036, 1937-2037, 1938-2038, 1939-2039, 1940-2040, 1941-2041, 1942-2042, 1943-2043, 1944-2044, 1945-2045, 1946-2046, 1947-2047, 1948-2048, 1949-2049, 1950-2050, 1951-2051, 1952-2052, 1953-2053, 1954-2054, 1955-2055, 1956-2056, 1957-2057, 1958-2058, 1959-2059, 1960-2060, 1961-2061, 1962-2062, 1963-2063, 1964-2064, 1965-2065, 1966-2066, 1967-2067, 1968-2068, 1969-2069, 1970-2070, 1971-2071, 1972-2072, 1973-2073, 1974-2074, 1975-2075, 1976-2076, 1977-2077, 1978-2078, 1979-2079, 1980-2080, 1981-2081, 1982-2082, 1983-2083, 1984-2084, 1985-2085, 1986-2086, 1987-2087, 1988-2088, 1989-2089, 1990-2090, 1991-2091, 1992-2092, 1993-2093, 1994-2094, 1995-2095, 1996-2096, 1997-2097, 1998-2098, 1999-2099, 2000-2100, 2001-2101, 2002-2102, 2003-2103, 2004-2104, 2005-2105, 2006-2106, 2007-2107, 2008-2108, 2009-2109, 2010-2110, 2011-2111, 2012-2112, 2013-2113, 2014-2114, 2015-2115, 2016-2116, 2017-2117, 2018-2118, 2019-2119, 2020-2120, 2021-2121, 2022-2122, 2023-2123, 2024-2124, 2025-2125, 2026-2126, 2027-2127, 2028-2128, 2029-2129, 2030-2130, 2031-2131, 2032-2132, 2033-2133, 2034-2134, 2035-2135, 2036-2136, 2037-2137, 2038-2138, 2039-2139, 2040-2140, 2041-2141, 2042-2142, 2043-2143, 2044-2144, 2045-2145, 2046-2146, 2047-2147, 2048-2148, 2049-2149, 2050-2150, 2051-2151, 2052-2152, 2053-2153, 2054-2154, 2055-2155, 2056-2156, 2057-2157, 2058-2158, 2059-2159, 2060-2160, 2061-2161, 2062-2162, 2063-2163, 2064-2164, 2065-2165, 2066-2166, 2067-2167, 2068-2168, 2069-2169, 2070-2170, 2071-2171, 2072-2172, 2073-2173, 2074-2174, 2075-2175, 2076-2176, 2077-2177, 2078-2178, 2079-2179, 2080-2180, 2081-2181, 2082-2182, 2083-2183, 2084-2184, 2085-2185, 2086-2186, 2087-2187, 2088-2188, 2089-2189, 2090-2190, 2091-2191, 2092-2192, 2093-2193, 2094-2194, 2095-2195, 2096-2196, 2097-2197, 2098-2198, 2099-2199, 2100-2200, 2101-2201, 2102-2202, 2103-2203, 2104-2204, 2105-2205, 2106-2206, 2107-2207, 2108-2208, 2109-2209, 2110-2210, 2111-2211, 2112-2212, 2113-2213, 2114-2214, 2115-2215, 2116-2216, 2117-2217, 2118-2218, 2119-2219, 2120-2220, 2121-2221, 2122-2222, 2123-2223, 2124-2224, 2125-2225, 2126-2226, 2127-2227, 2128-2228, 2129-2229, 2130-2230, 2131-2231, 2132-2232, 2133-2233, 2134-2234, or 2135-2235 of any one of SEQ ID NOS: 26-30.

Accordingly, provided herein are rAAV vectors comprising a polynucleotide comprising a nucleic acid encoding a human retinoschisin protein and a stuffer sequence. In some embodiments, the stuffer sequence has a length of between about 1000 nucleotides and about 4000 nucleotides. In some embodiments, the stuffer sequence has a length of between about 2000 nucleotides and about 3500, about 3200, about 3000, about 2900, or about 2800 nucleotides. In some embodiments, the stuffer sequence has a length of between about 2500 nucleotides and about 3000 nucleotides. In some embodiments, the stuffer sequence has a length of about 2800 nucleotides.

In some embodiments, the nucleic acid vector comprising the heterologous nucleic acid and stuffer sequence comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 16-17, 31-35. In some embodiments, the nucleic acid vector comprising the heterologous nucleic acid and stuffer sequence comprises a sequence of any one of SEQ ID NOs: 31-34.

In some embodiments, the AAV vector comprises a first ITR and a second ITR and the length of the AAV vector between the first ITR and the second ITR (i.e., “the length between the ITRs”) is between about 2000 nucleotides and about 6000 nucleotides. In some embodiments, such an AAV vector comprises a stuffer sequence. In some embodiments, the length of the AAV vector between the first ITR and the second ITR is between about 3000 nucleotides and about 5000 nucleotides. In some embodiments, the length of the AAV vector between the first ITR and the second ITR is between about 4000 nucleotides and about 5000 nucleotides. This length between the ITRs may be about 4500 nucleotides.

In some embodiments, the total length of the heterologous nucleic acid and the stuffer sequence is between about 2000 nucleotides and about 5000 nucleotides. In some embodiments, this total length is between about 2500 nucleotides and about 4500, about 4200, about 4000, about 3800, about 3700, about 3600, or about 3500 nucleotides. In some embodiments, the stuffer sequence has a length of about 3500 nucleotides.

In some embodiments, the stuffer sequence is positioned downstream (3′) of the heterologous nucleic acid encoding human retinoschisin protein. In some embodiments, the stuffer sequence is positioned upstream (5′) of the polyA sequence. In some embodiments, the stuffer sequence is positioned 3′ of the polyA sequence. In some embodiments, the stuffer sequence is positioned between two AAV ITR sequences.

### Stuffer Sequences

>stuffer 1 (2822 bp)
(SEQ ID NO: 26)
cgattcgagtgagcagaccacatgatgattggtattctccctgaagaaagaatgtcagcaaggagtatctgaagtccgaccaagccta
gaatccgcccggttcttatttgctgaagtaatactgttgtcaagtcagctacagggagtggtccctgcctggagtttgaagggctgtcaaa
gacattgtggtcacttactcaagtggtaaattccttacagactggactgcagttaagggtgataacgctactactaggacaagggtatcct
tgatcctgtggatctgtagtgacctgagactgtcaaggtatggtgagtatgacccttgaccctttccaacggttgtaaccgtgggcgctgt
caaatctgattaaccactactaggatagaatttggcagagctactgaaatggttacctggtcaagagactggcccacagcataagggcg
agcaagacgatgctcagattaactaagtgaccgaagtatcctagatggtccactaccagcaaatgtgtcagacctccctcagttggtagt
acaatgtatccttctagtcgcgacttattatatcgacgagcttatgtgtaagagaccagattctttgtaacctgacacttgagttcaaggtgt
gtagtaggagagtgcaaaccctgaagcaacagtagccgaaccgggtcgaggacctagcgttgattgactttgtctggtgtatcctgtaa
atttctccaatcgagatcgaggtaaagtggacctcacaggactttatcaaacaacagagttaatacctcaaatctctcgctgatatatgtga
caaattcattgcgtcacttatcgcagctatgttcggtcagcatattctggcgaacacccacgctgcagttcgacaaggatagtatctggcg
ctctcgcccaattcaagcacgcgaattactctattaacttgttgtctcttgtgtatatcataatcaattcctcctagttagtgattgaagacgac
cgtgcacttggctgccttgatgcagaccacttcttgtggccggcacaccctataatagagatgtgataaataacttagctggactgtagtt
atttcgtcctagggattctgacgggtgcaaatcactaagtgtctttggtattgggtctgtaacaatgttgagaactgtggtgtatgtacgaac
gatgtgatctgtagactacagcacagggtattgctgtttcgcaccagtgtgacacttgtgtggctgtcctctgtgctatagcgccgtatatc
ttatgcaggttgtatagcccaacgactgtttaggtacgtccgtataagaataggagtgctacactctacattgaatctatccagagagagtt
ctgtagaattaggtgacgaacaaatctacgcgataggaaggagagacctggttgtcccaagggccgtgggtatgatgtttagtacaact
aggaggctctaggtgtattggttgttggtcacgtagtgtccctacttggtcaggactaaagtttgtcagtgaactctctactattattcatcga
acctgtggtcgtcggcggtctgatatatgctcggtagattgcccgacaggacgtgaaggagcctaactattaaagcactctaactagcc
tatcgtaacaacttatattgggtatttgggtacgacggtgtactggaaatctatctaattcttctgatcgtatatcgggtcgtatattgatttatg
acggaccgaccactcacgcggtccgatacgcggtagcgctatccaggaccctcttatgaattggtatcaatagattgtggaacttgttgc
gtacactacagaatgtctttgcaaagaatgccaaattacaagctatagtcctcccactatactgcttcgtgactccaactagggtagttata
gttgttcaagactggtactctgttaccaattagtagcgatattgtgtgggatactggtgtctgtcatacgagaactgggagtagcgctgtaa
attctaaatagtcacactgttccagctggacgaccgctgtgtactctttgtagaaactcactgcggcttccacttatgagatttggttatagg
tcacaaagtggcagtccagttgcaggtctcttgttgtaatatgactggattgaattgactaggaagacgtagtctgttcttgttaggtccga
cgtggtcccaatttgcttatccaatatgaggtcctagttagcagtcgctccgaggtcgaaatagatacccttgtggtttcgtagattgtctga
ttcgcaacaatatcctgctaatcaatctacggaaacaccgcttcagaggaagattgcgacggacagaagagtcagtttcggctgcttgt
ggtttggcctaatataaagattacttacggcttaggtatactgcacatcctccggccttgtttggtgcttgagattggtgcgcatatatgtaa
ggtgctatcgtatccgcctccagtacttgatcttgtcctgcggctttagggctgcttatccctccagcggtccaacctctgaagggtcagg
ccatacttcacctcctttgtctaggattctgttccgaatttccggtctgggtcatgtactctcaccatcgagtgtagggagacgattctcgta
acagccgtagccgaatcactttcacttgagtctatgggttgtgcctgagttctagcttagtatttaacaacggcgtcggtccaaagacggt
acacacttaaccatagtgaacgaatgaaacagtaatcgctccaggttggcgcgccttgcatgctggggagagatctg
>stuffer 2 (2820 bp)
(SEQ ID NO: 27)
cagatctgaattcgattcgagtgagcagaccacatgatgattggtattctccctgaagaaagaatgtcagcaaggagtatctgaagtccg
accaagcctagaatccgcccggttcttatttgctgaagtaatactgttgtcaagtcagctacagggagtggtccctgcctggagtttgaag
ggctgtcaaagacattgtggtcacttactcaagtggtaaattccttacagactggactgcagttaagggtgataacgctactactaggac
aagggtatccttgatcctgtggatctgtagtgacctgagactgtcaaggtatggtgagtatgacccttgaccctttccaacggttgtaacc
gtgggcgctgtcaaatctgattaaccactactaggatagaatttggcagagctactgaaatggttacctggtcaagagactggcccaca
gcataagggcgagcaagacgatgctcagattaactaagtgaccgaagtatcctagatggtccactaccagcaaatgtgtcagacctcc
ctcagttggtagtacaatgtatccttctagtcgcgacttattatatcgacgagcttatgtgtaagagaccagattctttgtaacctgacacttg
agttcaaggtgtgtagtaggagagtgcaaaccctgaagcaacagtagccgaaccgggtcgaggacctagcgttgattgactttgtctg
gtgtatcctgtaaatttctccaatcgagatcgaggtaaagtggacctcacaggactttatcaaacaacagagttaatacctcaaatctctcg
ctgatatatgtgacaaattcattgcgtcacttatcgcagctatgttcggtcagcatattctggcgaacacccacgctgcagttcgacaagg
atagtatctggcgctctcgcccaattcaagcacgcgaattactctattaacttgttgtctcttgtgtatatcataatcaattcctcctagttagt
gattgaagacgaccgtgcacttggctgccttgatgcagaccacttcttgtggccggcacaccctataatagagatgtgataaataactta
gctggactgtagttatttcgtcctagggattctgacgggtgcaaatcactaagtgtctttggtattgggtctgtaacaatgttgagaactgtg
gtgtatgtacgaacgatgtgatctgtagactacagcacagggtattgctgtttcgcaccagtgtgacacttgtgtggctgtcctctgtgcta
tagcgccgtatatcttatgcaggttgtatagcccaacgactgtttaggtacgtccgtataagaataggagtgctacactctacattgaatct
atccagagagagttctgtagaattaggtgacgaacaaatctacgcgataggaaggagagacctggttgtcccaagggccgtgggtat
gatgtttagtacaactaggaggctctaggtgtattggttgttggtcacgtagtgtccctacttggtcaggactaaagtttgtcagtgaactct
ctactattattcatcgaacctgtggtcgtcggcggtctgatatatgctcggtagattgcccgacaggacgtgaaggagcctaactattaaa
gcactctaactagcctatcgtaacaacttatattgggtatttgggtacgacggtgtactggaaatctatctaattcttctgatcgtatatcggg
togtatattgatttatgacggaccgaccactcacgcggtccgatacgcggtagcgctatccaggaccctcttatgaattggtatcaataga
ttgtggaacttgttgcgtacactacagaatgtctttgcaaagaatgccaaattacaagctatagtcctcccactatactgcttcgtgactcca
actagggtagttatagttgttcaagactggtactctgttaccaattagtagcgatattgtgtgggatactggtgtctgtcatacgagaactgg
gagtagcgctgtaaattctaaatagtcacactgttccagctggacgaccgctgtgtactctttgtagaaactcactgcggcttccacttatg
agatttggttataggtcacaaagtggcagtccagttgcaggtctcttgttgtaatatgactggattgaattgactaggaagacgtagtctgtt
cttgttaggtccgacgtggtcccaatttgcttatccaatatgaggtcctagttagcagtcgctccgaggtcgaaatagatacccttgtggttt
cgtagattgtctgattcgcaacaatatcctgctaatcaatctacggaaacaccgcttcagaggaagattgcgacggacagaagagtcag
tttcggctgcttgtggtttggcctaatataaagattacttacggcttaggtatactgcacatcctccggccttgtttggtgcttgagattggtg
cgcatatatgtaaggtgctatcgtatccgcctccagtacttgatcttgtcctgcggctttagggctgcttatccctccagcggtccaacctct
gaagggtcaggccatacttcacctcctttgtctaggattctgttccgaatttccggtctgggtcatgtactctcaccatcgagtgtagggag
acgattctcgtaacagccgtagccgaatcactttcacttgagtctatgggttgtgcctgagttctagcttagtatttaacaacggcgtcggt
ccaaagacggtacacacttaaccatagtgaacgaatgaaacagtaatcgctccaggttaagaactattggtacc
>stuffer 3 (2249 bp)
(SEQ ID NO: 28)
cagtggtccctgcctggagtttgaagggctgtcaaagacattgtggtcacttactcaagtggtaaattccttacagactggactgcagtta
agggtgataacgctactactaggacaagggtatccttgatcctgtggatctgtagtgacctgagactgtcaaggtatggtgagtatgacc
cttgaccctttccaacggttgtaaccgtgggcgctgtcaaatctgattaaccactactaggatagaatttggcagagctactgaaatggtt
acctggtcaagagactggcccacagcataagggcgagcaagacgatgctcagattaactaagtgaccgaagtatcctagatggtcca
ctaccagcaaatgtgtcagacctccctcagttggtagtacaatgtatccttctagtcgcgacttattatatcgacgagcttatgtgtaagag
accagattctttgtaacctgacacttgagttcaaggtgtgtagtaggagagtgcaaaccctgaagcaacagtagccgaaccgggtcga
ggacctagcgttgattgactttgtctggtgtatcctgtaaatttctccaatcgagatcgaggtaaagtggacctcacaggactttatcaaac
aacagagttaatacctcaaatctctcgctgatatatgtgacaaattcattgcgtcacttatcgcagctatgttcggtcagcatattctggcga
acacccacgctgcagttcgacaaggatagtatctggcgctctcgcccaattcaagcacgcgaattactctattaacttgttgtctcttgtgt
atatcataatcaattcctcctagttagtgattgaagacgaccgtgcacttggctgccttgatgcagaccacttcttgtggccggcacaccct
ataatagagatgtgataaataacttagctggactgtagttatttcgtcctagggattctgacgggtgcaaatcactaagtgtctttggtattg
ggtctgtaacaatgttgagaactgtggtgtatgtacgaacgatgtgatctgtagactacagcacagggtattgctgtttcgcaccagtgtg
acacttgtgtggctgtcctctgtgctatagcgccgtatatcttatgcaggttgtatagcccaacgactgtttaggtacgtccgtataagaata
ggagtgctacactctacattgaatctatccagagagagttctgtagaattaggtgacgaacaaatctacgcgataggaaggagagacct
ggttgtcccaagggccgtgggtatgatgtttagtacaactaggaggctctaggtgtattggttgttggtcacgtagtgtccctacttggtca
ggactaaagtttgtcagtgaactctctactattattcatcgaacctgtggtcgtcggcggtctgatatatgctcggtagattgcccgacagg
acgtgaaggagcctaactattaaagcactctaactagcctatcgtaacaacttatattgggtatttgggtacgacggtgtactggaaatct
atctaattcttctgatcgtatatcgggtcgtatattgatttatgacggaccgaccactcacgcggtccgatacgcggtagcgctatccagg
accctcttatgaattggtatcaatagattgtggaacttgttgcgtacactacagaatgtctttgcaaagaatgccaaattacaagctatagtc
ctcccactatactgcttcgtgactccaactagggtagttatagttgttcaagactggtactctgttaccaattagtagcgatattgtgtgggat
actggtgtctgtcatacgagaactgggagtagcgctgtaaattctaaatagtcacactgttccagctggacgaccgctgtgtactctttgt
agaaactcactgcggcttccacttatgagatttggttataggtcacaaagtggcagtccagttgcaggtctcttgttgtaatatgactggatt
gaattgactaggaagacgtagtctgttcttgttaggtccgacgtggtcccaatttgcttatccaatatgaggtcctagttagcagtcgctcc
gaggtcgaaatagatacccttgtggtttcgtagattgtctgattcgcaacaatatcctgctaatcaatctacggaaacaccgcttcagagg
aagattgcgacggacagaagagtcaggcgcgccttgcatgctggggagagatctg
>stuffer 4 (2235 bp)
(SEQ ID NO: 29)
cagtggtccctgcctggagtttgaagggctgtcaaagacattgtggtcacttactcaagtggtaaattccttacagactggactgcagtta
agggtgataacgctactactaggacaagggtatccttgatcctgtggatctgtagtgacctgagactgtcaaggtatggtgagtatgacc
cttgaccctttccaacggttgtaaccgtgggcgctgtcaaatctgattaaccactactaggatagaatttggcagagctactgaaatggtt
acctggtcaagagactggcccacagcataagggcgagcaagacgatgctcagattaactaagtgaccgaagtatcctagatggtcca
ctaccagcaaatgtgtcagacctccctcagttggtagtacaatgtatccttctagtcgcgacttattatatcgacgagcttatgtgtaagag
accagattctttgtaacctgacacttgagttcaaggtgtgtagtaggagagtgcaaaccctgaagcaacagtagccgaaccgggtcga
ggacctagcgttgattgactttgtctggtgtatcctgtaaatttctccaatcgagatcgaggtaaagtggacctcacaggactttatcaaac
aacagagttaatacctcaaatctctcgctgatatatgtgacaaattcattgcgtcacttatcgcagctatgttcggtcagcatattctggcga
acacccacgctgcagttcgacaaggatagtatctggcgctctcgcccaattcaagcacgcgaattactctattaacttgttgtctcttgtgt
atatcataatcaattcctcctagttagtgattgaagacgaccgtgcacttggctgccttgatgcagaccacttcttgtggccggcacaccct
ataatagagatgtgataaataacttagctggactgtagttatttcgtcctagggattctgacgggtgcaaatcactaagtgtctttggtattg
ggtctgtaacaatgttgagaactgtggtgtatgtacgaacgatgtgatctgtagactacagcacagggtattgctgtttcgcaccagtgtg
acacttgtgtggctgtcctctgtgctatagcgccgtatatcttatgcaggttgtatagcccaacgactgtttaggtacgtccgtataagaata
ggagtgctacactctacattgaatctatccagagagagttctgtagaattaggtgacgaacaaatctacgcgataggaaggagagacct
ggttgtcccaagggccgtgggtatgatgtttagtacaactaggaggctctaggtgtattggttgttggtcacgtagtgtccctacttggtca
ggactaaagtttgtcagtgaactctctactattattcatcgaacctgtggtcgtcggcggtctgatatatgctcggtagattgcccgacagg
acgtgaaggagcctaactattaaagcactctaactagcctatcgtaacaacttatattgggtatttgggtacgacggtgtactggaaatct
atctaattcttctgatcgtatatcgggtcgtatattgatttatgacggaccgaccactcacgcggtccgatacgcggtagcgctatccagg
accctcttatgaattggtatcaatagattgtggaacttgttgcgtacactacagaatgtctttgcaaagaatgccaaattacaagctatagtc
ctcccactatactgcttcgtgactccaactagggtagttatagttgttcaagactggtactctgttaccaattagtagcgatattgtgtgggat
actggtgtctgtcatacgagaactgggagtagcgctgtaaattctaaatagtcacactgttccagctggacgaccgctgtgtactctttgt
agaaactcactgcggcttccacttatgagatttggttataggtcacaaagtggcagtccagttgcaggtctcttgttgtaatatgactggatt
gaattgactaggaagacgtagtctgttcttgttaggtccgacgtggtcccaatttgcttatccaatatgaggtcctagttagcagtcgctcc
gaggtcgaaatagatacccttgtggtttcgtagattgtctgattcgcaacaatatcctgctaatcaatctacggaaacaccgcttcagagg
aagattgcgacggacagaagagtcaggcgcgccttgcatgc
>stuffer 5 (2219 bp)
(SEQ ID NO: 30)
agtggtccctgcctggagtttgaagggctgtcaaagacattgtggtcacttactcaagtggtaaattccttacagactggactgcagttaa
gggtgataacgctactactaggacaagggtatccttgatcctgtggatctgtagtgacctgagactgtcaaggtatggtgagtatgaccc
ttgaccctttccaacggttgtaaccgtgggcgctgtcaaatctgattaaccactactaggatagaatttggcagagctactgaaatggtta
cctggtcaagagactggcccacagcataagggcgagcaagacgatgctcagattaactaagtgaccgaagtatcctagatggtccac
taccagcaaatgtgtcagacctccctcagttggtagtacaatgtatccttctagtcgcgacttattatatcgacgagcttatgtgtaagaga
ccagattctttgtaacctgacacttgagttcaaggtgtgtagtaggagagtgcaaaccctgaagcaacagtagccgaaccgggtcgag
gacctagcgttgattgactttgtctggtgtatcctgtaaatttctccaatcgagatcgaggtaaagtggacctcacaggactttatcaaaca
acagagttaatacctcaaatctctcgctgatatatgtgacaaattcattgcgtcacttatcgcagctatgttcggtcagcatattctggcgaa
cacccacgctgcagttcgacaaggatagtatctggcgctctcgcccaattcaagcacgcgaattactctattaacttgttgtctcttgtgta
tatcataatcaattcctcctagttagtgattgaagacgaccgtgcacttggctgccttgatgcagaccacttcttgtggccggcacacccta
taatagagatgtgataaataacttagctggactgtagttatttcgtcctagggattctgacgggtgcaaatcactaagtgtctttggtattgg
gtctgtaacaatgttgagaactgtggtgtatgtacgaacgatgtgatctgtagactacagcacagggtattgctgtttcgcaccagtgtga
cacttgtgtggctgtcctctgtgctatagcgccgtatatcttatgcaggttgtatagcccaacgactgtttaggtacgtccgtataagaatag
gagtgctacactctacattgaatctatccagagagagttctgtagaattaggtgacgaacaaatctacgcgataggaaggagagacctg
gttgtcccaagggccgtgggtatgatgtttagtacaactaggaggctctaggtgtattggttgttggtcacgtagtgtccctacttggtcag
gactaaagtttgtcagtgaactctctactattattcatcgaacctgtggtcgtcggcggtctgatatatgctcggtagattgcccgacagga
cgtgaaggagcctaactattaaagcactctaactagcctatcgtaacaacttatattgggtatttgggtacgacggtgtactggaaatctat
ctaattcttctgatcgtatatcgggtcgtatattgatttatgacggaccgaccactcacgcggtccgatacgcggtagcgctatccaggac
cctcttatgaattggtatcaatagattgtggaacttgttgcgtacactacagaatgtctttgcaaagaatgccaaattacaagctatagtcct
cccactatactgcttcgtgactccaactagggtagttatagttgttcaagactggtactctgttaccaattagtagcgatattgtgtgggata
ctggtgtctgtcatacgagaactgggagtagcgctgtaaattctaaatagtcacactgttccagctggacgaccgctgtgtactctttgta
gaaactcactgcggcttccacttatgagatttggttataggtcacaaagtggcagtccagttgcaggtctcttgttgtaatatgactggatt
gaattgactaggaagacgtagtctgttcttgttaggtccgacgtggtcccaatttgcttatccaatatgaggtcctagttagcagtcgctcc
gaggtcgaaatagatacccttgtggtttcgtagattgtctgattcgcaacaatatcctgctaatcaatctacggaaacaccgcttcagagg
aagattgcgacggacagaagagtcag

### Example AAV Vectors

In some embodiments, provided herein are nucleic acid vectors comprises a heterologous nucleic acid (e.g., encoding a retinoschisin protein) and one or more additional elements. In some embodiments, the additional element comprises an intron. In some embodiments, the additional element comprises a splice donor region. In some embodiments, the additional element comprises a splice acceptor region. In some embodiments, the additional element comprises a promoter. In some embodiments, the additional element comprises a polyA signal. In some embodiments, the additional element comprises one or more terminal repeats. In some embodiments, the additional element comprises a WPRE element. In some embodiments, the additional element comprises two or more of the elements described above.

Accordingly, exemplary rAAV vectors described in the disclosure may comprise any one of the following structures: AAV-hGRK1-hRS1syn, AAV-hGRK1-hRS1syn-WPREsf, AAV-hGRK1-GFP, AAV-pTR-X001-3p, AAV-pTR-X001-5p, AAV-pTR-X002-3p, AAV-pTR-X002-3pSR, AAV-pTR-X001, and AAV-pTR-X002. Exemplary vectors encoding an RS1 protein may comprise any of the following: AAV44.9(E531D)-hGRK1-hRS1syn, AAV44.9(E531D)-hGRK1-hRS1syn-WPREsf, AAV44.9(Y446F+T492V+E531D)-hGRK1-hRS1syn, AAV44.9(Y446F+T492V+E531D)-hGRK1-hRS1syn-WPREsf, AAV44.9(Y446F+E531D)-hGRK1-hRS1syn, AAV44.9(Y446F+E531D)-hGRK1-hRS1syn-WPREsf, AAV44.9(T492V+E531D)-hGRK1-hRS1syn, AAV44.9(T492V+E531D)-hGRK1-hRS1syn-WPREsf, AAV44.9-hGRK1-hRS1syn, AAV44.9-hGRK1-hRS1syn-WPREsf, AAV44.9(Y731F)-hGRK1-hRS1syn, AAV44.9(Y731F)-hGRK1-hRS1syn-WPREsf, AAV5-hGRK1-hRS1syn, AAV5-hGRK1-hRS1syn-WPREsf, AAV2(4pMut)ΔHS-hGRK1-hRS1syn, AAV2(4pMut)ΔHS-hGRK1-hRS1syn-WPREsf, AAV8(Y447F+Y733F+T494V)-hGRK1-hRS1syn, or AAV8(Y447F+Y733F+T494V)-hGRK1-hRS1syn-WPREsf. In certain embodiments, exemplary vectors comprise AAV44.9(E531D)-hGRK1-hRS1syn, AAV44.9(E531D)-hGRK1-hRS1syn-WPREsf, AAV44.9(Y446F+T492V+E531D)-hGRK1-hRS1syn, or AAV44.9(Y446F+T492V+E531D)-hGRK1-hRS1syn-WPREsf.

In certain embodiments, the vector comprises AAV44.9-pTR-X001-3p. In certain embodiments, the vector comprises AAV44.9-pTR-X001-5p. In certain embodiments, the vector comprises AAV44.9-pTR-X002-3p. In certain embodiments, the vector comprises AAV44.9-pTR-X002-3pSR. In certain embodiments, the vector comprises AAV44.9-pTR-X001. In certain embodiments, the vector comprises AAV44.9-pTR-X002.

In certain embodiments, the vector comprises AAV44.9(E531D)-pTR-X001-3p. In certain embodiments, the vector comprises AAV-44.9(E531D)-pTR-X001-5p. In certain embodiments, the vector comprises AAV44.9(E531D)-pTR-X002-3p. In certain embodiments, the vector comprises AAV44.9(E531D)-pTR-X002-3pSR. In certain embodiments, the vector comprises AAV44.9(E531D)-pTR-X001. In certain embodiments, the vector comprises AAV44.9(E531D)-pTR-X002.

In certain embodiments, the vector comprises AAV2-pTR-X001-3p. In certain embodiments, the vector comprises AAV2-pTR-X001-5p. In certain embodiments, the vector comprises AAV2-pTR-X002-3p. In certain embodiments, the vector comprises AAV2-pTR-X002-3pSR. In certain embodiments, the vector comprises AAV2-pTR-X001. In certain embodiments, the vector comprises AAV2-pTR-X002.

In certain embodiments, the vector comprises AAV2(4pMut)ΔHS-pTR-X001-3p. In certain embodiments, the vector comprises AAV2(4pMut)ΔHS-pTR-X001-5p. In certain embodiments, the vector comprises AAV2(4pMut)ΔHS-pTR-X002-3p. In certain embodiments, the vector comprises AAV2(4pMut)ΔHS-pTR-X002-3pSR. In certain embodiments, the vector comprises AAV2(4pMut)ΔHS-pTR-X001. In certain embodiments, the vector comprises AAV2(4pMut)ΔHS-pTR-X002.

Further exemplary vectors containing a reporter transgene (e.g., GFP) may comprise any of the following: AAV44.9(Y731F)-hGRK1-GFP, AAV44.9(E531D)-IRBP/GNAT2-hGFP, AAV44.9(Y731F)-IRBP/GNAT2-hGFP.

In some embodiments, the AAV vector comprises a sequence having about or at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 31. For example, the AAV vector comprises SEQ ID NO: 31.

In some embodiments, the AAV vector comprises a sequence having about or at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 32. For example, the AAV vector comprises SEQ ID NO: 32.

In some embodiments, the AAV vector comprises a sequence having about or at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 33. For example, the AAV vector comprises SEQ ID NO: 33.

In some embodiments, the AAV vector comprises a sequence having about or at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 34. For example, the AAV vector comprises SEQ ID NO: 34.

In some embodiments, the AAV vector comprises a sequence having about or at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 35. For example, the AAV vector comprises SEQ ID NO: 35.

In some embodiments, the AAV vector comprises a sequence having about or at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 33. For example, the AAV vector comprises SEQ ID NO: 33.

In some embodiments, the AAV vector has the following architecture: 5′ ITR—promoter—intron and/or splice donor and/or splice acceptor—hRS1 coding sequence—polyA—stuffer—3′ ITR. The promoter may be an hGRK1 promoter. The promoter may be a CBA or smCBA promoter. The polyA sequence may be a bGH polyA sequence. The intron and/or splice donor and/or splice acceptor may be a SV40 intron.

In some embodiments, the AAV vector has the following architecture: 5′ ITR—stuffer—promoter—intron and/or splice donor and/or splice acceptor—hRS1 coding sequence—polyA—3′ ITR. The promoter may be an hGRK1 promoter. The promoter may be a CBA or smCBA promoter. The polyA sequence may be a bGH polyA sequence. The intron and/or splice donor and/or splice acceptor may be a SV40 intron.

In some embodiments, the AAV vector has the following architecture: 5′ ITR—promoter—intron and/or splice donor and/or splice acceptor—hRS1 coding sequence—post-transcription regulatory element—polyA—stuffer—3′ ITR. The promoter may be an hGRK1 promoter. The promoter may be a CBA or smCBA promoter. The polyA sequence may be a bGH polyA sequence. The intron and/or splice donor and/or splice acceptor may be a SV40 intron. The post-transcription regulatory element may be a WPRE element.

In some embodiments, the AAV vector has the following architecture: 5′ ITR—promoter—intron and/or splice donor and/or splice acceptor—hRS1 coding sequence—polyA—3′ ITR. The promoter may be an hGRK1 promoter. The promoter may be a CBA or smCBA promoter. The polyA sequence may be a bGH polyA sequence. The intron and/or splice donor and/or splice acceptor may be a SV40 intron.

In some embodiments, the AAV vector has the following architecture: 5′ ITR—promoter—intron and/or splice donor and/or splice acceptor—hRS1 coding sequence—post-transcription regulatory element—polyA—3′ ITR. The promoter may be an hGRK1 promoter. The promoter may be a CBA or smCBA promoter. The polyA sequence may be a bGH polyA sequence. The intron and/or splice donor and/or splice acceptor may be a SV40 intron. The post-transcription regulatory element may be a WPRE element.

The disclosed rAAV vectors may comprise a nucleotide sequence having at least 75% identity, at least 80% identity, at least 85% identity, at least 90% identity, at least 95% identity, 98% identity, or 99% identity to the nucleotide sequence of SEQ ID NO: 16 or 17. In some embodiments, the vector comprises the nucleotide sequence of SEQ ID NO: 16 or 17. In particular embodiments, the disclosure provides rAAV vectors comprising the AAV-hGRK1-hRS1syn structure that comprises the nucleotide sequence of SEQ ID NO: 16, provided below. The length of SEQ ID NO: 16 is 1740 nucleotides (nt). In particular embodiments, the disclosure provides rAAV vectors comprising the AAV-hGRK1-hRS1syn-WPREsf structure that comprises the nucleotide sequence of SEQ ID NO: 17, provided below. The length of SEQ ID NO: 17 is 2320 nucleotides (nt).

In some embodiments, exemplary rAAV vectors of the disclosure contain the following architecture: 5′ ITR—promoter—SV40 intron—hRS1 coding sequence—polyA—stuffer—3′ ITR. In some embodiments, exemplary rAAV vectors of the disclosure contain the following architecture: 5′ ITR—promoter—SV40 intron—hRS1 coding sequence—polyA—3′ ITR. In some embodiments, exemplary rAAV vectors of the disclosure contain the following architecture: 5′ ITR—promoter—SV40 intron—hRS1 coding sequence—WPREsf—polyA—stuffer—3′ ITR. In some embodiments, exemplary rAAV vectors of the disclosure contain the following architecture: 5′ ITR—promoter—SV40 intron—hRS1 coding sequence WPREsf—polyA—3′ ITR. The promoter of a vector in accordance with any of these architectures may be an hGRK1 promoter. The promoter of a vector in accordance with any of these architectures may be a CBA or smCBA promoter. The polyA sequence of a vector in accordance with any of these architectures may be a bGH polyA sequence. In the below constructs, one or more modified Kozak sequences is indicated in italics.

pTR-X001-3p (ITR-ITR sequence - 4534 bp)
(SEQ ID NO: 31)
TTGGCCACTCCCTCTCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGGAGT
GGCCAACTCCATCACTAGGGGTTCCTCAGATCTGAATTCGGTACCGGGCCCCAGAAGCCT
GGTGGTTGTTTGTCCTTCTCAGGGGAAAAGTGAGGCGGCCCCTTGGAGGAAGGGGCCGG
GCAGAATGATCTAATCGGATTCCAAGCAGCTCAGGGGATTGTCTTTTTCTAGCACCTTCT
TGCCACTCCTAAGCGTCCTCCGTGACCCCGGCTGGGATTTAGCCTGGTGCTGTGTCAGCC
CCGGTCTCCCAGGGGCTTCCCAGTGGTCCCCAGGAACCCTCGACAGGGCCCGGTCTCTCT
CGTCCAGCAAGGGCAGGGACGGGCCACAGGCCAAGGGCTCTAGAGGATCCGGTACTCGA
GGAACTGAAAAACCAGAAAGTTAACTGGTAAGTTTAGTCTTTTTGTCTTTTATTTCAGGT
CCCGGATCCGGTGGTGGTGCAAATCAAAGAACTGCTCCTCAGTGGATGTTGCCTTTACTT
CTAGGCCTGTACGGAAGTGTTACTTCTGCTCTAAAAGCTGCGGAATTGTACCCGCGGCCG
CCACCATGTCACGCAAGATAGAAGGCTTTTTGTTATTACTTCTCTTTGGCTATGAAGCCAC
ATTGGGATTATCGTCTACCGAGGATGAAGGCGAGGACCCATGGTATCAAAAAGCCTGCA
AGTGCGATTGCCAAGGAGGACCCAATGCTCTGTGGTCTGCAGGTGCCACCTCCTTGGACT
GTATACCAGAATGCCCATATCACAAGCCTCTGGGTTTCGAGTCAGGGGAGGTCACACCG
GACCAGATCACCTGCTCTAACCCGGAGCAGTATGTGGGCTGGTATTCTTCGTGGACTGCA
AACAAGGCCCGGCTCAACAGTCAAGGCTTTGGGTGTGCCTGGCTCTCCAAGTTCCAGGAC
AGTAGCCAGTGGTTACAGATAGATCTGAAGGAGATCAAAGTGATTTCAGGCATCCTCAC
CCAGGGGCGCTGTGACATCGATGAGTGGATGACCAAGTACAGCGTGCAGTACAGGACCG
ATGAGCGCCTGAACTGGATTTACTACAAGGACCAGACTGGAAACAACCGGGTCTTCTAT
GGCAACTCGGACCGCACCTCCACGGTTCAGAACCTGCTGCGGCCCCCCATCATCTCCCGC
TTCATCCGCCTCATCCCGCTGGGCTGGCACGTCCGCATTGCCATCCGGATGGAGCTGCTG
GAGTGCGTCAGCAAGTGTGCCTGAGTCGACTAGAGCTCGCTGATCAGCCTCGACTGTGCC
TTCTAGTTGCCAGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGA
CAATAGCAGGCATCCGATTCGAGTGAGCAGACCACATGATGATTGGTATTCTCCCTGAAG
AAAGAATGTCAGCAAGGAGTATCTGAAGTCCGACCAAGCCTAGAATCCGCCCGGTTCTT
ATTTGCTGAAGTAATACTGTTGTCAAGTCAGCTACAGGGAGTGGTCCCTGCCTGGAGTTT
GAAGGGCTGTCAAAGACATTGTGGTCACTTACTCAAGTGGTAAATTCCTTACAGACTGGA
CTGCAGTTAAGGGTGATAACGCTACTACTAGGACAAGGGTATCCTTGATCCTGTGGATCT
GTAGTGACCTGAGACTGTCAAGGTATGGTGAGTATGACCCTTGACCCTTTCCAACGGTTG
TAACCGTGGGCGCTGTCAAATCTGATTAACCACTACTAGGATAGAATTTGGCAGAGCTAC
TGAAATGGTTACCTGGTCAAGAGACTGGCCCACAGCATAAGGGCGAGCAAGACGATGCT
CAGATTAACTAAGTGACCGAAGTATCCTAGATGGTCCACTACCAGCAAATGTGTCAGAC
CTCCCTCAGTTGGTAGTACAATGTATCCTTCTAGTCGCGACTTATTATATCGACGAGCTTA
TGTGTAAGAGACCAGATTCTTTGTAACCTGACACTTGAGTTCAAGGTGTGTAGTAGGAGA
GTGCAAACCCTGAAGCAACAGTAGCCGAACCGGGTCGAGGACCTAGCGTTGATTGACTT
TGTCTGGTGTATCCTGTAAATTTCTCCAATCGAGATCGAGGTAAAGTGGACCTCACAGGA
CTTTATCAAACAACAGAGTTAATACCTCAAATCTCTCGCTGATATATGTGACAAATTCAT
TGCGTCACTTATCGCAGCTATGTTCGGTCAGCATATTCTGGCGAACACCCACGCTGCAGT
TCGACAAGGATAGTATCTGGCGCTCTCGCCCAATTCAAGCACGCGAATTACTCTATTAAC
TTGTTGTCTCTTGTGTATATCATAATCAATTCCTCCTAGTTAGTGATTGAAGACGACCGTG
CACTTGGCTGCCTTGATGCAGACCACTTCTTGTGGCCGGCACACCCTATAATAGAGATGT
GATAAATAACTTAGCTGGACTGTAGTTATTTCGTCCTAGGGATTCTGACGGGTGCAAATC
ACTAAGTGTCTTTGGTATTGGGTCTGTAACAATGTTGAGAACTGTGGTGTATGTACGAAC
GATGTGATCTGTAGACTACAGCACAGGGTATTGCTGTTTCGCACCAGTGTGACACTTGTG
TGGCTGTCCTCTGTGCTATAGCGCCGTATATCTTATGCAGGTTGTATAGCCCAACGACTGT
TTAGGTACGTCCGTATAAGAATAGGAGTGCTACACTCTACATTGAATCTATCCAGAGAGA
GTTCTGTAGAATTAGGTGACGAACAAATCTACGCGATAGGAAGGAGAGACCTGGTTGTC
CCAAGGGCCGTGGGTATGATGTTTAGTACAACTAGGAGGCTCTAGGTGTATTGGTTGTTG
GTCACGTAGTGTCCCTACTTGGTCAGGACTAAAGTTTGTCAGTGAACTCTCTACTATTATT
CATCGAACCTGTGGTCGTCGGCGGTCTGATATATGCTCGGTAGATTGCCCGACAGGACGT
GAAGGAGCCTAACTATTAAAGCACTCTAACTAGCCTATCGTAACAACTTATATTGGGTAT
TTGGGTACGACGGTGTACTGGAAATCTATCTAATTCTTCTGATCGTATATCGGGTCGTAT
ATTGATTTATGACGGACCGACCACTCACGCGGTCCGATACGCGGTAGCGCTATCCAGGAC
CCTCTTATGAATTGGTATCAATAGATTGTGGAACTTGTTGCGTACACTACAGAATGTCTTT
GCAAAGAATGCCAAATTACAAGCTATAGTCCTCCCACTATACTGCTTCGTGACTCCAACT
AGGGTAGTTATAGTTGTTCAAGACTGGTACTCTGTTACCAATTAGTAGCGATATTGTGTG
GGATACTGGTGTCTGTCATACGAGAACTGGGAGTAGCGCTGTAAATTCTAAATAGTCACA
CTGTTCCAGCTGGACGACCGCTGTGTACTCTTTGTAGAAACTCACTGCGGCTTCCACTTAT
GAGATTTGGTTATAGGTCACAAAGTGGCAGTCCAGTTGCAGGTCTCTTGTTGTAATATGA
CTGGATTGAATTGACTAGGAAGACGTAGTCTGTTCTTGTTAGGTCCGACGTGGTCCCAAT
TTGCTTATCCAATATGAGGTCCTAGTTAGCAGTCGCTCCGAGGTCGAAATAGATACCCTT
GTGGTTTCGTAGATTGTCTGATTCGCAACAATATCCTGCTAATCAATCTACGGAAACACC
GCTTCAGAGGAAGATTGCGACGGACAGAAGAGTCAGTTTCGGCTGCTTGTGGTTTGGCCT
AATATAAAGATTACTTACGGCTTAGGTATACTGCACATCCTCCGGCCTTGTTTGGTGCTTG
AGATTGGTGCGCATATATGTAAGGTGCTATCGTATCCGCCTCCAGTACTTGATCTTGTCCT
GCGGCTTTAGGGCTGCTTATCCCTCCAGCGGTCCAACCTCTGAAGGGTCAGGCCATACTT
CACCTCCTTTGTCTAGGATTCTGTTCCGAATTTCCGGTCTGGGTCATGTACTCTCACCATC
GAGTGTAGGGAGACGATTCTCGTAACAGCCGTAGCCGAATCACTTTCACTTGAGTCTATG
GGTTGTGCCTGAGTTCTAGCTTAGTATTTAACAACGGCGTCGGTCCAAAGACGGTACACA
CTTAACCATAGTGAACGAATGAAACAGTAATCGCTCCAGGTTGGCGCGCCTTGCATGCTG
GGGAGAGATCTGAGGAACCCCTAGTGATGGAGTTGGCCACTCCCTCTCTGCGCGCTCGCT
CGCTCACTGAGGCCGGGCGACCAAAGGTCGCCCGACGCCCGGGCTTTGCCCGGGCGGCC
TCAGTGAGCGAGCGAGCGCGCAGAGAGGGAGTGGCCAA
pTR-X001-5p (ITR-ITR sequence - 4528 bp)
(SEQ ID NO: 32)
TTGGCCACTCCCTCTCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGGAGT
GGCCAACTCCATCACTAGGGGTTCCTCAGATCTGAATTCGATTCGAGTGAGCAGACCACA
TGATGATTGGTATTCTCCCTGAAGAAAGAATGTCAGCAAGGAGTATCTGAAGTCCGACC
AAGCCTAGAATCCGCCCGGTTCTTATTTGCTGAAGTAATACTGTTGTCAAGTCAGCTACA
GGGAGTGGTCCCTGCCTGGAGTTTGAAGGGCTGTCAAAGACATTGTGGTCACTTACTCAA
GTGGTAAATTCCTTACAGACTGGACTGCAGTTAAGGGTGATAACGCTACTACTAGGACA
AGGGTATCCTTGATCCTGTGGATCTGTAGTGACCTGAGACTGTCAAGGTATGGTGAGTAT
GACCCTTGACCCTTTCCAACGGTTGTAACCGTGGGCGCTGTCAAATCTGATTAACCACTA
CTAGGATAGAATTTGGCAGAGCTACTGAAATGGTTACCTGGTCAAGAGACTGGCCCACA
GCATAAGGGCGAGCAAGACGATGCTCAGATTAACTAAGTGACCGAAGTATCCTAGATGG
TCCACTACCAGCAAATGTGTCAGACCTCCCTCAGTTGGTAGTACAATGTATCCTTCTAGT
CGCGACTTATTATATCGACGAGCTTATGTGTAAGAGACCAGATTCTTTGTAACCTGACAC
TTGAGTTCAAGGTGTGTAGTAGGAGAGTGCAAACCCTGAAGCAACAGTAGCCGAACCGG
GTCGAGGACCTAGCGTTGATTGACTTTGTCTGGTGTATCCTGTAAATTTCTCCAATCGAG
ATCGAGGTAAAGTGGACCTCACAGGACTTTATCAAACAACAGAGTTAATACCTCAAATC
TCTCGCTGATATATGTGACAAATTCATTGCGTCACTTATCGCAGCTATGTTCGGTCAGCAT
ATTCTGGCGAACACCCACGCTGCAGTTCGACAAGGATAGTATCTGGCGCTCTCGCCCAAT
TCAAGCACGCGAATTACTCTATTAACTTGTTGTCTCTTGTGTATATCATAATCAATTCCTC
CTAGTTAGTGATTGAAGACGACCGTGCACTTGGCTGCCTTGATGCAGACCACTTCTTGTG
GCCGGCACACCCTATAATAGAGATGTGATAAATAACTTAGCTGGACTGTAGTTATTTCGT
CCTAGGGATTCTGACGGGTGCAAATCACTAAGTGTCTTTGGTATTGGGTCTGTAACAATG
TTGAGAACTGTGGTGTATGTACGAACGATGTGATCTGTAGACTACAGCACAGGGTATTGC
TGTTTCGCACCAGTGTGACACTTGTGTGGCTGTCCTCTGTGCTATAGCGCCGTATATCTTA
TGCAGGTTGTATAGCCCAACGACTGTTTAGGTACGTCCGTATAAGAATAGGAGTGCTACA
CTCTACATTGAATCTATCCAGAGAGAGTTCTGTAGAATTAGGTGACGAACAAATCTACGC
GATAGGAAGGAGAGACCTGGTTGTCCCAAGGGCCGTGGGTATGATGTTTAGTACAACTA
GGAGGCTCTAGGTGTATTGGTTGTTGGTCACGTAGTGTCCCTACTTGGTCAGGACTAAAG
TTTGTCAGTGAACTCTCTACTATTATTCATCGAACCTGTGGTCGTCGGCGGTCTGATATAT
GCTCGGTAGATTGCCCGACAGGACGTGAAGGAGCCTAACTATTAAAGCACTCTAACTAG
CCTATCGTAACAACTTATATTGGGTATTTGGGTACGACGGTGTACTGGAAATCTATCTAA
TTCTTCTGATCGTATATCGGGTCGTATATTGATTTATGACGGACCGACCACTCACGCGGTC
CGATACGCGGTAGCGCTATCCAGGACCCTCTTATGAATTGGTATCAATAGATTGTGGAAC
TTGTTGCGTACACTACAGAATGTCTTTGCAAAGAATGCCAAATTACAAGCTATAGTCCTC
CCACTATACTGCTTCGTGACTCCAACTAGGGTAGTTATAGTTGTTCAAGACTGGTACTCT
GTTACCAATTAGTAGCGATATTGTGTGGGATACTGGTGTCTGTCATACGAGAACTGGGAG
TAGCGCTGTAAATTCTAAATAGTCACACTGTTCCAGCTGGACGACCGCTGTGTACTCTTT
GTAGAAACTCACTGCGGCTTCCACTTATGAGATTTGGTTATAGGTCACAAAGTGGCAGTC
CAGTTGCAGGTCTCTTGTTGTAATATGACTGGATTGAATTGACTAGGAAGACGTAGTCTG
TTCTTGTTAGGTCCGACGTGGTCCCAATTTGCTTATCCAATATGAGGTCCTAGTTAGCAGT
CGCTCCGAGGTCGAAATAGATACCCTTGTGGTTTCGTAGATTGTCTGATTCGCAACAATA
TCCTGCTAATCAATCTACGGAAACACCGCTTCAGAGGAAGATTGCGACGGACAGAAGAG
TCAGTTTCGGCTGCTTGTGGTTTGGCCTAATATAAAGATTACTTACGGCTTAGGTATACTG
CACATCCTCCGGCCTTGTTTGGTGCTTGAGATTGGTGCGCATATATGTAAGGTGCTATCGT
ATCCGCCTCCAGTACTTGATCTTGTCCTGCGGCTTTAGGGCTGCTTATCCCTCCAGCGGTC
CAACCTCTGAAGGGTCAGGCCATACTTCACCTCCTTTGTCTAGGATTCTGTTCCGAATTTC
CGGTCTGGGTCATGTACTCTCACCATCGAGTGTAGGGAGACGATTCTCGTAACAGCCGTA
GCCGAATCACTTTCACTTGAGTCTATGGGTTGTGCCTGAGTTCTAGCTTAGTATTTAACAA
CGGCGTCGGTCCAAAGACGGTACACACTTAACCATAGTGAACGAATGAAACAGTAATCG
CTCCAGGTTAAGAACTATTGGTACCGGGCCCCAGAAGCCTGGTGGTTGTTTGTCCTTCTC
AGGGGAAAAGTGAGGCGGCCCCTTGGAGGAAGGGGCCGGGCAGAATGATCTAATCGGA
TTCCAAGCAGCTCAGGGGATTGTCTTTTTCTAGCACCTTCTTGCCACTCCTAAGCGTCCTC
CGTGACCCCGGCTGGGATTTAGCCTGGTGCTGTGTCAGCCCCGGTCTCCCAGGGGCTTCC
CAGTGGTCCCCAGGAACCCTCGACAGGGCCCGGTCTCTCTCGTCCAGCAAGGGCAGGGA
CGGGCCACAGGCCAAGGGCTCTAGAGGATCCGGTACTCGAGGAACTGAAAAACCAGAA
AGTTAACTGGTAAGTTTAGTCTTTTTGTCTTTTATTTCAGGTCCCGGATCCGGTGGTGGTG
CAAATCAAAGAACTGCTCCTCAGTGGATGTTGCCTTTACTTCTAGGCCTGTACGGAAGTG
TTACTTCTGCTCTAAAAGCTGCGGAATTGTACCCGCGGCCGCCACCATGTCACGCAAGAT
AGAAGGCTTTTTGTTATTACTTCTCTTTGGCTATGAAGCCACATTGGGATTATCGTCTACC
GAGGATGAAGGCGAGGACCCATGGTATCAAAAAGCCTGCAAGTGCGATTGCCAAGGAG
GACCCAATGCTCTGTGGTCTGCAGGTGCCACCTCCTTGGACTGTATACCAGAATGCCCAT
ATCACAAGCCTCTGGGTTTCGAGTCAGGGGAGGTCACACCGGACCAGATCACCTGCTCTA
ACCCGGAGCAGTATGTGGGCTGGTATTCTTCGTGGACTGCAAACAAGGCCCGGCTCAAC
AGTCAAGGCTTTGGGTGTGCCTGGCTCTCCAAGTTCCAGGACAGTAGCCAGTGGTTACAG
ATAGATCTGAAGGAGATCAAAGTGATTTCAGGCATCCTCACCCAGGGGCGCTGTGACAT
CGATGAGTGGATGACCAAGTACAGCGTGCAGTACAGGACCGATGAGCGCCTGAACTGGA
TTTACTACAAGGACCAGACTGGAAACAACCGGGTCTTCTATGGCAACTCGGACCGCACCT
CCACGGTTCAGAACCTGCTGCGGCCCCCCATCATCTCCCGCTTCATCCGCCTCATCCCGCT
GGGCTGGCACGTCCGCATTGCCATCCGGATGGAGCTGCTGGAGTGCGTCAGCAAGTGTG
CCTGAGTCGACTAGAGCTCGCTGATCAGCCTCGACTGTGCCTTCTAGTTGCCAGCCATCT
GTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATGCTGG
GGAGAGATCTGAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGCGGCCT
CAGTGAGCGAGCGAGCGCGCAGAGAGGGAGTGGCCAA
pTR-X002-3p (ITR-ITR sequence - 4549 bp)
(SEQ ID NO: 33)
TTGGCCACTCCCTCTCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGGAGT
GGCCAACTCCATCACTAGGGGTTCCTCAGATCTGAATTCGGTACCGGGCCCCAGAAGCCT
GGTGGTTGTTTGTCCTTCTCAGGGGAAAAGTGAGGCGGCCCCTTGGAGGAAGGGGCCGG
GCAGAATGATCTAATCGGATTCCAAGCAGCTCAGGGGATTGTCTTTTTCTAGCACCTTCT
TGCCACTCCTAAGCGTCCTCCGTGACCCCGGCTGGGATTTAGCCTGGTGCTGTGTCAGCC
CCGGTCTCCCAGGGGCTTCCCAGTGGTCCCCAGGAACCCTCGACAGGGCCCGGTCTCTCT
CGTCCAGCAAGGGCAGGGACGGGCCACAGGCCAAGGGCTCTAGAGGATCCGGTACTCGA
GGAACTGAAAAACCAGAAAGTTAACTGGTAAGTTTAGTCTTTTTGTCTTTTATTTCAGGT
CCCGGATCCGGTGGTGGTGCAAATCAAAGAACTGCTCCTCAGTGGATGTTGCCTTTACTT
CTAGGCCTGTACGGAAGTGTTACTTCTGCTCTAAAAGCTGCGGAATTGTACCCGCGGCCG
CCACCATGTCACGCAAGATAGAAGGCTTTTTGTTATTACTTCTCTTTGGCTATGAAGCCAC
ATTGGGATTATCGTCTACCGAGGATGAAGGCGAGGACCCATGGTATCAAAAAGCCTGCA
AGTGCGATTGCCAAGGAGGACCCAATGCTCTGTGGTCTGCAGGTGCCACCTCCTTGGACT
GTATACCAGAATGCCCATATCACAAGCCTCTGGGTTTCGAGTCAGGGGAGGTCACACCG
GACCAGATCACCTGCTCTAACCCGGAGCAGTATGTGGGCTGGTATTCTTCGTGGACTGCA
AACAAGGCCCGGCTCAACAGTCAAGGCTTTGGGTGTGCCTGGCTCTCCAAGTTCCAGGAC
AGTAGCCAGTGGTTACAGATAGATCTGAAGGAGATCAAAGTGATTTCAGGCATCCTCAC
CCAGGGGCGCTGTGACATCGATGAGTGGATGACCAAGTACAGCGTGCAGTACAGGACCG
ATGAGCGCCTGAACTGGATTTACTACAAGGACCAGACTGGAAACAACCGGGTCTTCTAT
GGCAACTCGGACCGCACCTCCACGGTTCAGAACCTGCTGCGGCCCCCCATCATCTCCCGC
TTCATCCGCCTCATCCCGCTGGGCTGGCACGTCCGCATTGCCATCCGGATGGAGCTGCTG
GAGTGCGTCAGCAAGTGTGCCTGAGTCGACCTCTGGATTACAAAATTTGTGAAAGATTGA
CTGGTATTCTTAACTATGTTGCTCCTTTTACGCTATGTGGATACGCTGCTTTAATGCCTTT
GTATCATGCTATTGCTTCCCGTATGGCTTTCATTTTCTCCTCCTTGTATAAATCCTGGTTGC
TGTCTCTTTATGAGGAGTIGTGGCCCGTTGTCAGGCAACGTGGCGTGGTGTGCACTGTGT
TTGCTGACGCAACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGCCTTGCCCGCTG
CTGGACAGGGGCTCGGCTGTTGGGCACTGACAATTCCGTGGTGTTGTCGGGGAAATCATC
GTCCTTTCCTTGGCTGCTCGCCTGTGTTGCCACCTGGATTCTGCGCGGGACGTCCTTCTGC
TACGTCCCTTCGGCCCTCAATCCAGCGGACCTTCCTTCCCGCGGCCTGCTGCCGGCTCTGC
GGCCTCTTCCGCGTCTTCGCCTTCGCCCTCAGACGAGTCGGATCTCCCTTTGGGCCGCCTC
CCCGCGTCGACTAGAGCTCGCTGATCAGCCTCGACTGTGCCTTCTAGTTGCCAGCCATCT
GTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATCCAGT
GGTCCCTGCCTGGAGTTTGAAGGGCTGTCAAAGACATTGTGGTCACTTACTCAAGTGGTA
AATTCCTTACAGACTGGACTGCAGTTAAGGGTGATAACGCTACTACTAGGACAAGGGTA
TCCTTGATCCTGTGGATCTGTAGTGACCTGAGACTGTCAAGGTATGGTGAGTATGACCCT
TGACCCTTTCCAACGGTTGTAACCGTGGGCGCTGTCAAATCTGATTAACCACTACTAGGA
TAGAATTTGGCAGAGCTACTGAAATGGTTACCTGGTCAAGAGACTGGCCCACAGCATAA
GGGCGAGCAAGACGATGCTCAGATTAACTAAGTGACCGAAGTATCCTAGATGGTCCACT
ACCAGCAAATGTGTCAGACCTCCCTCAGTTGGTAGTACAATGTATCCTTCTAGTCGCGAC
TTATTATATCGACGAGCTTATGTGTAAGAGACCAGATTCTTTGTAACCTGACACTTGAGT
TCAAGGTGTGTAGTAGGAGAGTGCAAACCCTGAAGCAACAGTAGCCGAACCGGGTCGAG
GACCTAGCGTTGATTGACTTTGTCTGGTGTATCCTGTAAATTTCTCCAATCGAGATCGAG
GTAAAGTGGACCTCACAGGACTTTATCAAACAACAGAGTTAATACCTCAAATCTCTCGCT
GATATATGTGACAAATTCATTGCGTCACTTATCGCAGCTATGTTCGGTCAGCATATTCTG
GCGAACACCCACGCTGCAGTTCGACAAGGATAGTATCTGGCGCTCTCGCCCAATTCAAGC
ACGCGAATTACTCTATTAACTTGTTGTCTCTTGTGTATATCATAATCAATTCCTCCTAGTT
AGTGATTGAAGACGACCGTGCACTTGGCTGCCTTGATGCAGACCACTTCTTGTGGCCGGC
ACACCCTATAATAGAGATGTGATAAATAACTTAGCTGGACTGTAGTTATTTCGTCCTAGG
GATTCTGACGGGTGCAAATCACTAAGTGTCTTTGGTATTGGGTCTGTAACAATGTTGAGA
ACTGTGGTGTATGTACGAACGATGTGATCTGTAGACTACAGCACAGGGTATTGCTGTTTC
GCACCAGTGTGACACTTGTGTGGCTGTCCTCTGTGCTATAGCGCCGTATATCTTATGCAG
GTTGTATAGCCCAACGACTGTTTAGGTACGTCCGTATAAGAATAGGAGTGCTACACTCTA
CATTGAATCTATCCAGAGAGAGTTCTGTAGAATTAGGTGACGAACAAATCTACGCGATA
GGAAGGAGAGACCTGGTTGTCCCAAGGGCCGTGGGTATGATGTTTAGTACAACTAGGAG
GCTCTAGGTGTATTGGTTGTTGGTCACGTAGTGTCCCTACTTGGTCAGGACTAAAGTTTGT
CAGTGAACTCTCTACTATTATTCATCGAACCTGTGGTCGTCGGCGGTCTGATATATGCTCG
GTAGATTGCCCGACAGGACGTGAAGGAGCCTAACTATTAAAGCACTCTAACTAGCCTAT
CGTAACAACTTATATTGGGTATTTGGGTACGACGGTGTACTGGAAATCTATCTAATTCTT
CTGATCGTATATCGGGTCGTATATTGATTTATGACGGACCGACCACTCACGCGGTCCGAT
ACGCGGTAGCGCTATCCAGGACCCTCTTATGAATTGGTATCAATAGATTGTGGAACTTGT
TGCGTACACTACAGAATGTCTTTGCAAAGAATGCCAAATTACAAGCTATAGTCCTCCCAC
TATACTGCTTCGTGACTCCAACTAGGGTAGTTATAGTTGTTCAAGACTGGTACTCTGTTAC
CAATTAGTAGCGATATTGTGTGGGATACTGGTGTCTGTCATACGAGAACTGGGAGTAGCG
CTGTAAATTCTAAATAGTCACACTGTTCCAGCTGGACGACCGCTGTGTACTCTTTGTAGA
AACTCACTGCGGCTTCCACTTATGAGATTTGGTTATAGGTCACAAAGTGGCAGTCCAGTT
GCAGGTCTCTTGTTGTAATATGACTGGATTGAATTGACTAGGAAGACGTAGTCTGTTCTT
GTTAGGTCCGACGTGGTCCCAATTTGCTTATCCAATATGAGGTCCTAGTTAGCAGTCGCT
CCGAGGTCGAAATAGATACCCTTGTGGTTTCGTAGATTGTCTGATTCGCAACAATATCCT
GCTAATCAATCTACGGAAACACCGCTTCAGAGGAAGATTGCGACGGACAGAAGAGTCAG
GCGCGCCTTGCATGCTGGGGAGAGATCTGAGGAACCCCTAGTGATGGAGTTGGCCACTC
CCTCTCTGCGCGCTCGCTCGCTCACTGAGGCCGGGCGACCAAAGGTCGCCCGACGCCCGG
GCTTTGCCCGGGCGGCCTCAGTGAGCGAGCGAGCGCGCAGAGAGGGAGTGGCCAA
pTR-X001 (ITR-ITR sequence - 1723 bp)
(SEQ ID NO: 34)
GGCCACTCCCTCTCTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAGAGGGAGTGG
CCAACTCCATCACTAGGGGTTCCTCAGATCTGAATTCGGTACCGGGCCCCAGAAGCCTGG
TGGTTGTTTGTCCTTCTCAGGGGAAAAGTGAGGCGGCCCCTTGGAGGAAGGGGCCGGGC
AGAATGATCTAATCGGATTCCAAGCAGCTCAGGGGATTGTCTTTTTCTAGCACCTTCTTG
CCACTCCTAAGCGTCCTCCGTGACCCCGGCTGGGATTTAGCCTGGTGCTGTGTCAGCCCC
GGTCTCCCAGGGGCTTCCCAGTGGTCCCCAGGAACCCTCGACAGGGCCCGGTCTCTCTCG
TCCAGCAAGGGCAGGGACGGGCCACAGGCCAAGGGCTCTAGAGGATCCGGTACTCGAG
GAACTGAAAAACCAGAAAGTTAACTGGTAAGTTTAGTCTTTTTGTCTTTTATTTCAGGTCC
CGGATCCGGTGGTGGTGCAAATCAAAGAACTGCTCCTCAGTGGATGTTGCCTTTACTTCT
AGGCCTGTACGGAAGTGTTACTTCTGCTCTAAAAGCTGCGGAATTGTACCCGCGGCCGCC
ACCATGTCACGCAAGATAGAAGGCTTTTTGTTATTACTTCTCTTTGGCTATGAAGCCACAT
TGGGATTATCGTCTACCGAGGATGAAGGCGAGGACCCATGGTATCAAAAAGCCTGCAAG
TGCGATTGCCAAGGAGGACCCAATGCTCTGTGGTCTGCAGGTGCCACCTCCTTGGACTGT
ATACCAGAATGCCCATATCACAAGCCTCTGGGTTTCGAGTCAGGGGAGGTCACACCGGA
CCAGATCACCTGCTCTAACCCGGAGCAGTATGTGGGCTGGTATTCTTCGTGGACTGCAAA
CAAGGCCCGGCTCAACAGTCAAGGCTTTGGGTGTGCCTGGCTCTCCAAGTTCCAGGACAG
TAGCCAGTGGTTACAGATAGATCTGAAGGAGATCAAAGTGATTTCAGGCATCCTCACCC
AGGGGCGCTGTGACATCGATGAGTGGATGACCAAGTACAGCGTGCAGTACAGGACCGAT
GAGCGCCTGAACTGGATTTACTACAAGGACCAGACTGGAAACAACCGGGTCTTCTATGG
CAACTCGGACCGCACCTCCACGGTTCAGAACCTGCTGCGGCCCCCCATCATCTCCCGCTT
CATCCGCCTCATCCCGCTGGGCTGGCACGTCCGCATTGCCATCCGGATGGAGCTGCTGGA
GTGCGTCAGCAAGTGTGCCTGAGTCGACTAGAGCTCGCTGATCAGCCTCGACTGTGCCTT
CTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGGGAAGACA
ATAGCAGGCATGCTGGGGAGAGATCTGAGGAACCCCTAGTGATGGAGTTGGCCACTCCC
TCTCTGCGCGCTCGCTCGCTCACTGAGGCCGGGCGACCAAAGGTCGCCCGACGCCCGGG
CTTTGCCCGGGCGGCCTCAGTGAGCGAGCGAGCGCGCAGAGAGGGAGTGGCC
pTR-X002 (ITR-ITR sequence - 2311 bp)
(SEQ ID NO: 35)
GGCCACTCCCTCTCTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAGAGGGAGTGG
CCAACTCCATCACTAGGGGTTCCTCAGATCTGAATTCGGTACCGGGCCCCAGAAGCCTGG
TGGTTGTTTGTCCTTCTCAGGGGAAAAGTGAGGCGGCCCCTTGGAGGAAGGGGCCGGGC
AGAATGATCTAATCGGATTCCAAGCAGCTCAGGGGATTGTCTTTTTCTAGCACCTTCTTG
CCACTCCTAAGCGTCCTCCGTGACCCCGGCTGGGATTTAGCCTGGTGCTGTGTCAGCCCC
GGTCTCCCAGGGGCTTCCCAGTGGTCCCCAGGAACCCTCGACAGGGCCCGGTCTCTCTCG
TCCAGCAAGGGCAGGGACGGGCCACAGGCCAAGGGCTCTAGAGGATCCGGTACTCGAG
GAACTGAAAAACCAGAAAGTTAACTGGTAAGTTTAGTCTTTTTGTCTTTTATTTCAGGTCC
CGGATCCGGTGGTGGTGCAAATCAAAGAACTGCTCCTCAGTGGATGTTGCCTTTACTTCT
AGGCCTGTACGGAAGTGTTACTTCTGCTCTAAAAGCTGCGGAATTGTACCCGCGGCCGCC
ACCATGTCACGCAAGATAGAAGGCTTTTTGTTATTACTTCTCTTTGGCTATGAAGCCACAT
TGGGATTATCGTCTACCGAGGATGAAGGCGAGGACCCATGGTATCAAAAAGCCTGCAAG
TGCGATTGCCAAGGAGGACCCAATGCTCTGTGGTCTGCAGGTGCCACCTCCTTGGACTGT
ATACCAGAATGCCCATATCACAAGCCTCTGGGTTTCGAGTCAGGGGAGGTCACACCGGA
CCAGATCACCTGCTCTAACCCGGAGCAGTATGTGGGCTGGTATTCTTCGTGGACTGCAAA
CAAGGCCCGGCTCAACAGTCAAGGCTTTGGGTGTGCCTGGCTCTCCAAGTTCCAGGACAG
TAGCCAGTGGTTACAGATAGATCTGAAGGAGATCAAAGTGATTTCAGGCATCCTCACCC
AGGGGCGCTGTGACATCGATGAGTGGATGACCAAGTACAGCGTGCAGTACAGGACCGAT
GAGCGCCTGAACTGGATTTACTACAAGGACCAGACTGGAAACAACCGGGTCTTCTATGG
CAACTCGGACCGCACCTCCACGGTTCAGAACCTGCTGCGGCCCCCCATCATCTCCCGCTT
CATCCGCCTCATCCCGCTGGGCTGGCACGTCCGCATTGCCATCCGGATGGAGCTGCTGGA
GTGCGTCAGCAAGTGTGCCTGAGTCGACCTCTGGATTACAAAATTTGTGAAAGATTGACT
GGTATTCTTAACTATGTTGCTCCTTTTACGCTATGTGGATACGCTGCTTTAATGCCTTTGT
ATCATGCTATTGCTTCCCGTATGGCTTTCATTTTCTCCTCCTTGTATAAATCCTGGTTGCTG
TCTCTTTATGAGGAGTTGTGGCCCGTTGTCAGGCAACGTGGCGTGGTGTGCACTGTGTTT
GCTGACGCAACCCCCACTGGTTGGGGCATTGCCACCACCTGTCAGCTCCTTTCCGGGACT
TTCGCTTTCCCCCTCCCTATTGCCACGGCGGAACTCATCGCCGCCTGCCTTGCCCGCTGCT
GGACAGGGGCTCGGCTGTTGGGCACTGACAATTCCGTGGTGTTGTCGGGGAAATCATCGT
CCTTTCCTTGGCTGCTCGCCTGTGTTGCCACCTGGATTCTGCGCGGGACGTCCTTCTGCTA
CGTCCCTTCGGCCCTCAATCCAGCGGACCTTCCTTCCCGCGGCCTGCTGCCGGCTCTGCG
GCCTCTTCCGCGTCTTCGCCTTCGCCCTCAGACGAGTCGGATCTCCCTTTGGGCCGCCTCC
CCGCGTCGACTAGAGCTCGCTGATCAGCCTCGACTGTGCCTTCTAGTTGCCAGCCATCTG
TTGTTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGGGGG
TGGGGTGGGGCAGGACAGCAAGGGGGAGGATTGGGAAGACAATAGCAGGCATGCTGGG
GAGAGATCTGAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGCCTC
AGTGAGCGAGCGAGCGCGCAGAGAGGGAGTGGCC
pTR-X002-3pSR (ITR-ITR sequence -4549 bp)
(SEQ ID NO: 33)
TTGGCCACTCCCTCTCTGCGCGCTCGCTCGCTCACTGAGGCCGCCCGGGCAAAGCCCGGG
CGTCGGGCGACCTTTGGTCGCCCGGCCTCAGTGAGCGAGCGAGCGCGCAGAGAGGGAGT
GGCCAACTCCATCACTAGGGGTTCCTCAGATCTGAATTCGGTACCGGGCCCCAGAAGCCT
GGTGGTTGTTTGTCCTTCTCAGGGGAAAAGTGAGGCGGCCCCTTGGAGGAAGGGGCCGG
GCAGAATGATCTAATCGGATTCCAAGCAGCTCAGGGGATTGTCTTTTTCTAGCACCTTCT
TGCCACTCCTAAGCGTCCTCCGTGACCCCGGCTGGGATTTAGCCTGGTGCTGTGTCAGCC
CCGGTCTCCCAGGGGCTTCCCAGTGGTCCCCAGGAACCCTCGACAGGGCCCGGTCTCTCT
CGTCCAGCAAGGGCAGGGACGGGCCACAGGCCAAGGGCTCTAGAGGATCCGGTACTCGA
GGAACTGAAAAACCAGAAAGTTAACTGGTAAGTTTAGTCTTTTTGTCTTTTATTTCAGGT
CCCGGATCCGGTGGTGGTGCAAATCAAAGAACTGCTCCTCAGTGGATGTTGCCTTTACTT
CTAGGCCTGTACGGAAGTGTTACTTCTGCTCTAAAAGCTGCGGAATTGTACCCGCGGCCG
CCACCATGTCACGCAAGATAGAAGGCTTTTTGTTATTACTTCTCTTTGGCTATGAAGCCAC
ATTGGGATTATCGTCTACCGAGGATGAAGGCGAGGACCCATGGTATCAAAAAGCCTGCA
AGTGCGATTGCCAAGGAGGACCCAATGCTCTGTGGTCTGCAGGTGCCACCTCCTTGGACT
GTATACCAGAATGCCCATATCACAAGCCTCTGGGTTTCGAGTCAGGGGAGGTCACACCG
GACCAGATCACCTGCTCTAACCCGGAGCAGTATGTGGGCTGGTATTCTTCGTGGACTGCA
AACAAGGCCCGGCTCAACAGTCAAGGCTTTGGGTGTGCCTGGCTCTCCAAGTTCCAGGAC
AGTAGCCAGTGGTTACAGATAGATCTGAAGGAGATCAAAGTGATTTCAGGCATCCTCAC
CCAGGGGCGCTGTGACATCGATGAGTGGATGACCAAGTACAGCGTGCAGTACAGGACCG
ATGAGCGCCTGAACTGGATTTACTACAAGGACCAGACTGGAAACAACCGGGTCTTCTAT
GGCAACTCGGACCGCACCTCCACGGTTCAGAACCTGCTGCGGCCCCCCATCATCTCCCGC
TTCATCCGCCTCATCCCGCTGGGCTGGCACGTCCGCATTGCCATCCGGATGGAGCTGCTG
GAGTGCGTCAGCAAGTGTGCCTGAGTCGACCTCTGGATTACAAAATTTGTGAAAGATTGA
CTGGTATTCTTAACTATGTTGCTCCTTTTACGCTATGTGGATACGCTGCTTTAATGCCTTT
GTATCATGCTATTGCTTCCCGTATGGCTTTCATTTTCTCCTCCTTGTATAAATCCTGGTTGC
TGTCTCTTTATGAGGAGTTGTGGCCCGTTGTCAGGCAACGTGGCGTGGTGTGCACTGTGT
TTGCTGACGCAACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGCCTTGCCCGCTG
CTGGACAGGGGCTCGGCTGTTGGGCACTGACAATTCCGTGGTGTTGTCGGGGAAATCATC
GTCCTTTCCTTGGCTGCTCGCCTGTGTTGCCACCTGGATTCTGCGCGGGACGTCCTTCTGC
TACGTCCCTTCGGCCCTCAATCCAGCGGACCTTCCTTCCCGCGGCCTGCTGCCGGCTCTGC
GGCCTCTTCCGCGTCTTCGCCTTCGCCCTCAGACGAGTCGGATCTCCCTTTGGGCCGCCTC
CCCGCGTCGACTAGAGCTCGCTGATCAGCCTCGACTGTGCCTTCTAGTTGCCAGCCATCT
GTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATCCAGT
GGTCCCTGCCTGGAGTTTGAAGGGCTGTCAAAGACATTGTGGTCACTTACTCAAGTGGTA
AATTCCTTACAGACTGGACTGCAGTTAAGGGTGATAACGCTACTACTAGGACAAGGGTA
TCCTTGATCCTGTGGATCTGTAGTGACCTGAGACTGTCAAGGTATGGTGAGTATGACCCT
TGACCCTTTCCAACGGTTGTAACCGTGGGCGCTGTCAAATCTGATTAACCACTACTAGGA
TAGAATTTGGCAGAGCTACTGAAATGGTTACCTGGTCAAGAGACTGGCCCACAGCATAA
GGGCGAGCAAGACGATGCTCAGATTAACTAAGTGACCGAAGTATCCTAGATGGTCCACT
ACCAGCAAATGTGTCAGACCTCCCTCAGTTGGTAGTACAATGTATCCTTCTAGTCGCGAC
TTATTATATCGACGAGCTTATGTGTAAGAGACCAGATTCTTTGTAACCTGACACTTGAGT
TCAAGGTGTGTAGTAGGAGAGTGCAAACCCTGAAGCAACAGTAGCCGAACCGGGTCGAG
GACCTAGCGTTGATTGACTTTGTCTGGTGTATCCTGTAAATTTCTCCAATCGAGATCGAG
GTAAAGTGGACCTCACAGGACTTTATCAAACAACAGAGTTAATACCTCAAATCTCTCGCT
GATATATGTGACAAATTCATTGCGTCACTTATCGCAGCTATGTTCGGTCAGCATATTCTG
GCGAACACCCACGCTGCAGTTCGACAAGGATAGTATCTGGCGCTCTCGCCCAATTCAAGC
ACGCGAATTACTCTATTAACTTGTTGTCTCTTGTGTATATCATAATCAATTCCTCCTAGTT
AGTGATTGAAGACGACCGTGCACTTGGCTGCCTTGATGCAGACCACTTCTTGTGGCCGGC
ACACCCTATAATAGAGATGTGATAAATAACTTAGCTGGACTGTAGTTATTTCGTCCTAGG
GATTCTGACGGGTGCAAATCACTAAGTGTCTTTGGTATTGGGTCTGTAACAATGTTGAGA
ACTGTGGTGTATGTACGAACGATGTGATCTGTAGACTACAGCACAGGGTATTGCTGTTTC
GCACCAGTGTGACACTTGTGTGGCTGTCCTCTGTGCTATAGCGCCGTATATCTTATGCAG
GTTGTATAGCCCAACGACTGTTTAGGTACGTCCGTATAAGAATAGGAGTGCTACACTCTA
CATTGAATCTATCCAGAGAGAGTTCTGTAGAATTAGGTGACGAACAAATCTACGCGATA
GGAAGGAGAGACCTGGTTGTCCCAAGGGCCGTGGGTATGATGTTTAGTACAACTAGGAG
GCTCTAGGTGTATTGGTTGTTGGTCACGTAGTGTCCCTACTTGGTCAGGACTAAAGTTTGT
CAGTGAACTCTCTACTATTATTCATCGAACCTGTGGTCGTCGGCGGTCTGATATATGCTCG
GTAGATTGCCCGACAGGACGTGAAGGAGCCTAACTATTAAAGCACTCTAACTAGCCTAT
CGTAACAACTTATATTGGGTATTTGGGTACGACGGTGTACTGGAAATCTATCTAATTCTT
CTGATCGTATATCGGGTCGTATATTGATTTATGACGGACCGACCACTCACGCGGTCCGAT
ACGCGGTAGCGCTATCCAGGACCCTCTTATGAATTGGTATCAATAGATTGTGGAACTTGT
TGCGTACACTACAGAATGTCTTTGCAAAGAATGCCAAATTACAAGCTATAGTCCTCCCAC
TATACTGCTTCGTGACTCCAACTAGGGTAGTTATAGTTGTTCAAGACTGGTACTCTGTTAC
CAATTAGTAGCGATATTGTGTGGGATACTGGTGTCTGTCATACGAGAACTGGGAGTAGCG
CTGTAAATTCTAAATAGTCACACTGTTCCAGCTGGACGACCGCTGTGTACTCTTTGTAGA
AACTCACTGCGGCTTCCACTTATGAGATTTGGTTATAGGTCACAAAGTGGCAGTCCAGTT
GCAGGTCTCTTGTTGTAATATGACTGGATTGAATTGACTAGGAAGACGTAGTCTGTTCTT
GTTAGGTCCGACGTGGTCCCAATTTGCTTATCCAATATGAGGTCCTAGTTAGCAGTCGCT
CCGAGGTCGAAATAGATACCCTTGTGGTTTCGTAGATTGTCTGATTCGCAACAATATCCT
GCTAATCAATCTACGGAAACACCGCTTCAGAGGAAGATTGCGACGGACAGAAGAGTCAG
GCGCGCCTTGCATGCTGGGGAGAGATCTGAGGAACCCCTAGTGATGGAGTTGGCCACTC
CCTCTCTGCGCGCTCGCTCGCTCACTGAGGCCGGGCGACCAAAGGTCGCCCGACGCCCGG
GCTTTGCCCGGGCGGCCTCAGTGAGCGAGCGAGCGCGCAGAGAGGGAGTGGCCAA
AAV-hGRK1-hRS1syn
(SEQ ID NO: 16)
GGTTGGCCACTCCCTCTCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAGAGGGA
GTGGCCAACTCCATCACTAGGGGTTCCTCAGATCTGAATTCGGTACCGGGCCCCAGAAGC
CTGGTGGTTGTTTGTCCTTCTCAGGGGAAAAGTGAGGCGGCCCCTTGGAGGAAGGGGCC
GGGCAGAATGATCTAATCGGATTCCAAGCAGCTCAGGGGATTGTCTTTTTCTAGCACCTT
CTTGCCACTCCTAAGCGTCCTCCGTGACCCCGGCTGGGATTTAGCCTGGTGCTGTGTCAG
CCCCGGTCTCCCAGGGGCTTCCCAGTGGTCCCCAGGAACCCTCGACAGGGCCCGGTCTCT
CTCGTCCAGCAAGGGCAGGGACGGGCCACAGGCCAAGGGCTCTAGAGGATCCGGTACTC
GAGGAACTGAAAAACCAGAAAGTTAACTGGTAAGTTTAGTCTTTTTGTCTTTTATTTCAG
GTCCCGGATCCGGTGGTGGTGCAAATCAAAGAACTGCTCCTCAGTGGATGTTGCCTTTAC
TTCTAGGCCTGTACGGAAGTGTTACTTCTGCTCTAAAAGCTGCGGAATTGTACCCGCGGC
CGCCACCATGTCACGCAAGATAGAAGGCTTTTTGTTATTACTTCTCTTTGGCTATGAAGCC
ACATTGGGATTATCGTCTACCGAGGATGAAGGCGAGGACCCATGGTATCAAAAAGCCTG
CAAGTGCGATTGCCAAGGAGGACCCAATGCTCTGTGGTCTGCAGGTGCCACCTCCTTGGA
CTGTATACCAGAATGCCCATATCACAAGCCTCTGGGTTTCGAGTCAGGGGAGGTCACACC
GGACCAGATCACCTGCTCTAACCCGGAGCAGTATGTGGGCTGGTATTCTTCGTGGACTGC
AAACAAGGCCCGGCTCAACAGTCAAGGCTTTGGGTGTGCCTGGCTCTCCAAGTTCCAGG
ACAGTAGCCAGTGGTTACAGATAGATCTGAAGGAGATCAAAGTGATTTCAGGCATCCTC
ACCCAGGGGCGCTGTGACATCGATGAGTGGATGACCAAGTACAGCGTGCAGTACAGGAC
CGATGAGCGCCTGAACTGGATTTACTACAAGGACCAGACTGGAAACAACCGGGTCTTCT
ATGGCAACTCGGACCGCACCTCCACGGTTCAGAACCTGCTGCGGCCCCCCATCATCTCCC
GCTTCATCCGCCTCATCCCGCTGGGCTGGCACGTCCGCATTGCCATCCGGATGGAGCTGC
TGGAGTGCGTCAGCAAGTGTGCCTGAGTCGACTAGAGCTCGCTGATCAGCCTCGACTGTG
CCTTCTAGTTGCCAGCCATCTGTTGTTTGCCCCTCCCCCGTGCCTTCCTTGACCCTGGAAG
GTGCCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGGGAA
GACAATAGCAGGCATGCTGGGGAGAGATCTGAGGAACCCCTAGTGATGGAGTTGGCCAC
TCCCTCTCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGAGAGGGAGTGGCCAACC
AAV-hGRK1-hRS1syn-WPREsf
(SEQ ID NO: 17)
GGTTGGCCACTCCCTCTCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGCAGAGAGGGA
GTGGCCAACTCCATCACTAGGGGTTCCTCAGATCTGAATTCGGTACCGGGCCCCAGAAGC
CTGGTGGTTGTTTGTCCTTCTCAGGGGAAAAGTGAGGCGGCCCCTTGGAGGAAGGGGCC
GGGCAGAATGATCTAATCGGATTCCAAGCAGCTCAGGGGATTGTCTTTTTCTAGCACCTT
CTTGCCACTCCTAAGCGTCCTCCGTGACCCCGGCTGGGATTTAGCCTGGTGCTGTGTCAG
CCCCGGTCTCCCAGGGGCTTCCCAGTGGTCCCCAGGAACCCTCGACAGGGCCCGGTCTCT
CTCGTCCAGCAAGGGCAGGGACGGGCCACAGGCCAAGGGCTCTAGAGGATCCGGTACTC
GAGGAACTGAAAAACCAGAAAGTTAACTGGTAAGTTTAGTCTTTTTGTCTTTTATTTCAG
GTCCCGGATCCGGTGGTGGTGCAAATCAAAGAACTGCTCCTCAGTGGATGTTGCCTTTAC
TTCTAGGCCTGTACGGAAGTGTTACTTCTGCTCTAAAAGCTGCGGAATTGTACCCGCGGC
CGCCACCATGTCACGCAAGATAGAAGGCTTTTTGTTATTACTTCTCTTTGGCTATGAAGCC
ACATTGGGATTATCGTCTACCGAGGATGAAGGCGAGGACCCATGGTATCAAAAAGCCTG
CAAGTGCGATTGCCAAGGAGGACCCAATGCTCTGTGGTCTGCAGGTGCCACCTCCTTGGA
CTGTATACCAGAATGCCCATATCACAAGCCTCTGGGTTTCGAGTCAGGGGAGGTCACACC
GGACCAGATCACCTGCTCTAACCCGGAGCAGTATGTGGGCTGGTATTCTTCGTGGACTGC
AAACAAGGCCCGGCTCAACAGTCAAGGCTTTGGGTGTGCCTGGCTCTCCAAGTTCCAGG
ACAGTAGCCAGTGGTTACAGATAGATCTGAAGGAGATCAAAGTGATTTCAGGCATCCTC
ACCCAGGGGCGCTGTGACATCGATGAGTGGATGACCAAGTACAGCGTGCAGTACAGGAC
CGATGAGCGCCTGAACTGGATTTACTACAAGGACCAGACTGGAAACAACCGGGTCTTCT
ATGGCAACTCGGACCGCACCTCCACGGTTCAGAACCTGCTGCGGCCCCCCATCATCTCCC
GCTTCATCCGCCTCATCCCGCTGGGCTGGCACGTCCGCATTGCCATCCGGATGGAGCTGC
TGGAGTGCGTCAGCAAGTGTGCCTGAGTCGACCTCTGGATTACAAAATTTGTGAAAGATT
GACTGGTATTCTTAACTATGTTGCTCCTTTTACGCTATGTGGATACGCTGCTTTAATGCCT
TTGTATCATGCTATTGCTTCCCGTATGGCTTTCATTTTCTCCTCCTTGTATAAATCCTGGTT
GCTGTCTCTTTATGAGGAGTIGTGGCCCGTTGTCAGGCAACGTGGCGTGGTGTGCACTGT
GTTTGCTGACGCAACCCCCACTGGTTGGGGCATTGCCACCACCTGTCAGCTCCTTTCCGG
GACTTTCGCTTTCCCCCTCCCTATTGCCACGGCGGAACTCATCGCCGCCTGCCTTGCCCGC
TGCTGGACAGGGGCTCGGCTGTTGGGCACTGACAATTCCGTGGTGTTGTCGGGGAAATCA
TCGTCCTTTCCTTGGCTGCTCGCCTGTGTTGCCACCTGGATTCTGCGCGGGACGTCCTTCT
GCTACGTCCCTTCGGCCCTCAATCCAGCGGACCTTCCTTCCCGCGGCCTGCTGCCGGCTCT
GCGGCCTCTTCCGCGTCTTCGCCTTCGCCCTCAGACGAGTCGGATCTCCCTTTGGGCCGCC
TCCCCGCGTCGACTAGAGCTCGCTGATCAGCCTCGACTGTGCCTTCTAGTTGCCAGCCAT
CTGTTGTTTGCCCCTCCCCCGTGCCTTCCTTGACCCTGGAAGGTGCCACTCCCACTGTCCT
TTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCATGCT
GGGGAGAGATCTGAGGAACCCCTAGTGATGGAGTTGGCCACTCCCTCTCTGCGCGCTCGC
TCGCTCACTGAGGCCGGGCGACCAAAGGTCGCCCGACGCCCGGGCTTTGCCCGGGCGGC
CTCAGTGAGCGAGCGAGCGCGCAGAGAGGGAGTGGCCAACC

### Capsid Variants

In some embodiments, the disclosure provides improved rAAV particles that have been derived from a number of different serotypes, including but not limited to AAV1, AAV2, AAV3, AAV4, AAV5, AAV6, AAV7, AAV8, AAV9, AAV10 and combinations thereof. In particular embodiments, the capsid comprises AAV5 or AAV44.9(E531D). In some embodiments, the capsid may comprise a variant of AAV2, a variant of AAV5, a variant of AAV7, a variant of AAV8, or a variant of AAV9. In some embodiments, the capsid comprises AAV44.9(E531D), AAV2(4pMut)ΔHS, AAV44.9, AAVrh.8, AAVrh.8R, AAVrh.10, AAVrh.74, AAV2TT, AAV2HBKO, AAV8(Y447F+Y733F+T494V), or AAVAnc80. In some embodiments, the capsid comprises AAV2, AAV6, or a capsid variant derived from AAV2 or AAV6. Accordingly, in some embodiments, the capsid comprises AAV2(7m8), AAV-DJ, AAV2/2-MAX, AAVSHh10, AAVSHh10Y, AAV3, AAV3b, AAVLK03, AAV7BP2, AAV1(E531K), AAV6(D532N), AAV6-3pmut, AAV2G9 or elements thereof.

In particular embodiments, the present disclosure provides rAAV particles comprising capsid variants of the capsid serotype AAV44.9. In some embodiments, the disclosed particles comprise the capsid protein AAV44.9(E531D). As described in the Examples herein, it was found that AAV44.9(E531D) mediates improved retinal transduction relative to unmodified AAV44.9 and AAVrh.8, and significantly higher transduction than benchmark capsids (e.g., AAV5- and AAV8-based vectors) in both species. Accordingly, the disclosure provides rAAV particles comprising a capsid protein of the AAV44.9(E531D) serotype, and related compositions and methods. In some embodiments, the rAAV particle comprises a heterologous nucleic acid, e.g., encoding a therapeutic or diagnostic agent. The heterologous nucleic acid may be in the form of a single-stranded (ss) or self-complementary (sc) AAV nucleic acid vector, such as single-stranded or self-complementary recombinant viral genome.

The disclosure further provides rAAV particles having AAV44.9 capsids that comprise the E531D substitution and one or more additional substitutions, such as a Y-F mutation at residue 446, a T-V mutation at residue 492, or both. Accordingly, the disclosure provides rAAV particles comprising an AAV44.9(T492V+E531D) capsid, an AAV44.9(Y446F+E531D) capsid, or an AAV44.9(Y446F+T492V+E531D) capsid, and related compositions and methods.

In some embodiments, the disclosure provides rAAV particles comprising an AAV44.9 capsid. This capsid may be highly suitable for mediating improved transduction of retinal tissues by subretinal injection. The inventors have discovered that intravitreal injection of AAV44.9 particles may not effectively transduce retinal cells.

In some embodiments, the disclosure provides rAAV particles comprising an AAV2(4pMut)ΔHS capsid. The AAV2(4pMut)ΔHS capsid was shown to mediate enhanced lateral spread in primate retina and display efficient photoreceptor transduction. The disclosure also provides particles comprising AAV8(Y733F) and AAV8(Y447F+Y733F+T494V) capsid variants.

Aspects of this disclosure relate to vectors comprising an AAV44.9(E531D) capsid that exhibits enhanced lateral spread after subretinal injection to the fovea, wherein detachment of the fovea (e.g., a temporary bullous detachment) is minimized. In some embodiments, the disclosure provides a capsid protein, e.g., a VP1, VP2 or VP3 capsid protein, comprising the amino acid sequence of SEQ ID NO: 1, 2, and/or 3.

In some embodiments, the disclosure provides an rAAV particle comprising a capsid comprising a VP1, VP2, and/or VP3 protein, wherein the rAAV particle further comprises a polynucleotide comprising a heterologous nucleic acid. In some embodiments, the rAAV particle comprises a capsid comprising a VP1, VP2, and/or VP3 protein, wherein the VP1 protein comprises the amino acid sequence of SEQ ID NO: 1, the VP2 protein comprises the amino acid sequence of SEQ ID NO: 2, and/or the VP3 protein comprises the amino acid sequence of SEQ ID NO: 3, and wherein the AAV further comprises a polynucleotide comprising a heterologous nucleic acid. The polynucleotide may be flanked by one or more inverted terminal repeat (ITR) sequences.

In some embodiments, the disclosure provides a capsid protein comprising an amino acid sequence having at least 80% identity, at least 85% identity, at least 90% identity, at least 92.5% identity, at least 95% identity, 98% identity, or 99% identity to any of SEQ ID NOs: 1, 2, or 3. In some embodiments, the disclosure provides a capsid protein comprising the amino acid sequence of SEQ ID NO: 1, 2, and/or 3. In particular embodiments, capsids comprising the amino acid sequence set forth as SEQ ID NO: 1 are provided (an AAV44.9(E531D) capsid VP1). In some embodiments, the disclosure provides a capsid protein comprising an amino acid sequence having at least 80% identity, at least 85% identity, at least 90% identity, at least 92.5% identity, at least 95% identity, 98% identity, or 99% identity to SEQ ID NO: 18. In particular embodiments, capsids comprising the amino acid sequence set forth as SEQ ID NO: 18 are provided (an AAV44.9(Y446F+T492V+E531D) capsid VP1). In some embodiments, the disclosed capsids may comprise a sequence having 1, 2, 3, 4, 5, 6, 7, 8, 9, 10, 11, 12, 13, 14, 15, 16, 17, or 18 amino acids that differ relative to the sequence of any one of SEQ ID NOs: 1-3 and 18. These differences may comprise amino acids that have been inserted, deleted, or substituted relative to the sequence of any one of SEQ ID NOs: 1-3 and 18.

In some embodiments, the disclosure provides a nucleic acid, e.g., a plasmid or viral vector, comprising the nucleic acid sequence of SEQ ID NO: 4 (which encodes AAV44.9(E531D) VP1). In some embodiments, the disclosure provides a nucleic acid, e.g., a plasmid or viral vector, comprising the nucleic acid sequence of SEQ ID NO: 5 (which encodes AAV44.9(E531D) VP2). In some embodiments, the disclosure provides a nucleic acid, e.g., a plasmid or viral vector, comprising the nucleic acid sequence of SEQ ID NO: 6 (which encodes AAV44.9(E531D) VP3). In some embodiments, the viral vector is a recombinant adeno-associated viral (rAAV) vector. In some embodiments, the rAAV vector is self-complementary. In some embodiments, the nucleic acid is comprised within a cell, e.g., a mammalian or insect cell.

All substitutions in the AAV44.9 capsid protein as described herein are based on the VP1 amino acid sequence set forth in SEQ ID NO: 1. As would be appreciated by one of skill in the art, all substitutions described herein in the VP1 sequence are equally applicable to the sequences of the VP2 and VP3 proteins. The sequences of SEQ ID NOs: 1-8 and 18 are provided below.

- AAV44.9(E531D) VP1 amino acid sequence
SEQ ID NO: 1
MAADGYLPDWLEDNLSEGIREWWDLKPGAPKPKANQQKQDDGRGLVLPGYKYLGPFNGL
DKGEPVNAADAAALEHDKAYDQQLKAGDNPYLRYNHADAEFQERLQEDTSFGGNLGRAVF
QAKKRVLEPLGLVEEGAKTAPGKKRPVEQSPQEPDSSSGIGKTGQQPAKKRLNFGQTGDTES
VPDPQPLGEPPAAPSGLGPNTMASGGGAPMADNNEGADGVGNSSGNWHCDSTWLGDRVIT
TSTRTWALPTYNNHLYKQISNGTSGGSTNDNTYFGYSTPWGYFDFNRFHCHFSPRDWQRLIN
NNWGFRPKRLNFKLFNIQVKEVTTNEGTKTIANNLTSTVQVFTDSEYQLPYVLGSAHQGCLP
PFPADVFMVPQYGYLTLNNGSQALGRSSFYCLEYFPSQMLRTGNNFQFSYTFEDVPFHSSYA
HSQSLDRLMNPLIDQYLYYLVRTQTTGTGGTQTLAFSQAGPSSMASQARNWVPGPSYRQQR
VSTTTNQNNNSNFAWTGAAKFKLNGRDSLMNPGVAMASHKDDDDRFFPSSGVLIFGKQGA
GNDGVDYSQVLITDEEEIKATNPVATEEYGAVAINNQAANTQAQTGLVHNQGVIPGMVWQ
NRDVYLQGPIWAKIPHTDGNFHPSPLMGGFGLKHPPPQILIKNTPVPADPPLTFNQAKLNSFIT
NL
- AAV44.9(E531D) VP2 amino acid sequence
SEQ ID NO: 2
MAPGKKRPVEQSPQEPDSSSGIGKTGQQPAKKRLNFGQTGDTESVPDPQPLGEPPAAPSGLGP
NTMASGGGAPMADNNEGADGVGNSSGNWHCDSTWLGDRVITTSTRTWALPTYNNHLYKQI
SNGTSGGSTNDNTYFGYSTPWGYFDFNRFHCHFSPRDWQRLINNNWGFRPKRLNFKLFNIQV
KEVTTNEGTKTIANNLTSTVQVFTDSEYQLPYVLGSAHQGCLPPFPADVFMVPQYGYLTLNN
GSQALGRSSFYCLEYFPSQMLRTGNNFQFSYTFEDVPFHSSYAHSQSLDRLMNPLIDQYLYYL
VRTQTTGTGGTQTLAFSQAGPSSMASQARNWVPGPSYRQQRVSTTTNQNNNSNFAWTGAA
KFKLNGRDSLMNPGVAMASHKDDDDRFFPSSGVLIFGKQGAGNDGVDYSQVLITDEEEIKAT
NPVATEEYGAVAINNQAANTQAQTGLVHNQGVIPGMVWQNRDVYLQGPIWAKIPHTDGNF
HPSPLMGGFGLKHPPPQILIKNTPVPADPPLTFNQAKLNSFITQYSTGQVSVEIEWELQKENSK
RWNPEIQYTSNYYKSTNVDFAVNTEGVYSEPRPIGTRYLTRNL
- AAV44.9(E531D) VP3 amino acid sequence
SEQ ID NO: 3
MASGGGAPMADNNEGADGVGNSSGNWHCDSTWLGDRVITTSTRTWALPTYNNHLYKQISN
GTSGGSTNDNTYFGYSTPWGYFDFNRFHCHFSPRDWQRLINNNWGFRPKRLNFKLFNIQVKE
VTTNEGTKTIANNLTSTVQVFTDSEYQLPYVLGSAHQGCLPPFPADVFMVPQYGYLTLNNGS
QALGRSSFYCLEYFPSQMLRTGNNFQFSYTFEDVPFHSSYAHSQSLDRLMNPLIDQYLYYLVR
TQTTGTGGTQTLAFSQAGPSSMASQARNWVPGPSYRQQRVSTTTNQNNNSNFAWTGAAKF
KLNGRDSLMNPGVAMASHKDDDDRFFPSSGVLIFGKQGAGNDGVDYSQVLITDEEEIKATNP
VATEEYGAVAINNQAANTQAQTGLVHNQGVIPGMVWQNRDVYLQGPIWAKIPHTDGNFHP
SPLMGGFGLKHPPPQILIKNTPVPADPPLTFNQAKLNSFITQYSTGQVSVEIEWELQKENSKRW
NPEIQYTSNYYKSTNVDFAVNTEGVYSEPRPIGTRYLTRNL
- AAV44.9(Y446F + T492V + E531D) VP1 amino acid sequence
SEQ ID NO: 18
MAADGYLPDWLEDNLSEGIREWWDLKPGAPKPKANQQKQDDGRGLVLPGYKYLGPFNGL
DKGEPVNAADAAALEHDKAYDQQLKAGDNPYLRYNHADAEFQERLQEDTSFGGNLGRAVF
QAKKRVLEPLGLVEEGAKTAPGKKRPVEQSPQEPDSSSGIGKTGQQPAKKRLNFGQTGDTES
VPDPQPLGEPPAAPSGLGPNTMASGGGAPMADNNEGADGVGNSSGNWHCDSTWLGDRVIT
TSTRTWALPTYNNHLYKQISNGTSGGSTNDNTYFGYSTPWGYFDFNRFHCHFSPRDWQRLIN
NNWGFRPKRLNFKLFNIQVKEVTTNEGTKTIANNLTSTVQVFTDSEYQLPYVLGSAHQGCLP
PFPADVFMVPQYGYLTLNNGSQALGRSSFYCLEYFPSQMLRTGNNFQFSYTFEDVPFHSSYA
HSQSLDRLMNPLIDQYLYFLVRTQTTGTGGTQTLAFSQAGPSSMASQARNWVPGPSYRQQR
VSTVTNQNNNSNFAWTGAAKFKLNGRDSLMNPGVAMASHKDDDDRFFPSSGVLIFGKQGA
GNDGVDYSQVLITDEEEIKATNPVATEEYGAVAINNQAANTQAQTGLVHNQGVIPGMVWQ
NRDVYLQGPIWAKIPHTDGNFHPSPLMGGFGLKHPPPQILIKNTPVPADPPLTFNQAKLNSFIT
QYSTGQVSVEIEWELQKENSKRWNPEIQYTSNYYKSTNVDFAVNTEGVYSEPRPIGTRYLTR
NL
- AAV44.9(E531D) VP1 nucleic acid sequence
SEQ ID NO: 4
ATGGCTGCCGATGGTTATCTTCCAGATTGGCTCGAGGACAACCTCTCTGAGGGCATTCGC
GAGTGGTGGGACTTGAAACCTGGAGCCCCGAAACCCAAAGCCAACCAGCAAAAGCAGG
ACGACGGCCGGGGTCTGGTGCTTCCTGGCTACAAGTACCTCGGACCCTTCAACGGACTCG
ACAAGGGGGAGCCCGTCAACGCGGCGGACGCAGCGGCCCTCGAGCACGACAAGGCCTA
CGACCAGCAGCTCAAAGCGGGTGACAATCCGTACCTGCGGTATAACCACGCCGACGCCG
AGTTTCAGGAGCGTCTGCAAGAAGATACGTCTTTTGGGGGCAACCTCGGGCGAGCAGTC
TTCCAGGCCAAGAAGCGGGTTCTCGAACCTCTCGGTCTGGTTGAGGAAGGCGCTAAGAC
GGCTCCTGGAAAGAAGAGACCGGTAGAGCAGTCACCCCAAGAACCAGACTCCTCATCGG
GCATCGGCAAGACAGGCCAGCAGCCCGCTAAAAAGAGACTCAATTTTGGTCAGACTGGC
GACACAGAGTCAGTCCCCGACCCACAACCTCTCGGAGAACCTCCAGCAGCCCCCTCAGG
TCTGGGACCTAATACAATGGCTTCAGGCGGTGGCGCTCCAATGGCAGACAATAACGAAG
GCGCCGACGGAGTGGGTAATTCCTCGGGAAATTGGCATTGCGATTCCACATGGCTGGGG
GACAGAGTCATCACCACCAGCACCCGAACCTGGGCCCTGCCCACCTACAACAACCACCT
CTACAAGCAAATCTCCAACGGCACCTCGGGAGGAAGCACCAACGACAACACCTACTTTG
GCTACAGCACCCCCTGGGGGTATTTTGACTTCAACAGATTCCACTGCCACTTTTCACCAC
GTGACTGGCAGCGACTCATCAACAACAATTGGGGATTCCGGCCCAAGAGACTCAACTTC
AAGCTCTTCAACATCCAGGTCAAGGAAGTCACGACGAACGAAGGCACCAAGACCATCGC
CAATAATCTCACCAGCACCGTGCAGGTCTTTACGGACTCGGAGTACCAGCTACCGTACGT
GCTAGGATCAGCTCACCAGGGATGTCTGCCTCCGTTCCCGGCGGACGTCTTCATGGTTCC
TCAGTACGGTTATCTAACTCTGAACAATGGCAGCCAGGCCCTGGGACGTTCCTCCTTCTA
CTGCCTGGAGTATTTCCCATCGCAGATGCTGAGAACCGGCAACAACTTTCAGTTCAGCTA
CACCTTCGAGGACGTGCCTTTCCACAGCAGCTACGCGCACAGCCAAAGCCTGGACAGGC
TGATGAATCCCCTCATCGACCAGTACCTGTATTACCTGGTCAGAACGCAGACAACCGGGA
CTGGAGGGACGCAGACTCTGGCATTCAGCCAAGCAGGCCCTAGCTCAATGGCCAGCCAG
GCTAGAAACTGGGTGCCCGGACCGAGCTACCGGCAGCAGCGCGTCTCCACGACAACCAA
CCAGAACAACAACAGCAACTTTGCCTGGACGGGAGCTGCCAAATTTAAACTGAACGGCC
GAGACTCTCTAATGAACCCCGGCGTGGCCATGGCTTCACACAAGGATGACGATGACCGG
TTCTTCCCTTCTAGCGGGGTCCTGATTTTCGGCAAGCAAGGAGCCGGGAATGATGGAGTG
GATTACAGCCAAGTGCTGATTACAGATGAGGAAGAAATCAAGGCTACCAACCCCGTGGC
AACAGAGGAATATGGAGCAGTGGCCATCAACAACCAGGCCGCTAATACGCAGGCGCAG
ACCGGACTCGTGCACAACCAGGGGGTGATTCCCGGCATGGTGTGGCAGAACAGAGACGT
GTACCTGCAGGGTCCCATCTGGGCCAAAATTCCTCACACGGACGGCAACTTTCACCCGTC
TCCCCTGATGGGCGGCTTTGGACTGAAGCACCCGCCTCCTCAAATTCTCATCAAGAACAC
ACCGGTTCCAGCGGACCCGCCGCTTACCTTCAACCAGGCCAAGCTGAACTCTTTCATCAC
GCAGTACAGCACCGGACAGGTCAGCGTGGAAATCGAGTGGGAGCTGCAGAAAGAAAAC
AGCAAACGCTGGAATCCAGAGATTCAGTACACTTCCAACTACTACAAATCTACAAATGT
GGACTTTGCTGTCAACACGGAAGGAGTGTATAGCGAGCCTCGCCCCATTGGCACGCGCT
ACCTCACCCGTAATCTGTAA
- AAV44.9(E531D) VP2 nucleic acid sequence
SEQ ID NO: 5
ACGGCTCCTGGAAAGAAGAGACCGGTAGAGCAGTCACCCCAAGAACCAGACTCCTCATC
GGGCATCGGCAAGACAGGCCAGCAGCCCGCTAAAAAGAGACTCAATTTTGGTCAGACTG
GCGACACAGAGTCAGTCCCCGACCCACAACCTCTCGGAGAACCTCCAGCAGCCCCCTCA
GGTCTGGGACCTAATACAATGGCTTCAGGCGGTGGCGCTCCAATGGCAGACAATAACGA
AGGCGCCGACGGAGTGGGTAATTCCTCGGGAAATTGGCATTGCGATTCCACATGGCTGG
GGGACAGAGTCATCACCACCAGCACCCGAACCTGGGCCCTGCCCACCTACAACAACCAC
CTCTACAAGCAAATCTCCAACGGCACCTCGGGAGGAAGCACCAACGACAACACCTACTT
TGGCTACAGCACCCCCTGGGGGTATTTTGACTTCAACAGATTCCACTGCCACTTTTCACC
ACGTGACTGGCAGCGACTCATCAACAACAATTGGGGATTCCGGCCCAAGAGACTCAACT
TCAAGCTCTTCAACATCCAGGTCAAGGAAGTCACGACGAACGAAGGCACCAAGACCATC
GCCAATAATCTCACCAGCACCGTGCAGGTCTTTACGGACTCGGAGTACCAGCTACCGTAC
GTGCTAGGATCAGCTCACCAGGGATGTCTGCCTCCGTTCCCGGCGGACGTCTTCATGGTT
CCTCAGTACGGTTATCTAACTCTGAACAATGGCAGCCAGGCCCTGGGACGTTCCTCCTTC
TACTGCCTGGAGTATTTCCCATCGCAGATGCTGAGAACCGGCAACAACTTTCAGTTCAGC
TACACCTTCGAGGACGTGCCTTTCCACAGCAGCTACGCGCACAGCCAAAGCCTGGACAG
GCTGATGAATCCCCTCATCGACCAGTACCTGTATTACCTGGTCAGAACGCAGACAACCGG
GACTGGAGGGACGCAGACTCTGGCATTCAGCCAAGCAGGCCCTAGCTCAATGGCCAGCC
AGGCTAGAAACTGGGTGCCCGGACCGAGCTACCGGCAGCAGCGCGTCTCCACGACAACC
AACCAGAACAACAACAGCAACTTTGCCTGGACGGGAGCTGCCAAATTTAAACTGAACGG
CCGAGACTCTCTAATGAACCCCGGCGTGGCCATGGCTTCACACAAGGATGACGATGACC
GGTTCTTCCCTTCTAGCGGGGTCCTGATTTTCGGCAAGCAAGGAGCCGGGAATGATGGAG
TGGATTACAGCCAAGTGCTGATTACAGATGAGGAAGAAATCAAGGCTACCAACCCCGTG
GCAACAGAGGAATATGGAGCAGTGGCCATCAACAACCAGGCCGCTAATACGCAGGCGC
AGACCGGACTCGTGCACAACCAGGGGGTGATTCCCGGCATGGTGTGGCAGAACAGAGAC
GTGTACCTGCAGGGTCCCATCTGGGCCAAAATTCCTCACACGGACGGCAACTTTCACCCG
TCTCCCCTGATGGGCGGCTTTGGACTGAAGCACCCGCCTCCTCAAATTCTCATCAAGAAC
ACACCGGTTCCAGCGGACCCGCCGCTTACCTTCAACCAGGCCAAGCTGAACTCTTTCATC
ACGCAGTACAGCACCGGACAGGTCAGCGTGGAAATCGAGTGGGAGCTGCAGAAAGAAA
ACAGCAAACGCTGGAATCCAGAGATTCAGTACACTTCCAACTACTACAAATCTACAAAT
GTGGACTTTGCTGTCAACACGGAAGGAGTGTATAGCGAGCCTCGCCCCATTGGCACGCG
CTACCTCACCCGTAATCTGTAA
- AAV44.9(E531D) VP3 nucleic acid sequence
SEQ ID NO: 6
ATGGCTTCAGGCGGTGGCGCTCCAATGGCAGACAATAACGAAGGCGCCGACGGAGTGGG
TAATTCCTCGGGAAATTGGCATTGCGATTCCACATGGCTGGGGGACAGAGTCATCACCAC
CAGCACCCGAACCTGGGCCCTGCCCACCTACAACAACCACCTCTACAAGCAAATCTCCA
ACGGCACCTCGGGAGGAAGCACCAACGACAACACCTACTTTGGCTACAGCACCCCCTGG
GGGTATTTTGACTTCAACAGATTCCACTGCCACTTTTCACCACGTGACTGGCAGCGACTC
ATCAACAACAATTGGGGATTCCGGCCCAAGAGACTCAACTTCAAGCTCTTCAACATCCAG
GTCAAGGAAGTCACGACGAACGAAGGCACCAAGACCATCGCCAATAATCTCACCAGCAC
CGTGCAGGTCTTTACGGACTCGGAGTACCAGCTACCGTACGTGCTAGGATCAGCTCACCA
GGGATGTCTGCCTCCGTTCCCGGCGGACGTCTTCATGGTTCCTCAGTACGGTTATCTAACT
CTGAACAATGGCAGCCAGGCCCTGGGACGTTCCTCCTTCTACTGCCTGGAGTATTTCCCA
TCGCAGATGCTGAGAACCGGCAACAACTTTCAGTTCAGCTACACCTTCGAGGACGTGCCT
TTCCACAGCAGCTACGCGCACAGCCAAAGCCTGGACAGGCTGATGAATCCCCTCATCGA
CCAGTACCTGTATTACCTGGTCAGAACGCAGACAACCGGGACTGGAGGGACGCAGACTC
TGGCATTCAGCCAAGCAGGCCCTAGCTCAATGGCCAGCCAGGCTAGAAACTGGGTGCCC
GGACCGAGCTACCGGCAGCAGCGCGTCTCCACGACAACCAACCAGAACAACAACAGCA
ACTTTGCCTGGACGGGAGCTGCCAAATTTAAACTGAACGGCCGAGACTCTCTAATGAACC
CCGGCGTGGCCATGGCTTCACACAAGGATGACGATGACCGGTTCTTCCCTTCTAGCGGGG
TCCTGATTTTCGGCAAGCAAGGAGCCGGGAATGATGGAGTGGATTACAGCCAAGTGCTG
ATTACAGATGAGGAAGAAATCAAGGCTACCAACCCCGTGGCAACAGAGGAATATGGAG
CAGTGGCCATCAACAACCAGGCCGCTAATACGCAGGCGCAGACCGGACTCGTGCACAAC
CAGGGGGTGATTCCCGGCATGGTGTGGCAGAACAGAGACGTGTACCTGCAGGGTCCCAT
CTGGGCCAAAATTCCTCACACGGACGGCAACTTTCACCCGTCTCCCCTGATGGGCGGCTT
TGGACTGAAGCACCCGCCTCCTCAAATTCTCATCAAGAACACACCGGTTCCAGCGGACCC
GCCGCTTACCTTCAACCAGGCCAAGCTGAACTCTTTCATCACGCAGTACAGCACCGGACA
GGTCAGCGTGGAAATCGAGTGGGAGCTGCAGAAAGAAAACAGCAAACGCTGGAATCCA
GAGATTCAGTACACTTCCAACTACTACAAATCTACAAATGTGGACTTTGCTGTCAACACG
GAAGGAGTGTATAGCGAGCCTCGCCCCATTGGCACGCGCTACCTCACCCGTAATCTGTAA

In some embodiments, an AAV-DJ capsid is used in conjunction with the rAAV vectors of the disclosure. AAV-DJ comprises the insertion of 7 amino acids into the HSPG binding domain of the AAV2 capsid and has high expression efficiency in Muller cells following intravitreal injection. In some embodiments, an AAV2(7m8) is used in conjunction with the rAAV vectors of the disclosure. The AAV2(7m8) capsid is closely related to AAV-DJ. In some embodiments, the AAV2/2-MAX capsid comprises five point mutations, Y272F, Y444F, Y500F, Y730F, T491V. In some embodiments, the AAVSHh10 and AAV6(D532N) capsids are derivatives of AAV6. In some embodiments, the AAV6-3pmut is (also known as AAV6(TM6) and AAV6(Y705+Y731F+T492V)).

In some embodiments, the capsid used in conjunction with the disclosed rAAV vectors is a capsid comprising non-native amino acid substitutions at amino acid residues of a wild-type AAV2 capsid. In some embodiments, the non-native amino acid substitutions comprise one or more of Y272F, Y444F, T491V, Y500F, Y700F, Y704F, Y730F or a combination thereof. In some embodiments, the capsids comprise non-native amino acid substitutions at amino acid residues of a wild-type AAV6 capsid as set forth in SEQ ID NO: 6. In some embodiments, the non-native amino acid substitutions comprise one or more of Y445F, Y705F, Y731F, T492V, S663V or a combination thereof. In some embodiments, the capsid comprises AAV2G9, a variant of AAV2.

In some embodiments, the capsid comprises a non-native amino acid substitution at amino acid residue 533 of a wild-type AAV8 capsid. In some embodiments, the non-native amino acid substitution is E533K, Y733F, or a combination thereof. In some embodiments, the capsid comprises AAV7BP2, a variant of AAV8.

In some embodiments, the capsid comprises non-native amino acid substitutions of a wild-type AAV2 capsid. In some embodiments, the capsid comprises one or more of:
(a) Y444F;
(b) Y444F+Y500F+Y730F;
(c) Y272F+Y444F+Y500F+Y730F;
(d) Y444F+Y500F+Y730F+T491V; or
(e) Y272F+Y444F+Y500F+Y730F+T491V, or at equivalent amino acid positions corresponding thereto in any one of the wild-type AAV1, AAV3, AAV4, AAV5, AAV7, AAV9, or AAV10 capsid proteins.

In some embodiments, the capsid comprises non-native amino acid substitutions of a wild-type AAV6 capsid. In some embodiments, the capsid comprises one or more of:
(a) Y445F;
(b) Y705F+Y731F;
(c) T492V;
(d) Y705F+Y731F+T492V;
(e) S663V; or
(f) S663V+T492V.

In various embodiments, the rAAV particles comprise one of the following capsids, i.e., capsid variants of AAV2: DGE-DF (also known as ‘V1V4 VR-V’), P2-V2, P2-V3, and ME-B(Y-F+T-V). The DGE-DF capsid variant contains aspartic acid, glycine, glutamic acid, aspartic acid, and phenylalanine at amino acid positions 492, 493, 494, 499, and 500 of wild-type AAV2 VP1. The P2-V2 capsid variant contains alanine, threonine, proline, aspartic acid, phenylalanine, and aspartic acid at positions 263, 490, 492, 499, 500, and 530 of AAV2 VP1. The P2-V3 capsid variant contains asparagine, alanine, phenylalanine, alanine, asparagine, valine, threonine, arginine, aspartic acid, and aspartic acid at positions 263, 264, 444, 451, 454, 455, 459, 527, 530, and 531 of AAV2 VP1. The ME-B(Y-F+T-V) capsid variant contains aspartic acid, glycine, glutamic acid, aspartic acid, and phenylalanine at positions 492, 493, 494, 499, and 500 of AAV2 VP1, respectively, SAAGADXAXDS at positions 546-556 of AAV2 VP1, and the following substitutions: Y272F, Y444F, and T491V. In some embodiments, the rAAV particles comprise a capsid selected from AAV6(3pMut), AAV2(quadYF+T-V), or AAV2(trpYF). In some embodiments, the rAAV particles comprise any of the capsid variants described in International Patent Publication No. WO 2018/156654, which is incorporated by reference herein.

In some embodiments, the AAV particles comprise one or more capsids from AAV2, e.g., AAV2(4pMut)ΔHS capsid. Example capsid sequences (SEQ ID NOs: 36-38) for AAV2(4pMut)ΔHS are provided below.

- AAV2(4pMut)ΔHS VP1 amino acid sequence
SEQ ID NO: 36
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYFLSRTNT
PSGTTTQSRLQFSQAGASDIRDQSRNWLPGPCYRQQGVSKVSADNNNSEF
SWTGATKYHLNGRDSLVNPGPAMASHKDDEEKFFPQSGVLIFGKQGSEKT
NVDIEKVMITDEEEIRTTNPVATEQYGSVSTNLQSGNTQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRFLTRNL
- AAV2(4pMut)ΔHS VP2 amino acid sequence
SEQ ID NO: 37
TAPGKKRPVEHSPVEPDSSSGTGKAGQQPARKRLNFGQTGDADSVPDPQP
LGQPPAAPSGLGTNTMATGSGAPMADNNEGADGVGNSSGNWHCDSTWMGD
RVITTSTRTWALPTYNNHLYKQISSQSGASNDNHYFGYSTPWGYFDFNRF
HCHFSPRDWQRLINNNWGFRPKRLNFKLFNIQVKEVTQNDGTTTIANNLT
STVQVFTDSEYQLPYVLGSAHQGCLPPFPADVFMVPQYGYLTLNNGSQAV
GRSSFYCLEYFPSQMLRTGNNFTFSYTFEDVPFHSSYAHSQSLDRLMNPL
IDQYLYFLSRTNTPSGTTTQSRLQFSQAGASDIRDQSRNWLPGPCYRQQG
VSKVSADNNNSEFSWTGATKYHLNGRDSLVNPGPAMASHKDDEEKFFPQS
GVLIFGKQGSEKTNVDIEKVMITDEEEIRTTNPVATEQYGSVSTNLQSGN
TQAATADVNTQGVLPGMVWQDRDVYLQGPIWAKIPHTDGHFHPNPEIQYT
SNYNKSVNVDFTVDTNGVYSEPRPIGTRFLTRNL
- AAV2(4pMut)ΔHS VP3 amino acid sequence
SEQ ID NO: 38
MATGSGAPMADNNEGADGVGNSSGNWHCDSTWMGDRVITTSTRTWALPTY
NNHLYKQISSQSGASNDNHYFGYSTPWGYFDFNRFHCHFSPRDWQRLINN
NWGFRPKRLNFKLFNIQVKEVTQNDGTTTIANNLTSTVQVFTDSEYQLPY
VLGSAHQGCLPPFPADVFMVPQYGYLTLNNGSQAVGRSSFYCLEYFPSQM
LRTGNNFTFSYTFEDVPFHSSYAHSQSLDRLMNPLIDQYLYFLSRTNTPS
GTTTQSRLQFSQAGASDIRDQSRNWLPGPCYRQQGVSKVSADNNNSEFSW
TGATKYHLNGRDSLVNPGPAMASHKDDEEKFFPQSGVLIFGKQGSEKTNV
DIEKVMITDEEEIRTTNPVATEQYGSVSTNLQSGNTQAATADVNTQGVLP
GMVWQDRDVYLQGPIWAKIPHTDGHFHPSPLMGGFGLKHPPPQILIKNTP
VPANPSTTFSAAKFASFITQYSTGQVSVEIEWELQKENSKRWNPEIQYTS
NYNKSVNVDFTVDTNGVYSEPRPIGTRFLTRNL

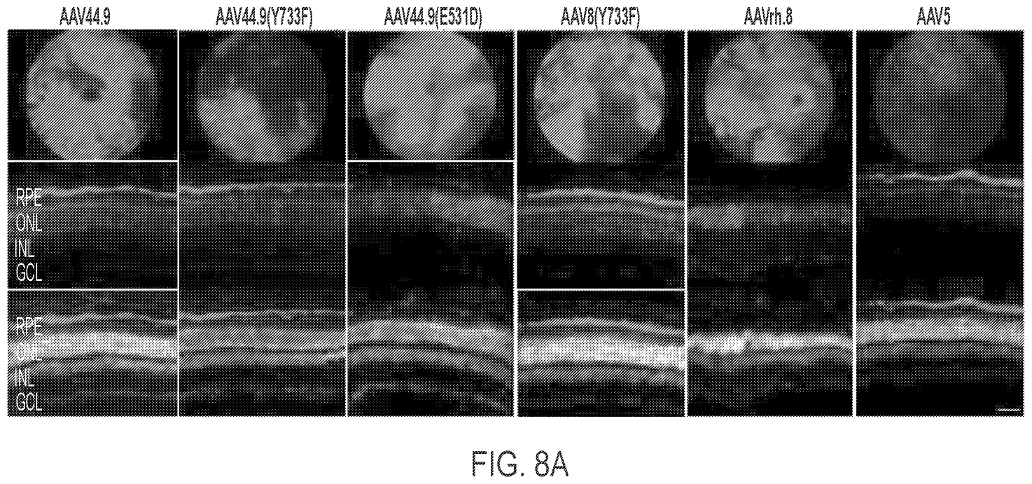

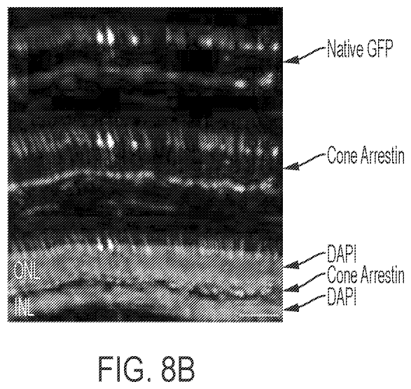

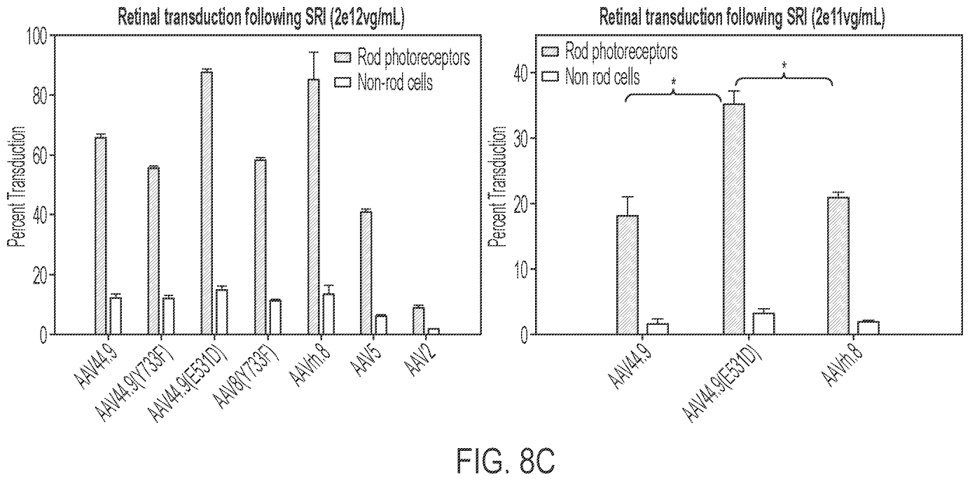

As shown in FIGS. 8A-8C, the transduction profile of subretinally injected rAAV vectors in Nrl-GFP mice retina at 4 and 6 weeks post-injection was evaluated. Flow cytometry was used to determine transduction efficiencies. As shown in FIG. 8C, cector-mediated GFP expression and cone arrestin colocalized, indicating that the evaluated AAV44.9, AAV44.9(Y731F), AAV44.9(E531D), AAVrh.8 and AAV8(Y733F) capsids efficiently transduces cone photoreceptors.

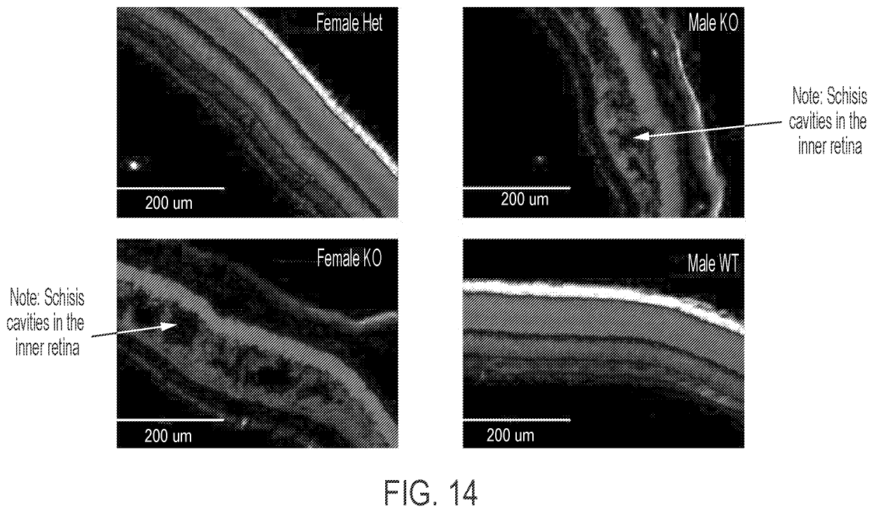

OCT measurements and schisis activity scores associated with the natural history study were evaluated. 1 month old mouse retinal sections stained with monoclonal antibody to RS1 (shaded), as shown in FIG. 14. RS1 expression localized to the inner/outer segment junction of photoreceptors in the female heterozygous and wildtype male mice and is absent in male and female RS1 KO mice. Schisis cavities were observed in the inner retina.

### Vectors

Recombinant AAV vectors are described herein. In some embodiments, the vector described herein is a self-complementary rAAV (scAAV) vector. In some embodiments, the vector is a single-stranded (ss) vector. In some embodiments, the vector is provided to the one or both eyes by one or more administrations of an infectious adeno-associated viral particle, an rAAV virion, or a plurality of infectious rAAV particles in an amount and for a time sufficient to treat or ameliorate one or more symptoms of the disease or condition being treated.

In some aspects, a method for providing a mammal in need thereof with a therapeutically-effective amount of a selected therapeutic agent (e.g., a synthetic human RS1) is described herein. In some embodiments, the therapeutic agent is encoded in a heterologous nucleic acid, or transgene, that is inserted into a recombinant AAV nucleic acid vector. In some embodiments, the nucleic acid vector comprises one or more heterologous nucleic acids comprising a sequence encoding a protein or polypeptide of interest operably linked to a promoter (e.g., an hGRK1 promoter), wherein the one or more transgenes are flanked on each side with an ITR sequence. The disclosed nucleic acid vectors may comprise AAV inverted terminal repeats flanking a polynucleotide comprising the RS1 heterologous nucleic acid (transgene) and other regulatory elements. In some embodiments, the disclosed nucleic acid vectors comprise AAV ITRs flanking a polynucleotide comprising the RS1 heterologous nucleic acid, SV40 intron, WPREsf element, and polyA signal sequence. In some embodiments, the vectors comprise AAV ITRs flanking a polynucleotide comprising the RS1 heterologous nucleic acid, SV40 intron, WPREsf element, stuff sequence, and polyA sequence.

The ITR sequences can be derived from any AAV serotype (e.g., 1, 2, 3, 4, 5, 6, 7, 8, 9, or 10) or can be derived from more than one serotype. In some embodiments, the ITR sequences of the first serotype are derived from AAV2, AAV5, AAV7 AAV8, or AAV9. In some embodiments, the ITR sequences are derived from AAV2 or AAV5. In some embodiments, the ITR sequences are the same serotype as the capsid (e.g., AAV5 ITR sequences and AAV5 capsid, etc.).

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. In some embodiments, the nucleic acid vector comprises a pTR-UF-11 plasmid backbone, which is a plasmid that contains AAV2 ITRs. This plasmid is commercially available from the American Type Culture Collection (ATCC MBA-331).

In some aspects, the rAAV vectors described herein may comprise multiple (two, three, four, five, six, seven, eight, nine, or ten) heterologous nucleic acids. In certain embodiments, the multiple heterologous nucleic acids are comprised on a single polynucleotide molecule. Multiple heterologous nucleic acids may be used, for example, to correct or ameliorate a gene defect caused by a multi-subunit protein. In various embodiments, a different heterologous nucleic acid may be used to encode each subunit of a protein, or to encode different peptides or proteins. This is desirable when the size of the nucleic acid encoding the protein subunit is large, e.g., for an immunoglobulin, the platelet-derived growth factor, or a dystrophin protein. In order for the cell to produce the multi-subunit protein, a cell is infected with the rAAV particle containing each of the different subunits. Alternatively, different subunits of a protein may be encoded by the same nucleic acid sequence. In various embodiments, a single heterologous nucleic acid includes the nucleic acid encoding each of the subunits, with the nucleic acid for each subunit separated by an internal ribozyme entry site (IRES). This is desirable when the size of the nucleic acid encoding each of the subunits is small, e.g., the total size of the nucleic acid encoding the subunits and the IRES is less than five kilobases.

As an alternative to an IRES, the nucleic acid may be separated by sequences encoding a 2A peptide, which self-cleaves in a post-translational event. This 2A peptide is significantly smaller than an IRES, making it well suited for use when space is a limiting factor. More often, when the heterologous nucleic acid is large, consists of multi-subunits, or two heterologous nucleic acids are co-delivered, or rAAV particle carrying the desired heterologous nucleic acid(s) or subunits are co-administered to allow them to concatamerize in vivo to form a single vector genome. In such an embodiment, a first rAAV particle may carry an expression cassette which expresses a single heterologous nucleic acid and a second rAAV particle may carry an expression cassette which expresses a different heterologous nucleic acid for co-expression in the host cell. However, the selected heterologous nucleic acid may encode any biologically active product or other product, e.g., a product desirable for study.

In some aspects, the rAAV vectors may be codon-optimized for human expression. In some embodiments, the rAAV vectors may have modified Kozak nucleic acid sequences that provide for enhanced transduction or fitness in the target cell, e.g., a PR cell. Kozak sequences include the translation initiation codon (ATG) and a stretch of nucleotides positioned 5′ of the initiation codon.

### Methods of Treatment and Formulations

In some embodiments, methods are provided involving providing a mammal in need thereof with a therapeutically effective amount of a selected therapeutic agent, the method comprising administering to one or both eyes of the mammal, an amount of the rAAV particles described herein; and for a time effective to provide the mammal with a therapeutically-effective amount of the selected therapeutic agent.

In certain embodiments, the mammal is suspected of having, is at risk for developing, or has been diagnosed with X-linked retinoschischis.

In some embodiments, methods are provided for transducing a mammalian photoreceptor cell or retinal pigment epithelium cell, the method comprising administering to one or both eyes of a mammal the rAAV particles described herein. In particular embodiments, methods are provided for expressing a polynucleotide in one or more photoreceptor cells of a mammal, the method comprising subretinally or intravitreally administering to one or both eyes of the mammal the rAAV particles described herein, or compositions thereof, wherein the rAAV particle comprises a polynucleotide comprising at least a first polynucleotide that comprises a PR- or an RPE-cell-specific promoter operably linked to at least a first hetereologous nucleic acid sequence that encodes a therapeutic agent, for a time effective to produce the therapeutic agent in the one or more PR or RPE cells of the mammal.

In particular embodiments, a replacement coding sequence is administered to the subject to provide a functional protein, e.g., RS1 protein, to restore, e.g., completely or partially, photoreceptor function to a subject (e.g., a human). In some embodiments, one or both alleles of a target coding sequence of the subject are silenced by administering an rAAV particle comprising a heterologous nucleic acid disclosed herein to the subject (e.g., to a human having dominant cone-rod dystrophy). In particular embodiments, the endogenous mutant alleles of one or more target coding sequences are silenced or suppressed by administering an rAAV particle disclosed herein.

In some embodiments, the mammal is a human subject. In some embodiments, the mammal is a non-human primate subject. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. Other exemplary subjects include domesticated animals such as dogs and cats; livestock such as horses, cattle, pigs, sheep, goats, and chickens; and other animals such as mice, rats, guinea pigs, and hamsters.

In certain embodiments, methods are provided for subretinally administering to a fovea (e.g., foveal cone cells) of the mammal the rAAV particles described herein or compositions thereof. In particular embodiments, detachment of the fovea is minimized during and/or after subretinal administration. In particular embodiments, subretinal administration of the rAAV particle is performed in the absence of any detachment of the fovea.

In some embodiments, the dose of rAAV particles administered to a cell or a subject may be on the order ranging from 10⁶ to 10¹⁴ vgs/mL (or particles/mL) or 10³ to 10¹⁵ vgs/mL, or any values therebetween for either range, such as for example, about 10⁶, 10⁷, 10⁸, 10⁹, 10¹⁰, 10¹¹, 10¹², 10¹³, or 10¹⁴ vgs/mL. In particular embodiments, rAAV particles in an amount of 5×10¹⁰ vgs/mL are be administered. In some embodiments, rAAV particles in an amount of 1×10¹¹ vgs/mL are be administered. In some embodiments, rAAV particles in an amount of 1×10¹² vgs/mL are be administered. In some embodiments, the dose of rAAV particles administered to a subject may be on the order ranging from 10⁶ to 10¹⁴ vgs/mL, or 10³ to 10¹⁵ vgs/mL, or any values therebetween for either range, such as for example, about 10⁶, 10⁷, 10⁸, 10⁹, 10¹⁰, 10¹¹, 10¹², 10¹³, or 10¹⁴ vgs/mL. In some embodiments, rAAV particles of higher than 10¹³ vgs/mL are be administered. The rAAV particles can be administered as a single dose, or divided into two or more administrations as may be required to achieve therapy of the particular disease, disorder or condition being treated.

In particular embodiments, rAAV particle titers range from 5×10¹⁰-1×10¹² vg/ml. In some embodiments, rAAV particle titers can be 1×10¹⁰, 2.5×10¹⁰, 5×10¹⁰, 1×10¹¹, 2.5×10¹¹, 5×10¹¹, 1×10¹², 2.5×10¹², 5×10¹², 1×10¹³, 2.5×10¹³, or 5×10¹³ vg/mL. In some embodiments, particle titers are less than 5×10¹⁰ vg/mL. In some embodiments, rAAV particles are administered via methods described herein (e.g., subretinally or intravitreally). In particular embodiments, particles are administered subretinally.

The rAAV particles can be administered as a single dose, or divided into two or more administrations (e.g., up to three subretinal injection blebs) as may be required to achieve therapy of the particular disease, disorder or condition being treated. In some embodiments, from 1 to 500 microliters of a composition (e.g., comprising an rAAV particle) described in this application is administered to one or both eyes of a subject. For example, in some embodiments, about 10, about 30, about 45, about 50, about 75, about 90, about 100, about 200 microliters can be administered to each eye. In particular embodiments, 3 subretinal injections ranging in bleb volumes of 10 μL to 100 μL each may be administered. However, it should be appreciated that smaller or larger volumes could be administered in some embodiments.

In some embodiments, the disclosure provides formulations of one or more rAAV-based compositions disclosed herein in pharmaceutically acceptable solutions for administration to a cell or an animal, either alone or in combination with one or more other modalities of therapy, and in particular, for therapy of human cells, tissues, and diseases affecting man.

The rAAV particles described herein may be administered in combination with other agents as well, such as, e.g., proteins or polypeptides or various pharmaceutically-active agents, including one or more systemic or topical administrations of therapeutic polypeptides, biologically active fragments, or variants thereof. In particular embodiments, the described rAAV particles may be administered in combination with one or more carbonic anhydrase inhibitors (CAIs). In some embodiments, they may be co-administered with any of the CAIs acetazolamide, dichlorphenamide (also known as diclofenamide), methazolamide, dorzolamide, brinzolamide, ethoxzolamide, and zonisamide. There is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized as described herein.

In some embodiments, described herein are uses of the described rAAV vectors, viral particles, compositions, and (host) cells described herein in the preparation of medicaments for diagnosing, preventing, treating or ameliorating at least one or more symptoms of XLRS. In some embodiments, the methods comprise direct administration to the vitreous of one or both eyes of a mammal in need thereof, one or more of the described vectors, viral particles, cells, compositions, or pluralities thereof, in an amount and for a time sufficient to diagnose, prevent, treat, or lessen one or more symptoms of such a disease, dysfunction, disorder, abnormal condition, deficiency, injury, or trauma in one or both eyes of the affected mammal. In various embodiments, the mammal is a human subject.

In some embodiments, described herein are compositions useful in treating XLRS in the preparation of medicaments to treat XLRS, comprising one or more of the described rAAV vectors, particles, compositions, and host cells. In some embodiments, the compositions comprise at least a first pharmaceutically-acceptable excipient for use in the manufacture of medicaments and methods involving therapeutic administration of such rAAV particles or vectors. In some embodiments, pharmaceutical formulations are suitable for intravitreal administration into one or both eyes of a human or other mammal.

In some embodiments, described herein are methods and uses of the described rAAV vectors and compositions for treating or ameliorating the symptoms of XLRS in human photoreceptors or RPE cells. In some embodiments, the disclosed methods and uses comprise intravitreal or subretinal administration to one or both eyes of a subject in need thereof, one or more of the described particles vectors, particles, host cells, or compositions, in an amount and for a time sufficient to treat or ameliorate the symptoms of such a deficiency in the affected mammal. In some embodiments, the methods comprise prophylactic treatment of an animals suspected of having such conditions, or administration of such compositions to those animals at risk for developing such conditions either following diagnosis, or prior to the onset of symptoms.

### Pharmaceutical Compositions and Cells

In some aspects, the disclosure contemplates host cells that comprise a particle that incorporates an AAV44.9(E531D) capsid, a nucleic acid encoding a AAV44.9(E531D) capsid or an rAAV particle as described herein. Such host cells include mammalian host cells, with human host cells being preferred, and may be isolated, e.g., in cell or tissue culture. In some embodiments, the host cell is a cell of the eye.

In some embodiments, a composition is provided which comprises an rAAV particle as described herein (e.g., comprising a AAV44.9(E531D) capsid) and optionally a pharmaceutically acceptable carrier, excipient, diluent and/or buffer. In some embodiments, the compositions described herein can be administered to a mammal (or subject) in need of treatment. In some embodiments, the subject has or is suspected of having retinoschisis. In some embodiments, the subject has one or more endogenous mutant alleles (e.g., associated with or that cause a disease, disorder or condition of the eye or retina, such as retinoschisis).

Formulation of pharmaceutically-acceptable excipients and carrier solutions is well-known to those of skill in the art, as is the development of suitable dosing and treatment regimens for using the particular compositions described herein in a variety of treatment regimens, including e.g., subretinal, intravitreal, parenteral, intravenous, intranasal, intra-articular, and intramuscular administration and formulation.

Typically, these formulations may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver an rAAV particle as described herein (e.g., comprising a AAV44.9(E531D) capsid) in suitably formulated pharmaceutical compositions disclosed herein either subretinally, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of compositions (e.g., comprising an rAAV particle as described herein) suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The term “carrier” refers to a diluent, adjuvant, excipient, or vehicle with which the rAAV particle as described herein is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

The compositions of the present disclosure can be delivered to the eye through a variety of routes. They may be delivered intraocularly, by topical application to the eye or by intraocular injection into, for example the vitreous (intravitreal injection) or subretinal (subretinal injection) inter-photoreceptor space. In some embodiments, they are delivered to rod photoreceptor cells. Alternatively, they may be delivered locally by insertion or injection into the tissue surrounding the eye. They may be delivered systemically through an oral route or by subcutaneous, intravenous or intramuscular injection. Alternatively, they may be delivered by means of a catheter or by means of an implant, wherein such an implant is made of a porous, non-porous or gelatinous material, including membranes such as silastic membranes or fibers, biodegradable polymers, or proteinaceous material. They can be administered prior to the onset of the condition, to prevent its occurrence, for example, during surgery on the eye, or immediately after the onset of the pathological condition or during the occurrence of an acute or protracted condition.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions may be prepared by incorporating an rAAV particle as described herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of composition (e.g., comprising an rAAV particle as described herein) and time of administration of such composition will be within the purview of the skilled artisan having benefit of the present teachings. It is likely, however, that the administration of therapeutically-effective amounts of the disclosed compositions may be achieved by a single administration, such as for example, a single injection of sufficient numbers of rAAV particles to provide therapeutic benefit to the patient undergoing such treatment. Alternatively, in some circumstances, it may be desirable to provide multiple, or successive administrations of the composition, either over a relatively short, or a relatively prolonged period of time, as may be determined by the medical practitioner overseeing the administration of such compositions.

In some embodiments, visual acuity can be maintained or restored (e.g., partially or completely) after administering one or more compositions described in this application. In some embodiments, one or more photoreceptor cells or one or more RPE cells may be preserved, partially or completely, and/or one or more rod- and/or cone-mediated functions may be restored, partially or completely, after administering one or more compositions described in this application.

To “treat” a disease as the term is used herein, means to reduce the frequency or severity of at least one sign or symptom of a disease, disorder or condition experienced by a subject (e.g., cone-rod dystrophy). The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

Methods of Producing rAAV Particles

Methods for producing and using pseudotyped rAAV particles are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001). Methods of producing rAAV particles and heterologous nucleic acids are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US 2007/0015238 and US 2012/0322861, which are incorporated herein by reference in their entireties; and plasmids and kits available from ATCC and Cell Biolabs, Inc.). For example, a plasmid containing the heterologous nucleic acid may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected or permanently integrated into a producer cell line such that the rAAV particle may be packaged and subsequently purified.

In some embodiments, the one or more helper plasmids include a first helper plasmid comprising a rep gene and a cap gene (e.g., encoding a rAAV capsid protein as described herein) and a second helper plasmid comprising a Ela gene, a E1b gene, a E4 gene, a E2a gene, and a VA gene. In some embodiments, the rep gene is a rep gene derived from AAV2 and the cap gene is derived from AAV44.9 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the heterologous nucleic acid sequence. As a further alternative, in another example HEK293 or BHK cell lines are infected with a HSV containing the heterologous nucleic acid sequence and optionally one or more helper HSVs containing rep and cap ORFs as described herein and the adenoviral VA, E2A (DBP), and E4 genes under the transcriptional control of their native promoters. The HEK293, BHK, or Sf9 cells may then incubated for at least 60 hours to allow for rAAV particle production. The rAAV particles can then be purified using any method known the art or described herein, e.g., by iodixanol step gradient, CsCl gradient, chromatography, polyethylene glycol (PEG) precipitation, and/or affinity capture.

In various embodiments, an iodixanol step gradient purification method is used. Vectors may be packaged into mammalian cells (e.g., HEK293T cells) and purified by iodixanol gradient centrifugation, followed by buffer exchange and concentration into BSS/Tween buffer. An affinity capture step may be added.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus may be considered to constitute preferred modes for its practice. However, those of skill in the art should, in light of the present disclosure, appreciate that many changes may be made in the specific embodiments which are disclosed and still obtain a like or similar result without departing from the spirit and scope of the invention.

### Example 1—Enhanced Lateral Spread and Foveal Transduction Following Subretinal Administration of an AAV Vector Encoding a Reporter Gene in Macaque

An evaluation of the performance of vectors and particles incorporating the AAV44.9(E531D) capsid variant in subretinally injected mice and macaques relative to benchmark vectors, the closely related AAVrh.8, and unmodified AAV44.9, was pursued. It was determined that the human rhodopsin kinase (hGRK1) promoter has exclusive activity in non-human primate rods and cones. As such, the hGRK1 promoter was evaluated for its ability to drive green fluorescent protein (GFP) reporter expression in macaque eyes in the improved AAV44.9(E531D) vector. The degree of lateral spread from the initial bleb boundaries was also evaluated.

The parafoveal region is the zone of the eye that circumscribes the fovea, approximately 4 degrees eccentricity from the central fixation point. The parafovea has the highest density of rods, while still also containing a large number of cones. It is a transitional zone between cone- and rod-dominant retina and is important in the context of diseases where degeneration proceeds from the outer to inner retina, such as retinitis pigmentosa (RP). The perifoveal region is the zone that circumscribes the parafovea, and represents the outermost band of the macula. Like the parafovea, the periovea has an important role in progression of diseases like RP, where retinal degeration starts in the periphery and progresses to the central retina. The perifovea is the first zone of the macula to undergo degeneration in RP.

Two rAAV vectors—AAV44.9-hGRK1-GFP and AAV44.9(E531D)-hGRK1-GFP—were subretinally administered to macaque eyes. Vectors were delivered at concentrations of 1×10¹² vg/mL. A control vector, AAV5-hGRK1-GFP, was also administered to the eyes.

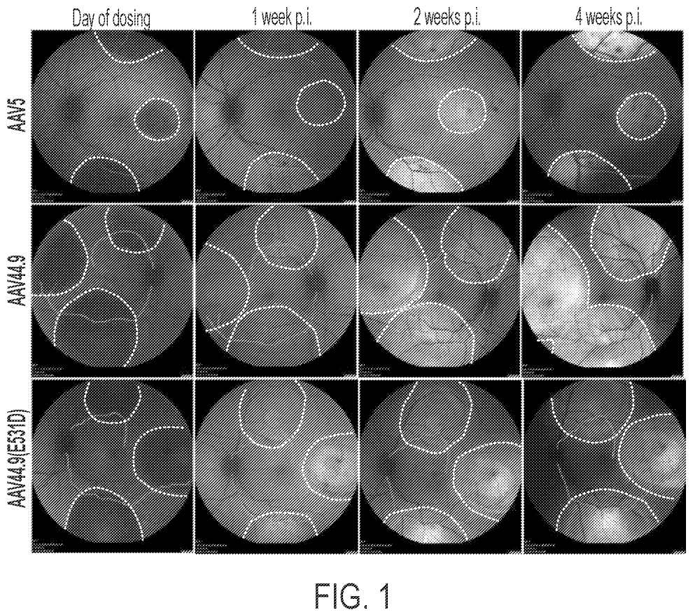

Particles incorporating both modified and unmodified AAV44.9 vectors exhibited enhanced lateral spread and potency in subretinally injected macaque subjects (see FIG. 1). Initial boundaries of the bleb and boundaries of resulting GFP expression are outlined in white dotted line in FIG. 1. Identical vasculature is highlighted in thickened dark line for reference. GFP expression mediated by AAV44.9(E531D) was visible at 1 week post injection. Both AAV44.9 and AAV44.9(E531D) were well tolerated in the primate retina at the 1×10¹² vg/mL dose. The control vector, AAV5, mediated GFP restriction that remained sequestered within the original injection bleb.

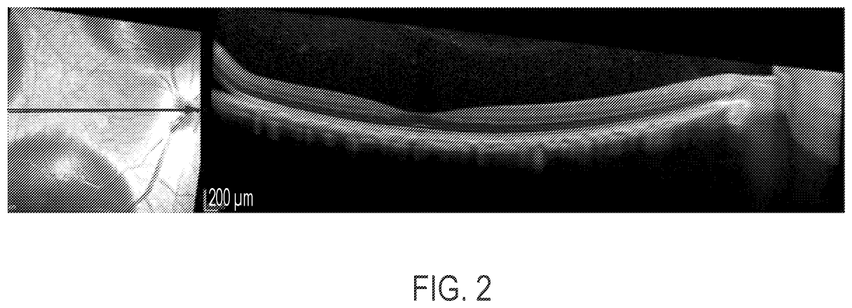

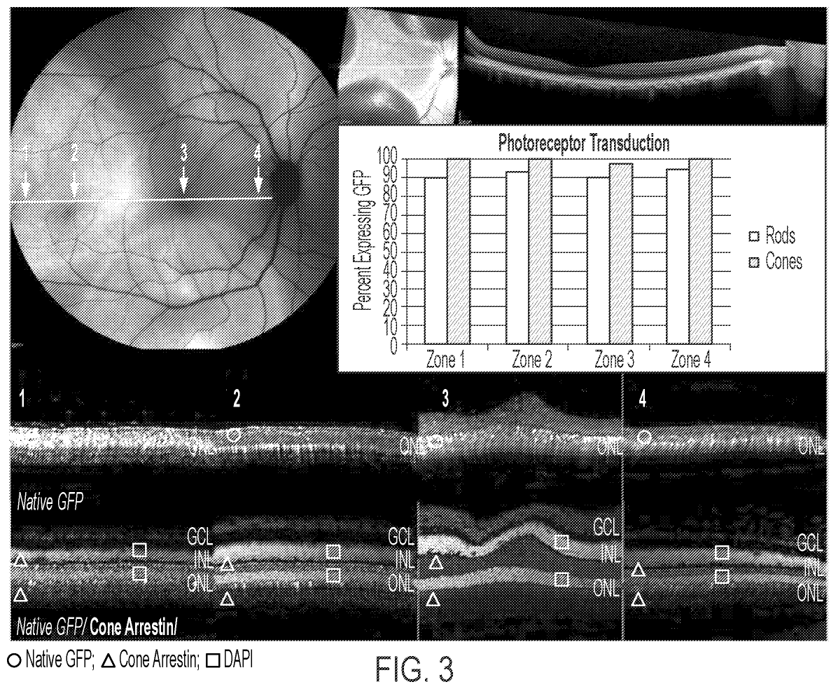

An extrafoveal subretinal injection of AAV44.9-hGRK1-GFP (concentration of 1×10¹² vg/mL) were performed in macaque subjects. Optical coherence tomography (OCT) scans revealed that the fovea was not detached during the injection (FIG. 2). This extrafoveal subretinal injection transduced 98% of foveal cones even and 100% of central rods, even though the fovea did not detach (see top right of FIG. 3). The capsid exhibited enhanced lateral spread, as bleb boundaries were expanded relative to the initial boundaries.

Images were also captured from macaque eyes injected with AAV44.9(E531D)-hGRK1-GFP. A qualitative analysis in a single eye revealed ˜50% of foveal cone transduction mediated by AAV44.9(E531D).

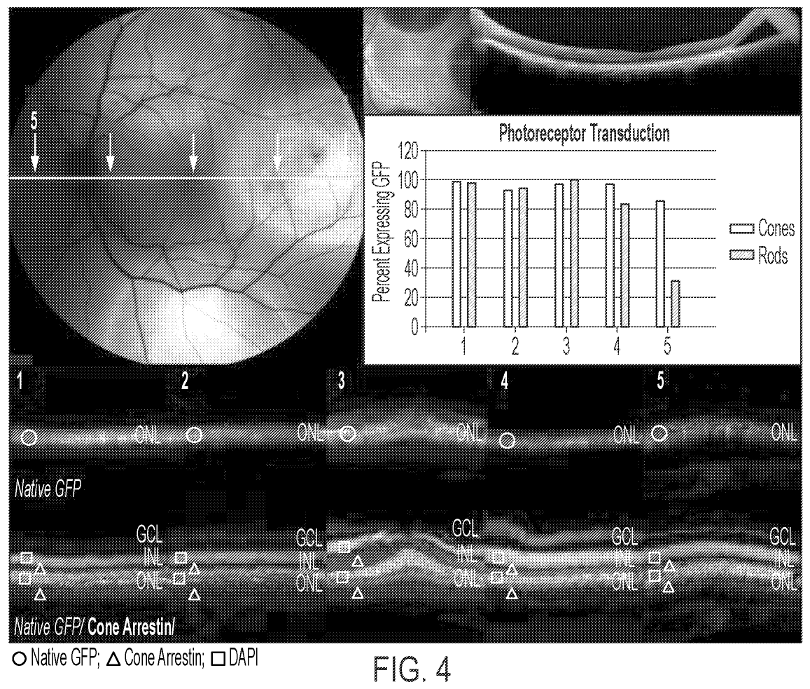

Three subretinal injections totalin a volume of 90 μL (30 μL each) of AAV44.9(E531D)-hGRK1-GFP were performed in the superior, temporal, and inferior retina outside the fovea of macaque eyes. Retinal sections were stained with an antibody directed against cone arrestin and three blinded observers counted the number of GFP positive cones and rods in 5 retinal regions across a single plane traversing the foveal pit. Results of this administration are shown in FIG. 4. OCT scans revealed that the fovea was not detached during the injection (see right panel of FIG. 4).

These results indicate that extrafoveal subretinal injection in macaque of AAV44.9(E531D)-hGRK1-GFP exhibited remarkable transduction of central cone and rod cells in the absence of foveal detachment. Peripheral rods and cones were also transduced very efficiently. Accordingly, extrafoveal subretinal injection resulted in highly efficient transduction across the foveal region.

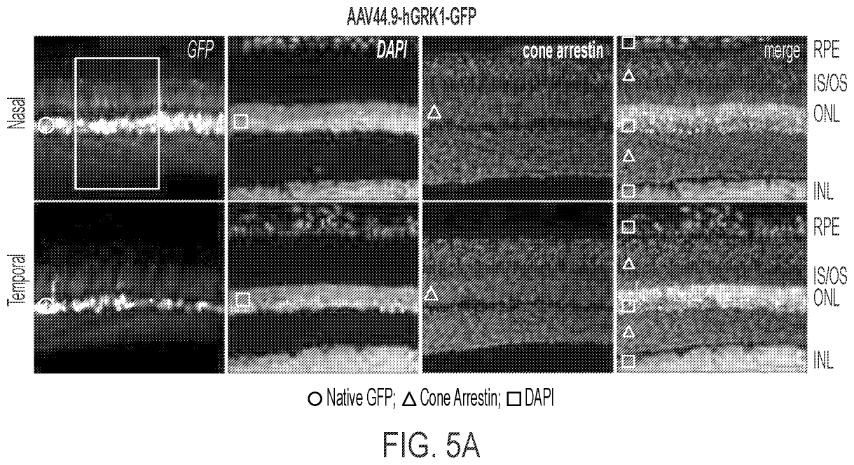

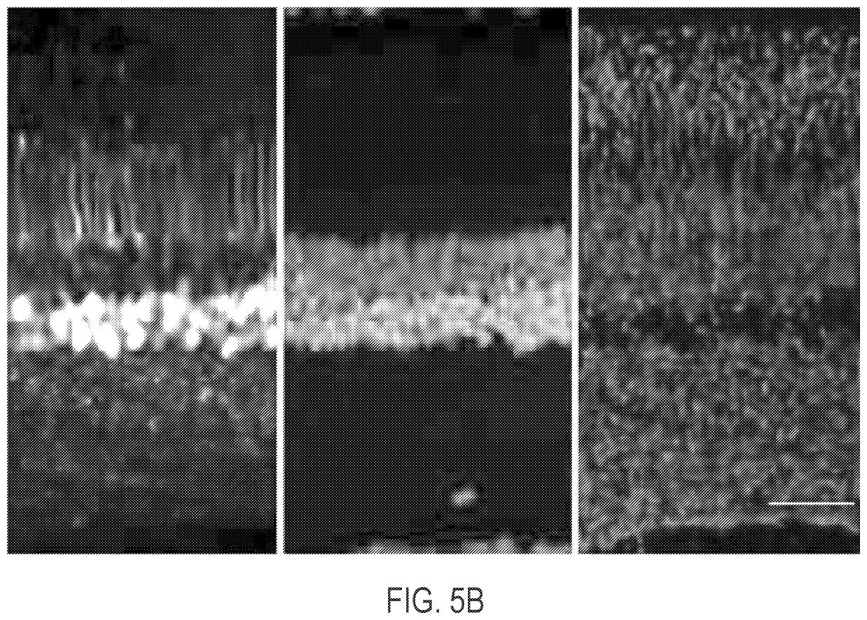

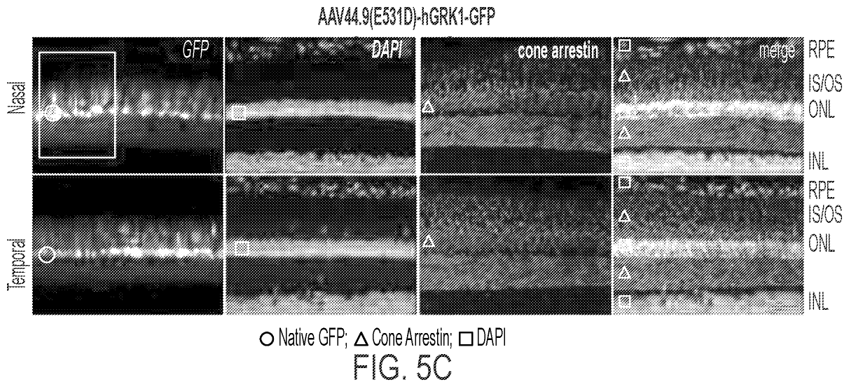

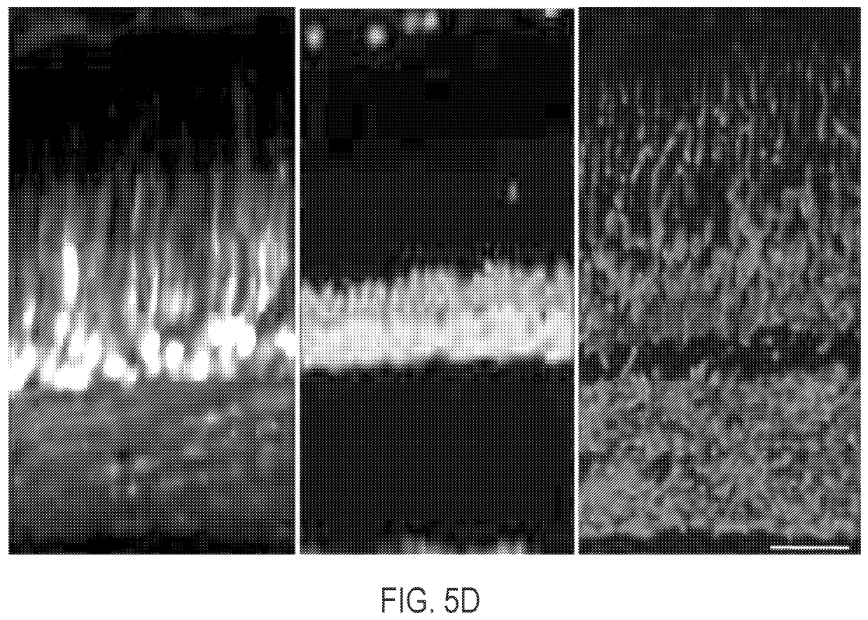

As shown in FIGS. 5A-5D, an examination of the parafovea following this injection revealed that the AAV44.9(E531D) particles transduced parafoveal cones located both nasal and temporal to the foveal pit. Notably, however, parafoveal cone transduction was not achieved with unmodified AAV44.9. This finding is of interest at least because i) parafoveal cones are refractory to transduction by a variety of AAV capsid variants, and ii) the earliest loss of structure and function due to aging and inherited retinal disease often occurs in the parafoveal region. Despite the dissimilarity in their ability to transduce cones in this region, the modified and unmodified AAV44.9 vectors efficiently transduced parafoveal rods to a substantially equal degree.

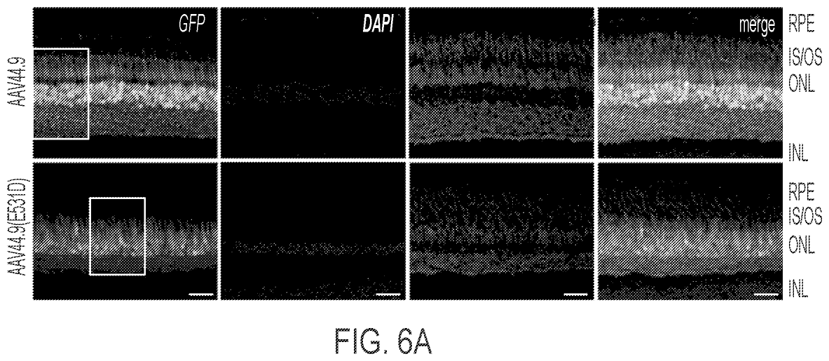

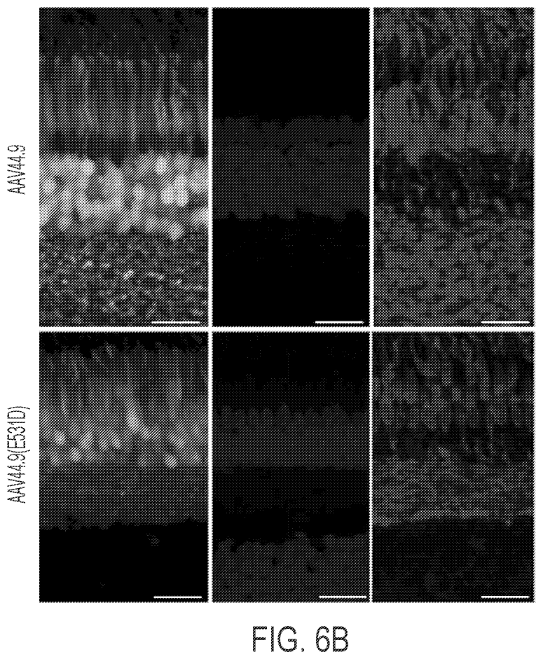

As shown in FIGS. 6A and 6B, an examination of the perifovea revealed a similar pattern: particles that incorporated the AAV44.9(E531D) capsid transduced perifoveal cones, but unmodified AAV44.9 did not. Perifoveal rods were efficiently transduced by both capsids in this region. The perifovea circumscribes the parafovea.

These results demonstrate that the enhanced lateral spread of transduction provided by the improved AAV44.9(E531D) capsid variant vectors may allow subretinal injection in the parafoveal region to produce transduction of the foveal cells while circumventing the deleterious effects of inducing a foveal detachment in human subjects. They further demonstrate that enhanced lateral spread and transduction may be achieved with a total injection volume of AAV particles of as low as 90 μL (3 injection blebs of 30 μL each).

### Materials and Methods

### Vector Production

A self-complementary AAV construct containing the truncated chimeric CMV-Chicken Beta Actin (smCBA) promoter driving mCherry (sc-smCBA-mCherry) was packaged into AAV44.9, AAV44.9(Y731F), AAV44.9(E531D), AAV5 and AAV8(Y733F) using a triple transfection-plasmid based system in adherent HEK293T seeded in double-stack cell factories (1,272 cm² cell growth area). Cells were harvested and lysed by successive freeze thaw cycles. Virus within the lysate was purified by iodixanol density gradient and was buffer exchanged into Alcon BSS supplemented with Tween 20 (0.014%). Virus was titered by qPCR relative to a standard and stored at −80 C. Addition of Y731F and E531D substitutions to the AAV44.9 capsid were accomplished by site-directed mutagenesis of the AAV2rep-44.9cap plasmid and confirmed by Sanger sequencing. An additional construct containing the cone-specific, IRBPe-GNAT2 chimeric promoter driving green fluorescent protein (GFP) was packaged in the AAV44.9 and AAV44.9 variant vectors.

### In-Vitro Transduction Assay

ARPE-19 (human retinal pigment epithelial cell line) and 661W (mouse cone cell line) cells were seeded in 96 well plates at a concentration of 1.0×10⁴ cells/well. The following day, cells were infected at 10,000 p/cell. Three days post-infection, fluorescent microscopy at a fixed exposure was performed, cells were detached and flow-cytometry was used to quantify reporter protein expression (mCherry) via fluorescence. mCherry expression was calculated by multiplying the mean mCherry fluorescence times the number of positive cells. Graphs represent expression levels minus the level of cells only.

### Injection

2×10⁹ vg in 1 μL of vector containing solution was delivered either intravitreally or subretinally to macaque retinas. A minimum of 6 eyes receiving successful injections were analyzed in each experiment.

### Fundoscopy

At 4 weeks post-injection, fundoscopy was performed using a Micron III camera (Phoenix Research Laboratories, Pleasanton, Calif.). Bright field and red fluorescent images were taken to visualize retinal health and mCherry expression, respectively. Exposure settings were constant between experiments and are indicated in the figure legends.

### Measurement of Retinal Transduction Via Flow-Cytometry

Neural retinas (with RPE manually stripped from retina) from between 4 to 6 Nrl-GFP eyes per cohort were harvested and dissociated with papain. Flow-cytometry was performed on treated, dissociated retinas and untreated controls to quantify the percentage of cells that were positive for GFP (rod photoreceptors), mCherry (non-rod retinal neurons transduced by rAAV), or both (rod photoreceptors transduced by rAAV). The percentage of rods and non-rod neural retinal cells transduced by each vector were separately averaged.

### Tissue Preparation and Immunostaining

Four weeks post-injection, the eyes were enucleated, fixed overnight at 4° C. in freshly prepared 4% paraformaldehyde (PFA) in phosphate-buffered saline (PBS). Cornea and lens were removed, and the eye cup was incubated in 30% sucrose solution overnight at 4° C. Eyes were embedded in cryostat compound and frozen at −80° C. Sections (12 μm thick) were cut using a cryostat (Leica Microsystem, Buffalo Grove, Ill.) and transferred to glass slides. Retinal cryosections were rinsed with 1× phosphate-buffered saline (PBS), blocked with 0.5% Triton-X100 and 1% bovine serum albumin (BSA) for 1 hour each and then incubated overnight at 4° C. with mouse monoclonal anti cone arrestin antibody (1:1000, generously provided by Dr Clay Smith). The following day slides were rinsed with 1×PBS and then incubated at room temperature for 1 hour with Alexa Fluor donkey-anti-mouse secondary antibody (1:500) in 1×PBS and counter-stained with DAPI. Images were acquired using confocal laser scanning microscope (Leica TCS SP8) and Fluorescence microscope (EVOS).

### Example 2—Natural History Study of Retinoschisis Mice

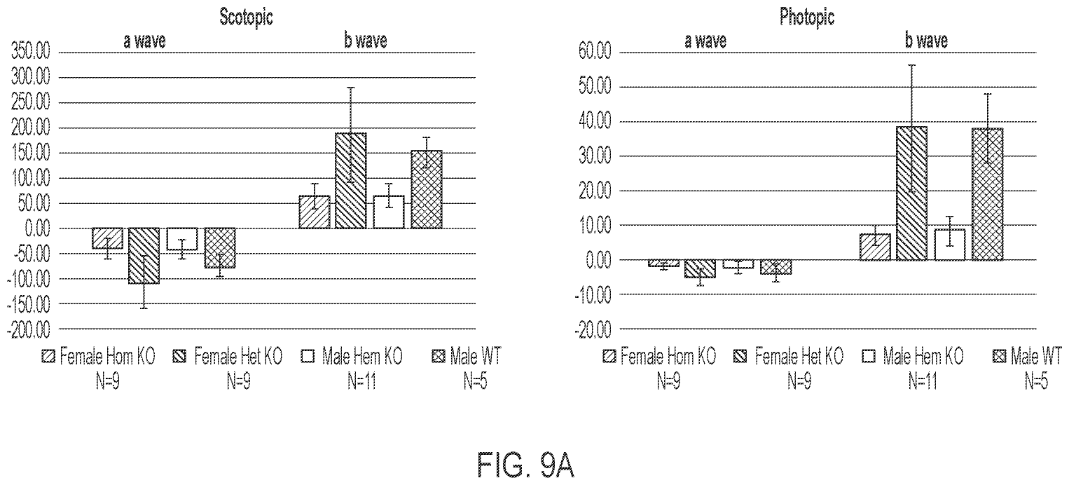

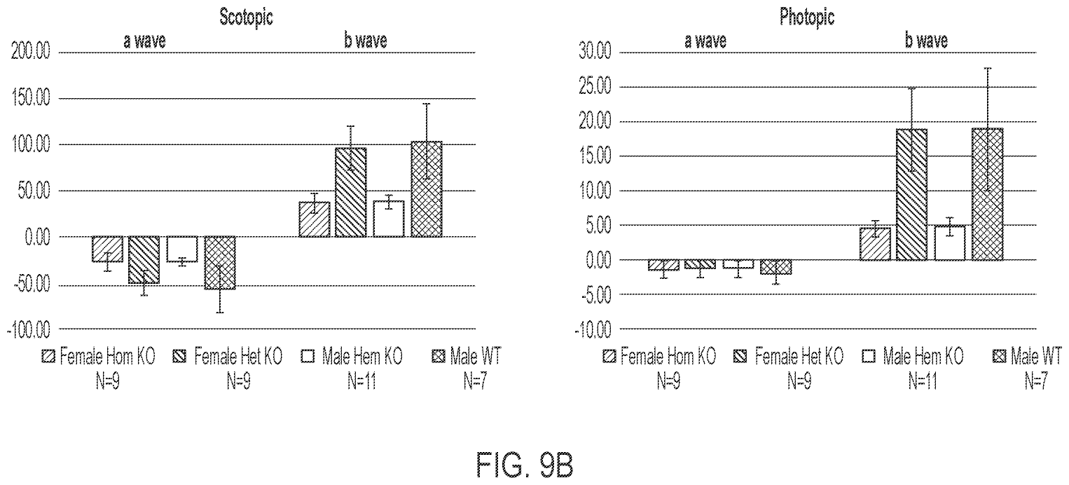

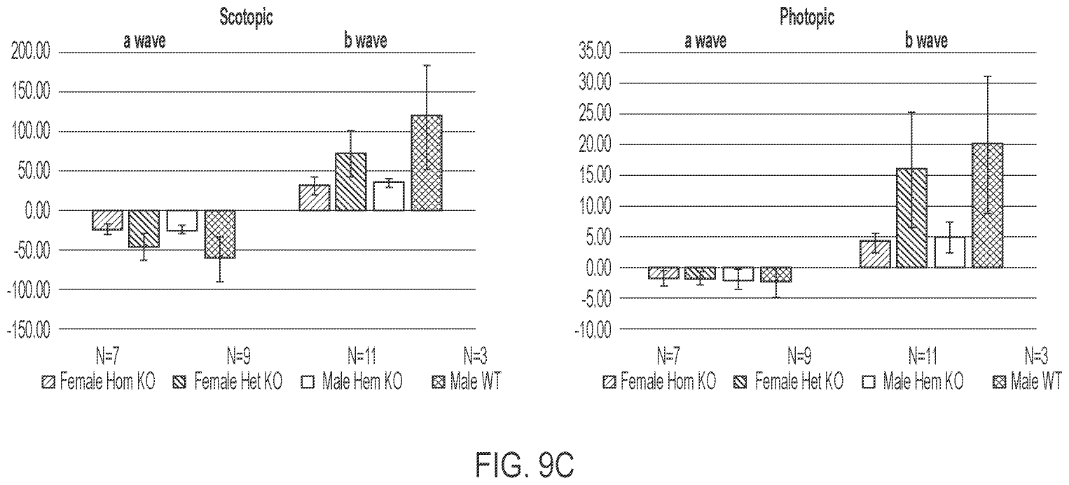

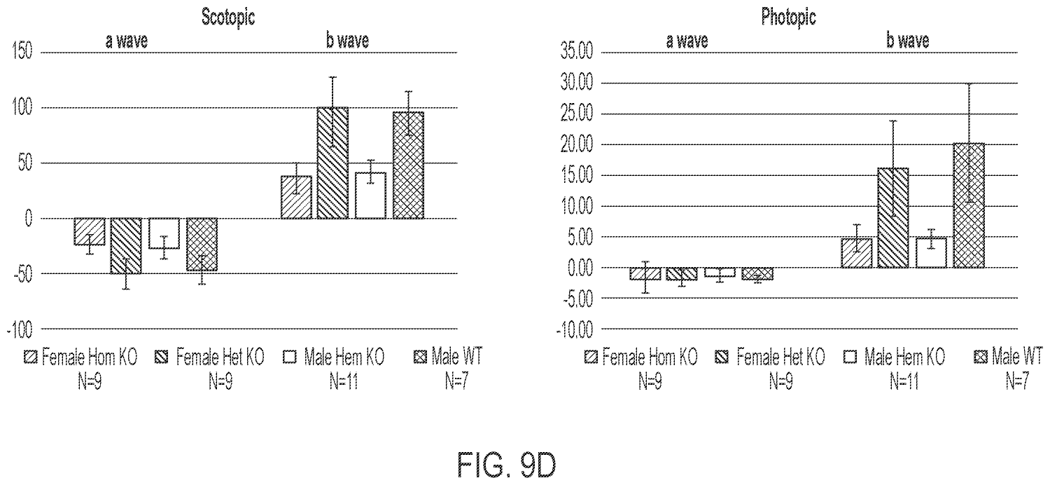

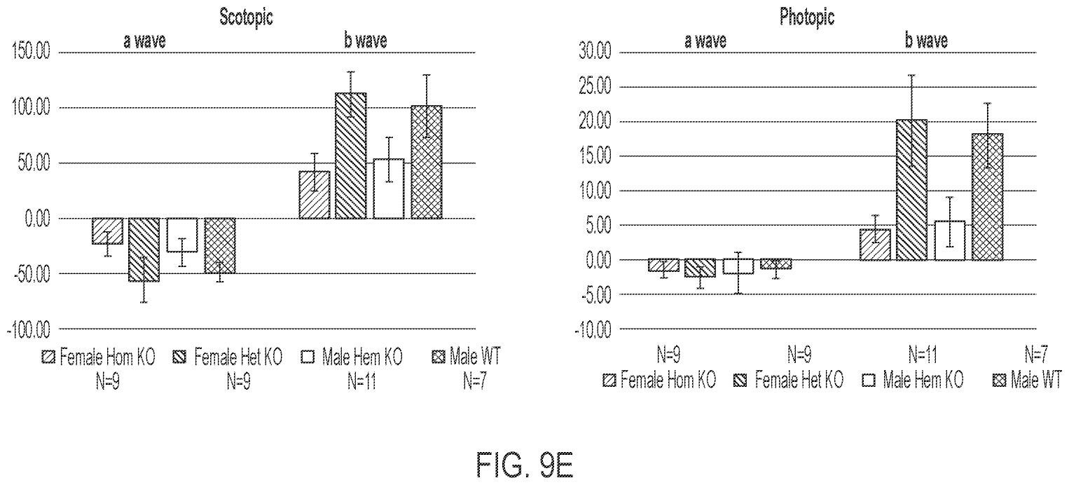

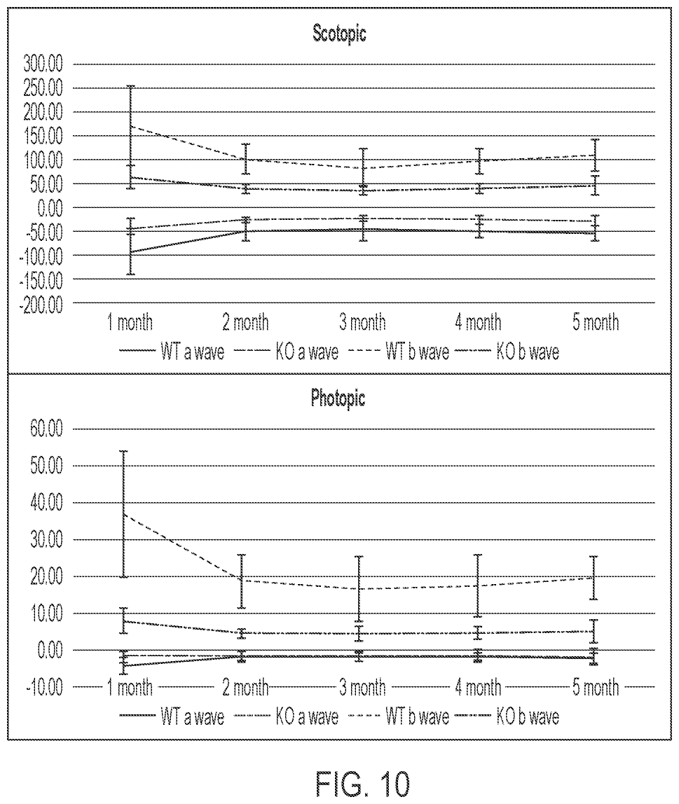

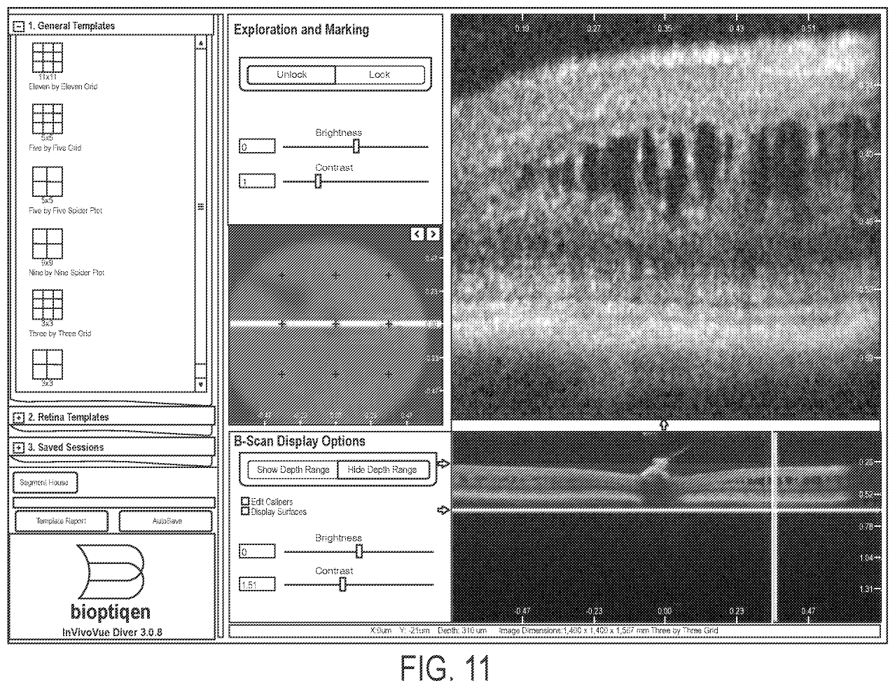

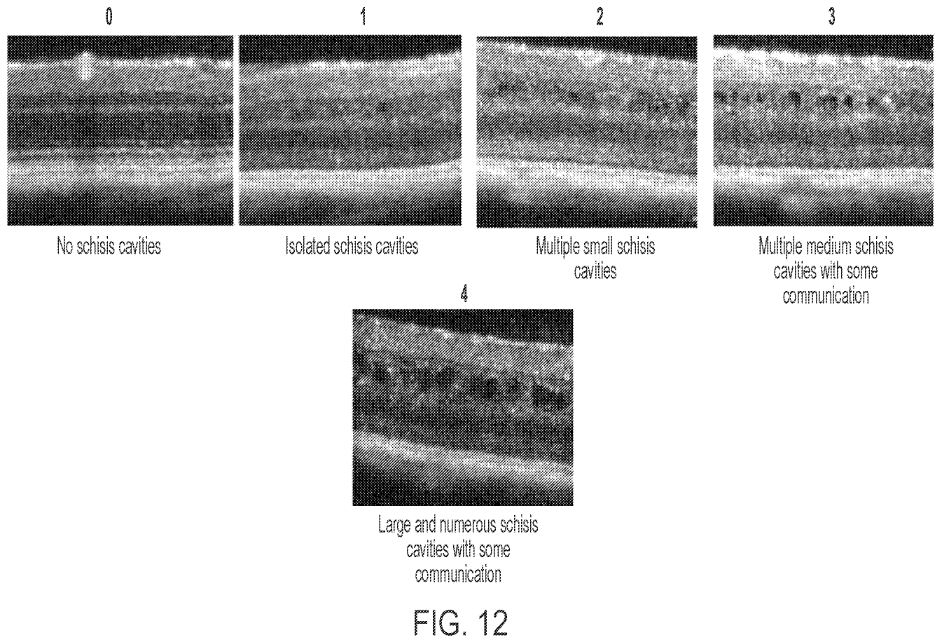

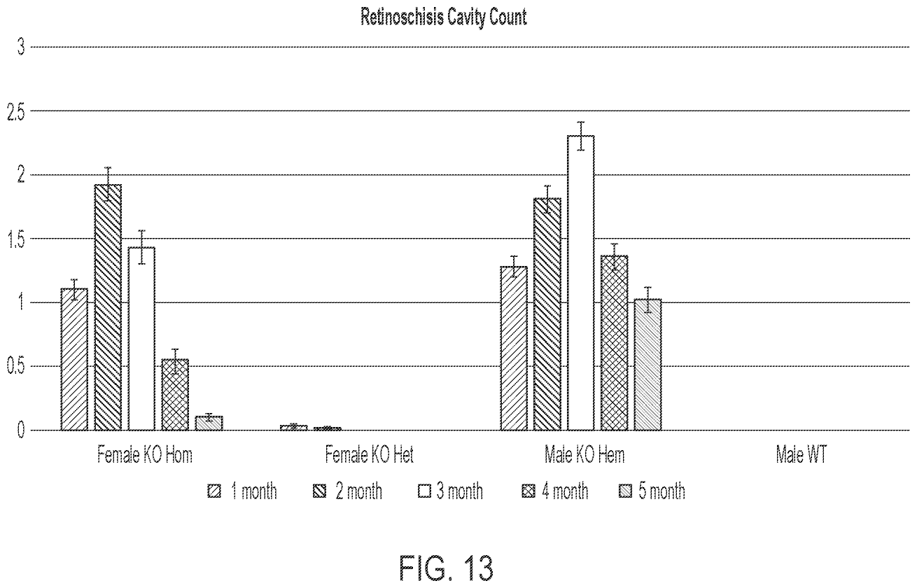

A 5-month natural history study of RS1 knockout (KO) mice was conducted to confirm that mice display the characteristic phenotypes of XLRS. Abnormal retinal function of the mice was observed by ERG, and schisis formations in retinal structure were measured by OCT. Measurements were taken at the end of each month for five months. See FIGS. 9A-9E and 10-13. Scores were averaged to generate an overall cavity score across individual retinas. Mice were grouped into the following cohorts:
Male Rs1 hemizygous (Rs1^{-/y}) KO (diseased)
Female Rs1 homozygous KO (diseased)
Male Rs1^{+/y} wild type (normal)
Female Rs1^{+/-} heterozygous (normal)

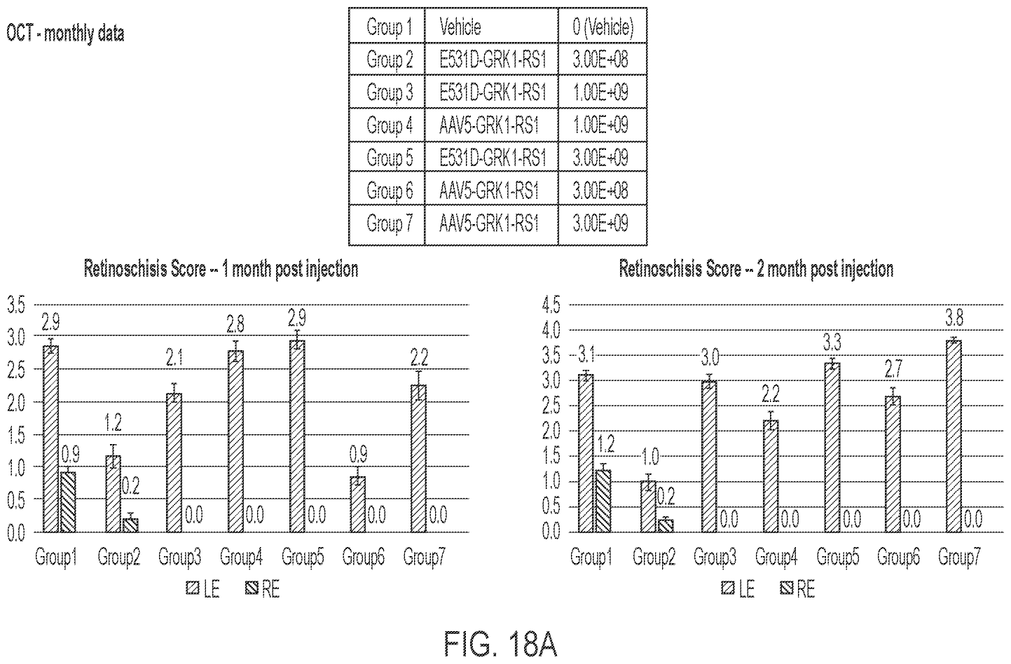

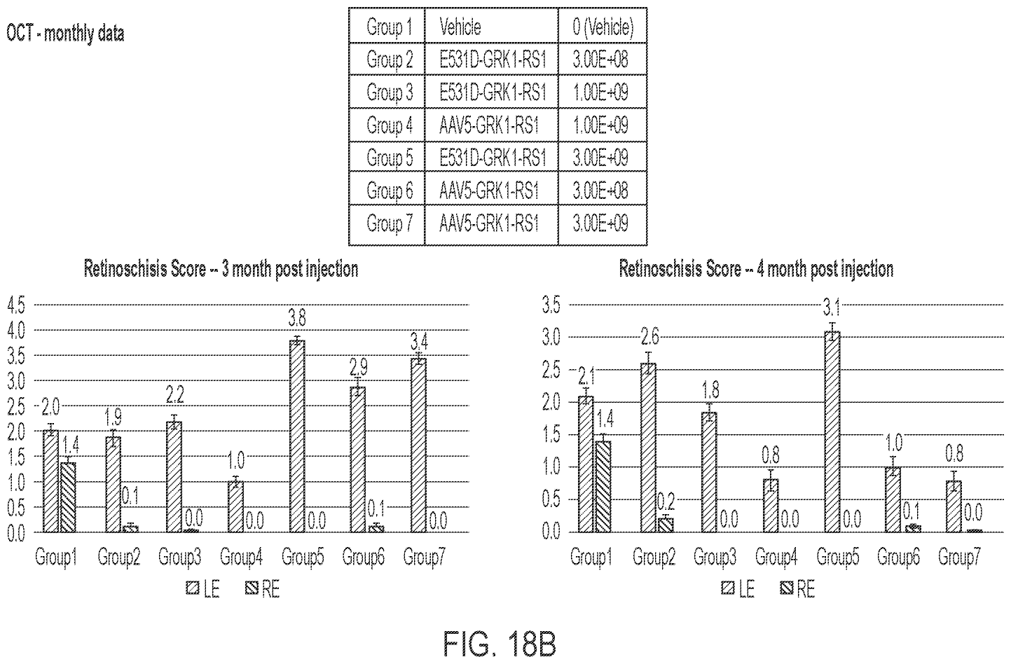

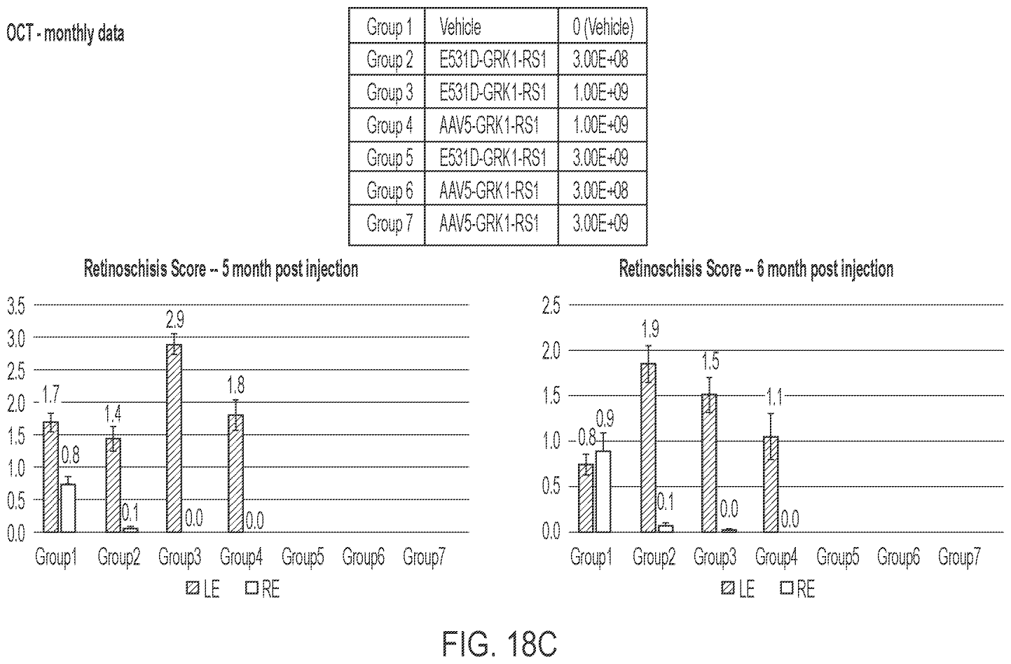

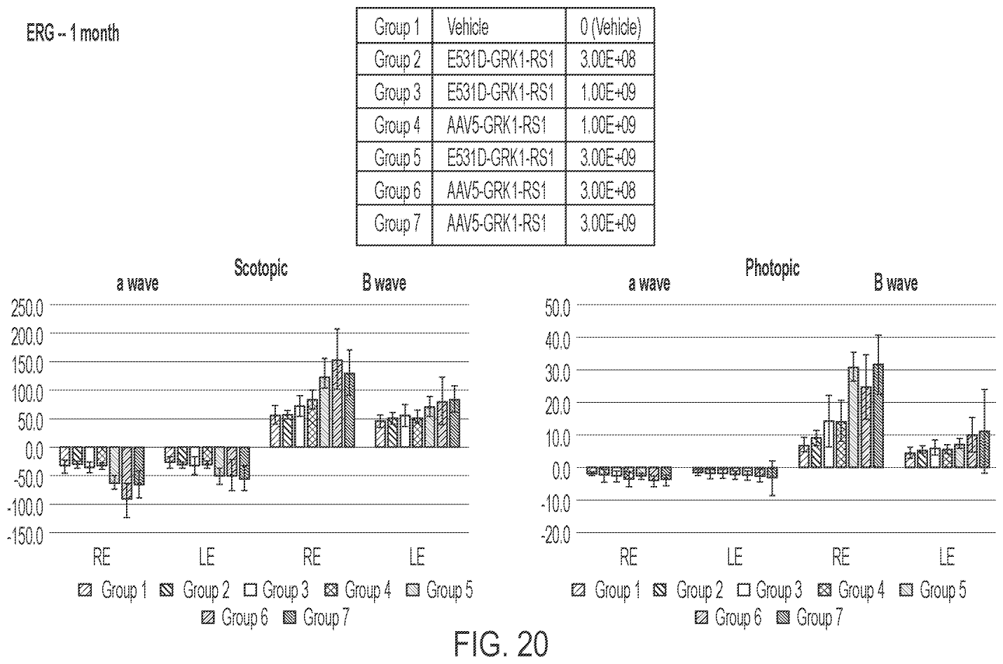

A single cohort of mice were sacrificed at 1 month of age, and their retinas were evaluated for expression of retinoschisin (RS1) by immunohistochemistry. Results are shown in FIGS. 18A-18C and 20.

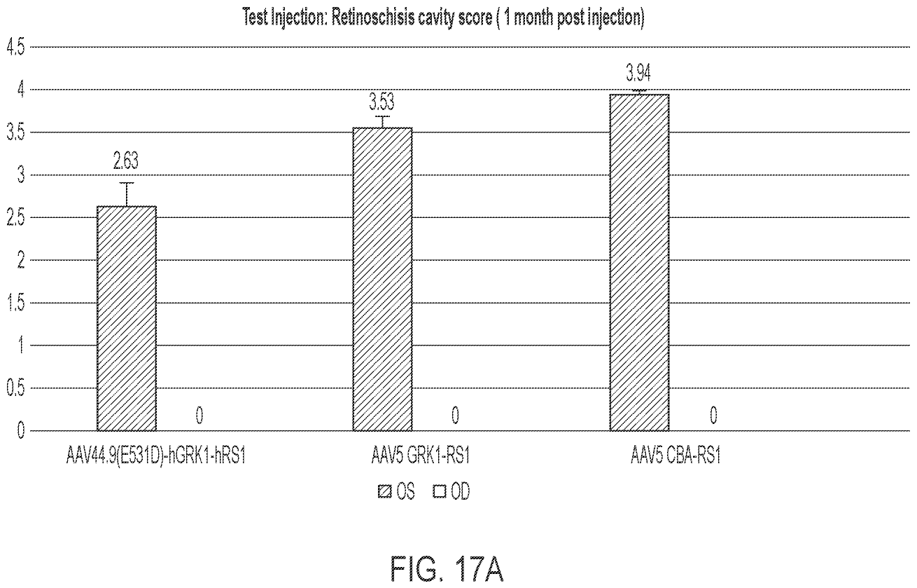

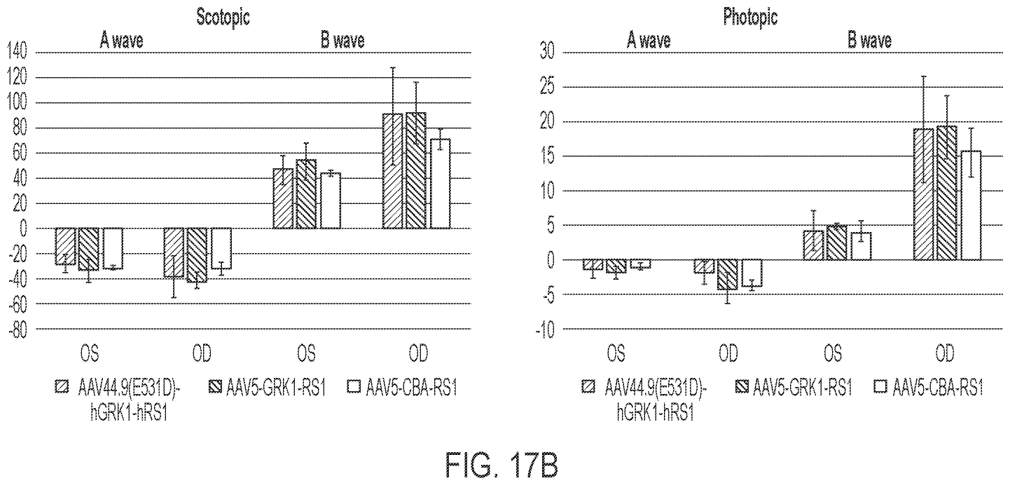

To interpret the OCT results, a grading system for severity of schisis cavities (0-4), with 0 equaling no schisis cavities and 4 being the highest cavity count. OCT measurements and schisis cavity scoring charts are shown in FIGS. 17A and 18A-18C for each of 5 months of the study. The grading system is defined as follows:
0: No schisis cavities; 1: Isolated schisis cavities; 2: Multiple small schisis cavities; 3: Multiple medium schisis cavities with some communication; 4: Large and numerous schisis cavities with some communication

The natural history study showed that the cavity score of RS1 KO mice peaked at 2 to 3 months, and then improved with time. IHC measurements showed that RS1 expression localized to the inner/outer segment junction of photoreceptors in the female heterozygous and wildtype male mice and is absent in male and female Rs1 KO mice.

### Example 3—Evaluation of Therapeutic Efficacy of AAV Capsids in Subretinal Delivery of Synthetic hRS1 to Retinoschisis Mice

The biodistribution of various AAV capsids, including AAV44.9(E531D), in retina and RPE relative to AAVS capsids across multiple doses of rAAV particles were evaluated over a five-month study. rAAV particles containing one of the evaluated capsids were administered to Rs1 knockout mice.

Particles were administered to four mice in each of four cohorts in a dose of 1 μl of 5×10¹² vector genomes (vgs)/mL. Accordingly, a total of 5×10⁹ vector genomes were delivered to each animal. Particles were administered by subretinal injection into the OD eye, and contralateral eyes were not treated.

It was evaluated whether subretinal injections of rAAV vectors expressing a synthetic human a synthetic human RS1 transgene could provide therapeutic efficacy to Rs1 knockout (Rs1-KO) mice. This synthetic transgene was truncated, lacking 5′ and 3′ untranslated regions (UTRs), such that it contained only the coding region of RS1. The nucleotide sequence of this transgene is provided above as SEQ ID NO: 8. In some experiments, the transgene was myc-tagged (see the nucleotide sequence of SEQ ID NO: 10).

In other experiments, the synthetic transgene also had four CpG islands have been removed from the coding sequence and was codon-optimized for human expression (see the nucleotide sequence of SEQ ID NO: 10).

Several different capsids were evaluated for inducing strongest biodistribution in PRs. rAAV44.9(E531D)-hGRK1-hRS1 and rAAV5-hGRK1-hRS1 vectors were administered to Rs1 KO mice restored retinal function and structure. Both of these capsids have the hGRK1 promoter operably controlling the hRS1 transgene. Results were compared to those in Rs1 KO mice treated with benchmark vector rAAV5-CBA-hRS1, and those of contralateral (OS) untreated eyes (in all groups).

Viral particles comprising the AAV44.9(E531D) capsid carrying hGRK1-hRS1 successfully transduced photoreceptor cells and provided therapeutic levels of human retinoschisin in these cells. Surprisingly, retinoschisis cavities were completely resolved in all treated eyes as early as a single month after rAAV injection. Likewise, retinal function was substantially improved in all treated eyes as early as a single month after rAAV injection. Table 1 (below) shows 6-month ERG and OCT data from mice in Groups 1-4, and 4-month ERG and OCT data in mice from Groups 6 and 7. “vg”=vector geomes; “—N*” signifies the number (N) of animals dead or sacrificed for IHC.

The human rhodopsin kinase promoter (hGRK1) was confirmed as the strongest evaluated promoter for mediating expression of therapeutic levels of human RS1 in PRs of treated eyes.

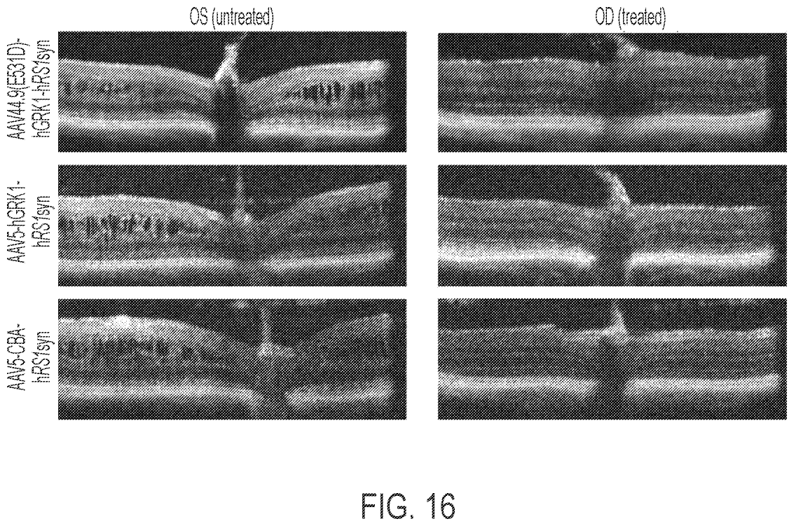

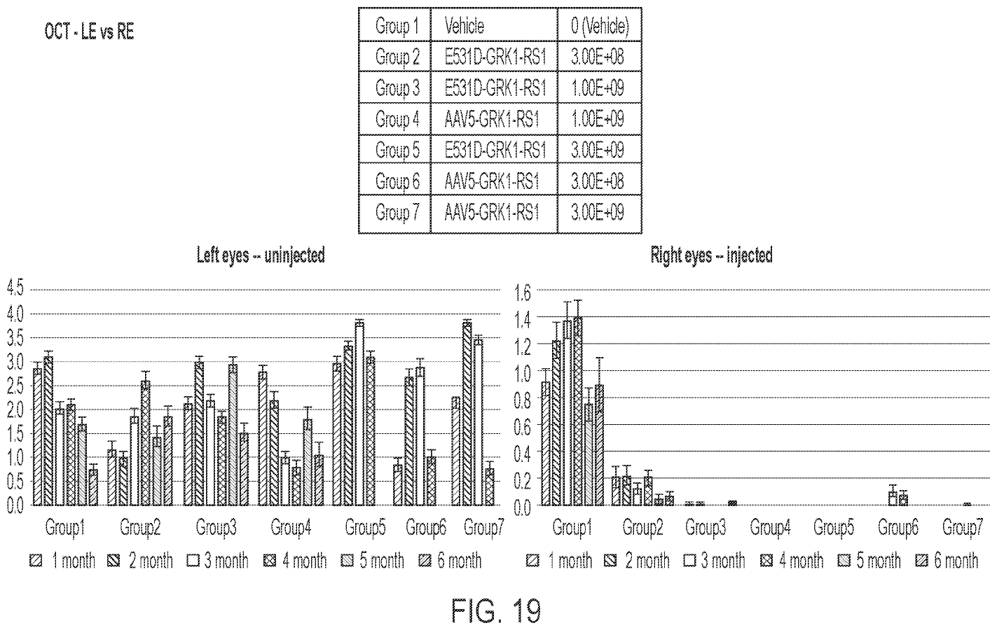

In addition, a 1-month proof-of-concept (POC) study evaluating the impact of subretinally delivered AAV5 or AAV44.9(E531D), containing hGRK1 or the ubiquitious CBA promoter driving the hRS1syn gene, on retinal structure and function was performed in RS1-KO deficient mice (hemizygous Rs1^{-/y} males). A single dose (2.3×10¹² vg/mL; 2.3×10⁹ vg/eye) of rAAV44.9(E531D)-hGRK1-hRS1syn, rAAV5-hGRK1-hRS1syn, or AAV5-CBA-hRS1syn was administered a postnatal day 25 (P25). The P25 time point was chosen because it corresponds to an immature adult (early teens), a time point where it is expected that XLRS patients will pursue treatment. The contralateral eye remained un-injected. At 1-month post-injection, retinal structure and function were assessed via optical coherence tomography (OCT) and electroretinogram (ERG), respectively. Mice were euthanized immediately thereafter and both eyes from mice in all groups were collected, fixed, cryo-protected and evaluated with immunohistochemistry. FIG. 16 shows restoration of retinal structure in rAAV-treated RS1-KO mice at 1 month post-injection by OCT analysis (complete resolution of schisis cavities in RS1-KO mice treated with all three vectors). Further, rod- and cone-mediated retinal function was improved in RS1-KO mouse eyes treated with all vectors relative to un-injected controls. Immunohistochemical analysis revealed the presence of RS1 expression in photoreceptor inner segments in cross sections of treated retinas. RS1 expression was absent from contralateral untreated eyes. These results suggested that both capsids rAAV44.9(E531D)-hGRK1-hRS1syn, rAAV5-hGRK1-hRS1syn, were efficacious, and outcomes were similar following treatment with vectors containing the photoreceptor-specific hGRK1 promoter and the ubiquitous CBA promoter. Further studies were performed using hGRK1 for limited expression to the intended target (photoreceptors), thus providing an additional safety feature by avoiding off-target expression. Associated ERG measurements at 1 month are shown in FIG. 17B. A comparison of OCT data of the left eye and right eye are shown in FIG. 19.

Following the POC study, a six-month, pharmacology study was conducted to evaluate dose responsive improvements in retinal structure and function following a single subretinal administration of an AAV5 or AAV44.9(E531D) capsid containing the hGRK1 promoter driving the hRS1syn gene (rAAV5-hGRK1-hRS1syn or rAAV44.9(E531D)-hGRK1-hRS1syn, respectively) in RS1-KO mice. Mice were subretinally injected between P24 and P26 in one eye with either vehicle, AAV5 or AAV44.9(E531D) at low (1.4×10¹¹ vg/ml; 1.4×10⁸ vg/eye), middle (4.7×10¹¹ vg/ml; 4.7×10⁸ vg/ml), or high (1.4×10¹² vg/ml; 1.4×10⁹ vg/eye) dose. The contralateral eyes remained un-injected. Retinal structure and function were assessed via OCT and ERG, respectively, monthly over the course of 6 months. Following euthanasia at 6 months post-injection, retinas were cryosectioned and evaluated for RS1 expression via immunohistochemistry.

OCT analysis revealed stable resolution of schisis cavities in RS1-KO mice treated with both vectors at all doses starting at one-month post-injection. An apparent dose-response was observed, as lower retinoschisis scores were observed in the two higher dose cohorts than in the low dose group. Significant improvements in both rod- and cone-mediated ERG function were also observed following treatment with both vectors at all doses, including the low dose. For scotopic b-wave amplitudes, the number of timepoints at which a significant difference between rAAV44.9(E531D)-hGRK1-hRS1syn-treated and vehicle-treated animals was observed, increased from 3 timepoints (2, 5, 6 months) at the low dose, 4 timepoints (2, 3, 5, 6 months) at the mid dose, to all 6 timepoints in the high dose-treated animals. A similar observation was made for photopic b-wave amplitudes. These data suggest a dose response of rAAV44.9(E531D)-hGRK1-hRS1syn in correcting retinal function in RS1-KO mice. Similar observations were seen in animals injected with rAAV5-hGRK1-hRS1syn. Immunohistochemical analysis revealed the presence of RS1 expression in photoreceptor inner segments in cross sections of retinas from treated eyes of RS1-KO mice. RS1 expression was absent from contralateral untreated eyes and those treated with vehicle alone.

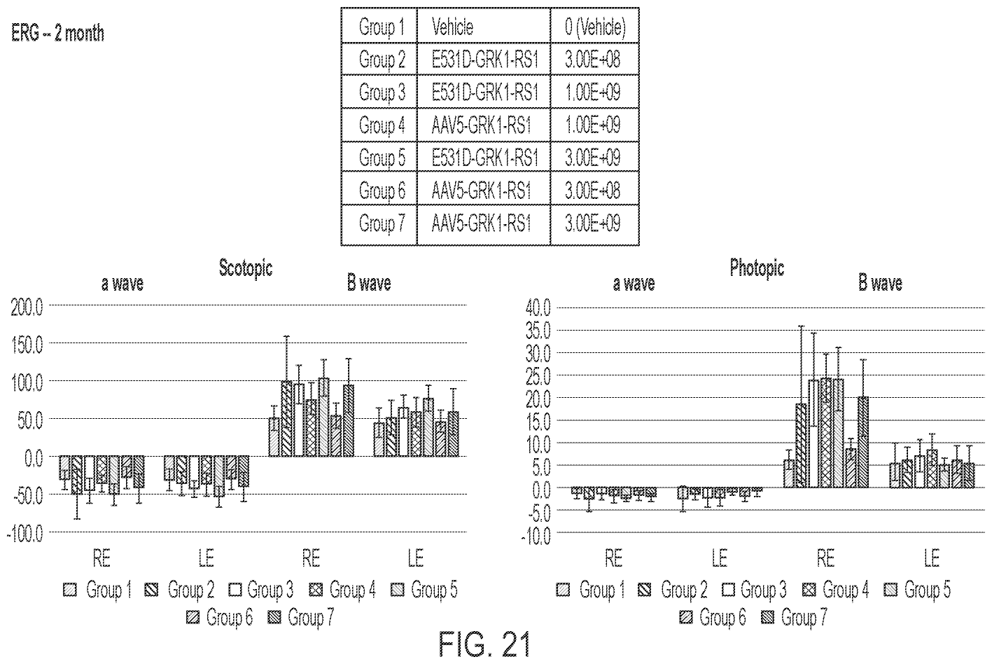

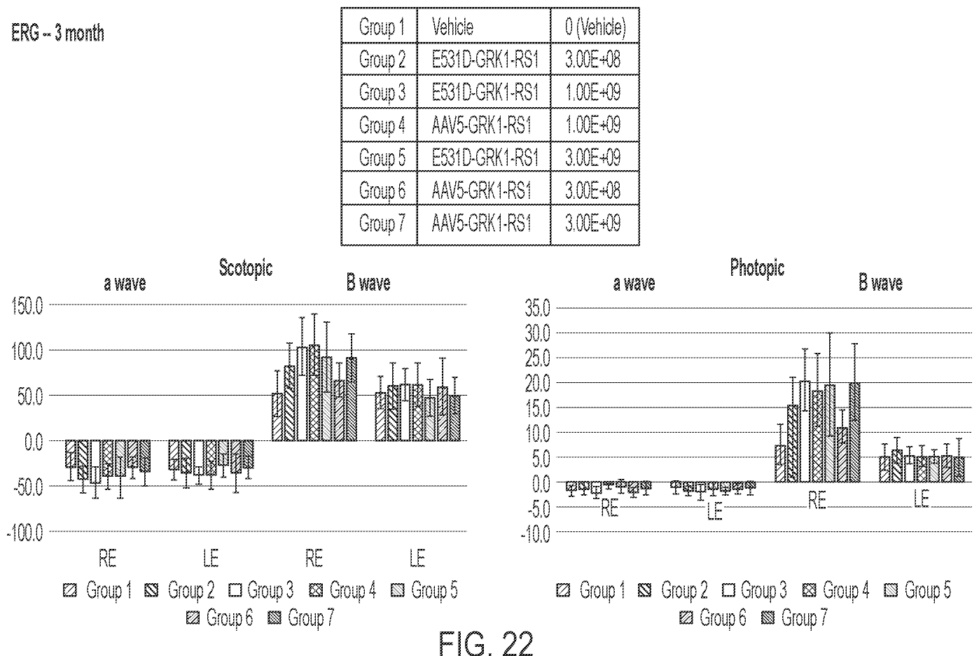

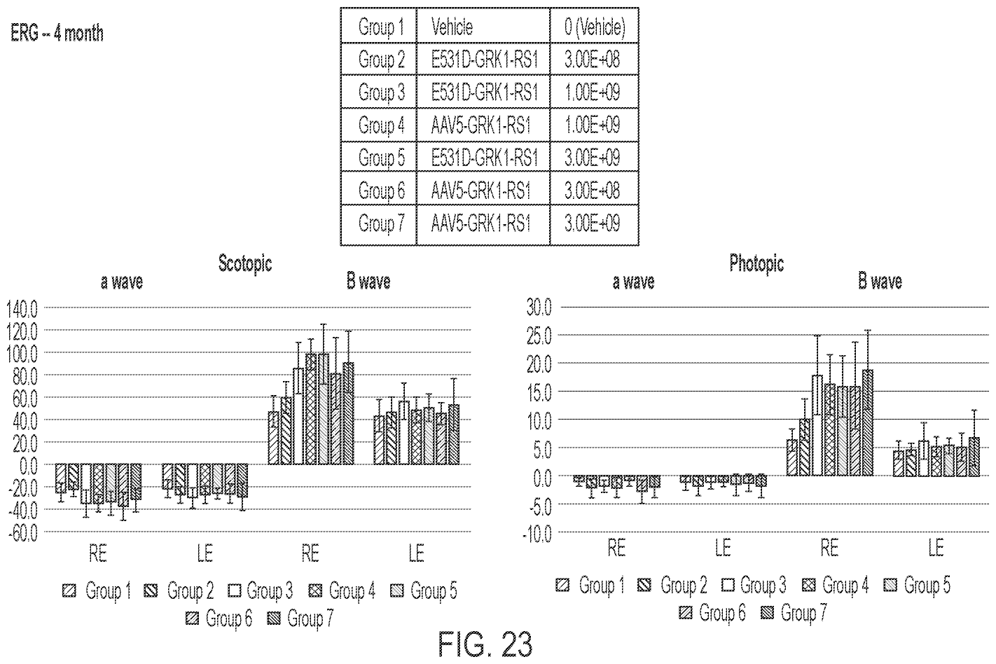

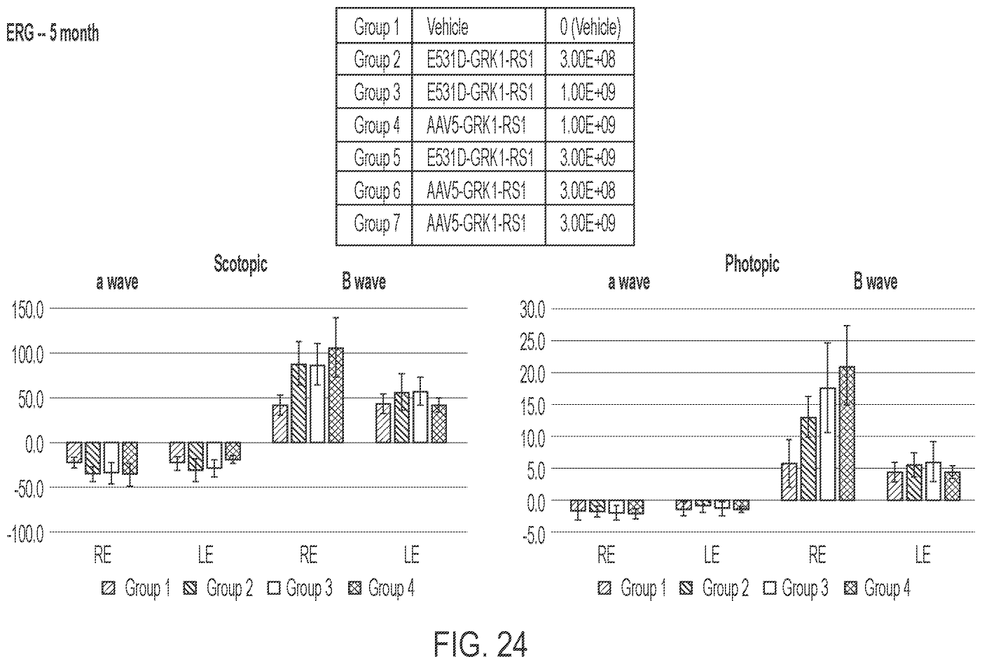

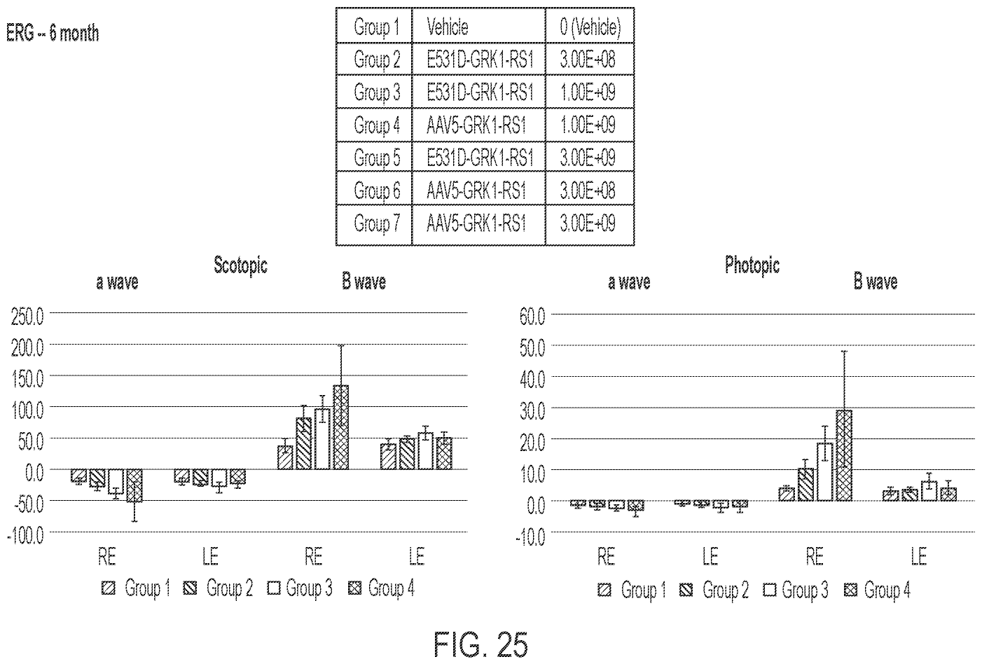

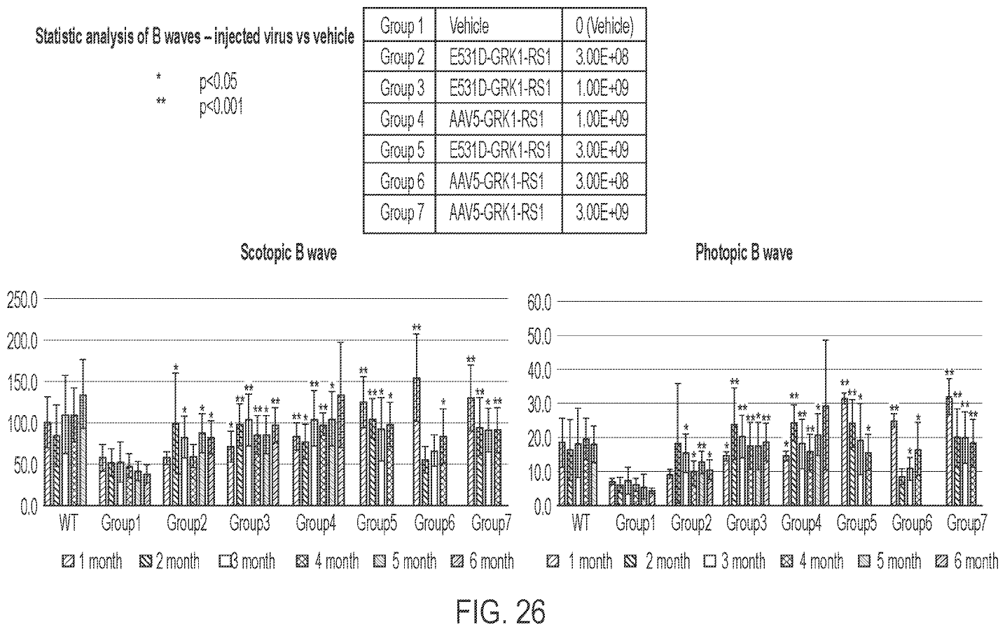

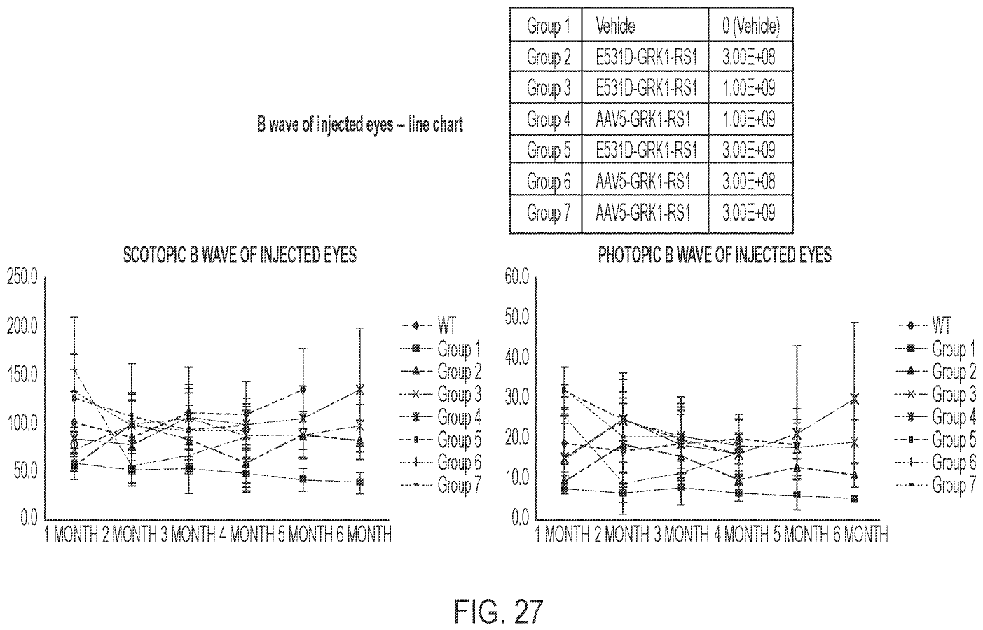

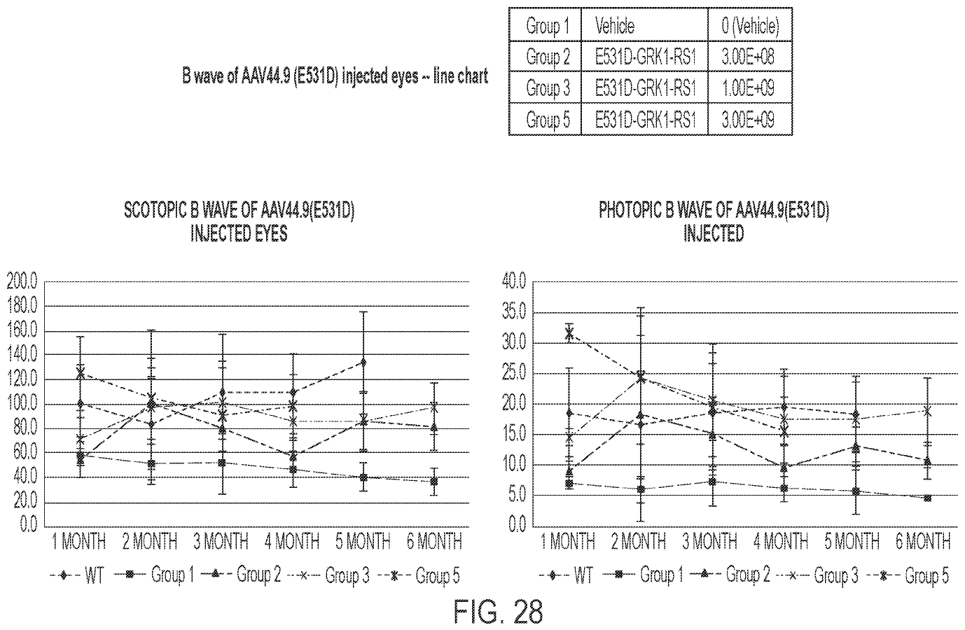

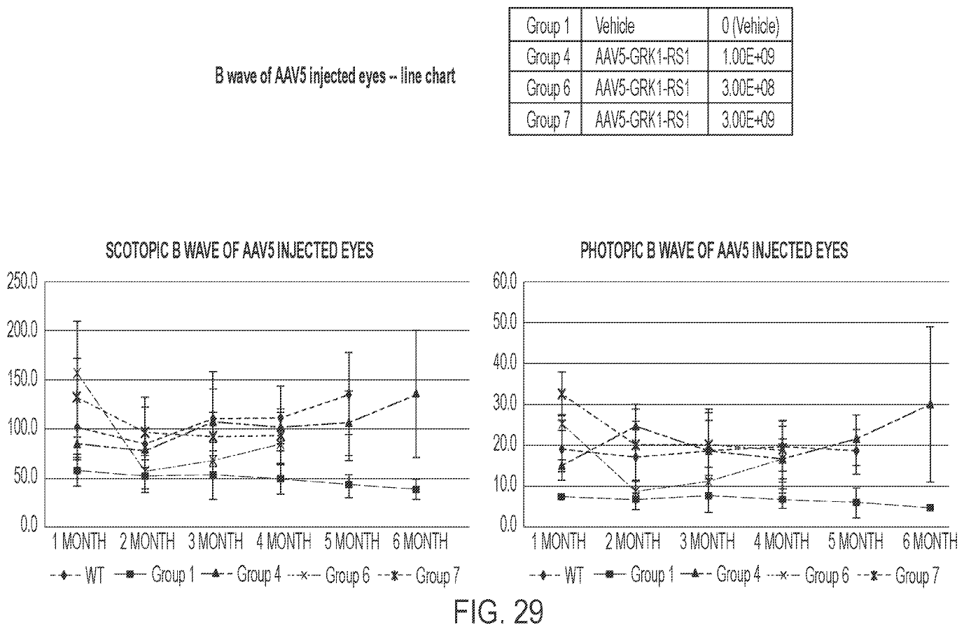

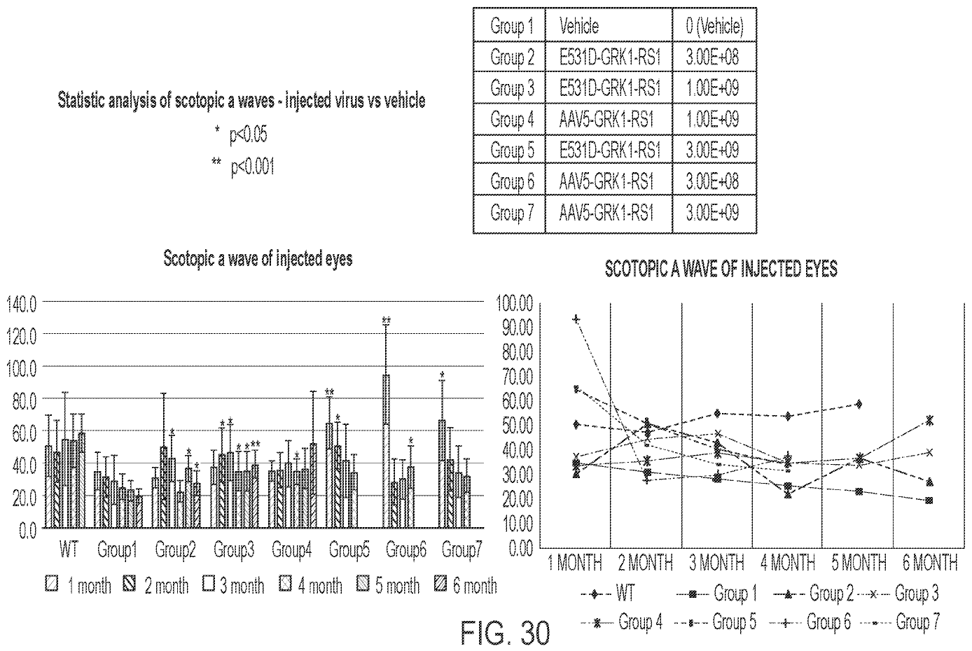

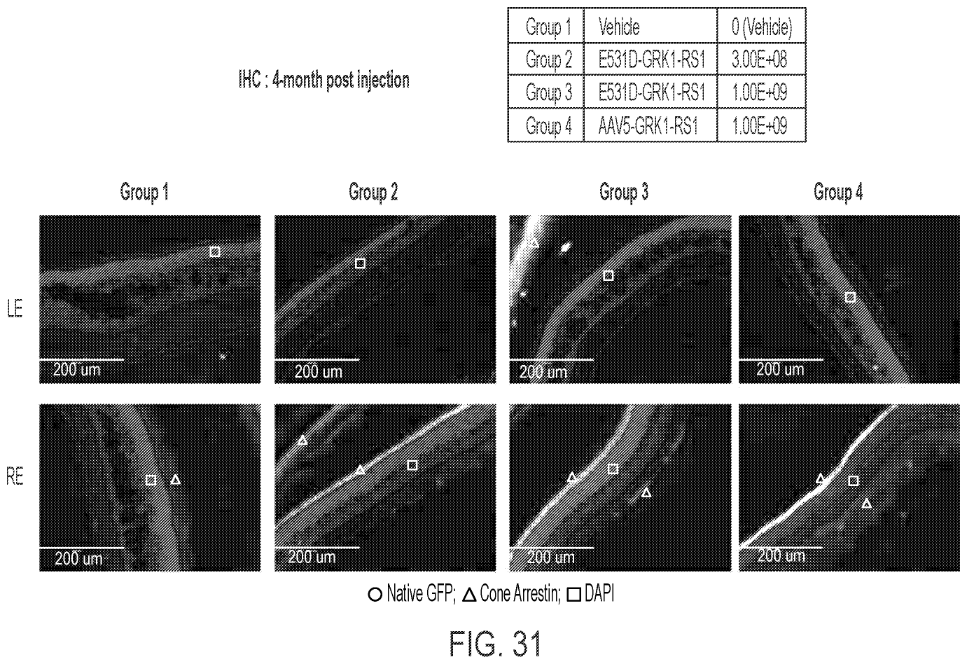

The ERG measurement results at each of months 1-6 are shown in FIGS. 20-25, respectively. Statistical analyses of b waves of the injected virus versus vehicle, as determined by ERG measurements of Group 6 and 7 are shown in FIGS. 26-30. IHC measurements at the 4-month timepoint after injection are shown in FIG. 31.

In conclusion, these study results show that 1) subretinal injections of rAAV5 and rAAV44.9(E531D) vectors have comparable potency across a range of doses in the RS1-KO mice, 2) a dose response relationship was observed between both rAAV44.9(E531D)-hGRK1-hRS1syn and rAAV5-hGRK1-hRS1syn and correction of the structural and functional deficits in RS1-KO mice. A minimal effective dose (MED) of approximately 1.4×10¹¹ vg/ml (1.4×10⁸ vg/eye) was identified. Further, 3) the photoreceptor-specific hGRK1 promoter drives therapeutic levels of RS1 expression in treated mice.

Additional studies evaluated dose ranging transduction and biodistribution of rAAV particles having optimal capsids in non-human primates with and without induced retinoschisis. Additional studies will involve administration of Cloning AAV-hGRK1-hRS1 vectors having a woodchuck hepatitis virus post-transcription regulatory element (WPRE), wherein the WPRE is safe for administration (“WPREsf”). It is envisioned that this construct will lead to higher expression of RS1 allowing for reduction of vector dose to further improve the safety profile of these ocular gene therapies.

TABLE 1
ERG/OCT data in Mice

| Group of | | | Injection 1 | | Injection 2 | | Injection 3 | | |
|---|---|---|---|---|---|---|---|---|---|
| mice | Vector | Dose (vg) | Date | Number | Date | Number | Date | Number | Total |
| Group 1 | Vehicle | 0 | Dec. 18, 2019 | 10 | Jan. 7, 2020 | 7 | Feb. 26, 2020 | 3 | 20 - 3* |
| | (BSS + Pluronic) | | | | | | | | |
| Group 2 | AAV44.9(E531D)- | 3.00 × 10⁸ | Dec. 18, 2019 | 9 | | | | | 9 - 2* |
| | hGRK1-RS1 | | | | | | | | |
| Group 3 | AAV44.9(E531D)- | 1.00 × 10⁹ | Dec. 18, 2019 | 10 | Feb. 4, 2020 | 7 | | | 17 - 5* |
| | hGRK1-RS1 | | | | | | | | |
| Group 4 | AAV5-hGRK1- | 1.00 × 10⁹ | Dec. 18, 2019 | 10 | | | | | 10 - 6* |
| | RS1 | | | | | | | | |
| Group 5 | AAV44.9(E531D)- | 3.00 × 10⁹ | Feb. 4, 2020 | 9 | | | | | 9 - 2* |
| | hGRK1-RS1 | | | | | | | | |
| Group 6 | AAV5-hGRK1- | 3.00 × 10⁸ | Feb. 24, 2020 | 10 | | | | | 10 - 1* |
| | RS1 | | | | | | | | |
| Group 7 | AAV5-hGRK1- | 3.00 × 10⁹ | Feb. 26, 2020 | 10 | | | | | 10 - 1* |
| | RS1 | | | | | | | | |

### Example 4—Evaluation of hRS1-Containing rAAV Vectors with Stuffer Sequences

This study was conducted to evaluate optimized hRS1-containing AAV44.9(E531D) vectors with genome sizes conducive to efficient packaging. The goal was to identify a construct that was at least as effective as rAAV44.9(E531D)-X001.

Due to the small packaging size of pTR-X001 (1723 bp), and the possibility for heterologous genome packaging, several new constructs were designed with cassette sizes approaching the natural carrying capacity of AAV (˜4.7 Kb ITR to ITR cassette). This was accomplished by addition of an inert stuffer sequence inserted within the vector cassette either 5′ to (X001-5p), or 3′ to (X001-3p) hGRK1-hRS1syn-bGH polyA (pTR-X001-5p and pTR-X001-3p, respectively). The stuffer DNA was de novo synthesized (Genscript, NJ). Additionally, a version was created incorporating the woodchuck hepatitis virus post-transcriptional regulatory element (WPRE) positioned between hRS1syn and bGH polyA (pTR-X002-3p). The mutant version of WPRE used has previously been incorporated in AAV vectors used in other ocular gene therapy clinical trials. All constructs contained the same hGRK1 promoter, SV40 SD/SA and bGH polyA signal sequence. rAAV44.9(E531D) vectors were produced by packaging these expression cassettes, and the vector genomes ranged in size from 4534-4549 nucleotides. The multiple constructs evaluated are represented in FIGS. 15D-15F.

A 3-month nonclinical study using two vector doses was performed to test restoration of retinal structure/function in RS1-KO mice. The ‘unstuffed’ vector pTR-X001 (rAAV44.9(E531D)-X001), which rescued retinal structure/function in the earlier described RS1-KO mouse studies, was included as the comparator control.

A summary of the cassette selection study design is presented in Table 2. RS1-KO mice were subretinally injected in one eye with either vehicle (Group 1), rAAV44.9(E531D)-X001 (Groups 2 and 3), rAAV44.9(E531D)-X001-3p (Groups 4 and 5), rAAV44.9(E531D)-X001-5p (Groups 6 and 7), or rAAV44.9(E531D)-X002-3p (Groups 8 and 9) vectors. Vectors were delivered at either 1.0×10¹¹ vg/mL; 1.0×10⁸ vg/eye (Groups 2,4,6,8) or 5.0×10¹¹ vg/mL; 5.0×10⁸ vg/eye (Groups 3,5,7,9). The contralateral eyes remained un-injected. Retinal structure and function were assessed via OCT and ERG, respectively, at approximately 1-, and 2-months post-injection. Animals were euthanized at approximately 3 months post-injection. Following euthanasia, retinas were cryosectioned and evaluated for RS1 expression via immunohistochemistry.

TABLE 1
Study Study Design

| | Number | | | |
|---|---|---|---|---|
| | of | | Vector Dose | |
| Group | Animals | Test Material | vg/mL | vg/eye |
| 1 | 10 | Vehicle | N/A | N/A |
| | | (BSS + 0.014% Tween-20) | | |
| 2 | 10 | rAAV44.9(E531D) -X001 | 1.0 × 10¹¹ | 1.0 × 10⁸ |
| 3 | 10 | rAAV44.9(E531D)-X001 | 5.0 × 10¹¹ | 5.0 × 10⁸ |
| 4 | 10 | rAAV44.9(E531D)-X001-3p | 1.0 × 10¹¹ | 1.0 × 10⁸ |
| 5 | 10 | rAAV44.9(E531D)-X001-3p | 5.0 × 10¹¹ | 5.0 × 10⁸ |
| 6 | 10 | rAAV44.9(E531D)-X001-5p | 1.0 × 10¹¹ | 1.0 × 10⁸ |
| 7 | 10 | rAAV44.9(E531D)-X001-5p | 5.0 × 10¹¹ | 5.0 × 10⁸ |
| 8 | 10 | rAAV44.9(E531D)-X002-3p | 1.0 × 10¹¹ | 1.0 × 10⁸ |
| 9 | 10 | rAAV44.9(E531D)-X002-3p | 5.0 × 10¹¹ | 5.0 × 10⁸ |

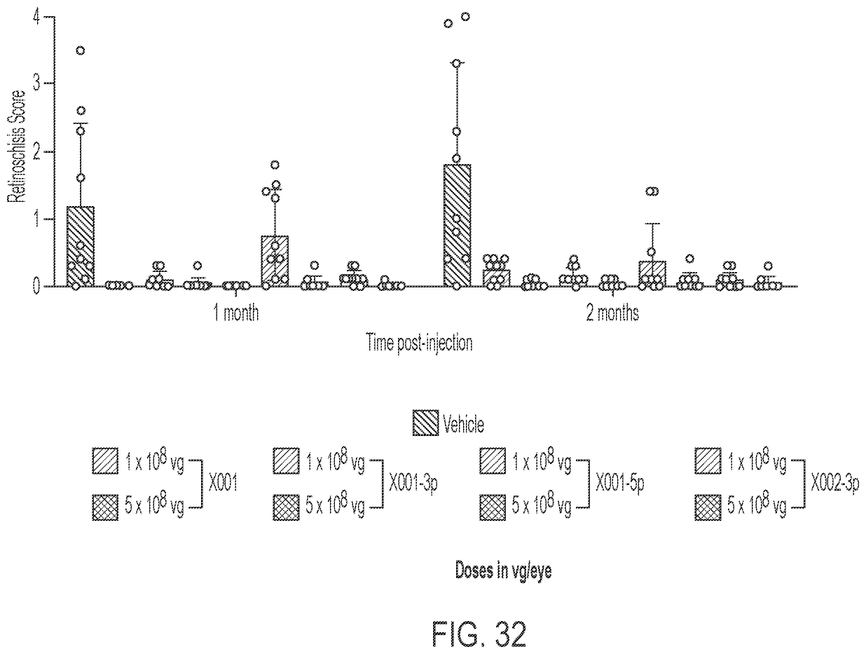

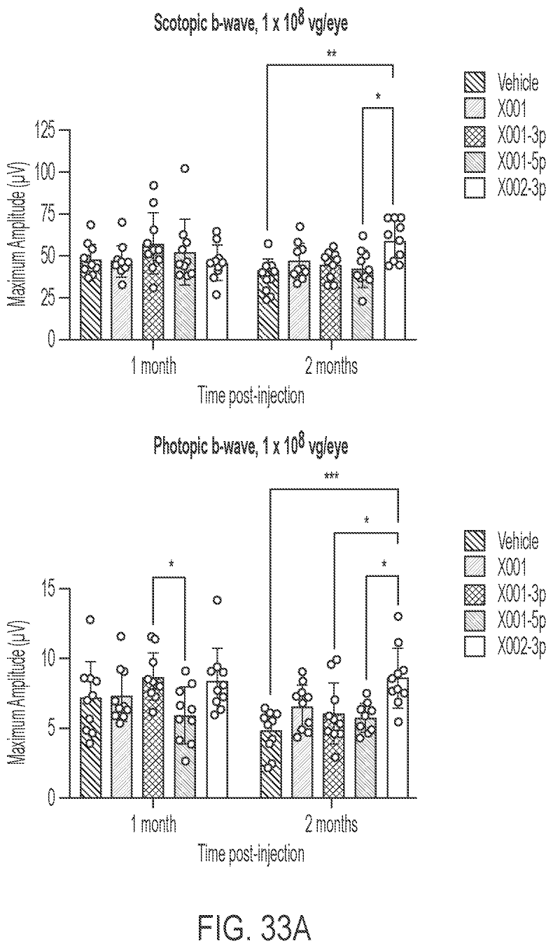

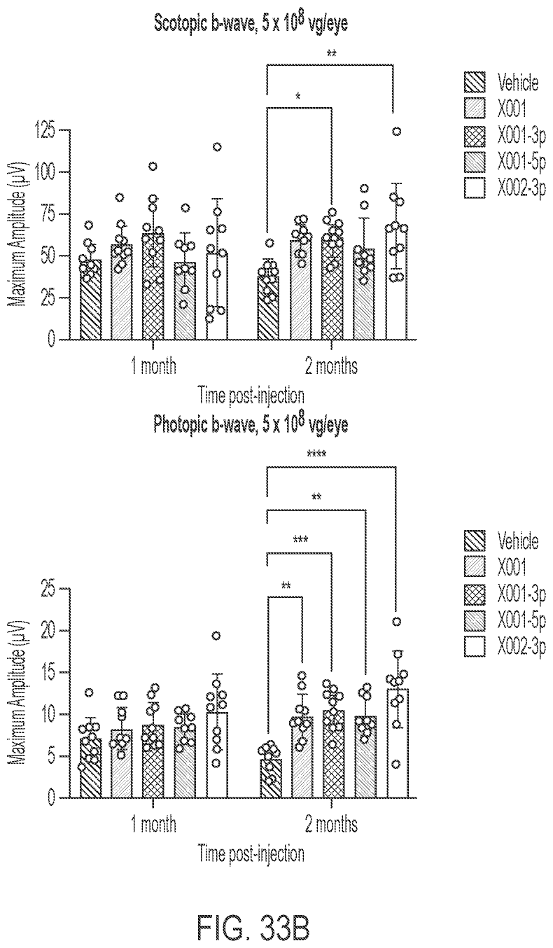

OCT analysis revealed resolution of schisis cavities in vector-treated eyes (FIG. 32). With the exception of eyes treated with the low dose of rAAV44.9(E531D)-X001-5p, and evaluated at 1-month post-injection, there was a statically significant difference in the retinoschisis score between vehicle and vector-treated eyes at both timepoints. At a concentration of 1×10¹¹ vg/mL (1.0×10⁸ vg/eye), rAAV44.9(E531D)-X002-3p led to significant improvements in rod- and cone-mediated function relative to eyes injected with vehicle alone (FIG. 33A). At the higher concentration of 5×10¹¹ vg/mL (5.0×10⁸ vg/eye), all three stuffer constructs conferred significant improvements in retinal function relative to vehicle-injected controls (FIG. 33B). No significant differences in ERG amplitudes between eyes injected with rAAV44.9(E531D) carrying stuffed vs. unstuffed constructs were observed at this higher dose.

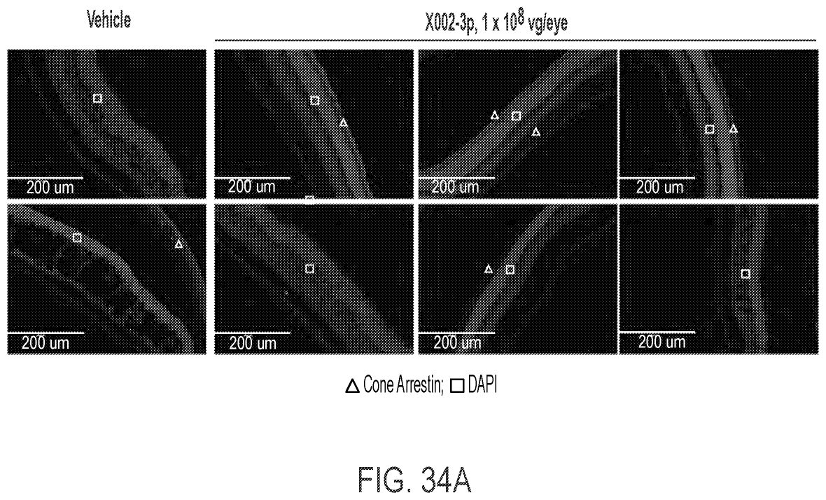

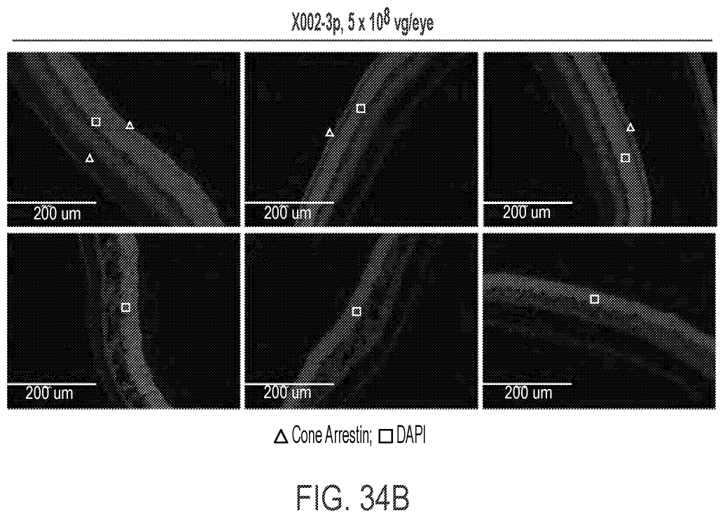

Immunohistochemical analysis revealed the presence of RS1 expression in photoreceptor inner segments of retinas from RS1-KO mouse eyes treated with rAAV44.9(E531D)-X002-3p. RS1 expression was absent from contralateral untreated eyes and those treated with vehicle alone (FIGS. 34A and 34B).

### Example 5—Treatment of XLRS with pTR-X002-3pSR

Multiple pharmacology studies in RS1-KO mice with vectors of the present application, including pTR-X002-3pSR, were performed. These studies established that photoreceptor transduction mediated by the AAV44.9(E531D) capsid and RS1 expression driven by the photoreceptor-specific hGRK1 promoter stably restored retinal structure and function to RS1-KO mice in a dose-dependent fashion.

pTR-X002-3pSR contains RS1 cDNA under the transcriptional control of the human rhodopsin kinase (hGRK1) promoter for specific expression in rod and cone photoreceptors. The hGRK1 promoter is coupled to the SV40 splice donor/splice acceptor, which promotes mRNA transport to the cytoplasm following removal of the SV40 intron. Translation is enhanced by incorporation of a consensus Kozak sequence immediately preceding the hRS1syn start codon. The synthetic human RS1 gene contains four silent changes relative to NCBI reference sequence and is restricted to coding region only. The vector also contains the mut6 version of the woodchuck hepatitis virus posttranslational regulatory element (WPRE) to enhance expression of the RS1 protein. This WPRE contains mutations designed to ablate expression of the putative X protein ORF. The expression cassette also includes a bovine growth hormone poly adenylation signal (bGH poly A). Due to the small packaging size of the promoter-therapeutic vector (1723 bp), which has the potential for heterologous packaging, an inert vector cassette stuffer sequence was inserted to achieve a cassette size of 4549 bp (ITR to ITR). This cassette was packed into pTR-X002-3pSR and demonstrated improved efficacy compared to an unstuffed cassette in an RS1-KO mouse model. A schematic of the vector genome is present in FIG. 15F, and the sequence from ITR to ITR is provided as SEQ ID NO: 33. A description of the sequence elements is shown in Table 3.

TABLE 3
Description of pTR-X002-3pSR cassette elements

| # | Feature | Start | End | Name | Description |
|---|---|---|---|---|---|
| 1 | Region | 1 | 145 | ITR | AAV2 Inverted terminal repeat |
| 2 | Region | 165 | 456 | hGRK1 promoter | Human rhodopsin kinase promoter |
| 3 | Region | 457 | 620 | SV40 SD/SA | Simian Virus 40 Splice Acceptor/Donor and Intron |
| 4 | Region | 654 | 662 | Kozak | Consensus Kozak sequence |
| 5 | Gene | 663 | 1337 | hRS1syn | Human retinoschisin 1 open reading frame |
| 6 | Region | 1339 | 1925 | WPRE | Woodchuck hepatitis virus |
| | | | | | posttranscriptional regulatory element |
| 7 | Region | 1927 | 2155 | bGH PolyA | Bovine growth hormone poly adenylation signal |
| 8 | Region | 2156 | 4389 | Vector Stuffer | Inert vector stuffer DNA sequence |
| 9 | Region | 4405 | 4549 | ITR | AAV2 Inverted terminal repeat |

The pTR-X002-3pSR is administered with an AAV capsid variant AAV44.9(E531D) to introduce human retinoschisin (RS1) gene to photoreceptors in the eye to restore or attenuate the deterioration of vision in patients with X-linked Retinoschisis (XLRS). AAV44.9(E531D) is more potent than benchmark capsids, and unlike other AAVs, it effectively transduces parafoveal cones in subretinally injected cynomolgus monkeys and enables transduction of the macula/fovea without the need for detachment. In addition, a helper plasmid may be utilized to provide E2a, E4, and VA RNA helper genes from Adenovirus Type 5 to the cell the support vector production without the need for wild type viral co-infection. An example helper plasmid may be sourced commercially, e.g., from Aldevron (Fargo, N. Dak.).

### Additional Embodiments

It should be understood that the examples and embodiments described herein are for illustrative purposes only and that various modifications or changes in light thereof may be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein. The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein may be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved. All such similar substitutes and modifications apparent to those skilled in the art are deemed to be within the spirit, scope and concept of the invention as defined by the appended claims.

The following numbered embodiments are intended to be exemplary:
1. An adeno-associated virus (AAV) vector comprising a polynucleotide that comprises a heterologous nucleic acid encoding a retinoschisin protein and an SV40 intron.
2. The AAV vector of embodiment 1, wherein the SV40 intron comprises a splice donor region and/or a splice acceptor region.
3. The AAV vector of embodiment 1 or 2, wherein the SV40 intron comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the nucleic acid sequence of SEQ ID NO: 20.
4. The AAV vector of embodiment 1 or 2, wherein the SV40 intron comprises the nucleic acid sequence of SEQ ID NO: 20.
5. The AAV vector of any one of embodiments 1-4, wherein the retinoschisin protein is a human retinoschisin protein.
6. The AAV vector of any one of embodiments 1-5, wherein the heterologous nucleic acid does not comprise the 5′ untranslated region of human retinoschisin.
7. The AAV vector of embodiment 6, wherein the 5′ untranslated region comprises the nucleic acid sequence of SEQ ID NO: 39.
8. The AAV vector of any one of embodiments 1-7, wherein the nucleic acid does not comprise the 3′ untranslated region of human retinoschisin.
9. The AAV vector of embodiment 8, wherein the 3′ untranslated region comprises the nucleic acid sequence of SEQ ID NO: 40.
10. A recombinant AAV (rAAV) vector comprising a polynucleotide that comprises a heterologous nucleic acid comprising a sequence having at least 80%, 81%, 82%, 83%, 84%, 85%, 86%, 87%, 88%, 89%, 90%, 91%, 92%, 92.5%, 93%, 94%, 95%, 96%, 97%, 98%, or 99% identity to the nucleic acid sequence of SEQ ID NO: 8.
11. The rAAV vector of any one of embodiments 1-10, wherein the heterologous nucleic acid comprises the nucleic acid sequence set forth as SEQ ID NO: 8, 9 or 10.
12. The rAAV vector of any one of embodiments 1-11, wherein the heterologous nucleic acid is operably linked to one or more regulatory elements that direct expression of the heterologous nucleic acid in a photoreceptor cell or retinal pigment epithelium cell.
13. The rAAV vector of any one of embodiments 1-12, wherein the retinoschisin protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of SEQ ID NO: 12.
14. The rAAV vector of any one of embodiments 1-13, wherein the retinoschisin protein comprises the amino acid sequence of SEQ ID NO: 12.
15. The rAAV vector of any one of embodiments 1-14, wherein the polynucleotide comprises a promoter.
16. The rAAV vector of embodiment 15, wherein the promoter is selected from a rhodopsin kinase promoter, a rhodopsin promoter, an IRBP promoter, a chimeric human Retinoschisin-IRBP enhancer (RS/IRPB); a red/green cone opsin promoter, a Cone Arrestin promoter, a chimeric IRBP enhancer-cone transducin promoter, a chicken beta actin promoter, and a truncated chimeric chicken beta actin (smCBA) promoter.
17. The rAAV vector of embodiment 15 or 16, wherein the promoter is a rhodopsin kinase promoter.
18. The rAAV vector of any one of embodiments 15-17, wherein the promoter is a human rhodopsin kinase (hGRK1) promoter.
19. The rAAV vector of any one of embodiments 15-18, wherein the promoter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 7.
20. The rAAV vector of any one of embodiments 15-18, wherein the promoter comprises the nucleic acid sequence of SEQ ID NO: 7.
21. The rAAV vector of any one of embodiments 1-20, wherein the polynucleotide comprises a WPRE element.
22. The rAAV vector of embodiment 21, wherein the WPRE element comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 15, or comprises the nucleotide sequence of SEQ ID NO: 15.
23. The rAAV vector of embodiment 21 or 22, wherein the WPRE element is positioned 3′ of the heterologous nucleic acid.
24. The rAAV vector of any one of embodiments 1-23, wherein the vector is self-complementary.
25. The rAAV vector of any one of embodiments 1-24, wherein the polynucleotide is flanked by one or more inverted terminal repeats (ITRs).
26. The rAAV vector of embodiment 25, wherein the one or more ITR sequences comprises a first ITR sequence and a second ITR sequence.
27. The rAAV vector of embodiment 26, wherein the first ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 22-23.
28. The rAAV vector of embodiment 26 or 27, wherein the first ITR sequence comprises the nucleic acid sequence of any one of SEQ ID NOs: 22-23.
29. The rAAV vector of any one of embodiments 26-28, wherein the second ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 24-25.
30. The rAAV vector of any one of embodiments 26-29, wherein the second ITR sequence comprises the nucleic acid sequence of any one of SEQ ID NOs: 24-25.
31. The rAAV vector of any one of embodiments 1-30, wherein the polynucleotide further comprises a polyadenylation signal.
32. The rAAV vector of embodiment 31, wherein the polyadenylation signal is selected from a bovine growth factor hormone polyadenylation signal, an SV40 polyadenylation signal, a human growth factor hormone polyadenylation signal, and an rbGlob polyadenylation signal.
33. The rAAV vector of embodiment 32, wherein the polyadenylation signal comprises a bovine growth factor polyadenylation signal.
34. The rAAV vector of any one of embodiments 31-33, wherein the polyadenylation signal comprises a sequence having least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19.
35. The rAAV vector of any one of embodiments 31-34, wherein the polyadenylation signal comprises SEQ ID NO: 19.
36. The rAAV vector of any one of embodiments 1-35, wherein the vector comprises a nucleotide sequence having at least 85%, 86%, 87%, 88%, 89%, 90%, 91%, 92%, 93%, 94%, 95%, 96%, 97%, 98%, or 99% identity to the nucleotide sequence of any one of SEQ ID NOS: 16, 17, and 31-35.
37. The rAAV vector of any one of embodiments 1-36, wherein the vector comprises any one of the nucleotide sequences of any one of SEQ ID NOS: 16, 17, and 31-35.
38. The rAAV vector of any one of embodiments 1-37, wherein the SV40 intron is positioned 5′ of the heterologous nucleic acid.
39. The rAAV vector of any one of embodiments 1-38, wherein the SV40 intron is positioned 3′ of the promoter.
40. The rAAV vector of any one of embodiments 1-39, wherein the SV40 intron is positioned between the promoter and heterologous nucleic acid.
41. The rAAV vector of any one of embodiments 1-40, wherein the SV40 intron is positioned 3′ of the heterologous nucleic acid.
42. An AAV vector comprising a polynucleotide comprising a heterologous nucleic acid encoding a retinoschisin protein operably linked to a rhodopsin kinase promoter.
43. The rAAV vector of embodiment 42, wherein the promoter is a human rhodopsin kinase (hGRK1) promoter.
44. The AAV vector of embodiment 42 or embodiment 43, wherein the promoter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 7.
45. The AAV vector of any one of embodiments 42-44, wherein the promoter comprises the nucleic acid sequence of SEQ ID NO: 7.
46. The AAV vector of any one of embodiments 42-45, comprising a polyadenylation signal.
47. The AAV vector of embodiment 46, wherein the polyadenylation signal is selected from a bovine growth factor hormone polyadenylation signal, an SV40 polyadenylation signal, a human growth factor hormone polyadenylation signal, and an rbGlob polyadenylation signal.
48. The AAV vector of embodiment 47, wherein the polyadenylation signal comprises a bovine growth factor polyadenylation signal.
49. The AAV vector of any one of embodiments 46-48, wherein the polyadenylation signal comprises a sequence having least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19.
50. The AAV vector of any one of embodiments 46-49, wherein the polyadenylation signal comprises SEQ ID NO: 19.
51. The AAV vector of any one of embodiments, 42-50, comprising one or more ITRs.
52. The AAV vector of embodiment 51, wherein the one or more ITRs comprises a first ITR sequence and a second ITR sequence.
53. The AAV vector of embodiment 52, wherein the first ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NOs: 22-23.
54. The AAV vector of embodiment 52 or 53, wherein the first ITR sequence comprises the nucleic acid sequence of SEQ ID NOs: 22-23.
55. The AAV vector of any one of embodiments 52-54, wherein the second ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NOs: 24-25.
56. The AAV vector of any one of embodiments 52-55, wherein the second ITR sequence comprises the nucleic acid sequence of SEQ ID NOs: 24-25.
57. The rAAV vector of any one of embodiments 42-56, comprising a WPRE element.
58. The rAAV vector of embodiment 57, wherein the WPRE element comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 15.
59. The rAAV vector of embodiment 58, wherein the WPRE element comprises the nucleotide sequence of SEQ ID NO: 15.
60. The rAAV vector of any one of embodiments 57-59, wherein the WPRE element is positioned 3′ of the heterologous nucleic acid.
61. An AAV vector comprising a polynucleotide comprising a heterologous nucleic acid encoding a retinoschisin protein and a polyadenylation signal.
62. The AAV vector of embodiment 61, wherein the polyadenylation signal is selected from a bovine growth factor hormone polyadenylation signal, an SV40 polyadenylation signal, a human growth factor hormone polyadenylation signal, and an rbGlob polyadenylation signal.
63. The AAV vector of embodiment 62, wherein the polyadenylation signal comprises a bovine growth factor polyadenylation signal.
64. The AAV vector of any one of embodiments 61-63, wherein the polyadenylation signal comprises a sequence having least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19.
65. The AAV vector of any one of embodiments 61-64, wherein the polyadenylation signal comprises SEQ ID NO: 19.
66. The AAV vector of any one of embodiments, 61-65, comprising one or more ITRs.
67. The AAV vector of embodiment 66, wherein the one or more ITRs comprises a first ITR sequence and a second ITR sequence.
68. The AAV vector of embodiment 67, wherein the first ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NOs: 22-23.
69. The AAV vector of embodiment 67 or 68, wherein the first ITR sequence comprises the nucleic acid sequence of SEQ ID NOs: 22-23.
70. The AAV vector of any one of embodiments 67-69, wherein the second ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NOs: 24-25.
71. The AAV vector of any one of embodiments 67-70, wherein the second ITR sequence comprises the nucleic acid sequence of SEQ ID NOs: 24-25.
72. The rAAV vector of any one of embodiments 61-71, comprising a WPRE element.
73. The rAAV vector of embodiment 72, wherein the WPRE element comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 15.
74. The rAAV vector of embodiment 73, wherein the WPRE element comprises the nucleotide sequence of SEQ ID NO: 15.
75. The rAAV vector of any one of embodiments 72-74, wherein the WPRE element is positioned 3′ of the heterologous nucleic acid.
76. The rAAV vector of any one of embodiments 61-75, wherein the polynucleotide comprises a promoter.
77. The rAAV vector of embodiment 76, wherein the promoter is selected from a rhodopsin kinase promoter, a rhodopsin promoter, an IRBP promoter, a RS/IRPB promoter; a red/green cone opsin promoter, a Cone Arrestin promoter, a chimeric IRBP enhancer-cone transducin promoter, a chicken beta actin promoter, and a smCBA promoter.
78. The AAV vector of embodiment 76, wherein the promoter is a rhodopsin kinase promoter.
79. The rAAV vector of embodiment 78, wherein the promoter is a human rhodopsin kinase (hGRK1) promoter.
80. The AAV vector of any one of embodiments 76-79, wherein the promoter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 7.
81. The AAV vector of any one of embodiments 76-80, wherein the promoter comprises the nucleic acid sequence of SEQ ID NO: 7.
82. An AAV vector comprising i) a polynucleotide comprising a heterologous nucleic acid encoding a retinoschisin protein and ii) one or more ITRs.
83. The AAV vector of embodiment 82, wherein the one or more ITRs comprises a first ITR sequence and a second ITR sequence.
84. The AAV vector of embodiment 83, wherein the first ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NOs: 22-23.
85. The AAV vector of embodiment 83 or 84, wherein the first ITR sequence comprises the nucleic acid sequence of SEQ ID NOs: 22-23.
86. The AAV vector of any one of embodiments 83-85, wherein the second ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NOs: 24-25.
87. The AAV vector of any one of embodiments 83-86, wherein the second ITR sequence comprises the nucleic acid sequence of SEQ ID NOs: 24-25.
88. The AAV vector of any one of embodiments 82-87, comprising a polyadenylation signal.
89. The AAV vector of embodiment 88, wherein the polyadenylation signal is selected from a bovine growth factor hormone polyadenylation signal, an SV40 polyadenylation signal, a human growth factor hormone polyadenylation signal, and an rbGlob polyadenylation signal.
90. The AAV vector of embodiment 89, wherein the polyadenylation signal comprises a bovine growth factor polyadenylation signal.
91. The AAV vector of any one of embodiments 88-90, wherein the polyadenylation signal comprises a sequence having least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19.
92. The AAV vector of any one of embodiments 88-91, wherein the polyadenylation signal comprises SEQ ID NO: 19.
93. The rAAV vector of any one of embodiments 82-92, comprising a WPRE element.
94. The rAAV vector of embodiment 93, wherein the WPRE element comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 15.
95. The rAAV vector of embodiment 94, wherein the WPRE element comprises the nucleotide sequence of SEQ ID NO: 15.
96. The rAAV vector of any one of embodiments 93-95, wherein the WPRE element is positioned 3′ of the heterologous nucleic acid.
97. The rAAV vector of any one of embodiments 82-96, wherein the polynucleotide comprises a promoter.
98. The rAAV vector of embodiment 97, wherein the promoter is selected from a rhodopsin kinase promoter, a rhodopsin promoter, an IRBP promoter, a RS/IRPB promoter; a red/green cone opsin promoter, a Cone Arrestin promoter, a chimeric IRBP enhancer-cone transducin promoter, a chicken beta actin promoter, and a smCBA promoter.
99. The AAV vector of embodiment 97, wherein the promoter is a rhodopsin kinase promoter.
100. The rAAV vector of embodiment 99, wherein the promoter is a human rhodopsin kinase (hGRK1) promoter.
101. The AAV vector of any one of embodiments 97-100, wherein the promoter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 7.
102. The AAV vector of any one of embodiments 97-101, wherein the promoter comprises the nucleic acid sequence of SEQ ID NO: 7.
103. An AAV vector comprising i) a polynucleotide comprising a heterologous nucleic acid encoding a retinoschisin protein and ii) a WPRE element.
104. The rAAV vector of embodiment 103, wherein the WPRE element comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 15.
105. The rAAV vector of embodiment 104, wherein the WPRE element comprises the nucleotide sequence of SEQ ID NO: 15.
106. The rAAV vector of any one of embodiments 103-105, wherein the WPRE element is positioned 3′ of the heterologous nucleic acid.
107. The AAV vector of any one of embodiments, 103-106, comprising one or more ITRs.
108. The AAV vector of embodiment 107, wherein the one or more ITRs comprises a first ITR sequence and a second ITR sequence.
109. The AAV vector of embodiment 108, wherein the first ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NOs: 22-23.
110. The AAV vector of embodiment 108 or 109, wherein the first ITR sequence comprises the nucleic acid sequence of SEQ ID NOs: 22-23.
111. The AAV vector of any one of embodiments 108-110, wherein the second ITR sequence comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NOs: 24-25.
112. The AAV vector of any one of embodiments 108-111, wherein the second ITR sequence comprises the nucleic acid sequence of SEQ ID NOs: 24-25.
113. The AAV vector of any one of embodiments 103-112, comprising a polyadenylation signal.
114. The AAV vector of embodiment 113, wherein the polyadenylation signal is selected from a bovine growth factor hormone polyadenylation signal, an SV40 polyadenylation signal, a human growth factor hormone polyadenylation signal, and an rbGlob polyadenylation signal.
115. The AAV vector of embodiment 114, wherein the polyadenylation signal comprises a bovine growth factor polyadenylation signal.
116. The AAV vector of any one of embodiments 113-115, wherein the polyadenylation signal comprises a sequence having least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 19.
117. The AAV vector of any one of embodiments 113-116, wherein the polyadenylation signal comprises SEQ ID NO: 19.
118. The rAAV vector of any one of embodiments 103-117, wherein the polynucleotide comprises a promoter.
119. The rAAV vector of embodiment 118, wherein the promoter is selected from a rhodopsin kinase promoter, a rhodopsin promoter, an IRBP promoter, a RS/IRPB promoter; a red/green cone opsin promoter, a Cone Arrestin promoter, a chimeric IRBP enhancer-cone transducin promoter, a chicken beta actin promoter, and a smCBA promoter.
120. The AAV vector of embodiment 118, wherein the promoter is a rhodopsin kinase promoter.
121. The rAAV vector of any one of embodiments 118-120, wherein the promoter is a human rhodopsin kinase (hGRK1) promoter.
122. The AAV vector of any one of embodiments 118-121, wherein the promoter comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 7.
123. The AAV vector of any one of embodiments 118-122, wherein the promoter comprises the nucleic acid sequence of SEQ ID NO: 7.
124. The AAV vector of any one of embodiments 42-123, wherein the polynucleotide further comprises an SV40 intron.
125. The AAV vector of embodiment 124, wherein the SV40 intron comprises a splice donor region and/or a splice acceptor region.
126. The AAV vector of embodiment 124 or 125, wherein the SV40 intron comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the nucleic acid sequence of SEQ ID NO: 20.
127. The AAV vector of embodiment 126, wherein the SV40 intron comprises the nucleic acid sequence of SEQ ID NO: 20.
128. The AAV vector of any one of embodiments 42-127, wherein the retinoschisin protein is a human retinoschisin protein.
129. The AAV vector of any one of embodiments 42-128, wherein the heterologous nucleic acid does not comprise the 5′ untranslated region of human retinoschisin.
130. The AAV vector of embodiment 129, wherein the 5′ untranslated region comprises the nucleic acid sequence of SEQ ID NO: 39.
131. The AAV vector of any one of embodiments 42-130, wherein the nucleic acid does not comprise the 3′ untranslated region of human retinoschisin.
132. The AAV vector of embodiment 131, wherein the 3′ untranslated region comprises the nucleic acid sequence of SEQ ID NO: 40.
133. The rAAV particle of any one of embodiments 42-132, wherein the heterologous nucleic acid comprises the nucleic acid sequence set forth as SEQ ID NO: 8, 9 or 10.
134. The AAV vector of any one of embodiments 42-133, wherein the retinoschisin protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of SEQ ID NO: 12.
135. The AAV vector of any one of embodiments 42-134, wherein the retinoschisin protein comprises the amino acid sequence of SEQ ID NO: 12.
136. The AAV vector of any one of embodiments 42-135, wherein the polynucleotide comprises a splice donor region and/or a splice acceptor region.
137. The AAV vector of any one of embodiments 1-136, wherein the AAV vector comprises a first ITR and a second ITR and the length of the AAV vector between the first ITR and the second ITR is between about 2000 nucleotides and about 6000 nucleotides.
138. The AAV vector of embodiment 137, wherein the length of the AAV vector between the first ITR and the second ITR is between about 3000 nucleotides and about 5000 nucleotides.
139. The AAV vector of embodiment 138, wherein the length of the AAV vector between the first ITR and the second ITR is between about 4000 nucleotides and about 5000 nucleotides.
140. The AAV vector of embodiment 139, wherein the length of the AAV vector between the first ITR and the second ITR is about 4500 nucleotides.
141. The AAV vector of any one of embodiments 1-140, wherein the AAV vector comprises a stuffer sequence.
142. An AAV vector comprising a polynucleotide comprising a nucleic acid encoding human retinoschisin protein and a stuffer sequence.
143. The AAV vector of embodiment 141 or 142, wherein the stuffer sequence has a length of between about 1000 nucleotides and about 4000 nucleotides.
144. The AAV vector of any one of embodiments 141-143, wherein the stuffer sequence has a length of between 2000 nucleotides and about 3000 nucleotides.
145. The AAV vector of any one of embodiments 141-144, wherein the stuffer sequence has a length of about 2800 nucleotides.
146. The AAV vector of any one of embodiments 141-144, wherein the stuffer sequence has a total length of about 2200 nucleotides.
147. The AAV vector of any one of embodiments 141-146, wherein the stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of any one of SEQ ID NOS: 26-30.
148. The AAV vector of any one of embodiments 141-147, wherein the stuffer sequence is positioned 3′ of the heterologous nucleic acid.
149. The AAV vector of any one of embodiments 141-147, wherein the stuffer sequence is positioned 5′ of the heterologous nucleic acid.
150. The AAV vector of any one of embodiments 141-149, wherein the stuffer sequence is positioned between ITR sequences.
151. A nucleic acid stuffer sequence comprising a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least a 100 contiguous bases of any one of SEQ ID NOS: 26-30.
152. An AAV vector comprising the nucleic acid stuffer sequence of embodiment 151.
153. An AAV particle that comprises the AAV vector of any one of embodiments 1-152 and a capsid.
154. The AAV particle of embodiment 153, wherein the capsid is selected from AAV44.9(E531D), AAV44.9(T492V+E531D), AAV44.9(Y446F+E531D), and AAV44.9(Y446F+T492V+E531D), AAVS and variants thereof, AAV7 and variants thereof, AAV8 and variants thereof, AAVS and variants thereof, AAV2(4pMut)ΔHS, AAV44.9, AAV8(Y447F+Y733F+T494V), AAVrh.8, AAVrh.8R, AAVrh.10, AAVrh.74, AAV2TT, AAV2HBKO, AAVAnc80, DGE-DF, P2-V2, P2-V3, and ME-B(Y-F+T-V).
155. The AAV particle of embodiment 153, wherein the AAV capsid is selected from AAV44.9(E531D), AAV44.9(T492V+E531D), AAV44.9(Y446F+E531D), and AAV44.9(Y446F+T492V+E531D).
156. The AAV particle of embodiment 153, wherein the AAV capsid is AAV44.9(E531D).
157. The AAV particle of embodiment 153, wherein the AAV capsid is selected from DGE-DF, P2-V2, P2-V3, and ME-B(Y-F+T-V).
158. The AAV particle of embodiment 153, wherein the AAV capsid comprises an AAVS capsid protein, or a variant thereof.
159. A composition comprising the AAV particle of any one of embodiments 153-158 and a pharmaceutically acceptable carrier, buffer, diluent, or excipient, or any combination thereof.
160. A cell comprising the AAV vector of any one of embodiments 1-152, the AAV particle of any one of embodiments 153-158, or the composition of embodiment 159.
161. A method for transducing a cell, the method comprising administering to the cell the AAV vector of any one of embodiments 1-152, the AAV particle of any one of embodiments 153-158, or the composition of embodiment 159.
162. The method of embodiment 160 or 161, wherein the cell is a photoreceptor cell.
163. The method of embodiment 160 or 161, wherein the cell is a retinal pigment epithelial (RPE) cell.
164. A method for treating X-linked retinoschisis (XLRS) in a mammal, the method comprising administering to the mammal the AAV vector of any one of embodiments 1-152, the AAV particle of any one of embodiments 153-158, the composition of embodiment 159, or the cell of embodiment 160.
165. The method of embodiment 164, wherein the administration is to one or both eyes of the mammal.
166. The method of embodiment 164 or 165, wherein the step of administering comprises intravitreal administration or subretinal administration to one or both eyes of the mammal.
167. The method of any one of embodiments 164-166, wherein a volume of between 30 μL and 150 μL of the vector, particle or the composition is administered.
168. The method of embodiment 167, wherein a volume of about 90 μL of the vector, particle or the composition is administered.
169. The method of any one of embodiments 164-168, wherein the particle is administered in an amount of between 5×10¹⁰ and 1×10¹² vector genomes (vgs)/mL.
170. The method of any one of embodiments 164-169, wherein step of administering a) preserves one or more photoreceptor cells, b) restores laminar retinal structure, c) restores one or more rod- and/or cone-mediated functions, d) restores completely or partially visual behavior in one or both eyes, or e) any combination thereof.
171. The method of any one of embodiments 164-170, wherein the mammal is human.

What is claimed is:

1. An adeno-associated virus (AAV) vector comprising a polynucleotide that comprises (i) a heterologous nucleic acid encoding a retinoschisin protein, and (ii) an intron, splice donor region, splice acceptor region, or any combination of two or more thereof.

2. The AAV vector of claim 1, comprising the intron, wherein the intron comprises a splice donor region and/or a splice acceptor region.

3. The AAV vector of claim 1 or 2, comprising the intron, wherein the intron comprises a sequence having at least at least 80%, 81%, 82%, 83%, 84%, 85%, 86%, 87%, 88%, 89%, 90%, 91%, 92%, 93%, 94%, 95%, 96%, 97%, 98%, or 99% identity to the nucleic acid sequence of SEQ ID NO: 20 or 21.

4. The AAV vector of claim 3, wherein the intron comprises the nucleic acid sequence of SEQ ID NO: 20 or 21.

5. The AAV vector of any one of claims 1-4, comprising the intron, wherein the intron comprises an SV40 intron.

6. The AAV vector of any one of claims 1-5, comprising a post-transcription regulatory element.

7. An AAV vector comprising a polynucleotide that comprises (i) a heterologous nucleic acid encoding a retinoschisin protein and (ii) a post-transcription regulatory element.

8. The AAV vector of claim 6 or claim 7, wherein the post-transcription regulatory element comprises a woodchuck hepatitis virus post-transcription regulatory element (WPRE).

9. The rAAV vector of any one of claims 6-8, wherein the post-transcription regulatory element comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 15.

10. The rAAV vector of any one of claims 6-9, wherein the post-transcription regulatory element is positioned 3′ of the heterologous nucleic acid.

11. The AAV vector of any one of claims 1-10, wherein the retinoschisin protein is a human retinoschisin protein.

12. The AAV vector of any one of claims 1-11, wherein the heterologous nucleic acid does not comprise the 5′ untranslated region of human retinoschisin.

13. The AAV vector of claim 12, wherein the 5′ untranslated region comprises the nucleic acid sequence of SEQ ID NO: 39.

14. The AAV vector of any one of claims 1-13, wherein the nucleic acid does not comprise the 3′ untranslated region of human retinoschisin.

15. The AAV vector of claim 14, wherein the 3′ untranslated region comprises the nucleic acid sequence of SEQ ID NO: 40.

16. The AAV vector of any one of claims 1-15, wherein the heterologous nucleic acid comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the nucleotide sequence of SEQ ID NO: 8.

17. A recombinant AAV (rAAV) vector comprising a polynucleotide that comprises a heterologous nucleic acid comprising a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to the nucleotide sequence of SEQ ID NO: 8.

18. The rAAV vector of claim 17, wherein the polynucleotide comprises a post-transcription regulatory element.

19. The rAAV vector of claim 18, wherein the post-transcription regulatory element comprises a sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 15.

20. The rAAV vector of claim 18 or 19, wherein the post-transcription regulatory element is positioned 3′ of the heterologous nucleic acid.

21. The rAAV vector of any one of claims 1-20, wherein the heterologous nucleic acid comprises a sequence that differs by 5, 10, 15, 20, 25, or more than 25 nucleotides from the nucleotide sequence of SEQ ID NO: 8.

22. The rAAV vector of any one of claims 1-21, wherein the heterologous nucleic acid comprises the sequence set forth as SEQ ID NO: 8.

23. The rAAV vector of any one of claims 1-22, wherein the heterologous nucleic acid comprises the sequence set forth as SEQ ID NO: 9.

24. The rAAV vector of any one of claims 1-23, wherein the polynucleotide comprises the sequence of SEQ ID NO: 10.

25. The rAAV vector of any one of claims 1-24, wherein the heterologous nucleic acid is operably linked to one or more regulatory elements that direct expression of the heterologous nucleic acid in a photoreceptor cell or retinal pigment epithelium cell.

26. The rAAV vector of any one of claims 1-25, wherein the polynucleotide comprises a promoter that is capable of expressing the nucleic acid sequence in one or more photoreceptors or retinal pigment epithelial cells of a mammalian eye.

27. The rAAV vector of claim 26, wherein the promoter is selected from a rhodopsin kinase promoter, a rhodopsin promoter, an IRBP promoter, a chimeric human Retinoschisin-IRBP enhancer (RS/IRPB); a red/green cone opsin promoter, a Cone Arrestin promoter, a chimeric IRBP enhancer-cone transducin promoter, a chicken beta actin promoter, and a truncated chimeric chicken beta actin (smCBA) promoter.

28. The rAAV vector of claim 26 or 27, wherein the promoter is a human rhodopsin kinase (hGRK1) promoter.

29. The rAAV vector of any one of claims 1-28, wherein the vector is self-complementary.

30. The rAAV vector of any one of claims 1-29, wherein the polynucleotide is flanked by one or more inverted terminal repeat (ITR) sequences.

31. The AAV vector of claim 30, wherein the one or more ITR sequences comprises a nucleic acid sequence having at least 80%, 85%, 90%, 92%, 95%, 98%, or 99% identity to SEQ ID NOs: 22-25.

32. The AAV vector of claim 30 or 31, wherein the one or more ITR sequences comprises the nucleic acid sequence of SEQ ID NOs: 22-25.

33. The AAV vector of any one of claims 1-32 further comprising a polyadenylation signal.

34. The AAV vector of claim 33, wherein the polyadenylation signal comprises a bovine growth factor polyadenylation signal.

35. The rAAV vector of any one of claims 1-34, wherein the vector comprises a nucleotide sequence having at least 95%, 98%, or 99% identity to the nucleotide sequence of any one of SEQ ID NOs: 16, 17, and 31-35.

36. The rAAV vector of any one of claims 1-35, wherein the vector comprises the nucleotide sequence of any one of SEQ ID NOs: 16, 17, and 31-35.

37. A recombinant adeno-associated viral (rAAV) particle comprising a capsid, wherein the rAAV particle further comprises the rAAV vector of any one of claims 1-36.

38. The rAAV particle of claim 37, wherein the capsid comprises an AAV44.9(E531D) capsid.

39. The rAAV particle of claim 37, wherein the capsid comprises a protein that comprises the amino acid sequence of any one of SEQ ID NOs: 1-6, 18, and 36-38.

40. A recombinant adeno-associated viral (rAAV) particle comprising an AAV44.9(E531D) capsid and the rAAV vector of any one of claims 1-36.

41. The rAAV particle of claim 37, wherein the capsid comprises any of the following capsid proteins: AAV44.9(T492V+E531D), AAV44.9(Y446F+E531D), and AAV44.9(Y446F+T492V+E531D).

42. The rAAV particle of claim 37, wherein the capsid comprises any of the following capsid proteins: AAVS and variants thereof, AAV7 and variants thereof, AAV8 and variants thereof, AAV9 and variants thereof, AAV2(4pMut)ΔHS, AAV44.9, AAV8(Y447F+Y733F+T494V), AAVrh.8, AAVrh.8R, AAVrh.10, AAVrh.74, AAV2TT, AAV2HBKO, and AAVAnc80.

43. The rAAV particle of claim 37, wherein the capsid comprises an AAV5 capsid protein, or a variant thereof.

44. The rAAV particle of claim 37, wherein the capsid protein comprises AAV44.9(Y446F+T492V+E531D).

45. A composition comprising a plurality of the rAAV particle of any one of claims 37-44 further comprising one or more pharmaceutically acceptable carriers, buffers, diluents or excipients.

46. A cell comprising the rAAV vector of any one of claims 1-36 or the rAAV particle of any one of claims 37-44.

47. The cell of claim 46, wherein the cell is a mammalian cell.

48. A method for transducing a mammalian photoreceptor cell or retinal pigment epithelium cell, the method comprising administering to one or both eyes of a mammal the rAAV particle of any one of claims 37-44 or the composition of claim 45.

49. A method for treating or ameliorating X-linked retinoschisis (XLRS) in a mammal, the method comprising subretinally administering to one or both eyes of the mammal the rAAV particle of any one of claims 37-44 or the composition of claim 45 in an amount sufficient to treat or ameliorate the one or more symptoms of the retinoschisis in the mammal.

50. The method of claim 49, wherein the mammal is human.

51. The method of any one of claims 48-50, wherein the particle is administered in an amount of between 5×10¹⁰ and 1×10¹² vector genomes (vgs)/mL.

52. The method of any one of claims 48-51, wherein step of administering a) preserves one or more photoreceptor cells, b) restores laminar retinal structure, c) restores one or more rod- and/or cone-mediated functions, d) restores completely or partially visual behavior in one or both eyes, or e) any combination thereof.

53. The method of any one of claims 48-52, wherein the step of administering restores laminar retinal structure.

54. The method of any one of claims 48-53, wherein the step of administering comprises subretinal administration to a fovea of one or both eyes of the mammal.

55. The method of claim 54, wherein detachment of the fovea is minimized.


SEQUENCE LISTING (40 sequences)

SEQ ID NO: 1 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMASQARNWVPGPSYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 2 - protein, 599 aa, Artificial Sequence
  Synthetic
MAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMASQARNWVPGPSYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 3 - protein, 534 aa, Artificial Sequence
  Synthetic
MASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMASQARNWVPGPSYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 4 - DNA, 2211 bp, Artificial Sequence
  Synthetic
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcaccccaagaaccagactcctcatcgggcatcggcaagacaggccagcagcccgctaaaaagagactcaattttggtcagactggcgacacagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctactttggctacagcaccccctgggggtattttgacttcaacagattccactgccacttttcaccacgtgactggcagcgactcatcaacaacaattggggattccggcccaagagactcaacttcaagctcttcaacatccaggtcaaggaagtcacgacgaacgaaggcaccaagaccatcgccaataatctcaccagcaccgtgcaggtctttacggactcggagtaccagctaccgtacgtgctaggatcagctcaccagggatgtctgcctccgttcccggcggacgtcttcatggttcctcagtacggttatctaactctgaacaatggcagccaggccctgggacgttcctccttctactgcctggagtatttcccatcgcagatgctgagaaccggcaacaactttcagttcagctacaccttcgaggacgtgcctttccacagcagctacgcgcacagccaaagcctggacaggctgatgaatcccctcatcgaccagtacctgtattacctggtcagaacgcagacaaccgggactggagggacgcagactctggcattcagccaagcaggccctagctcaatggccagccaggctagaaactgggtgcccggaccgagctaccggcagcagcgcgtctccacgacaaccaaccagaacaacaacagcaactttgcctggacgggagctgccaaatttaaactgaacggccgagactctctaatgaaccccggcgtggccatggcttcacacaaggatgacgatgaccggttcttcccttctagcggggtcctgattttcggcaagcaaggagccgggaatgatggagtggattacagccaagtgctgattacagatgaggaagaaatcaaggctaccaaccccgtggcaacagaggaatatggagcagtggccatcaacaaccaggccgctaatacgcaggcgcagaccggactcgtgcacaaccagggggtgattcccggcatggtgtggcagaacagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcagtacacttccaactactacaaatctacaaatgtggactttgctgtcaacacggaaggagtgtatagcgagcctcgccccattggcacgcgctacctcacccgtaatctgtaa

SEQ ID NO: 5 - DNA, 1800 bp, Artificial Sequence
  Synthetic
acggctcctggaaagaagagaccggtagagcagtcaccccaagaaccagactcctcatcgggcatcggcaagacaggccagcagcccgctaaaaagagactcaattttggtcagactggcgacacagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctactttggctacagcaccccctgggggtattttgacttcaacagattccactgccacttttcaccacgtgactggcagcgactcatcaacaacaattggggattccggcccaagagactcaacttcaagctcttcaacatccaggtcaaggaagtcacgacgaacgaaggcaccaagaccatcgccaataatctcaccagcaccgtgcaggtctttacggactcggagtaccagctaccgtacgtgctaggatcagctcaccagggatgtctgcctccgttcccggcggacgtcttcatggttcctcagtacggttatctaactctgaacaatggcagccaggccctgggacgttcctccttctactgcctggagtatttcccatcgcagatgctgagaaccggcaacaactttcagttcagctacaccttcgaggacgtgcctttccacagcagctacgcgcacagccaaagcctggacaggctgatgaatcccctcatcgaccagtacctgtattacctggtcagaacgcagacaaccgggactggagggacgcagactctggcattcagccaagcaggccctagctcaatggccagccaggctagaaactgggtgcccggaccgagctaccggcagcagcgcgtctccacgacaaccaaccagaacaacaacagcaactttgcctggacgggagctgccaaatttaaactgaacggccgagactctctaatgaaccccggcgtggccatggcttcacacaaggatgacgatgaccggttcttcccttctagcggggtcctgattttcggcaagcaaggagccgggaatgatggagtggattacagccaagtgctgattacagatgaggaagaaatcaaggctaccaaccccgtggcaacagaggaatatggagcagtggccatcaacaaccaggccgctaatacgcaggcgcagaccggactcgtgcacaaccagggggtgattcccggcatggtgtggcagaacagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcagtacacttccaactactacaaatctacaaatgtggactttgctgtcaacacggaaggagtgtatagcgagcctcgccccattggcacgcgctacctcacccgtaatctgtaa

SEQ ID NO: 6 - DNA, 1605 bp, Artificial Sequence
  Synthetic
atggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctactttggctacagcaccccctgggggtattttgacttcaacagattccactgccacttttcaccacgtgactggcagcgactcatcaacaacaattggggattccggcccaagagactcaacttcaagctcttcaacatccaggtcaaggaagtcacgacgaacgaaggcaccaagaccatcgccaataatctcaccagcaccgtgcaggtctttacggactcggagtaccagctaccgtacgtgctaggatcagctcaccagggatgtctgcctccgttcccggcggacgtcttcatggttcctcagtacggttatctaactctgaacaatggcagccaggccctgggacgttcctccttctactgcctggagtatttcccatcgcagatgctgagaaccggcaacaactttcagttcagctacaccttcgaggacgtgcctttccacagcagctacgcgcacagccaaagcctggacaggctgatgaatcccctcatcgaccagtacctgtattacctggtcagaacgcagacaaccgggactggagggacgcagactctggcattcagccaagcaggccctagctcaatggccagccaggctagaaactgggtgcccggaccgagctaccggcagcagcgcgtctccacgacaaccaaccagaacaacaacagcaactttgcctggacgggagctgccaaatttaaactgaacggccgagactctctaatgaaccccggcgtggccatggcttcacacaaggatgacgatgaccggttcttcccttctagcggggtcctgattttcggcaagcaaggagccgggaatgatggagtggattacagccaagtgctgattacagatgaggaagaaatcaaggctaccaaccccgtggcaacagaggaatatggagcagtggccatcaacaaccaggccgctaatacgcaggcgcagaccggactcgtgcacaaccagggggtgattcccggcatggtgtggcagaacagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcagtacacttccaactactacaaatctacaaatgtggactttgctgtcaacacggaaggagtgtatagcgagcctcgccccattggcacgcgctacctcacccgtaatctgtaa

SEQ ID NO: 7 - DNA, 292 bp, Artificial Sequence
  Synthetic
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggc

SEQ ID NO: 8 - DNA, 675 bp, Artificial Sequence
  Synthetic
atgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctga

SEQ ID NO: 9 - DNA, 675 bp, Artificial Sequence
  Synthetic
atgtcaagaaagatagaaggctttttgttattacttctctttggctatgaagccacattgggattatcgtctacagaggatgaaggagaggacccatggtatcaaaaagcctgcaagtgtgattgccaaggaggacccaatgctctgtggtctgcaggtgccacctccttggactgtataccagaatgcccatatcacaagcctctgggttttgagtcaggggaggtcacacctgaccagatcacctgctctaaccctgagcagtatgtgggctggtattcttcttggactgcaaacaaggccagactcaacagtcaaggctttgggtgtgcctggctctccaagttccaggacagtagccagtggttacagatagatctgaaggagatcaaagtgatttcagggatcctcacccaggggagatgtgacattgatgagtggatgaccaagtactctgtgcagtacaggacagatgagcgcctgaactggatttactacaaggaccagactggaaacaacagagtcttctatggcaactctgacagaacctccacagttcagaacctgctgagaccccccatcatctccagattcatcagactcatcccactgggctggcatgtcagaattgccatcaggatggagctgctggagtgtgtcagcaagtgtgcctga

SEQ ID NO: 10 - DNA, 705 bp, Artificial Sequence
  Synthetic
atgtcacgcaagatagaaggctttttgttattacttctctttggctatgaagccacattgggattatcggagcagaaattaatcagtgaggaagatctgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctga

SEQ ID NO: 11 - skipped

SEQ ID NO: 12 - protein, 224 aa, Artificial Sequence
  Synthetic
MSRKIEGFLLLLLFGYEATLGLSSTEDEGEDPWYQKACKCDCQGGPNALWSAGATSLDCIPECPYHKPLGFESGEVTPDQITCSNPEQYVGWYSSWTANKARLNSQGFGCAWLSKFQDSSQWLQIDLKEIKVISGILTQGRCDIDEWMTKYSVQYRTDERLNWIYYKDQTGNNRVFYGNSDRTSTVQNLLRPPIISRFIRLIPLGWHVRIAIRMELLECVSKCA

SEQ ID NO: 13 - protein, 224 aa, Artificial Sequence; identical to SEQ ID NO: 12
  Synthetic

SEQ ID NO: 14 - protein, 234 aa, Artificial Sequence
  Synthetic
MSRKIEGFLLLLLFGYEATLGLSEQKLISEEDLSTEDEGEDPWYQKACKCDCQGGPNALWSAGATSLDCIPECPYHKPLGFESGEVTPDQITCSNPEQYVGWYSSWTANKARLNSQGFGCAWLSKFQDSSQWLQIDLKEIKVISGILTQGRCDIDEWMTKYSVQYRTDERLNWIYYKDQTGNNRVFYGNSDRTSTVQNLLRPPIISRFIRLIPLGWHVRIAIRMELLECVSKCA

SEQ ID NO: 15 - DNA, 587 bp, Artificial Sequence
  Synthetic
tcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 16 - DNA, 1731 bp, Artificial Sequence
  Synthetic
ggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacc

SEQ ID NO: 17 - DNA, 2319 bp, Artificial Sequence
  Synthetic
ggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacc

SEQ ID NO: 18 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYFLVRTQTTGTGGTQTLAFSQAGPSSMASQARNWVPGPSYRQQRVSTVTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 19 - DNA, 228 bp, Artificial Sequence
  Synthetic
tcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcat

SEQ ID NO: 20 - DNA, 164 bp, Artificial Sequence
  Synthetic
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttac

SEQ ID NO: 21 - DNA, 92 bp, Artificial Sequence
  Synthetic
aagaggtaagggtttaagggatggttggttggtggggtattaatgtttaattacctggagcacctgcctgaaatcactttttttcaggttgg

SEQ ID NO: 22 - DNA, 143 bp, Artificial Sequence
  Synthetic
ggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 23 - DNA, 145 bp, Artificial Sequence
  Synthetic
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 24 - DNA, 143 bp, Artificial Sequence
  Synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 25 - DNA, 145 bp, Artificial Sequence
  Synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 26 - DNA, 2822 bp, Artificial Sequence
  Synthetic
cgattcgagtgagcagaccacatgatgattggtattctccctgaagaaagaatgtcagcaaggagtatctgaagtccgaccaagcctagaatccgcccggttcttatttgctgaagtaatactgttgtcaagtcagctacagggagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcagtttcggctgcttgtggtttggcctaatataaagattacttacggcttaggtatactgcacatcctccggccttgtttggtgcttgagattggtgcgcatatatgtaaggtgctatcgtatccgcctccagtacttgatcttgtcctgcggctttagggctgcttatccctccagcggtccaacctctgaagggtcaggccatacttcacctcctttgtctaggattctgttccgaatttccggtctgggtcatgtactctcaccatcgagtgtagggagacgattctcgtaacagccgtagccgaatcactttcacttgagtctatgggttgtgcctgagttctagcttagtatttaacaacggcgtcggtccaaagacggtacacacttaaccatagtgaacgaatgaaacagtaatcgctccaggttggcgcgccttgcatgctggggagagatctg

SEQ ID NO: 27 - DNA, 2820 bp, Artificial Sequence
  Synthetic
cagatctgaattcgattcgagtgagcagaccacatgatgattggtattctccctgaagaaagaatgtcagcaaggagtatctgaagtccgaccaagcctagaatccgcccggttcttatttgctgaagtaatactgttgtcaagtcagctacagggagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcagtttcggctgcttgtggtttggcctaatataaagattacttacggcttaggtatactgcacatcctccggccttgtttggtgcttgagattggtgcgcatatatgtaaggtgctatcgtatccgcctccagtacttgatcttgtcctgcggctttagggctgcttatccctccagcggtccaacctctgaagggtcaggccatacttcacctcctttgtctaggattctgttccgaatttccggtctgggtcatgtactctcaccatcgagtgtagggagacgattctcgtaacagccgtagccgaatcactttcacttgagtctatgggttgtgcctgagttctagcttagtatttaacaacggcgtcggtccaaagacggtacacacttaaccatagtgaacgaatgaaacagtaatcgctccaggttaagaactattggtacc

SEQ ID NO: 28 - DNA, 2249 bp, Artificial Sequence
  Synthetic
cagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcaggcgcgccttgcatgctggggagagatctg

SEQ ID NO: 29 - DNA, 2235 bp, Artificial Sequence
  Synthetic
cagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcaggcgcgccttgcatgc

SEQ ID NO: 30 - DNA, 2219 bp, Artificial Sequence
  Synthetic
agtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcag

SEQ ID NO: 31 - DNA, 4534 bp, Artificial Sequence
  Synthetic
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatccgattcgagtgagcagaccacatgatgattggtattctccctgaagaaagaatgtcagcaaggagtatctgaagtccgaccaagcctagaatccgcccggttcttatttgctgaagtaatactgttgtcaagtcagctacagggagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcagtttcggctgcttgtggtttggcctaatataaagattacttacggcttaggtatactgcacatcctccggccttgtttggtgcttgagattggtgcgcatatatgtaaggtgctatcgtatccgcctccagtacttgatcttgtcctgcggctttagggctgcttatccctccagcggtccaacctctgaagggtcaggccatacttcacctcctttgtctaggattctgttccgaatttccggtctgggtcatgtactctcaccatcgagtgtagggagacgattctcgtaacagccgtagccgaatcactttcacttgagtctatgggttgtgcctgagttctagcttagtatttaacaacggcgtcggtccaaagacggtacacacttaaccatagtgaacgaatgaaacagtaatcgctccaggttggcgcgccttgcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 32 - DNA, 4528 bp, Artificial Sequence
  Synthetic
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcgattcgagtgagcagaccacatgatgattggtattctccctgaagaaagaatgtcagcaaggagtatctgaagtccgaccaagcctagaatccgcccggttcttatttgctgaagtaatactgttgtcaagtcagctacagggagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcagtttcggctgcttgtggtttggcctaatataaagattacttacggcttaggtatactgcacatcctccggccttgtttggtgcttgagattggtgcgcatatatgtaaggtgctatcgtatccgcctccagtacttgatcttgtcctgcggctttagggctgcttatccctccagcggtccaacctctgaagggtcaggccatacttcacctcctttgtctaggattctgttccgaatttccggtctgggtcatgtactctcaccatcgagtgtagggagacgattctcgtaacagccgtagccgaatcactttcacttgagtctatgggttgtgcctgagttctagcttagtatttaacaacggcgtcggtccaaagacggtacacacttaaccatagtgaacgaatgaaacagtaatcgctccaggttaagaactattggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 33 - DNA, 4549 bp, Artificial Sequence
  Synthetic
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatccagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcaggcgcgccttgcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 34 - DNA, 1723 bp, Artificial Sequence
  Synthetic
ggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 35 - DNA, 2311 bp, Artificial Sequence
  Synthetic
ggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 36 - protein, 735 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQGVSKVSADNNNSEFSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 37 - protein, 598 aa, Artificial Sequence
  Synthetic
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQGVSKVSADNNNSEFSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 38 - protein, 533 aa, Artificial Sequence
  Synthetic
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQGVSKVSADNNNSEFSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 39 - DNA, 40 bp, Artificial Sequence
  Synthetic
agttcagtaaggtagagctttggccgaggacgaggggaag

SEQ ID NO: 40 - DNA, 2316 bp, Artificial Sequence
  Synthetic
tgcctgcctcagctcggcgcctgccagggggtgactggcacagagcgggccgtaggggaccccctcacacaccaccgagatggacagggctatatttcgcaaagcaattgtaactgcagtgctgggtagataatttttttttttttaagatatagctttctgatttcaatgaaataaaaatgaacttattccccactcagggccagagaaagtcagaacaaagaaaatgtccccgaaacgaattttcttacaaaagcctaagtagcaggggtaattttctgctcattttttgtctcagtgatactgtgaaaggtgcagtctcaggggaacacaaagcagccctgataatttgaaaattcatttgctttaccacattcaagacagaaacatacagtttcctaaagcctggctttgaatgcagaagggagcagctcctcctagttaagtttccactaaatcatcgccaaagaggacttcagagccctggggaggcagctgagggtctcaagggtgactgggtggcagggatgagtgcggtgggtgagaatcccggtgccctgagaggctatacgtgacaaatgaccaaaagcccaaggtaggggagtttcctctgctcacagttcttaccttcaaggcggatctgggcttccaccctcatgaacacagggattggggagggaccagagcgcccaatacacacagctccattatgcaatccattccagcaaattccccgtgtctgtggtcaccatttaggtgatcatacaggacaggctgcacatctcagtatatgtagggaccccaaatgaccacaacacagtacaattgccctttacctagggctaccatttcctagcaaaccaaacatagttcgagaacagctggcccaggagctaccactggctactcagaggaggctcattagctggctacatgcttcgcaggaagtgggaaggactcacatcataaaaaggaccatgtagctttttccctgaaagcttctcaccctccaccctctgccttgcaatacgcaaactgcgcctgctcctgaaaagctctctgggaaggaatgggcctggctttccgttcctggaggcggcgccttagattgggaggcctcattggccacttagagcgcagcctgagtttccaggccccttcctgggagaggctgttaacacgggggaggggcaggagagggatatggagagcaggtggtggaatcagaggacgaggctgctctaaagactgttctggccccagacacagggtagtctttgctagcagctcatttccgagttacttttcattttcaaatgccaaggcaagtgactagactcgcgctaatacagtgctggacaacacattcaccttttctgtgaacaggcagccttctaaaagccccaaacatccttcttgatgctttgggggctcaattattttatatccaacccagcatctttctagtccctatgctgtatgcttgaacttcggaaaatgcttttccccgcccaatcttctctcaaatataaacacatcacacagggtgttgggggtggggggggggggtggggggacttatccctggccttaggacacaggacaaatctattttggatagaaatgcctgaacagagacccttatttggaaaggtgaattaactttggtcacgacatggactgtcagacaaaatggcagtatcctaagagttaaggcacatcaaacacaggagtcgagagagtgcagttcagggaaaaaggagaggaggaaacagtgaggcagggagaaaggctttccaaataagagttcatgttggaaacttttgtcacggctttattgagattaagttcacatacaatttgtatccatttaaagtgtacaatttgatgacttttggtatattcagagttgtgcaaccattatcactagatcaattttagaaagtttatcaccccaaagagaaatcctgcacccatcagccaacactccccaacccatcggccaccccaagccctctgcaaccacgaatcgactgtctctgtagattggccttctggacgttctacataaatgaaatcatatagtatgtggtatttcgtgactggcttctttcacttagcatagtgttttaaagttcatccacgttataacatgtgtatcactatgtcacttgtcactcctttttattgctgaacatcattgttcagtatcatgtcaagagcacattgttatttatccattcatccattgatggatatttgggtttccactctttagctattatgaataatgctgctatgaacatttgtgtataa